SEQUENCE LISTING (139 sequences)

SEQ ID NO: 1 - protein, 407 aa, Trichoderma reesei
MQTFGAFLVSFLAASGLAAALPTEGQKTASVEVQYNKNYVPHGPTALFKAKRKYGAPISDNLKSLVAARQAKQALAKRQTGSAPNHPSDSADSEYITSVSIGTPAQVLPLDFDTGSSDLWVFSSETPKSSATGHAIYTPSKSSTSKKVSGASWSISYGDGSSSSGDVYTDKVTIGGFSVNTQGVESATRVSTEFVQDTVISGLVGLAFDSGNQVRPHPQKTWFSNAASSLAEPLFTADLRHGQNGSYNFGYIDTSVAKGPVAYTPVDNSQGFWEFTASGYSVGGGKLNRNSIDGIADTGTTLLLLDDNVVDAYYANVQSAQYDNQQEGVVFDCDEDLPSFSFGVGSSTITIPGDLLNLTPLEEGSSTCFGGLQSSSGIGINIFGDVALKAALVVFDLGNERLGWAQK

SEQ ID NO: 2 - protein, 417 aa, Myceliophthora thermophila
MHLTPALVAATCAVEVCAGVLPRSSSTPTTFGSGTLSLKQVRNPNFVRNGPVQLARIYHKYGVPLPHDLREAVARFRAEIRKRSNGSTETNPETNDVEYLTPVSIGTPPQVLNLDFDTGSSDLWVFSSETRSSDVQGQTIYDPNESSTAQKLQGYSWQISYGDGSSSSGDVYTDAVTVGGLTVPSQAVEVARRVSDEFTSDPNNDGLLGLGFSSINTVQPVPQKTFFDNAKADLDAPIFTADLKASAPGFFNFGYIDHGAYTGEITYMPVDSSDGFWAWTSPGYAVGSGSFKRTTIQGIADTGTSLFLLPSSVVSAYYGQISGAKYDSIQGGYTLPCSGSVPDFAFGIGDSNTTISVPGDYVRYAATDSSGIICFGGIQANTGIGFSIFGDVALKAAFVVFDGAKQQLGWASKPLPS

SEQ ID NO: 3 - protein, 417 aa, Myceliophthora heterothallica
MHLTPALVAATCAVEVCAGVLPRSSSTPTSFGSGTLSLKQVRNPNFVPNGPVQLARIYHKYGVPLPHDLREAVARFRADTRKRSNGSTETNPEANDVEYLTPVSIGTPPQVLNLDFDTGSSDLWVFSSETRSSDVQGQTIYDPNESSTARKLQGYSWQISYGDGSSSSGDVYTDAVTVGGLTVPSQAVEIARRVSDEFTSDPNNDGLLGLGFSSINTVQPVPQKTFFDNAKADLDAPIFTADLKASAPGYFNFGYIDPGAYTGEITYMPVDSSDGFWAWTSPGYAVGSGSFKSTTIQGIADTGTSLFLLPSSVVSAYYGHISGAKYDSIQGGYTLPCSGSVPDFAFGIGDSNSTITVPGDYVRYAATDSSGITCFGGIQANTGIGFSIFGDVALKAAFVVFDGDKQQLGWASKPLPS

SEQ ID NO: 4 - protein, 399 aa, Trichoderma reesei
MQPSFGSFLVTVLSASMAAGSVIPSTNANPGSFEIKRSANKAFTGRNGPLALARTYAKYGVEVPKTLVDAIQLVKSIQLAKRDSATVTATPDHDDIEYLVPVKIGTPPQTLNLDFDTGSSDLWVFSSDVDPTSSQGHDIYTPSKSTSSKKLEGASWNITYGDRSSSSGDVYHDIVSVGNLTVKSQAVESARNVSAQFTQGNNDGLVGLAFSSINTVKPTPQKTWYDNIVGSLDSPVFVADLRHDTPGSYHFGSIPSEASKAFYAPIDNSKGFWQFSTSSNISGQFNAVADTGTTLLLASDDLVKAYYAKVQGARVNVFLGGYVFNCTTQLPDFTFTVGEGNITVPGTLINYSEAGNGQCFGGIQPSGGLPFAIFGDIALKAAYVIFDSGNKQVGWAQKK

SEQ ID NO: 5 - protein, 454 aa, Myceliophthora thermophila
MKFAALALAASLVAAAPRVVKVDPSDIKPRRLGGTKFKLGQIHNDLFRQHGRGPRALAKAYEKYNIELPPNLLEVVQRILKDLGIEPHSKKIPGSKSSYGNGAPYTNETDDSGEVSAIPQLFDVEYLAPVQIGTPPQTLMLNFDTGSSDLWVFSSETPSRQQNGQKIYKIEESSTARRLSNHTWSIQYGDGSRSAGNVYLDTVSVGGVNVFNQAVESATFVSSSFVTDAASSGLLGLGFDSINTVKPTKQKTFISNALESLEMGLFTANLKKAEPGNYNFGFIDETEFVGPLSFIDVDSTDGFWQFDATGYSIQLPEPSGNITGTPFRAVAHTAIADTGTTLLLLPPGIAQAYYWQVQGARQAPEVGGWVMPCNASMPDLTLHIGTYKAVIPGELIPYAPVDTDDMDTATVCYGGIQSASGMPFAIYGDIFFKAQFTVFDVENLKLGFAPKPEL

SEQ ID NO: 6 - protein, 454 aa, Myceliophthora heterothallica
MKFAALALAASLVAAAPRVVKVDPSDIKPRRLGGTKFKLGQIRNDLFRQHGRGPRALAKAYEKYNIELPPDLLEVVQKILKDLGIEPHSKKIPGFKSSYGNGAPYTNETDDSGEVSAIPQLFDVEYLAPVQIGTPPQTLMLNFDTGSSDLWVFSSETPSRQQNGQKIYKIEESSTARRLSNHTWSIQYGDGSRSSGNVYLDTVSVGGVNVFNQAVESATFVSSSFVTDAASSGLLGLGFDSINTVKPTKQKTFISNALESLEMGLFSANLKKAEPGNYNFGFIDETEFVGPLSFVDVDSTDGFWQFEATGYSIQLPEPSGNVTGTPFRAVAHTAIADTGTTLLLLPPGIAQAYYWQVQGARQAPEVGGWVMPCNASMPDLTLHIGTYKAVIPGELIPYAPVDTDDMDTATVCYGGIQSASGMPFAIYGDIFFKAQFTVFDVENLKLGFAPKPEL

SEQ ID NO: 7 - protein, 452 aa, Trichoderma reesei
MEAILQAQAKFRLDRGLQKITAVRNKNYKRHGPKSYVYLLNRFGFEPTKPGPYFQQHRIHQRGLAHPDFKAAVGGRVTRQKVLAKKVKEDGTVDAGGSKTGEVDAEDQQNDSEYLCEVTIGTPGQKLMLDFDTGSSDLWVFSTELSKHLQENHAIFDPKKSSTFKPLKDQTWQISYGDGSSASGTCGSDTVTLGGLSIKNQTIELASKLAPQFAQGTGDGLLGLAWPQINTVQTDGRPTPANTPVANMIQQDDIPSDAQLFTAAFYSERDENAESFYTFGYIDQDLVSASGQEIAWTDVDNSQGFWMFPSTKTTINGKDISQEGNTAIADTGTTLALVSDEVCEALYKAIPGAKYDDNQQGYVFPINTDASSLPELKVSVGNTQFVIQPEDLAFAPADDSNWYGGVQSRGSNPFDILGDVFLKSVYAIFDQGNQRFGAVPKIQAKQNLQPPQ

SEQ ID NO: 8 - protein, 466 aa, Myceliophthora thermophila
MDALFETHAKLRKRMALYRVRAVPNQNYQRDGTKSYVSVLNRFGFQPTKPGPYFQIFEESEEAPSMSAAPGVKPGHVWQGLFKKLKDQEEPGEVTAEDQQNDSEYLCEVMIGTAWTAERQIVKMDFDTGLADFWVSQKSFDPKKSVTWQLAKDKSWKVQYGDGSSASGIVGHDILIIGGIQIKRQAIEIATEMSAQFSEGTMDGILGLAFSKLNTVQTDGKPDPQRTVVDNMMAQDDIPPEAELFSTALYSNREDDQRSFYTFGWIDEDLVKASGEEIVWTDVDNSEGFWMFSSEHVTIDGQQVRIEGNKAIADTGTSLVLVSDQVCDALYAHIPSAEYSEEYQGWTFPQETEVDKLPEFSIAIGDKEFVLQKEDLIFAPADERVFYGSVQSRGENPFDILGIAFLKSIYAIWDQGHKRFGAVPKMEAFVPPTKYDRPRLTDQDRKDLGVTIGYGDISSTFFEKRA

SEQ ID NO: 9 - protein, 466 aa, Myceliophthora heterothallica
MDALFETHAKLRKRMALYRVRAVPNQNYQRDGTKSYVSALNRFGFQPTKPGPYFQIFEESEEAPSMPAAPGVKPGHVWQGLFKKLKDQEEPGEVTAEDQQNDSEYLCEVMIGTAWTAERQIVKMDFDTGSADFWVSHKSFDPKKSVTWQLAKDKSWKVQYGDGSSASGIVGNDILIIGGIQIKRQAIEIATEMSAQFSEGTMDGILGLAFSKLNTVQTDGKPDPQRTVVDNMMAQDDIPPEAELFSTALYSNREDDQRSFYTFGWIDEDLVKASGEEIVWTDVDNSEGFWMFSSEHVTIDGQQVRIEGNKAIADTGTSLVLVSDQVCDALYAHIPGAEYSEEYQGWTFPQETEVDKLPDFSIAIGDKEFVLQKEDLIFAPADERVFYGSVQSRGENPFDILGIAFLKSTYAIWDQGHKRFGAVPKMEAFVPPTKYDRPRLTDQDRKDLGVMIGYGDISSTFFEKRA

SEQ ID NO: 10 - protein, 395 aa, Trichoderma reesei
MKSALLAAAALVGSAQAGIHKMKLQKVSLEQQLEGSSIEAHVQQLGQKYMGVRPTSRAEVMFNDKPPKVQGGHPVPVTNFMNAQYFSEITIGTPPQSFKVVLDTGSSNLWVPSQSCNSIACFLHSTYDSSSSSTYKPNGSDFEIHYGSGSLTGFISNDVVTIGDLKIKGQDFAEATSEPGLAFAFGRFDGILGLGYDTISVNGIVPPFYQMVNQKLIDEPVFAFYLGSSDEGSEAVFGGVDDAHYEGKIEYIPLRRKAYWEVDLDSIAFGDEVAELENTGAILDTGTSLNVLPSGLAELLNAEIGAKKGFGGQYTVDCSKRDSLPDITFSLAGSKYSLPASDYIIEMSGNCISSFQGMDFPEPVGPLVILGDAFLRRYYSVYDLGRDAVGLAKAK

SEQ ID NO: 11 - protein, 397 aa, Myceliophthora thermophila
MKDAFLLTAAVLLGSAQGAVHKMKLQKIPLSEQLEAVPINTQLEHLGQKYMGLRPRESQADAIFKGMVADVKGNHPIPISNFMNAQYFSEITIGTPPQSFKVVLDTGSSNLWVPSVECGSIACYLHSKYDSSASSTYKKNGTSFEIRYGSGSLSGFVSQDTVSIGDITIQGQDFAEATSEPGLAFAFGRFDGILGLGYDRISVNGIVPPFYKMVEQKLIDEPVFAFYLADTNGQSEVVFGGVDHDKYKGKITTIPLRRKAYWEVDFDAISYGDDTAELENTGIILDTGTSLIALPSQLAEMLNAQIGAKKSYTGQYTIDCNKRDSLKDVTFNLAGYNFTLGPYDYVLEVQGSCISTFMGMDFPAPTGPLAILGDAFLRRYYSIYDLGADTVGLAEAK

SEQ ID NO: 12 - protein, 397 aa, Myceliophthora heterothallica
MKGAFLLTAAVLLGSAQGAVHKMKLQKIPLSEQLEAVPINTQLEHLGQKYMGLRPRESQADAIFKGMVADVKGNHPIPISNFMNAQYFSEITIGTPPQSFKVVLDTGSSNLWVPSVECGSIACYLHSKYDSSASSTYKKNGTSFEIRYGSGSLSGFVSQDTVSIGDITIQGQDFAEATSEPGLAFAFGRFDGILGLGYDRISVNGIVPPFYKMVEQKLIDEPVFAFYLADTKGQSEVVFGGVDHDKYKGKITTIPLRRKAYWEVDFDAISYGDDTAELENTGIILDTGTSLIALPSQLAEMLNAQIGAKKSYTGQYTIDCNKRDSLKDVTFNLAGYNFTLGPYDYILEVQGSCISTFMGMDFPAPTGPLAILGDAFLRRYYSIYDLGADTVGLAEAK

SEQ ID NO: 13 - protein, 426 aa, Trichoderma reesei
MKFHAAALTLACLASSASAGVAQPRADEVESAEQGKTFSLEQIPNERYKGNIPAAYISALAKYSPTIPDKIKHAIEINPDLHRKFSKLINAGNMTGTAVASPPPGADAEYVLPVKIGTPPQTLPLNLDTGSSDLWVISTDTYPPQVQGQTRYNVSASTTAQRLIGESWVIRYGDGSSANGIVYKDRVQIGNTFFNQQAVESAVNISNEISDDSFSSGLLGAASSAANTVRPDRQTTYLENIKSQLARPVFTANLKKGKPGNYNFGYINGSEYIGPIQYAAINPSSPLWEVSVSGYRVGSNDTKYVPRVWNAIADTGTTLLLVPNDIVSAYYAQVKGSTFSNDVGMMLVPCAATLPDFAFGLGNYRGVIPGSYINYGRMNKTYCYGGIQSSEDAPFAVLGDIALKAQFVVFDMGNKVVGFANKNTNV

SEQ ID NO: 14 - protein, 454 aa, Myceliophthora heterothallica; identical to SEQ ID NO: 6

SEQ ID NO: 15 - protein, 488 aa, Trichoderma reesei
MRASPLAVAGVALASAAQAQVVQFDIEKRHAPRLSRRDGTIDGTLSNQRVQGGYFINVQVGSPGQNITLQLDTGSSDVWVPSSTAAICTQVSERNPGCQFGSFNPDDSDTFDEVGQGLFDITYVDGSSSKGDYFQDNFQINGVTVKNLTMGLGLSSSIPNGLIGVGYMNDEASVSTTRSTYPNLPIVLQQQKLINSVAFSLWLNDLDASTGSILFGGIDTEKYHGDLTSIDIISPNGGKTFTEFAVNLYSVQATSPSGTDTLSTSEDTLIAVLDSGTTLTYLPQDMAEEAWNEVGAEYSNELGLAVVPCSVGNTNGFFSFTFAGTDGPTINVTLSELVLDLFSGGPAPRFSSGPNKGQSICEFGIQNGTGSPFLLGDTFLRSAFVVYDLVNNQIAIAPTNFNSTRTNVVAFASSGAPIPSATAAPNQSRTGHSSSTHSGLSAASGFHDGDDENAGSLTSVFSGPGMAVVGMTICYTLLGSAIFGIGWL

SEQ ID NO: 16 - protein, 501 aa, Myceliophthora thermophila
MRGYAAVAFGAILAGAVHASAGNGVVQWDIRRTQRQEELQRLNRRLRKRANPVLEVITNEKIRGGYFATCKIGTPGQDLTLQLDTGSSDIWVPDSAAQVCREIGTEGCALGTFNPNRSSSFEVIGEGQFDIEYVDGSSSKGDYFTDVFQIGDISVQNMTMGLGLHTDIAYGLVGVGYAINEAIVATTQSRDSVYPNLPVQMVDQGLINTVAYSLWLNDLDASSGSILFGGIDTEKYQGELTRIDIYPTSQGDFSSFVVALTSLEARSPSGQDTLTSQEFPIPVVLDSGTTLSYLPTDLATQAWKEVGAFYLPEVGAAVLPCDMENSKGSFSFGFAGPDGPRITVGMDELVLDMTDGQAPQFLSGPYKGRDVCQFGIQNFTSAPFLLGDTFLRSAYVVYDLVNNQIGIAATDFNSTDSNIVPFPSMGAPIPSATVAANQREVTRVPTVTEPAYSASQGFMESASGEESLAPGMPAAWGMGQLLVVGVTMALTALGSGLFFVL

SEQ ID NO: 17 - protein, 501 aa, Myceliophthora heterothallica
MRGYAAVAFGALLAGAVHASAGNGVVQWDIRRTQRQEELQRLNRRLRKRANTVLEVITNEKIRGGYFATCKIGTPGQDLTLQLDTGSSDIWVPDSAAQVCREIGTEGCALGTFDPNRSSSFEVIGEGLFDIEYVDGSSSKGDYFTDVFQIGDISVQNMTMGLGLHTDIAYGLVGVGYAINEAIVAKTQSRDSVYPNLPVQMVDQGLINTVAYSLWLNDLDASSGSILFGGIDTQKYQGELTRIDIYPTSQGDFSSFVVALTSLEARSPSGQDTLTSQEFPIPVVLDSGTTLSYLPTDLATQAWKEVGAFYSSEVGAAVLPCDMENSKGTFSFGFAGPDGPRITVGMDELVLDMTDGQAPQFLSGPYKGRDVCQFGIQNFTSAPFLLGDSFLRSAYVVYDLVNNQIGIAATDFNSTDSNIVPFPSMGAPIPSATVAANQREVTRVPTVTEPAYSASQGFMESASGEKSLAPDMPAAWGMGQMLVVGVTMALTALGSGLFFVL

SEQ ID NO: 18 - protein, 516 aa, Trichoderma reesei
MRPNSVLLAPLALYASGALAFYPYTPPWLKELEEHNAGEAKRSADNGLTFDIKRRASRRAPASQEEKAAWQAALLSHKYSESVTPSPSPDTTLSKRDNQFSILKAVDPDAPNTAGLAQDGTDYSYFVQASLGSKKTKLYMLLDTGAGSSWVMGTDCVSEACSLHDSFGPEDSDTLKTSTKDFSIAYGSGAVSGSLVNDTIEVAGMSLTYQFGLAHNTSSDFVHFAFDGILGMSMNSGANENFLSALEGAGLLDKSIFSVALARASDGHNDGEVTFGATNPSRYTGDITYTPIPSGTDWSIPLDDMSYNGKKGNVGGINAYIDTGTSYMFGPSKNVKALHAVIDGAKSSDGITWTVPCDTTTPLVVTFSGVDFAISPKDWISPKDSSGKCTSNVYGYEVVSGSWLFGDTFLKNVYAVFDKEQMRIGFAKRSSSDVGSQSFVTATSTDTAVASATAASTGTATFGAPSSAQPTMGLDGQETSGAAQPTQTTGSSGAVSLRSGSAALVLCILPFLVVMV

SEQ ID NO: 19 - protein, 545 aa, Myceliophthora thermophila
MSRVAALLLQLTLWAATARAFFPFVPVGHCAPDDDCAHLGLGARRNDDSQGPPSGGVTVELYHRPTGPHDAPKFDPIAEALGRVTRKFGRPSLFPVPRSNEKRKNNYSVSKPADPKTPNSAGIYQYGPDYSYFIKVLVGSAQQPIFMLLDTGAANTWLMGSDCTSAACQLHDTFDPSTSKTWKGGKNGFEIFYGTGDVRGTVGQDTFVFAGITHDLSFGLANHTSDDFKHFAFDGILGLGMSKSVTGTFLQTLREQKVLDSLVVSMSLNRDSDGSNDGQVTFGGIDESQFTGELTYHSIPPEHEKKGEWAITMDGASLNGKPAGIANKLAYIDTGTSFIFAPSADLDALFKAIPGVTTSQDGPYTEYWVPCDTTVPVTLTFSGVDYNVSARDWVVPGGGEDGKCLSNLYGFEISAGTWLVGDTFLKNVYSVFDADEMRIGFAAKPAPPPRPTSSSTSASASASVTLSPGAAETTSPAAGEDGESALPFMPGSSSGREGSPESSQTTGSSVPASSSKATQVNSGSRPSSGFYLPALCLAALLAVAG

SEQ ID NO: 20 - protein, 546 aa, Myceliophthora heterothallica
MSRVAALLLQLTLWAATARAFFPFVPEGHCAPDEDCAHLGLGARRNDDSQGPPSGGVTVELYHRPTGSHDAPKFDPIAEALGRVTRKFGRPSLFPVPRSNEKRKNNYSVSKPADPKTPNSAGIYQYGPDYSYFIKILVGSARQPIFMLLDTGAANTWLMGSDCTSAACQLHDTFDPSTSKTWKGGKNGFEIFYGTGDVRGTVGQDTFVFAGITHDLGFGLANHTSDDFKHFAFDGILGLGMSKSVTGTFLQTLREQNVLDSLVVSMSLNRDSDGSNDGQVTFGGIDESQFTGELTYHSIPPEQEKKGEWAITMDGASLNGKPAGFANKLAYIDTGTSFIFAPSDDLDALFKAIPGVTTSQDGPYTEYWVPCDTTLPVTLTFSGVDYNVSARDWVVPGGGEDGKCLSNLYGFEINAGTWLVGDTFLKNVYSVFDADEMRIGFAAKPAPPPTRPTSSTSEASASASVTLLPGAAETPSPAAGADGESARPFMPGSSGGRDGSPESSQTTGSSGPASSSKATQVNSGSRPSSGFYVPALCLAALLAVAG

SEQ ID NO: 21 - protein, 761 aa, Trichoderma reesei
MRSTLYGLAALPLAAQALEFIDDTVAQQNGIMRYTLTTTKGATSKHLHRRQDSADLMSQQTGYFYSIQLEIGTPPQAVSVNFDTGSSELWVNPVCSKATDPAFCKTFGQYNHSTTFVDAKAPGGIKYGTGFVDFNYGYDYVQLGSLRINQQVFGVATDSEFASVGILGAGPDLSGWTSPYPFVIDNLVKQGFIKSRAFSLDIRGLDSDRGSVTYGGIDIKKFSGPLAKKPIIPAAQSPDGYTRYWVHMDGMSITKEDGSKFEIFDKPNGQPVLLDSGYTVSTLPGPLMDKILEAFPSARLESTSGDYIVDCDIIDTPGRVNFKFGNVVVDVEYKDFIWQQPDLGICKLGVSQDDNFPVLGDTFLRAAYVVFDWDNQEVHIAANEDCGDELIPIGSGPDAIPASAIGKCSPSVKTDTTTSVAETTATSAAASTSELAATTSEAATTSSEAATTSAAAETTSVPLNTAPATTGLLPTTSHRFSNGTAPYPIPSLSSVAAAAGSSTVPSESSTGAAAAGTTSAATGSGSGSGSGDATTASATYTSTFTTTNVYTVTSCPPSVTNCPVGHVTTEVVVAYTTWCPVENGPHPTAPPKPAAPEITATFTLPNTYTCSQGKNTCSNPKTAPNVIVVTPIVTQTAPVVIPGIAAPTPTPSVAASSPASPSVVPSPTAPVATSPAQSAYYPPPPPPEHAVSTPVANPPAVTPAPAPFPSGGLTTVIAPGSTGVPSQPAQSGLPPVPAGAAGFRAPAAVALLAGAVAAALL

SEQ ID NO: 22 - protein, 668 aa, Myceliophthora thermophila
MSFALYAAALLPVAVLGAGLSVPEDNRMVQQDGLLRYPLMPRLGNLLFGKHANITRRQIDTGIFDPLSGTLYTIELTLGTPGQTVPVQFDTGSDMLWVNPVCSKAAEPEFCAAQPRFTDSSTLVDFGEQGNITYGTGYAYYEYVADYVAIGSARITQQVFGVALDSAHADVGIFGAGPNLDGWDSAYPLVVDSLAQQGYTSSRAFSMDLKGFESARGSVIFGGIDTKKYRGSLIKRLIIPAAESPDGYTRFWIYLDGISVNQPDGDVVTVFSTPDGGKGQPVLLDSGYTLSALPRPIFQKLVAAFPSAQYVSSADVYVVDCVDHGEGGSLDFIFGGKTINVPYHEFVWAQPESNTCVLGAFEDDFPVLGDTFLRSAYVVYDWDNRNIYLAQSDDCGSNLVAIGSGPDAVPSIVGECGKPKPTSTSTFSKTSSKTSTASKTSSTSDSTSSSSSHVTTSSSSTTATTLSTHKPPFPTASGNFTTTRSPTTTTASSTISKSTLTITSATTYTITSCPPTVTRCPAHEVTTEIITKTTAVCPETTATYTIPRTITCPGSGGGDDCPPGATRTTTLTVTLSPVGPTDRTTHVVPGVTTTTPTTITAPPTGQTTTTLVPALPPTTTTMSGHRGINGTVTATSKPPAVTAGSAKVGLVSGATAIVAGVMAVLMAL

SEQ ID NO: 23 - protein, 572 aa, Myceliophthora heterothallica
MSFALYAVALLPVAVLGAGLSVPEDNRMVQQDGLLRYPLMPRLGNLLFGKHSNITRRQIDTGIFDPLSGTLYTIELTLGTPGQTVAVQFDTGSDMLWVNPFCSKAAEPEFCAAQPRFTDSSTLVDFGEQGNITYGIGYAYYEYVADYVAIGSARITQQVFGVALDSAHADVGIFGAGPNLDGWDSAHPLVDGYTRFWIYLDGISVNQPDGDVVTVFSAPDGGKGQPKLVAAFPSAQYVSSADMYVVDCADHGEGGSLDFTFGGKTINVPYHEFVWPQPESNTCSDDCGSNLVAIGSGPDAVPSIVGECGKPEPTSTTSSKTSSKTSTASKTSSTSDSISSSSSSSSSSSSSSSHVTTSSSSTTATTLSTHKPPFPTASGNFTTTRFPTTTTTTSSTISKSTLTITSATTYTITSCPPTVTRCPAHEVTTEIITKTTAVCPETTATYTIPRTITCPGSGGDSCPPGATRTTSLTVTLSPVGPTDRTAHVVPGVTTTTSTAITAAPTGQTTTTLVPALPPPPTTTTTTSGHWGINGTVTATSKPPAVTAGSAKVGLVSGAMAIVAGVMAVLMAL

SEQ ID NO: 24 - protein, 477 aa, Trichoderma reesei
MRFVQYVSLAGLFAAATVSAGVVTVPFEKRNLNPDFAPSLLRRDGSVSLDAINNLTGGGYYAQFSVGTPPQKLSFLLDTGSSDTWVNSVTADLCTDEFTQQTVGEYCFRQFNPRRSSSYKASTEVFDITYLDGRRIRGNYFTDTVTINQANITGQKIGLALQSVRGTGILGLGFRENEAADTKYPTVIDNLVSQKVIPVPAFSLYLNDLQTSQGILLFGGVDTDKFHGGLATLPLQSLPPSIAETQDIVMYSVNLDGFSASDVDTPDVSAKAVLDSGSTITLLPDAVVQELFDEYDVLNIQGLPVPFIDCAKANIKDATFNFKFDGKTIKVPIDEMVLNNLAAASDEIMSDPSLSKFFKGWSGVCTFGMGSTKTFGIQSDEFVLLGDTFLRSAYVVYDLQNKQIGIAQATLNSTSSTIVEFKAGSKTIPGPASTGDDSDDSSDDSDEDSAGAALHPTFSIALAGTLFTAVSMMMSVL

SEQ ID NO: 25 - protein, 475 aa, Myceliophthora thermophila
MRSSSLLVALATLIQITHGLALPERRHGPSVLGLEMQRRAPRNPLHRDKRRRKRGTLEVGLDNEETLYFINGTVGTPPKPVRLHIDTGSSDLWVNTPASELCASANDPCAFAGTYSANSSSTYQYISSNFNISYVDGSGASGDYVSDTVTIGSQKIDRLQFGVGYSSTNDQGILGIGYPLNEVQVGRAGLRPYNNLPAQLVADGVIRSMAYSIWLNDLDANTGNILFGGVDTEKYAPPLLSLPVESASGVFSEFMITLTGLKLGSQTIGPSDLAIAVLLDTGSSLTYLPDALVSDIYAAVGAVFDGDANAAYVPCSLARDASAPPLTFTFSEPAIAVGMDELVLDLVTASGRRPTFDNGTPACLFGIGPAGAGTYVLGDTFLRSAYVVYDLDNNEIALAPTRFNSSATRVVEIGTGQDAVPGATRVSNPVKATEGLRGMNAKKNAAAAVAGLGSGGMRLMVACATLVVVVVGGLV

SEQ ID NO: 26 - protein, 478 aa, Myceliophthora heterothallica
MRSSSLLVALATLIQITHGLALPERRHGPRVLGLEMQRRAPRNPLHRDKRRRKRGTLEVGLDNEETLYFINGTVGTPPKPVRLHVDTGSSDLWVNTPASELCASANNPCAFAGTYSANSSSTYQYISSNFNISYVDGSGASGDYVSDTVTIGSQKIDRLQFGIGYSSTNDQGILGIGYPLNEVQVGRAGLRPYNNLPAQLVADGVIRSMAYSIWLNDLDANTGNILFGGVDTEKYAPPLLSLPVESASGVFSEFMITLTGLKLGSQTIGPSDLAIAVLLDTGSSLTYLPDALVSEIYAAVGAVFDGDANAAYVPCSLARDASAPPLTFTFSEPAIAVGMDELVLDLVTASGRRPTFDNGTPACLFGIGPAGAGTYVLGDTFLRSAYVVYDLDNNEIALAPTRFNSSATRVVEIGTGQDAVPGATRVSNPVQATEGLRGMNANKNAAAPAAAVAGLGSGGMRLMVACATLVVVVVGGLV

SEQ ID NO: 27 - protein, 451 aa, Trichoderma reesei
MRPPTSLSAALTLLLATDYIEAIATPVFPRAKSGDGYLAIPVGTIPRPRKVGKRSAIDATLQNMDFFYAIQVGMGTPPQNVTVLVDTGSSELWVNPDCSTAPSQQQADMCENLGQYNPRRSRTPPVGPFGREEINYGDPTDQSTQTSVDITYYADTLSFGSIQVKNQTFGVVTASDGQAQGIMGLAPDVRGGFPGDEPYSLLLNTMAEQGVIASRVFALDLRHSDSETGAIIYGGLDRSKFVGSLEARPIVRGIKGETRLAVNLTTLGLTLGRSQSFRLSAADTNVMLDSGTTLSRMHEAAAIPILEALGAQDAGEGYFYVPCSTRNAGGSVDFGFGNKVVRVPFSDFILTGEDSSDSDYCFVGIVITTDQQILGDTVLRAGYFVFDWDNREVHIAQAADCGSSDIVVVGRGSDAVPSVQGNCNENEAFPTGSGSPTVSISFVQTAFTAND

SEQ ID NO: 28 - protein, 444 aa, Myceliophthora thermophila
MEIEIGTPPQKVMLIVDTGSPNTWVNPQCETSNTPSDCAKYPQFDYTESSSINITDYVDVLRYGSGSATVQYVYETVSIGSATLKDQIIGIALESEDIPLGILGLSPPVRGVNQYPYILDTMVDQGLIKSRAFSLDLRGVDNPTGAVIFGGIDTGKYIGTLAKLPIIAPSSAPGGADRYYITMTGVGLTLPDGTMVRSEELDVPVFLDSGSTLSRLPTVIHQALAASFTEAMLDQESGLFILPCEYTDMAGSIDFYFAGKTIRVPLREFIWRSGDYCILGVAPEDDEPILGDTFLRAAYVVYDQDNRNVHLAQAADCGTNLVAIGSGEDAVPSSTGRCTELPTPTGDPTRTRAGSSNLDMTATRPPANTFTGRLPTGIAGGPGPARDGSTTTVTGGGLQPMLPTGSPKGSEGTEQNAAGRGVDSGLGAAVAAVLGVVSLLVLML

SEQ ID NO: 29 - protein, 444 aa, Myceliophthora heterothallica
MEIELGTPPQKVTLIVDTGSPNTWVNPQCETSNTPSDCAKYPQFDYAKSSSINITDYVDVLRYGSGSATVQYVYETVSIGSATLKDQIIGIALESEDIPLGILGLSPPVRGVNQYPYILDTMVDQGLIKSRAFSLDLRGVDNPTGAVIFGGIDTGKYIGTLAKLPIIAPSSAPGGADRYYITMTGVGLTLPDGTMVRSEELDVPVFLDSGSTLSRLPTVIHQALAASFTEAMLDQESGLFILPCEYTDMAGSIDFYFAGKTIRVPLREFIWRSGDYCILGVAPEDDEPILGDTFLRAAYVVYDQDNRNVHLAQAADCGTNLVAIGSGEDAVPSSTGRCTELPTPTGDPTRTRAGSSNLDMTATRPPANTFTGRLPTGIVGGPGPARDGSTTTVTGGGLQPMLPTGSPKGSEGTEQNAAGRAVDSGLGAAVAAVLGAVSLLVLML

SEQ ID NO: 30 - protein, 372 aa, Trichoderma reesei
MHAALVSLSIASLAAAALPEGVVEVPLQRIKNQTAYGVEFQVGNPPQKAVISADTGSPTYAFESPRNTVCQQGLCSAYGTYDNTTSTTSKWLSDGYSDMLIDHGFGSFINDTLRIGGVTLNDMMFGVVEQNFASFPINTQQTAIFGLGAFCQTPACDTYSTFLNQLYEHGAIARRAFSVYLGPNDPDATGSLLFGGIDLAKRQGPVHKLKVLDPTVTAANLQPNWVSLSSIELQLSNGTTITSTYDNGTYALWDTGSPGWYVQQDMFDALTSYWGLTNPNPNNDIIVDCKFREPSDDSLAVNIAPGVTINVPLSSLPIDNGDGTCTARVSPWGRVMGDTFLRNVYFTFDYEDLTVEFALVKYTDETNIVKIE

SEQ ID NO: 31 - protein, 566 aa, Myceliophthora thermophila
MKPSSAILLALAPGSSSKNVVEFSVSRGLPGNRTPLSFPPLTRRETYSERLINNIAGGGYYVQVQVGTPPQNLTMLLDTGSSDAWVLSHEADLCISPALQDFYGMPCTDTYDPSKSSSKKMVEEGGFKITYLDGGTASGDYITDHFTIGGVTVQSLQMACVTKAVRGTGILGLGFSISERASTKYPNIIDEMYSQGLIKSKAFSLYLNDRRADSGTLLFGGIDTDKFIGPLGVLPLHKPPGDRDYSSFEVNFTSVSLTYTNGSRHTIPTAILNHPAPAVLDSGTTLSYLPDELADPINTALDTFYDDRLQMTLIDCSHPLLRTDPDFHLAFTFTPTTSITVPLGDLVLDILPPTYPQSNSNNNNEVEDDDDDDDDDDDDDKVPPATERRWCVFGIQSTTRFAASSGQSEANFTLLGDTFLRSAYVVYDLSHYQIGLAQANLNSSSSSTNTNTIVELTADNHDDGASERGEGAGAGADAGTRTVIAGGLPSGLMGVEAQQTTFTPTATANGHPGYGGGPGGSTRPGSERNAAAGGFTAVRTGLLGELVGVAAVTALFILLGGALIAV

SEQ ID NO: 32 - protein, 562 aa, Myceliophthora heterothallica
MKPSSAILLALAPGSSSKNVVEFSVSRSLPGNRTPLSFPPLTRRETYSERLINNIAGGGYYVQVQVGTPPQNLTMLLDTGSSDAWVLSHEADLCISPALQDFYGMPCTDTYDPSKSSSKKMVKEAGFKITYLDGGTASGDYITDHFTIGGVTVRSLQMACVTKAVRGTGILGLGFNISERASTKYPNIIDEMYSQGLIKSKAFSLYLNDRRADSGTLLFGGIDTDKFIGPLGVLPLQKPPGGRDYSSFEVNFTSVSLTYANGSRHTIPTAILNHPAPAVLDSGTTLSYLPNELADPINTALDTFYDDRLQMTLIDCSHPLLRTDPDFHLTFTFTPTTSITVPLGDLILDILPPTYPQSNSKNNNEVEDDDDDDDDDDDDDKVPPATERKWCVFGIQSTTRFAASSSQSEANFTLLGDTFLRSAYVVYDLSHYQIGLAQANLNSSSSSTNTIVELTAAADDDDGASERGEGAGADAGTRTVIGGGLPSGLMGVEAQQTTFTPTATANGHPGYGGPGGSSRPGSEQNAAAGAFTALRTGLLGELVGVAAVTALFILLGGALIAV

SEQ ID NO: 33 - protein, 256 aa, Trichoderma reesei
MFIAGVALSALLCADTVLAGVAQDRGLAARLARRAGRRSAPFRNDTSHATVQSNWGGAILEGSGFTAASATVNVPRGGGGSNAAGSAWVGIDGASCQTAILQTGFDWYGDGTYDAWYEWYPEFAADFSGIDIRQGDQIAMSVVATSLTGGSATLENLSTGQKVTQNFNRVTAGSLCETSAEFIIEDFEECNSNGSNCQPVPFASFSPAITFSSATATRSGRSVSLSGAEITEVIVNNQDLTRCSVSGSSTLTCSYV

SEQ ID NO: 34 - protein, 243 aa, Myceliophthora thermophila
MKLAVLIATTAGLAAALPQGVARRGVGRPLHHSGPNIRNTTYPQYSSNWAGAVQIGTGFTSVYGTITVPSVHDRNPNAAASAWVGIDGDTCQQAILQTGVSFYGDGSFDAWYEWIPDYAYSFSNFRLSAGDQIRMSVEASSKRAGVATLENLSTGQKVSHTFTSTPSTLCETNAEWIVEDFQEGSSLVPFADFGTVTFTDAYATGSSGTVTPSGATIIDIKQGNEVLTNCATSGSDLTCSYTG

SEQ ID NO: 35 - protein, 243 aa, Myceliophthora heterothallica
MKLAILIAATAGLAAALPQGVARRGVGRPLHHSGPNLRNATYPEYSSNWAGAVQIGTGFTSVYGTITVPSVHDRNPNAAASAWVGIDGDTCQQAILQTGVSFYGDGSFDAWYEWIPDYAYSFSNFRLSAGDQIRMSVEASSKRAGVATLENLSTGQKVSHTFINTPSTLCETNAEWIVEDFQEGSSLVPFADFGTVTFTDAYATGSSGSVTPSGATIIDIKQGNEVLTSCATSGSDLTCSYTG

SEQ ID NO: 36 - protein, 236 aa, Trichoderma reesei
MDAIRARSAARRSNRFQAGSSKNVNGTADVESTNWAGAAITTSGVTEVSGTFTVPRPSVPAGGSSREEYCGAAWVGIDGYSDADLIQTGVLWCVEDGEYLYEAWYEYLPAALVEYSGISVTAGSVVTVTATKTGTNSGVTTLTSGGKTVSHTFSRQNSPLPGTSAEWIVEDFTSGSSLVPFADFGSVTFTGATAVVNGATVTAGGDSPVIIDLEDSRGDILTSTTVSGSTVTVEYE

SEQ ID NO: 37 - protein, 541 aa, Trichoderma reesei
MRSVVALSMAAVAQASTFQIGTIHEKSAPVLSNVEANAIPDAYIIKFKDHVGEDDASKHHDWIQSIHTNVEQERLELRKRSNVFGADDVFDGLKHTFKIGDGFKGYAGHFHESVIEQVRNHPDVEYIERDSIVHTMLPLESKDSIIVEDSCNGETEKQAPWGLARISHRETLNFGSFNKYLYTADGGEGVDAYVIDTGTNIEHVDFEGRAKWGKTIPAGDEDEDGNGHGTHCSGTVAGKKYGVAKKAHVYAVKVLRSNGSGTMSDVVKGVEYAALSHIEQVKKAKKGKRKGFKGSVANMSLGGGKTQALDAAVNAAVRAGVHFAVAAGNDNADACNYSPAAATEPLTVGASALDDSRAYFSNYGKCTDIFAPGLSIQSTWIGSKYAVNTISGTSMASPHICGLLAYYLSLQPAGDSEFAVAPITPKKLKESVISVATKNALSDLPDSDTPNLLAWNGGGCSNFSQIVEAGSYTVKPKQNKQAKLPSTIEELEEAIEGDFEVVSGEIVKGAKSFGSKAEKFAKKIHDLVEEEIEEFISELSE

SEQ ID NO: 38 - protein, 534 aa, Myceliophthora thermophila
MRGLVAFSLAACVSAAPSFKTETINGEHAPILSSSNAEVVPNSYIIKFKKHVDESSASAHHAWIQDIHTSREKVRQDLKKRGQVPLLDDVFHGLKHTYKIGQEFLGYSGHFDDETIEQVRRHPDVEYIERDSIVHTMRVTEETCDGELEKAAPWGLARISHRDTLGFSTFNKYLYAAEGGEGVDAYVIDTGTNIEHVDFEGRAKWGKTIPAGDADVDGNGHGTHCSGTIAGKKYGVAKKANVYAVKVLRSNGSGTMADVVAGVEWAAKSHLEQVQAAKDGKRKGFKGSVANMSLGGGKTRALDDTVNAAVSVGIHFAVAAGNDNADACNYSPAAAEKAVTVGASAIDDSRAYFSNYGKCTDIFAPGLSILSTWIGSKYATNTISGTSMASPHIAGLLAYYLSLQPATDSEYSVAPITPEKMKSNLLKIATQDALTDIPDETPNLLAWNGGGCNNYTAIVEAGGYKAKKKTTTDKVDIGASVSELEKLIEHDFEVISGKVVKGVSSFADKAEKFSEKIHELVDEELKEFLEDIAA

SEQ ID NO: 39 - protein, 534 aa, Myceliophthora heterothallica
MRGLVAFSLAACVSAAPSFKTETINGDHAPILSSSNAEVVPNSYIIKFKKHVDESSASAHHAWIQDIHTSREKTRQDLRKRGQVPLLDDVFHGLKHTYKIGQEFLGYSGHFDDETIEQVRRHPDVEYIERDSIVHTMRVTEETCDGELEKAAPWGLARISHRDALGFSTFNKYLYAAEGGEGVDAYVIDTGTNIEHVDFEGRAKWGKTIPAGDADVDGNGHGTHCSGTIAGKKYGVAKKANVYAVKVLRSNGSGTMADVVAGVEWAAKSHLEQVQAAKDGKRKGFKGSVANMSLGGGKTRALDDTVNAAVSVGIHFAVAAGNDNADACNYSPAAAEKAVTVGASAIDDSRAYFSNYGKCTDIFAPGLSILSTWIGSKYATNTISGTSMASPHIAGLLAYYLSLQPATDSEYSVAPITPEKMKSNLLKIATQDALTDIPDETPNLLAWNGGGCNNYTAIVEAGGYKANKKTTTDKIDIGASVSELEKLIEHDFEVISGKVVKGVSSFADKAEKFSEKIHELVDEELKEFLEDIAA

SEQ ID NO: 40 - protein, 409 aa, Trichoderma reesei
MASLRRLALYLGALLPAVLAAPAVNYKLPEAVPNKFIVTLKDGASVDTDSHLTWVKDLHRRSLGKRSTAGVEKTYNIDSWNAYAGEFDEETVKQIKANPDVASVEPDYIMWLSDIVEDKRALTTQTGAPWGLGTVSHRTPGSTSYIYDTSAGSGTFAYVVDSGINIAHQQFGGRASLGYNAAGGDHVDTLGHGTHVSGTIGGSTYGVAKQASLISVKVFQGNSASTSVILDGYNWAVNDIVSRNRASKSAINMSLGGPASSTWATAINAAFNKGVLTIVAAGNGDALGNPQPVSSTSPANVPNAITVAALDINWRTASFTNYGAGVDVFAPGVNILSSWIGSNTATNTISGTSMATPHVVGLALYLQALEGLSTPTAVTNRIKALATTGRVTGSLNGSPNTLIFNGNSA

SEQ ID NO: 41 - protein, 392 aa, Myceliophthora thermophila
MHFSTALLAFLPAALAAPTAETLDKRAPILTARAGQVVPGKYIIKLRDGASDDVLEAAIGKLRSKADHVYRGKFRGFAGKLEDDVLDAIRLLPEVEYVEEEAIFTINAYTSQSNAPWGLARLSSKTAGSTTYTYDTSAGEGTCAYVIDTGIYTSHSDFGGRATFAANFVDSSNTDGNGHGTHVAGTIGGTTYGVAKKTKLYAVKVLGSDGSGTTSGVIAGINFVADDAPKRSCPKGVVANMSLGGSYSASINNAAAALVRSGVFLAVAAGNENQNAANSSPASEASACTVGATDRNDAKASYSNYGSVVDIQAPGSNILSTWIGSTSATNTISGTSMASPHIAGLGAYLLALEGSKTPAELCNYIKSTGNAAITGVPSGTTNRIAFNGNPSA

SEQ ID NO: 42 - protein, 392 aa, Myceliophthora heterothallica
MHFSTALLAFLPAALAAPTAETLDKRAPILTARAGQVVPGKYIIKLRDGASDDVLEAAIGKLRSKADHVYRGKFRGFAGKLEDDVLDAIRLLPEVEYVEEEAIFTINAYTSQSNAPWGLARLSSKTTGATTYTYDTSAGEGTCAYVIDTGIYTSHSDFGGRATFAANFVDSSNTDGNGHGTHVAGTIGGTTYGVAKKTKLYAVKVLGSDGSGTTSGVIAGINFVADDAPKRSCPKGVVANMSLGGSYSASINNAAAALVRSGVFLAVAAGNENQNAANTSPASEASACTVGATDRNDAKASYSNYGSVVDIQAPGSNILSTWIGSTSATNTISGTSMASPHIAGLGAYLLALEGSKTPAELCNYIKSTGNAAITGVPSGTTNRIAFNGNPSA

SEQ ID NO: 43 - protein, 1419 aa, Trichoderma reesei
MKSALLWAAPLSLLAGLGACGKNFDELLEVPEGWTQLNDAVNPSQHIRLSIAVKQPYIDSLEARMAEKGNRLSMQEVRELQTPAKKDIDNVLHWLSQNNLYGVVEKDFIRVWTTVAKAEPLLKMKLSRFSYEGKPAVLRTTKYTIPDSVADSISFINPINNFMSARHRERGLTFLLPPSKGAVLPGNTTAYCAGSVTPSCLSKLYNINYSPANTSSPVIFGVAGFLEENANLQDLRQFLNQSAPEVAKTGRTINVELVNGGVNSQELSESGIEAALDVDYAVSLGFPTNVTFYSTGGRGVKLNDDGQPIEGEDDDNEPYLEFFQYLLAKPDGQVPHVLSLSYSDDELSVPRDYAKRVCSLFGLLTARGTSIIFSSGDGGARGGRASSCLTNDGTKRQVTMATFPPTCPWVTSIGAVTNIAEPPNGARFSTGGFSQYFAQPRWQNEAVEGYVKALGGHLDGYYNESMRAIPDVSAVGTAFSIISGGYPRSVQGTSASAPVFAAMIALINDARLRAGKKSLGFLNQHLYSSEVRAVLQDITAGQSASCIWNDADIPGGWPAAEGWDAITGLGVPKRFDKLMEVLVNHLPRLATGFARFKMPTQHVEPESHALLQDVANSLLKARKVVVVTGAGISTNSGIPDFRSENGLYSLIQAQFDAANQPTRPAERSKADGTADSGEEPRPTKRGMTSREASPDLDEVTRQLRDDIEARAESQRPAASSRAAGTQPAVAASDANASAEGVCLSTPRRKPALPSTPLPTTSPLSSPPREDFLIPLPSWSSSSILRTEDRKRITDASHNVVSSPLSSPPPVLFDPFHPSSPSDEDRSRRSSTTPSEAGENPPNAMPASQTSSFGKANLPNMKGKDLFDASIWSDPTRTSVFYQFATSLRQKVRDAEPTSSHKFISHLRDRGKLVRCYTQNIDQIEEKVGLSTSLEDGPGSRGRFSRKATANASQLNKMVQEVSSGEGGASSHVNASSQSSNGSSEQSSAESSQANDRTEEEDSAGSSSTTAATTTTTTTPPDRPKPAPRKEPPQSGVECVFLHGSLQLLRCFLCGQVCSWDDDDREVETLSGLQPECPHCVGATEARQERGKRALGVGKLRPDIVLYGEEHPSAHLISPIVTHDLALYPDMLLILGTSLRVHGLKVLVREFAKTVHSRGGKVVFVNFTKPPESSWGDIIDYWIQWDCDAWVSDLQVRIPKLWQDPEPPKPKKKRDSGGAADDNREEKKRPPAQNPVALRDTKVNGAYCTLKILKELRRITYTRDSAAIGNPIITAPEPPTTTEAISRAVAMDSSRTSTPRGKSKRSRRSATGAIDRPKRTPSTLNPNHGRSKKPTAEAKKKQQEEEEVPETPSQPPASTVEEFSSILHSVKSNPRIRKRKMIDGEEFVFPAVGKKRGAVDSLYKGPGDDTKELPPLRPMP

SEQ ID NO: 44 - protein, 464 aa, Myceliophthora thermophila
MRYSVPAPLLGLVIPLVHAAPAVILEETKGIPPGWVFLGNASASDRMTLFVALKEPGIEELKAKLNQRRDVDHPSFGQHLSRDEVAQYRQPGDQSSRAVLGWLRSEGIRNIHNQGSLISFEASAQTVKTLFHAELGYYAYEESNAAPVLRAQSYTIPAWLCDYIDFKPFSTITSTTTTTTRSTTSAISSSSSTLAPKPTPDTPDNTINPFPNLPCLAATVPDCIKKLYNITYSPSPPGSPSPVRFGVAGFLEQWVLHSDVDLFLDTLAPTVPRRMNNNNNNNNSYYNFTVELVNNGTNPQDSPRNAGLEASLDVEYAMALAYPANVVFYSTGGRGTMLDAGTGTGVALPPVSQSDNEPFLELLQALLAKPDGELPHVLSISYADDEVGVPRAYARRVCDQFAALAARGTTVLAATGDGGAAGTGRTRCVDRETGARRFVATFPAGCPYVTAVGAVSNVGPPVAG

SEQ ID NO: 45 - protein, 648 aa, Myceliophthora heterothallica
MRYSVPAALLSLVIPLVHAAPAVILEETKGIPPGWVFLRNASASDRMTLFVALKEPGIEELKAKFSQRRDVDHPSFGQHLSRDEVAQYRQPGEQSSRAVLGWLRSEGIRNMHNQGSLISFEASAQTVKTLFHAELGYYAYEESNAAPVLRAQSYTIPAWLRDYIDFVHPISSFMPPRPRPRPHPKPTKKPFSMITSTTTTTTTRSTTSAISSSSSSSSSSITLAPKPTPDIPDNMINPFPDLPCLAATVPDCIKKLYNISYSPSPPGSPSPVRFGVAGFLEQWVLHSDVDLFLDTLAPTLPRRTNDDDKNSHNFTVELVNDGTNPQDSPRNAGLEASLDVEYAMALGYPANVVFYSTGGRGAMLDAGTGLALPASQSDNEPFLELLQALLAKPDGELPHVLSISYADDEVGVPRAYARRVCDQFAALAARGTTVLAATGDGGAAGTGRTQCVDRETGARRFVATFPAGCPYVTAVGAVSNVGPPVAGAAYSSGGFSDFFGRPAWQDEAVEPYLAALVRGNDTRIGLFNHGGRAVPDISAVGSAFQIVLGGDNSQVLGTSASTPVVAAMVALINDARMRAGKPSLGWLNPLLYSAKVRNVLRDVTVGESMGCRFPDGVRSVGWSAVRGYDLVTGLGVVDDFNDFLEALL

SEQ ID NO: 46 - protein, 421 aa, Trichoderma reesei
QWTGTVDVTPSHRLFFWFFNSRNDPVRDPVIVWMNGGPGGSSMMGLFNELGACWLDPGANATVPNPFAWNNNASVLFLDQPAGVGLSSRSPGSPLQAHDVDGAADFQAFLNIFFGKIFPDRAHLPIHIAAESYGGHYGPVYLKHILDSRRFDSPDAFRGNISSLILVDAVIDFAAPAVGTYEFLCRGYGHHGKIMNDSTCDALRRSVPKVFELDRNCDAGNDGYECWGMTTFLEEVVHPYYYAEVEAGKRNPFNVHIPCPNWPWCADIRKGNITAYLNQPAVKAALGFPPSFTFEDVDIPLNAAYTLSKDPFRPTARQVAAVLDAGIRVLVLQGNEDYIVNTPGNMWAYDNLRWSGLADYRLAHWRPLPEGVAATGTWKAGGEGGRLAFVAVDGAGHTVPGDVREGSWRIVKEWLEGGWRG

SEQ ID NO: 47 - protein, 444 aa, Myceliophthora thermophila
MRWSLASVVLAYLGSVFGYHKSRAFDMRYGPAPDWTQSPFLNERSQLYFWFFPSTDPVARQNKEILIYLSGGPGCSSIGELVQMNGPISWLPGTNQPIPNRWSWHHLTNVVWVDQPVGTGFSQGTPTATNEVDVAEQFLGFWQNFVDTFALRGYQVYVTGSSYAGMYAPFITSAMLDWNGTANFNVSGMAIWAGLYSKIPLTMDIPAARYVDKWKMAFPLNDTSRAGIKAIDQRCGFTAYLDEFLVFPAKGPQPSTLPGEDPATGLVRPECALYAGAWLAAHELNPCFTLFDVTSRCPILFDPLGLASGRYILPASFNTSYFNLPAVKAALNAPRNSTWAFCPKVFSNPVFVNGVDTSLRAGPGSQPAIPRVIDGTRNVILGHGSRDFLILPDATLLTIQNMTWQGQRGFRTRPRSPLFVPYHDNRDPLTAAGAGVKQSCDEGE

SEQ ID NO: 48 - protein, 444 aa, Myceliophthora heterothallica
MRWSLASLVLAYLASVFGYHESRASDTRYGPAPDWTQSPFLNERSQLYFWFFPSTDPVARHNKEILIYLSGGPGCSSIGELVQMNGPISWLPGTNKPIPNRWSWHHLTNVVWVDQPVGTGFSQGTPTATNEVDVAEQFLGFWQNFVNTFALRGYQVYVTGSSYAGMYAPFISSAMLDRNGTANFNVSGMAIWAGLYSKVPLTMDIPAARYVGKWKMAFPLNDTSRAGIEAIDQRCGFTAYLDEFLVFPAKGPQPSTLPGEDPVTGLVRPECALYAGAWLAAHELNPCFTLFDVTSRCPILFDPLGLASGRYILPASFNTSYFNLPAVKAALNAPRNSTWAFCPKVFSNPVFVNGVDTSLRDGPGSQPAIPRVIDGTRNVILGHGSRDFLVLPDATLLTIQNMTWQGQRGFRTRPRSPLFVPYHDNRDPLSAAGAGVKQSCDEGE

SEQ ID NO: 49 - protein, 612 aa, Trichoderma reesei
MAKLSTLRLASLLSLVSVQVSASVHLLESLEKLPHGWKAAETPSPSSQIVLQVALTQQNIDQLESRLAAVSTPTSSTYGKYLDVDEINSIFAPSDASSSAVESWLQSHGVTSYTKQGSSIWFQTNISTANAMLSTNFHTYSDLTGAKKVRTLKYSIPESLIGHVDLISPTTYFGTTKAMRKLKSSGVSPAADALAARQEPSSCKGTLVFEGETFNVFQPDCLRTEYSVDGYTPSVKSGSRIGFGSFLNESASFADQALFEKHFNIPSQNFSVVLINGGTDLPQPPSDANDGEANLDAQTILTIAHPLPITEFITAGSPPYFPDPVEPAGTPNENEPYLQYYEFLLSKSNAEIPQVITNSYGDEEQTVPRSYAVRVCNLIGLLGLRGISVLHSSGDEGVGASCVATNSTTPQFNPIFPATCPYVTSVGGTVSFNPEVAWAGSSGGFSYYFSRPWYQQEAVGTYLEKYVSAETKKYYGPYVDFSGRGFPDVAAHSVSPDYPVFQGGELTPSGGTSAASPVVAAIVALLNDARLREGKPTLGFLNPLIYLHASKGFTDITSGQSEGCNGNNTQTGSPLPGAGFIAGAHWNATKGWDPTTGFGVPNLKKLLALVRF

SEQ ID NO: 50 - protein, 621 aa, Myceliophthora thermophila
MLRNIFLTAALAAFGQCGSTVFESVPAKPRGWTRLGDASADQPLRLRIALQQPNEDLFERTLYEVSDPSHARYGQHLSRDELSALLAPRAESTAAVLNWLRDAGIPSDKIEEDGEWINLRVTVREASELLDADFGVWAYEGTNVKRVRALQYSVPEEIAPHIRMVAPVVRFGQIRPERSQVFEVVETAPSQVKVAAAIPPQDLDVKACNTSITPECLRALYKVGSYQAEPSKKSLFGVAGYLEQWAKYDQLELFASTYAPYAADANFTSVGVNGGENNQGPSDQGDIEANLDIQYAVALSYKTPITYYITGGRGPLVPDLDQPDPNDVSNEPYLEFFSYLLKLPDSELPQTLTTSYGEDEQSVPRPYAEKVCQMIGQLGARGVSVIFSSGDTGVGSACQTNDGKNTTRFLPIFPGACPYVTSIGATRYVEPEQAAAFSSGGFSDIFKRPAYQEAAVSTYLHKHLGSRWKGLYNPQGRGFPDVSAQGVAYHVFSQDKDIKVSGTSASAPLFAALVSLLNNARLAQGRPPLGFLNPWLYSEKVQKAGALTDIVHGGSSGCTGKDMYSGLPTPYVPYASWNATPGWDPVTGLGTPVFDKLLELSSPGKKLPHIGGGHGHGAGGH

SEQ ID NO: 51 - protein, 621 aa, Myceliophthora heterothallica
MLRNIFLTAALAAFGQCGSTVFESVPTKPRGWTRLGDASADQPLRLRIALQQPNEDLFESTLYEVSDPSHARYGQHLSRDELSALLAPRAESTAAVLNWLRDAGIPSDKIEEDGEWINLRVTVREASELLDADFGVWAHEGTNVKRVRALQYSVPEEIAPHIRMVAPVVRFGQIRPERSQVFEVVDTVPSQVKVAAAIPPQDLDVKVCNTSITPECLRALYKVGSYQAEPSKKSLFGVAGYLEEWAKYDQLELFASTYAPYAADANFTSVGVNGGENNQSPSDQDDIEANLDIQYAVALSYKTPITYYITGGRGPLVPDLDQPDPNDVSNEPYLEFFSYLLKLPDSELPQTLTTSYGEDEQSVPRPYAEKVCQMIGQLGARGVSVIFSSGDTGVGSACQTNDGKNTTRFLPTFPGACPYVTSIGATRYVEPEQAAAFSSGGFSDIFKRPAYQEAAVSTYLRKHLGNRWKGLYNPQGRGFPDVSAQGVAYHVFSQDKDIKVSGTSASAPLFAALVSLLNNARLAQGRPPLGFLNPWLYSEKVQKAGALTDIVHGGSNGCTGKDMYSGLPTPYVPYASWNATPGWDPVTGLGTPVFDKLLELSSPGQKLPHIGGGYGHGAGGH

SEQ ID NO: 52 - protein, 538 aa, Trichoderma reesei
MHAVNLHARHDPEQRVSVRQYEDRYPNIKAYNFSVPIDHFHNESRYEPHSGDSFPLRYWLDISNYKPGGPVIVLHSGEFDSTERLPYLQHGIVPILTKATGGVGLIMEHRYYGTSFPVPDLTTESLRFLTTEQALADTAYFAKHVRFPGLEHLDLTASKTPWVIYGGSYAGAFAAFARKLYSEVFWGAISSSGVTAAVEDLWQYFEAARRYAPGDCGPTTQKLTHVVDTVLFGDDRAKVLGFKQLFGLGDLDDDDFATTITRGLYGLQSTNWDPEEDVSSLGIYCAVVTSNFQLFASTTHLIPTVQDLVAKAGFAAEAEVLSVRMLNYIGYVRDYVKTDLAKGCKGKSVSECFSPRYSTPNDTSLNAGWLRSWSWQVCTQWGYFVTGSGTPRDQLPMISRAITLESASSHCKRLFNITTSPDVESINKLGGFNFSYPRLAILDGLQDPWRSATPHASGLPDRESTTSEPFMLIDWGVHHWDEFGLPEDVHVEGLPPPQVAEAHMAEIEFVKAWLADWKEENGISMLQSMDEEETMGEL

SEQ ID NO: 53 - protein, 561 aa, Myceliophthora thermophila
MLRGTIAVGVACLAQLVAGLDGPLFRTSLTLRDFREQLERRQARDGAALEARSSDLQDLYPAHTLQVPVDHFHNDSLYEPHSSETFPLRYWFDASHYKKGGPIIVLQSGETDGVGRLPFLQKGIVAQLARATNGLGVILEHRYYGESIPTPDFSTEKLRFLTTDQALADMAYFARHVVFKGLEHLDLTSAKNPYIAYGGSYAGAFVAFLRKLYPDVYWGAISSSGVPEAIYDYWQYYEAARIYAPHDCVVATQKLTHIVDNILLDKADTDYVRRLKTGFGLGGVTRNDDFANAISWGIGGLQGLNWDPALNDTGFGEYCNNLTATKPLYPTSPALEQEARELVKAGGYGKEADTLTTQLLNYMGYVNATTVQTCHKDSQDECFTNYNSTFYQQDDKTQDWRLWPYQYCFEWGYLQTGSGVPANQLPLISRLIDLNFTSVVCREAFNITTPSQVERINKLGGVNISYPRLAFVDGERDPWRYASPHRIGLPERKNTISEPFILIKDGVHHWDENGLFPNETRPGLPPKPVADAQRAEVKFVKAWLKEWKEKEKCRGRKFCWP

SEQ ID NO: 54 - protein, 561 aa, Myceliophthora heterothallica
MLRGTIAVGAACLAQLVAGLDGPLFRTSLTLRDFRQQLERRQARDGAALEARSSDLQDLYPAHTLQVPVDHFHNDSLYEPHSSETFPLRYWFDASHYKKGGPIIVLQSGETDGVGRLPFLQKGIVAQLARATNGLGVILEHRYYGESIPTPDFSTENLRFLTTDQALADMAYFARHVVFKGLEHLDLTSAKNPYIAYGGSYAGAFVAFLRKLYPDVYWGAISSSGVPEAIYDYWQYYEAARIYAPHDCVVATQKLTHIVDTILLDKADTDYVRRLKTGFGLGGVTRNDDFANAISWGIGGLQGLNWDPALNDTGFGEYCNNLTATKPLYPTSPALEQEARELVKAGGYGEEADTLTIQLLNYMGYVNATTVQTCHNDSQDECFTNYNSTFYQQDDKTQDWRLWPYQYCFEWGFLQTGSGVPANQLPLISRLIDLNFTSVVCREAFNITTPSQVERINKLGGVNISYPRLAFVDGERDPWRYASPHRIGLPERKNTISEPFILIKDGVHHWDENGLFPNETRPGLPPKPVADAQRAEVKFVKAWLKEWKEKEKCRGRKFCWP

SEQ ID NO: 55 - protein, 910 aa, Trichoderma reesei
MAPRASDRLERSPSTDSLSSVSTTSIVFDRIQEEMEKGEVPPKARARLGYSDVYAEDPLKDEASGDAETEAFLGSTGAALQRRPMDRKLRRFLCIGAVIFAAAWLASLAVFVSTGSYKHASDVEHDPDADFRGSGKPVTLEQLYDRFWSPKQKSISWIAGPHGEDGLLLERGASGKDYLIVEDVRSAVGNATAAAAAAADETAQLAKSRTLMKHADFEFDGKTYQPQWLQPSPDQNKVLIGVAQEKVWRHSFTAIYFIFDVETQTAEPLVPSEAQARVQLAGWSPRSDSVSFTRDNNLYIRRLTGDGGIVQITKDGGPEYFYGIPDWVYEEEVFSGNSATWWSDDGKYLAFLRTNETGVPEYPVQFFISRPSGHTPPSGEESYPEVQEIKYPKAGAHNPVVDLHFYDVSKGDVFSVSTGDEFPDDDRIINNLVWAGDKVIVKQTNRVSDILKVILIDVNKRQGSTINTIDVNKIDGGWFEISHSMTYVPADPENGRPEDGYVDTVIHNGYEHIGYFTPLNNSEPIMLTSGSWEVDDAPSAVDLANNLVYFVATKESSIQRHVYSVKLDGSELKPLTNTSAEAYYRASFSSGAGFALLTYRGPKTPYQKVISTPSTTIAYDVVVEDNAELADRAKKHELPVLKYGTIDLDTGVKVNYLERRPPHWDPKKKYPILFQQYSGPGSQQVTKAFTVDFQSYVASNLGYIVVTVDPRGTGFLGRKHRVPVRSQLGLLEAQDHIAAARHFASLDYVDASRLAIWGWSYGGFTTLKTLEQDAGRTFSYGMAVAPVTDWRFYDSIYTERYMRTPQENDKGYALSSIANATALGENKRFLLMHGVADDNVHFQNSLTLLDKLDLAGVENYDVHVFPDSDHGIYFHGANRIVYDKLNNWLINAFNGEWLKVSHPKPIGKKA

SEQ ID NO: 56 - protein, 955 aa, Myceliophthora thermophila
MAPYRDSPQSSLAHSPNRERSQSPARMSYESGSSVSTTSIVFDRISERVAAASKAAPEKHRRGDNGESDDEDLKDETAYEDLETGPFLGSGNGNPTQHAEVKGKGMDRGLRRALLIAAGLLVSAWVAGLFVYVATKSYKPASEIAHDPQATIVRGSEKAITLDQVLNSFWRPEVRSIEWIAGPDGEDGLLLERGEGKDFLVVKDIRAQNGAGVAADAPVADSRTLMKEARFEHGQKGYSVGKVVPSRDLQKVLVATNEKSNWRHSSYAAYWIFDVKTQTAEPLVPGEPDARLQLAQWSPTSDAVAFTRDNNLYLRKVGSDSVVQITQDGGSEVFNGVPDWVYEEEVFSGPSATWWSEDGKYIAFLRTNETGVPEYPVQYFLSRPSGTEPEPGEELYPEVRNIKYPKAGTHNPVVELKFYDVERGDVFSVDTSGGFADDDRLITEVVWAGGQVLVKETNRVSDVMRVVLVDVAARTGKAVRTTDVRALDGGWFEITHQTKYIPADPSKGRSHDGYIDLIIHGDGNHLAYFTPLDNPEPVMLTSGDWEVVDSPYAVDLDKNVVYFVATKESSIQRHVYQVGLTGDGLAPVSDTSSEGYYAASFSIGGGYVLLTYEGPGIPWQKVVSTPSNPRRYEYVVEENQDLAERAKKHELPIKIYGTINVDGVELNYIERRPPHFDASKKYPVLFYQYSGPGSQTVSKKFLVDFQSYVASGLGYICVTVDGRGTGYIGRKNRVLIRGKLGQWESHDQIAAAKIWAGKKYVDETRLAIWGWSFGGYNTLKTLEQDGGQTFRYGMAVAPVTDWRFYDSIYTERYMLTPQANEKGYDGSAISNSTALSQNVRFLIMHGVADDNVHFQNSLTLLDKLDLAQVENYDVHVFPDSDHSIYFHGANRVVYDSKLSPPDKSTIVGFALTNDYDRTDELAHQRFQRRVDQSHEPNTEGQKEVGLKGPVTRCAS

SEQ ID NO: 57 - protein, 924 aa, Myceliophthora heterothallica
MAPYRDSPQSSLAHGPNRERSQSPARMSYESGSSVSTTSIVFDRISERVAAVSKAAPEKHRRGDNGESDDEDLKDETRYEDLETGPFLGSGNGNPTQHADVKGKGMDRGLRRALLIAAGLLVSAWVAGLFVYVATKSYKPASEIAHDPQATIVRGSAKAITLDQVLNSFWRPEVRSIEWIAGPDGEDGLLLERGEGKDFLVVKDIRAQNGAGVAADASVADSRTLMKEARFEHGQKGYSAGKVVPSRDLQKVLVATNEKSNWRHSSYAAYWIFDVKTQTAEPLVPGEPEARLQLAQWSPTSDAVAFTRDNNLYLRKVGSDSVVQITQDGGSEVFNGVPDWVYEEEVFSGPSATWWSEDGKYIAFLRTNETGVPEYPVQYFLSRPSGTEPEPGEELYPEVRNIKYPKAGTRNPVVELKFYDVARGDVFSVDTSGGFADDDRLITEVVWAGGQVLVKETNRVSDVMRVVLVDVAARTGKVVRTTDLRALDGGWFEITHQTKYIPADPSKGRGHDGYIDLIIHGDGNHLAYFTPLDNPEPVMLTSGDWEVVDSPYAVDLDKNVVYFVATKESSIQRHVYQVGLTGEGLAPVSDTSSEGYYAASFSIGGGYVLLTYEGPGIPWQKVISTPSNPRRYEYVVEENQDLAEKAKRHELPIKIYGTIDVDGVELNYVERRPPHFDAGKKYPVLFYQYSGPGSQTVSKKFLVDFQSYVASGLGYICVTVDGRGTGYIGRKNRVPIRGKLGQWESHDQIAAAKIWAGKKYVDEARLAIWGWSFGGYNTLKTLEQDGGQTFRYGMAVAPVTDWRFYDSIYTERYMLTPQANEKGYDGSAISNTTALSQNVRFLIMHGVADDNVHFQNSLTLLDKLDLAQVENYDVHVFPDSDHSIYFHGANRVVYDKLTNWLINAFNGEWIKVTNPTPKAKKRSA

SEQ ID NO: 58 - protein, 690 aa, Trichoderma reesei
MTITAQKLTPEVLLAAPRRSPGVPNATGELVLYTVSTYSFDSHSKTAQIRVLNLKEGTSHLVSEDSAASEPIWIAEQEIAYVKSLDHGASALVAQHVFNPNESNTIQRFGGSINSLKAKPLSTDKVAFCCAALTTPDGRMYSPAAEPKSYTSAKIYTSLFVRHWDSWNTENKNSLWYGQLNKVDGKWTLGNSELTNLLAGTRLHSPVPPFGGTGDFDISTTGIVFVAKDPDLNHARTTKTDLYFVPLNSYLDQPTFPQIVKTGALRGYSLSPVFSNDGKQVAFLRMKSQQYEADKTRLLLIPDVTDLSNVQEFYATEDGKGGWDYKPDWLIWSHDDKELYVAAEKHARVVLWKLPSSPLEAKSLPTPIHEDGSVAEARVLGKGSSLLITTRSRVESSNYSILDPASKSTTIISSSSRQGKTFALSKSQCQEIWFNGSKGYPIHALVTLPSTFDSSKKYPLAFFIHGGPQGAWGDDWSTRWNPAVFAEQGYVVVSPNPTGSTGYGQDHTDAIQNNWGGDPYIDLVKCFEFLEEEVNYIDTTRAVALGASYGGYMINWIQGHDLGRKFKALVCHDGVFSTLNQWSTEELFFPEHDFGGALWENREGYEKWDPAKHVGNWATPQLVIHNELDYRLPISEGLAMFNVLQARGVPSKFVMFPDEHHWVLKPENSLVWHREVLNWINKYSGISEKN

SEQ ID NO: 59 - protein, 725 aa, Myceliophthora thermophila
MTLTATKFTPEVMLSAPRRSPGVPNPSGTLALYTVSTYSFKDHEKTTNIRVLDINTGTATTPVDEAGASNPVWLNDDEILYVRSGDKGSSTLVVRNVTASASSSFYEAGTIQGPIGDLKVKALADGTVALACSASATPDGNLFNPETEVKPHSTARVYTSLFVRHWDTWNSEKRSSIFYGVLDKKDGKYALRDGRLTNALAGTRLSSPVPPFGGTGDFDIGPEGLVFVAKDPDLDPAIYTKTDLYFIPLKTFAESSPPPPKIVKTPGLEGYSNSPVFSHCGRKVAFTRMRSNQYESDKPRLLLVRDIGNPDETEEFFATPDGDGAWDSRPESIAWGHDDTQLYVTAEHRARTLIFTLPSTPSRATSLPTPLPTPDGSVADFRLYAASSPSSSSKTDHLFVTTTSLIDNSCYSLTHPATATSTVISSASKQGRSFGLSRAQVDSITVPGPDYDVHALVVRPSTWVSDAATGSTTGSRRATKKYPLCMLVHGGPQGAWQDSWSTRWNPAVFAEQGYVVVCPNPTGSTGYGMALEDGIRGQWGGRPYEDLERCFDWVVANMPEVDGERAVALGASYGGFMINWIQGHPLGRKFKALVCHDGVFSTLNQWATEELFFPIHDFEGTLYENREGYEKWDPARFINEWATPQLIIHSELDYRLPVTEGLAAFNVLQAKKIPSKLLVFPDENHWVLKHENSLVWHREVLGWINKYSGIDDEQKLAEETEALKV

SEQ ID NO: 60 - protein, 725 aa, Myceliophthora heterothallica
MTLTATKFTPEVMLSAPRRSPGVPNPSGKLALYTVSTYSFKDHEKTTNIRVLDINTGTATTPVDEAGASNPVWLNDDEILYVRSGDKGSSTLVVRNVTASASSSFYEAGTIQGPIGDLKVKALADGTVALACSASATPDGNLFNPETEVKPHSTARVYTSLFVRHWDTWNSEKRSSIFYGVLDKKDGKYALRDGRLTNALAGTRLSSPVPPFGGTGDFDIGPEGLVFVAKDPDLDPAIYTKTDLYFIPLKTFAESSPPPPKMVKTPGLEGYSNSPVFSHCGRKVAFTRMRSNQYESDKPRLLLVRDIGNPDETEEFFATPDGDGAWDSRPESIAWGHDDTQLYVTAEHRARTLIFTLPSTPSRATSLPTPLPTPDGSVADFRLFAASSPSSSSKTDHLFVTTTSLIDNSCYSLTHPATATSTVISSASKQGRSFGLSRAQVDSITVPGPDYDVHALVVRPSTWVSDAATGSTTGSRTATKKYPLCMLVHGGPQGAWQDSWSTRWNPAVFAEQGYVVVCPNPTGSTGYGMALEDGIRGQWGGRPYEDLERCFDWIVANMPEVDGERAVALGASYGGFMINWIQGHPLGRKFKALVCHDGVFSTLNQWATEELFFPIHDFEGTLYENREGYEKWDPARFINEWATPQLIIHSELDYRLPVTEGLAAFNVLQAKKIPSKLLVFPDENHWVLKHENSLVWHREVLGWINKYSGIDDEQKLAEETEALKV

SEQ ID NO: 61 - protein, 533 aa, Trichoderma reesei
MWSLAVAAELLLLAGTASALLFQDLRPPKPDVDLDNNFRIAAAASTPTYGTFQQLIDHDHPELGTFSQRYVWNDEFYAGPGSPIILMGPNESALDGYERYTTNLTLPGVMAQELGAGALIIEHRYWGQSSPFDSLTTENMRYLTLEQSVQDLVYFAQNVVLPFDQNRTSTPDKAPWVLVGCSYSGALAAWVQDLAPGTFWAYQCSSPVVEAIGPLWKYFEQVKLAMPQNCTADYARIVQYIDGVLLGHNQKAKDELKASFNLGDVEHDDDFAAALVSGLYTWQSVLFSDGYSATNRMCDYVENQWPGSTAPAPGKNGVGLTKALAGYAKWFTTLSLPGSCASYGYWTNPNDTSCYDTYNASSPLFTDLSPENTGNRQWWWFLCNEPFKWWQVRGLGNLVSRLVNQPYFERQCGLIFPREGKYTYGIAQGATTARVNRVTGGWYNVNTHRLAWTAGEYDPWRPATVMAVDRPGGPLRSTKQHPVWVVPGAAHCGDMLVRNRNANEGVRNVFDSEVANIKQWVQEFYEEKGKKGN

SEQ ID NO: 62 - protein, 544 aa, Myceliophthora thermophila
MRLSITALAAGLAFAPLAAAFGRGLIMDIGPDDDTVSETELQKRAETVNGWGTFDQLIDHANPSLGTFKQRYWYGTEYWKGPGSPIYLVSPGEQSGEGFNRTWLTTARLSGVMANQTGGAVIVLEHRYWGQSSPYDELTVENLRYLTLDNSLKDLVYFAKNFAPPFDPSGSSSADKAPWIFAGGSYSGALAGWLAAREPGTFWAYYSSSGVVEAIGDFWQYFVPVQEATPKNCSADVGAVINYVDLVLSFGSKKSKQALKDKFGLGELEDADFAAALEWGPWEWQSGQFYSFNTTGYNPFYRFCDYVENVWPNSTNKVPGPRGVGLTKALEGYAKYVKEQVIPGFCESAGYPEWEGENNIACFKNLDPNNAAYKDLSLDNWINRQWNWMLCNEPFEWWQDGAPLTRPTLVSRLVNADYWRKQCPLHFPEGGYGIAAGKRAKDVNRWTGGWSVTNTTRAMHTNGQYDPWRDATLSSKFRPGGPVQSTERLPVRLVKGGTHCSDLYGQNWAVNEDVKKLAQDAADEMTGWVGEWYEEKGLPKPWAA

SEQ ID NO: 63 - protein, 544 aa, Myceliophthora heterothallica
MRLSITALAAGLALAPLAAAFGRGLVMDIGPDDDTVSETDLQKRAETVNGWGTFDQLIDHANPSLGTFKQRYWYGTQYWKGPGSPVYLVSPGEQSGEGFNRTWLTTARLSGVMANQTGGAVIVLEHRYWGESSPYDELTVENLQYLTLDNSLKDLVYFAKNFVPPFDPSGSSSADKAPWIFAGGSYSGALAGWLAAREPGTFWAYYSSSGVVEAIGDFWQYFVPVQEATPKNCSADVGAVINYVDLVLSFGSKKSKQALKDKFGLGELEDADFAAALEWGPWEWQSGQFYSFNTTGYNPFYRFCDYVENVWPNSTNKVPGPGGVGLTKALDGYAKYVKEQVIPGFCESAGYPEWEGENNIACFKNLDPNNAAYKDLSLDNWINRQWNWMLCNEPFEWWQDGAPLTRPTLVSRLVNADYWRKQCPLHFPEGGYGIAAGKRAKDVNRWTGGWSVTNTTRAMHTNGQYDPWRDATLSSKFRPGGPVQSTKRLPVRLVKGGTHCSDLYGQNWAVNEDVKKLAQDAADEMTGWVGEWYEEKGLPKPWES

SEQ ID NO: 64 - protein, 882 aa, Trichoderma reesei
MVRSALFVSLLATFSGVIARVSGHGSKIVPGAYIFEFEDSQDTADFYKKLNGEGSTRLKFDYKLFKGVSVQLKDLDNHEAKAQQMAQLPAVKNVWPVTLIDAPNPKVEWVAGSTAPTLESRAIKKPPIPNDSSDFPTHQMTQIDKLRAKGYTGKGVRVAVIDTGIDYTHPALGGCFGRGCLVSFGTDLVGDDYTGFNTPVPDDDPVDCAGHGSHVAGIIAAQENPYGFTGGAPDVTLGAYRVFGCDGQAGNDVLISAYNQAFEDGAQIITASIGGPSGWAEEPWAVAVTRIVEAGVPCTVSAGNEGDSGLFFASTAANGKKVIAVASVDNENIPSVLSVASYKIDSGAAQDFGYVSSSKAWDGVSKPLYAVSFDTTIPDDGCSPLPDSTPDLSDYIVLVRRGTCTFVQKAQNVAAKGAKYLLYYNNIPGALAVDVSAVPEIEAVGMVDDKTGATWIAALKDGKTVTLTLTDPIESEKQIQFSDNPTTGGALSGYTTWGPTWELDVKPQISSPGGNILSTYPVALGGYATLSGTSMACPLTAAAVALIGQARGTFDPALIDNLLATTANPQLFNDGEKFYDFLAPVPQQGGGLIQAYDAAFATTLLSPSSLSFNDTDHFIKKKQITLKNTSKQRVTYKLNHVPTNTFYTLAPGNGYPAPFPNDAVAAHANLKFNLQQVTLPAGRSITVDVFPTPPRDVDAKRLALWSGYITVNGTDGTSLSVPYQGLTGSLHKQKVLYPEDSWIADSTDESLAPVENGTVFTIPAPGNAGPDDKLPSLVVSPALGSRYVRVDLVLLSAPPHGTKLKTVKFLDTTSIGQPAGSPLLWISRGANPIAWTGELSDNKFAPPGTYKAVFHALRIFGNEKKKEDWDVSESPAFTIKYA

SEQ ID NO: 65 - protein, 897 aa, Myceliophthora thermophila
MVRLDWAAVLLAATAVAKAVTPHTPSFVPGAYIVEYEEDQDSHAFVNKLGGKASLRKDLRFKLFKGASIQFKDTETADQMVAKVAEMPKVKAVYPVRRYPVPNHVVHSTGNVADEVLVKRQAAGNDTFSTHLMTQVNKFRDAGITGKGIKIAVIDTGIDYLHEALGGCFGPDCLVSYGTDLVGDDFNGSNTPKPDPDPIDNCQGHGTHVAGIIAAQTNNPFGIIGAATDVTLGAYRVFGCNGDTPNDVLIAAYNMAYEAGSDIITASIGGPSGWSEDPWAAVVTRIVENGVPCVVSAGNDGDAGIFYASTAANGKKVTAIASVDNIVTPALLSNASYTLNGTDDFFGFTAGDPGSWDDVNLPLWAVSFDTTDPANGCNPYPDSTPDLSGYIVLIRRGTCTFVEKASYAAAKGAKYVMFYNNVQQGTVTVSAAEAKGIEGVAMVTAQQGEAWVRALEAGSEVVLHMKDPLKAGKFLTTTPNTATGGFMSDYTSWGPTWEVEVKPQFGTPGGSILSTYPRALGSYAVLSGTSMACPLAAAIYALLINTRGTKDPKTLENLISSTARPNLFRLNGESLPLLAPVPQQGGGIVQAWDAAQATTLLSVSSLSFNDTDHFKPVQTFTITNTGKKAVTYSLSNVGAATAYTFADAKSIEPAPFPNELTADFASLTFVPKRLTIPAGKRQTVTVIAKPSEGVDAKRLPVYSGYIAINGSDSSALSLPYLGVVGSLHSAVVLDSNGARISLASDDTNKPLPANTSFVLPPAGFPNDTSYANSTDLPKLVVDLAMGSALLRADVVPLSGGAATATARLTRTVFGTRTIGQPYGLPARYNPRGTFEYAWDGRLDDGSYAPAGRYRFAVKALRIFGDAKRAREYDAAETVEFNIEYLPGPSAKFRRRLF

SEQ ID NO: 66 - protein, 882 aa, Myceliophthora heterothallica
MVRLDWAAVLLAATAVAKAVTPHTPSFVPGAYIVEYEEDQDSHAFVNKLGGKASLRKDLRFKLFKGASIQFKDTETADQMVAKVAEMPKVKAVYPVRRYPVPNHVVHSTGNVADEVLVKRQAAGNDTFSTHLMTQVNKFRDAGITGKGIKIAVIDTGIDYLHEALGGCFGPDCLVSYGTDLVGDDFNGSNTPKPDPDPIDNCQGHGTHVAGIIAAQTNNPFGIIGAATDVTLGAYRVFGCKGDTPNDVLIAAYNMAYEAGSDIITASIGGPSGWSEDPWAAVVTRIVESGVPCVVSAGNDGDAGIFYASTAANGKKVTAIASVDNIVTPALLSNASYTLNGTDDFFGFTAGDPGSWDNVNLPLWAVSFDTTDPANGCNPYPDSTPDLSGYIVLIRRGTCTFVEKASYAAAKGAKYVVFYNNVQQGTVTVSAAEAEGIEGVAMVTAQQGEAWVRALEAGSEVVLHMKDPLKAGKFLTTTPNTATGGFMSDYTSWGPTWEVEVKPQFGTPGGSILSTYPRALGSYAVLSGTSMACPLAAAIYALLINTRGTKDPETLENLISSTARPNLFRLNGESLPLLAPVPQQGGGIVQAWDAAQATTLLGVSSLSFNDTDHFKPVQTFSITNTGKKAVTYSLSNVGAATAYTFEDAKSIEPAPFPNELTADFASLTFVPQRLTIPAGKRRTVTVIAKPPKGVDAKRLPVYSGYVAINGSDGSALSLPYLGVAGSLHSAVVLGSNGARISLASDDTNTPLPANTSFVLPPAGLANDTSYANNTDLPKLVVDLAMGSALLRADIVPLSGGARAIGQPYGLPARYNPRGAFEYAWDGRLDDGSYAPAGRYRFAVRALRIFGDARRAREYDAAETVEFNIDYLPGPSAKFRRLF

SEQ ID NO: 67 - protein, 395 aa, Trichoderma reesei
MKFLQTSLVAALPAALVSGRFVAENEGDHVILEEPAKYLIELGPGETQWVTEEDKWDLRRNGQRFMDITDTRDLGSLRAQFRAATVQFPDKCVKQKEVGQLAGNLSKDEMLKNLEKLTSFHTRYYKSDYGRQSSEWVLDKVNQIIKDAGASDTVTASPFAHSWPQSSVIARIPGKSNTTVVIGAHQDSINLWLPAILAAPGADDDGSGTVTILEVFRTLLGDKDVVGGSARNTVEFHWYSAEEAGLLGSQAIFQAYEKAGRDVKAMLQQDMTGYAQGTIDAGKPVSVGVITDYVDPGLTAFIKKVIEEYCSIPWVETKCGYACSDHASASKAGYPSAFVIESAFENSDQHIHGTEDLIKYLSFDHMLEHAKMTLGLVYELAYHDFSSARDEPSEL

SEQ ID NO: 68 - protein, 405 aa, Myceliophthora thermophila
MKISGASLLALLLPAVSGRFVEPGEPDRVLLYPDGVPQPSESSEKYHIELAPGETRWVTEEEKWELRRQGRRFFDITDHPGLGTLRARDTFKKRSVFPKAPKLQDEVNPLLANLSKTEMKDHLTTFTSFHTRYYKSDYGRQSSEWLLGQVRQTIKEAGAEDYVHAEHFKHPWGQHSIIATIPGKTNSTVVIGAHQDSINLWLPSILAAPGADDDGSGTVTILEAFRVLLQSEDVVKGNHQNTLEFHWYSAEEGGLLGSQAIFSAYEKEGRDVKAMLQQDMTGFVARTLDAGKPESVGVIVDYVDPDLTQFIKVIIDEYCDIPYVETECGYACSDHASASKAGYPSAFVIESAFEYSDNHIHSTEDLIKYLSFDHMLQHARMTLAFAYELAFTDFDSLEGPDHSDA

SEQ ID NO: 69 - protein, 406 aa, Myceliophthora heterothallica
MKISGASLLALLLPAVSGRFVEPGEPDRVLLYPDGVPQPSESSEKYHIELAPGETKWVTEEEKWELRRQGRRFFDITDHPGLGTLRARNTFKKRSVFPKAPKLQDEVNPLLANLSKTEMKDHLTTFTSFHTRYYKSDYGRQSSEWLLGQVRQTIKEAGAGDYVRAEHFKHPWGQHSIIATIPGKTNSTVVIGAHQDSINLWLPSILAAPGADDDGSGTVTILEAFRVLLQSEDVVKGNHQNTLEFHWYSAEEGGLLGSQAIFSAYEKEGREVKAMLQQDMTGFVARTLDAGKPESVGVIVDYVDPGLTQFIKVIIDEYCDIPYVETECGYACSDHASASKAGYPSAFVIESAFEYSDNHIHSTEDLIKYLSFDHMLQHARMTLAFGYELAFTDFDSLEGPDHSDAL

SEQ ID NO: 70 - protein, 513 aa, Trichoderma reesei
MAVLSRLALTASFALCGVSAAGIQQPLTAPESLPPSHEAVADYGSKPIIDSEALQSAISIDTLVKRAESFYKFAKASEEEYGHPTRVIGSAGHEQTLNYITNTLLDHGDYYNVSVQEFPVTLSNVFQFRLVLADEVSKSAIPMGLTPPTKDKEPVHGDLVLVQNSGCDASDYPQNVKGNIAFIRRGACSFGDKSIGAGKAGAKAAVIYNTDPEELHGTLGLPVEDHIATFGIDGVEGKKILAKLSNGESVDAIAYIDAEVKQIQTVNVLAQTEEGDPDNCVMLGGHSDGVAEGPGINDDGSGSISVLEVAVQLTKFKVNNCVRFAWWAAEEEGLLGSDFYAASLSDEENQKIRLFMDYDMMASPNFAYQIYNATNAESPAGSEELRNLYVDWYKSQGLNYTFIPFDGRSDYDGFIRAGIPAGGIATGAEAVKTKEEAEMFGGRAGEWLDPCYHQLCDDLGNLNHTAWEVNTKLIAHSVATYALSFDGFPKRKLETEMSAYSQTTKHHGPKLIL

SEQ ID NO: 71 - protein, 511 aa, Myceliophthora thermophila
MRTPAASLLAFALPALATAGGHGGSSGLGCDSQRPLVSSEKLQSLIKKEDLLAGSQELQDIATAHGGHRAFGSSGHNATVDFLYYTLKALDYYNVTKQPFKEIFSSGTGSLTVDGEDIEAETLTYTPSGSATDKPVVVVANVGCDAADYPAEVAGNIALIKRGTCTFSQKSVNAKAAGAVAAIIYNNAEGKLSGTLGQPFLDYAPVLGITLEAGEALLAKLAGGPVTATLQIDALVEERVTYNVIAETKEGDHSNVLVLGGHTDSVPAGPGINDDGSGTIGMLTVAKALTKFRVKNAVRFAFWSAEEYGLLGSYAYIKSINSSAAELSKIRAYLNFDMIASPNYIYGIYDGDGNAFNLTGPAGSDVIERNFENFFKRKHTPSVPTEFSGRSDYAAFIENGIPSGGLFTGAEVLKTEREAELFGGRAGVAYDVNYHQAGDTVDNLALDAFLLNTKAIADSVATYALSFDGLPRVDGKKRRWDAHRARMLKRSAGSHGHAHLHSGPCGGGASI

SEQ ID NO: 72 - protein, 516 aa, Myceliophthora heterothallica
MRFNHASGIAGVATLGVVSALGGQKPLTDTSLSQVPLDGDGPNPKSLVTSEALQATISIDALFSRAKQLFKIAHLSEDEYGHPTRVIGSKGHQGTLEYIYNSLAALGDYYVLSTQPLPAVTGTVFESRLLIDLTVAESASPMSLTPPTKEKRPVDGKLVLVANEGCDAADYPSDLKGSIAFVKRGTCPFGTKSELAGRAGAVAAVVFNNENGPLSGTLGTPSDDHVATFGLSGSEAAPFVRRLEAGETVEASAYIDSEVKSIETTNIIAQTRDGDPDNCVMLGGHSDSVTEGPGINDDGSGSLTLLEVATQLTNFTVSNCVRFAWWSGEEEGLLGSDYYVSVLSPEENRKIRLFMDYDMLASPNFAYQVYDARNHVNPVGSQELRDLYVDWYIAHGLNYTFIPFDGRSDYDGFIKNGIPGGGIATGAEGIKTEEEAAFFGGEAGVPYDVCYHAICDTISNLNLTAWELNSKLVAHSVATYARSFEGFPERTHDEFSVEVSNAYSENVKYHGSKLFI

SEQ ID NO: 73 - protein, 605 aa, Trichoderma reesei
MAATRDPTTLSNYGAWRTKHTSASFNIDFDNKRLVGSVVLQLESQTDKQSTEIVLDSRFVKVSSVAVDGAAAQWELKPHSDPFGAPLHIQVPHGAAKGDVVRVAIELETTSKCTALQWLTPAQTSNKKHPYMFSQCQAINARSIFPCQDTPDVKSTISFKFTSTLPVVASGVAVGDHTATPGVEKVYEFEQKVPIPSYLFAVASGDIVAAPIGSRSKVVTGPNELKGCQWELERDMEKFLEIAESIVFPYRWGEYNVLVLPPSFPYGGMENPIYTFATPTIISGDRQNVDVIAHELAHSWSGNLVSNASWEHFWLNEGWTVYLERRIEAAVHGEPQFDFSSIIGWKALEDAVAHFGKDHEYTKLIISHDNVDPEDVYSTVAYEKGFHFLYYLDRLVGRDNFNKFIPHYFTKWAGKSLDSFEFRDTFVDFFNNLGDEEVKQKVATIDWEGRFYTPGLPPKPEFDTTLASQCYELAEKWKDASYEPSPKDVEGFSSNQKIVFLEKLQQFEPLSTDRAQLLGKVYDLLNTQNVELKFAYLQLALKANDASSYQTTADLLGKVGRMKFVRPLFRSLNKVDRNLALETFAKNKDFYHPICRGMVEKDLGV

SEQ ID NO: 74 - protein, 613 aa, Myceliophthora thermophila
MAPVRDPNTLSNYDQWRTRHTTANFKVDFAAKCLRGSVILELESQTDKASREIILDSSYVDVSAIKLNSTPSQWVIKDRTGPNGSPVHVAVPNGAAKGEVVKLEIELATTEKCTALQWLTPAQTSNKKAPFMFSQCQAIHARSIFPCQDTPDVKSTYEFNIRSPHVVVASGVPVPGATVDAGEDKIYKFEQKVPIPSYLFAVASGDIASAPIGRCSSVATGPNELKASQWELQDDMDKFLDAAEKIVFPYQWGEYNVLVLPPSFPYGGMENPIFTFATPTIISGDRQNIDVIAHELAHSWSGNLVTSCSWEHFWLNEGWTVYLERRILAAIHKNDAYFDFSAIIGWKHLEEAIEGFGKDHEYTKLSIKHDGIDPDDAFSTVPYEKGFHFIWSLDRLVGRENFDKFIPYYFKKWQNKSLDSYEFKDTFLEFFGAPEYAGLKDKLAEIDWEGRFFNTGLPPKPEFNTSLVDVCFQLAEKWKQKDYTPSPSDISSWTGNQVLVFLNAVQDFEEPLTVGQSQNLGKIYGLADSKNAELKSAYYQIAMKAKDTSSYPGVAELLGNVGRMKFVRPLFRSLNKVDRDLALGTFEKNRDFYHPICRQLVEKDLGVGEAKSS

SEQ ID NO: 75 - protein, 659 aa, Myceliophthora heterothallica
MWGFLRLGRARLAQETANSTIAQNLKTTAAAASFCQRRWFGRSFARMAPVRDPNTLSNYDQWRTRHTTANFKVDFAAKCLRGSVILELESQTDKASREIILDSSYVDVSAIKLNSTPSQWVIKDRTGPNGSPVHVAVPNGAGKGEVVKLEIELATTEKCTALQWLTPAQTSNKKAPFMFSQCQAIHARSIFPCQDTPDVKSTYEFNIRSPHVVVASGVPVPGATVDAGEDKIYKFEQKVPIPSYLFAVASGDIASAPIGRCSSVATGPNELKASQWELQDDMDKFLDAAEKIVFPYQWGEYNVLVLPPSFPYGGMENPIFTFATPTIISGDRQNIDVIAHELAHSWSGNLVTSCSWEHFWLNEGWTVYLERRILAAIHKNDAYFDFSAIIGWKHLEEAIEGFGKDHEYTKLSIKHDGIDPDDAFSTVPYEKGFHFIWSLDRLVGRENFDKFIPYYFKKWQNKSLDSYEFKDTFLEFFGAPEYAGLKDKLAEIDWEGRFFNTGLPPKPEFNTSLVDVCFQLAEKWKQKDYTPSPSDISSWTGNQVLVFLNAVQDFEEPLTVEQSQNLGKIYGLADSKNAELKSAYYQIAMKAKDTSSYPGVAELLGNVGRMKFVRPLFRSLNKVDRELALKTFEKNRDFYHPICRQLVEKDLGVGEAKSS

SEQ ID NO: 76 - protein, 419 aa, Trichoderma reesei
MKCLALLSAFLASASAAAVQPHRVSYNGYKVFRVSVEESNIGRINDVVDRLNLQTWKPAKKAGSFADIVVHPDQLEAFQQETQDLKPVVMHEDLGLSIEAESSSQNFAINAVNSTWFNSYHAYADHLQWLRDLSAQFPNTSEIVTSGNSLNGNPITGIHFWGSSGKGKKPAVILHGTVHAREWITTLVVEYFAYTLLTSTDATTKGLLDKYDFYFFPVVNVDGFLYTQSTDRLWRKNRQTNSGSSCIGRDINRNWNYMWNVPGGASTNPCAEDYKGAAAGDSVEFKALSAFIQKIKTAQGVKLYIDYHSYSQLFMTPYGYSCTAVAPNNSELQSLAKGAVAAIRAVHGTSFDYGPICSTIYQATGSSVDYVSDVIKADYTFTSELRDTGRYGFVLPADQILPSGEEAYAGFKYLLQNMK

SEQ ID NO: 77 - protein, 421 aa, Myceliophthora thermophila
MKLNLLVSSALLALASAAAVQSPQQRKSYDGYKVVRLTVGKDAAKVNSIIDRLGLSTWKGKVRAGAVADIVIPPSQVDAFAAETAGMEAITMHEDLGASIAAESTFQAYAAGSANATWFQSYHSFNDHLQFLNDLQQQFSTQAEIVSSGKSLNGRDITGIHFWGKSGKGKPAVVFHGTVHAREWISTMVVEYLAYYLLNNQDSAEVAGFLDKYDFFFFPFVNPDGFVYTQTNDRLWRKNRQSTSGSSCIGHDINRNWPYKWDVSGGASTNPCAEDFKGTKEADAPETQALSAWLKQTKESQGIKLFIDWHSYSQLFMTPYGYSCTAVAPNNSELQSLAQGAAAAIKAVHGTSFKTGPICSTIYKATGSSVDYANDVVGADYVFTAELRDTGNYGFVLPASQILPSGEETYAGVRYLLQNMK

SEQ ID NO: 78 - protein, 421 aa, Myceliophthora heterothallica
MKLNLLVSSALLALASAAAVQSPQQRKSYDGYKVVRLTVGKDAAKVNSIIDRLGLSTWKGKVRAGAVADIVIPPSQVDEFAAETAGMEAITMHEDLGASITAESTFQVYAAGSANATWFQSYHSFDDHVQFLNDLQQQFSTQAEIVSSGKSLNGRDITGIHFWGKSGKGKPAVVFHGTVHAREWISTMVVEYLAYYLLNNQDSAEVAGFLDKYDFFFFPFVNPDGFVYTQTNDRLWRKNRQSTSGSSCIGHDINRNWPYKWDISGGASTNPCAEDFKGTKEADAPETQALSAWLKKTKESQGIKLFIDWHSYSQLFMTPYGYSCTAVAPNNSELQSLAQGAAAAIKAVHGTSFKTGPICSTIYKATGSSVDYANDVVGADYVFTAELRDTGNYGFVLPASQILPSGEETYAGVRYLLQNMK

SEQ ID NO: 79 - protein, 442 aa, Trichoderma reesei
MKTFIPVALLALSQATSACLLPHERDEASGLKPVVRRQSSNGTPIGTGDRFSGGSTAPRGLGTQSSSTSFSTILNVKEVTSGLQGLANTYGVQTFNTPYKTAQGATVVGAKVGGTGGNCTDAYRVFFNGNIHARERGSADSVLYFISDLLYANAHNTGLTYGSKTYSNAQVKTALAAGIVFIPLSNPDGVAYDQSTNSCWRKNRNPNSGQSPGVDLNRNFDFLWDFRNLFASSAQSSVGSTSPSSETYHGASAFSEPETKNIKWVFDTYSKVRWFVDLHSYAGDVLWNWGSDENQVDYPTMNFLNGTYNKVRGILTDTPSPGRGYGEYVPQADLDVKEAAAKRVASALTAGGGRSYTAFQSAQLYPTSGASDDYAYSRHFSDPTKNLIHSYTIEFGFANNAASCPFYPSVSQYNSNLRATSAGFMELLLAATDYGLGDATTC

SEQ ID NO: 80 - protein, 548 aa, Trichoderma reesei
MRLSTSALVLGAASSAMAFQDQKVLSDPSSKPAVKFGSDFESLASSLKESFGELTADAKAVWDEVAMLAPDAMAAFKEQVKGIKPKKHDRKPDGDWDHVVKGADVQKLWVEDESGEPRRLVGGKLSNYSLRAKKVNPEKLGVDKVKQFSGYLDDDEKDKHLFYWFFESRNDPKNDPVILWLNGGPGCSSLTGLFFELGPASINKKIEVVHNPHAWNNNASVIFLDQPVNVGYSYGSGSVSDTVAAGKDVYALMTLFFHQFPEYSHQDFHIAGESYGGHYVPTFASEILSHKDRNINLKSIAVGNGLTDEFTQYAYYRPMACGEGGYPAVLSESQCNAMDNALPRCQSLIKNCYDSESAWLCVPAAIYCNNAFIGPYQQTGYNPYDIRSKCEDSGNLCYEGLGYISEYLNKPEVMEALGAEVSSYESCNFQINRDFLMRGDWMKPIYRLVPDLLKQIPVLIYAGDADFICNWLGNKAWVTQLEWEHGDSFRSAEAKDLTVGDKTYGNVLSSHNLTWIQVYGAGHMTPTDEPEGSINFINRWIAGEWVAK

SEQ ID NO: 81 - protein, 554 aa, Myceliophthora thermophila
MRIAASTVLLGAASAASFQQQAQHVLSDGFGKAQEAMKPLSDALADAAGRPIENFEEAFSGMTAEAKALWEEIKLLVPDSAFKNPSWFSKPKPHRRRDDWDHVVKGADVQKIWVQDANGESHRQVGGRIEDYNLRVKTVDPSKLGVDSVKQFSGYLDDEANDKHLFYWFFESRNDPKNDPVVLWLNGGPGCSSLTGLFLELGPSSIDKNLKVVNNEFSWNNNASVIFLDQPVNVGYSYSGSSVSNTIAAGKDVYALLTLFFHQFPEYAKQDFHIAGESYAGHYIPVFASEILSHKNRNINLKSILIGNGLTDGLTQYEYYRPMACGEGGYPAVLSESECRSMDNALPRCQSLIRNCYDSGSVWSCVPASIYCNNALIGPYQRTGQNVYDIRGKCEDSSNLCYSALGYISDYLNQQSVMDALGVEVSSYESCNFDINRNFLFQGDWMQPFHRLVPNILKEIPVLIYAGDADYICNWLGNRAWTEKLEWPGQKAFNQAKVHDLKLAGADEEYGKVKASGNFTFMQIYQAGHMVPMDQPENSLDFLNRWLSGEWFAK

SEQ ID NO: 82 - protein, 554 aa, Myceliophthora heterothallica
MRIAASTVLLGAASAASFQQQAQHVLSDGFGKAQEAMKPLSDALADAAGRPIENFEEAFSGMTAEAKALWEEIKLLVPDSAFKNPSWFSKPKPHRRRDDWDHVVKGADVQNIWVQDANGESHRQVGGRIEDYNLRVKTVDPSKLGVDSVKQFSGYLDDEANDKHLFYWFFESRNDPKNDPVVLWLNGGPGCSSLTGLFLELGPSSIDKNLKVVNNEFSWNNNASVIFLDQPVNVGYSYSGSSVSNTIAAGKDVYALLTLFFHQFPEYAKQDFHIAGESYAGHYIPVFASEILSHKNRNINLKSILIGNGLTDGLTQYEYYRPMACGEGGYPAVLSESECRSMDNALPRCQSLIRNCYDSGSVWSCVPASIYCNNALIGPYQRTGQNVYDIRGKCEDSSNLCYSALGYISDYLNQQSVMDALGVEVSSYESCNFDINRNFLFQGDWMQPFHRLVPNILKEIPVLIYAGDADYICNWLGNRAWTEKLEWPGQKAFNQAKVHDLKLAGADEEYGKVKASGNFTFMQIYQAGHMVPMDQPENSLDFLNRWLSGEWFAK

SEQ ID NO: 83 - protein, 707 aa, Trichoderma reesei
MMKPEELKHYLADQPPSVVRLEIEKHFALLTEKQKRYAHFISKAAFAGSRIVLRQLSPESEPIFDLIITLHKSANGDWNALAKKAGVDEAELNLFLEYVAMFLGNMGNYKSFGDSKFIPRCSETTVAALAATSPEAQKFYDATKGAIFSHDDPAAMHLGFLDAGHMTTYYPDSKDITKSEIEAVSTWMEEKRLLPENTRLRKTADGFDILIASAVTSVPPEGGDIGKETEFTVENGPLKGKTIRLVYGDHAEEMKNITAYIKQAAANADNENQKNMHLAYAKSFETGSLEAFKESQRWWIKDKGPSVECNIGFIETYRDPAGVRGEWEGFASMVNQERTRAFGELVNASPSLIPLLPWGKEFEKDKFLSPDFTSLEVLTFANSGIPAGINIPNYDDIRQTEGFKNVSLGNVLSAKAPNEKIPFIREEDLAVYQANRDAAFEVQVGLHELTGHGCGKLLQETSPGVYNFDKENPPVSPVTQKPISTWYKPGQTWGSVFGPIAASYEECRAELVAMYLSCEFPVLKIFGFGDGSEDINGPAGDVLYASYLSMARAGLASLEMWDPKSQKWGQAHSQARFSILKSFLEAGDDFCKLDYTKDDLSDLTIKLDRSKILTAGRDAVAKYLQKLHVYKSTADVETGTKFYTDMSTVGLDFWGTKVRQVVLDNKQPRKVFVQANTTLDEATDSVSIKHYDATLLGMIQSWADRNL

SEQ ID NO: 84 - protein, 704 aa, Myceliophthora thermophila
MDAKELSQYLADAPPSVVRLEIEKHFNALSDKQKRYAHFISKASFAGNRIVLRQVSPESEDIYDFILALYRTAGGDWKALADKAGVDEQGLTAFLQYAAQLLGNSGNYKSFGDSKFLPRCDESVFAALASTSPEAEKHYKATNGAIFASDKQGLLHLGFTDEGHLTTYYPDSPSITKDEVVAVSKWMEDKGLLPENTRLRKTEKGVYELLIASATTSIPSQGGDIGKDTEYVISEGPLAGKTVRLVYGDYAREMNTIAGFIRKAAENAENETQKKMHLAYAESFETGSLLAFKDSQRHWIRDQGPMVECNIGFIETYRDPAGVRGEWEGFAAVERTRAFGELVRSAPELIPLLPWSKDFEKDKFLSPDFTSLEVLTFCGSGIPAGVNIPNYDDIRQQEGFKNVSLGNVLSAKAPDEKIPFIAESDLELYQKYRDAAFEVQVGLHELTGHGCGKLLQETSPGVYNFDVKNPPISPLTGKPITTWYKPGQTWGSVFGGVAGAYEECRAELVAMHLSCEFAALKIFGFGDGTVDMDGPAGDVLYTSYLSMARAGLTSIEFWDPKSQKWGQPHCQARFAILKAFLEAGDDFCKLEYKNEDLSDLTIRLDRSKILTSGRKAVADFLQKIHIYKSTADVEAGTKFFNEMSSVGLEYWGTKVRDVVLKNKQPRKVFVQANTFLDEATGKVTLKQYEPSPEGLIQSWAERDV

SEQ ID NO: 85 - protein, 707 aa, Myceliophthora heterothallica
MDAKELSQYLADAPPSVVRLEIEKHFNALSDKQKRYAHFISKASFAGNRIVLRQVSPESENIYDFILALYRTAGGDWKALADKAGVDEQGLIAFLQYAAQFLGNSGNYKSFGDSKFLPRCDESVFAALASTSPEAEKHYKATNGAIFASDKQGLLHLGFTDEGHLTTYYPDSPSITKDEVVAVSKWMEDKGLLPENTRLRKTEEGVYELLIASATTSIPSQGGDIGKDTEYVISEGPLAGKTVRLVYGDYAREMNTIAGFIRKAAENAENETQKKMHLAYAESFETGSLLAFKDSQRHWIRDQGPMVECNIGFIETYRDPAGVRGEWEGFAAVVNLERTRAFGELVRSAPELIPLLPWSKDFEKDKFLSPDFTSLEVLTFCGSGIPAGINIPNYDDIRQQEGFKNVSLGNVLSAKAPDEKIPFIAESDLELYQKYRDAAFEVQVGLHELTGHGCGKLLQETSPGVYNFDVKNPPISPLTGKPITTWYKPGQTWGSVFGGVAGAYEECRAELVAMHLSCEFAALKIFGFGDGTVDMDGPAGDVLYTSYLSMARAGLTSIEFWDPKSQKWGQPHCQARFAILKAFLEAGDDFCKLEYKNEDLSDLTIRLDRSKILTSGRKAVADFLQKIHIYKSTADVEAGTKFFNEMSSVGLEYWGTKVRDVVLKNKQPRKVFVQANTFLDEATGKVTLKQYEPSPEGLIQSWAERDV

SEQ ID NO: 86 - protein, 463 aa, Trichoderma reesei
MVAQDFEAVLKGKYPGKSHASRVVDLIRKKDPNANGVLYLEGRMTKLLEDNDSPEPFRQRRYFYYLTGCNLADCYFTYDIQAAKSTLFIPPIDPHDVIWSGLPTTIDDALRLYDVDEVKLTTEVNATLAHLANATSSSTIYAINGQVSDHITFIGFENKDFALLKEAIEVSRVVKDEFEVAMIRKANHVSSLAHKAVVEKAKQASNEMELEAVFLERCVAKGAKNMAYHPIVASGTAAATLHYVANDAPLAGKQNLLIDAGAEWNNYAADITRTFPLNGKFTKEGREIYDIVYKMQQECIAVIKAGMYWEDAHILAHKIAIDGLLALGILKGDKDEIFKARTSQAFFPHGLGHYLGMDTHDTGGNPNRQDPDVIFQNLRLRGQVPAGGVVTVEPGVYFCKFIIDPYLENPEHSKYIDADVLAKYWDVGGVRIEDNVLVTETGYENLTTAIKEPGELEALMAAS

SEQ ID NO: 87 - protein, 460 aa, Myceliophthora thermophila
MDYDRILQGKYPAKRHAKRVVDYIRTKVPDATGVIYLESRMTKLLEDNDEPEPFRQRRFFYYLTGCPLADSFLVHDMDSAKTTLFIPPIDPDSVIWSGLPVSAQEALEKWDVDDVKYTNEINAELAHLAKQKSKATVFAIPGQVSDQVTFLGFGETNFDVLKQAIEVCRVVKDEFELALMAKANAVSADAHHAVMKQVRHAKNERELEATFLASSISAGAREQAYHGIFASGRAAATLHYVHNDKPLAGKLNLLLDAGAEWNCYASDITRTFPINGKFSKESREIYDIVLKMQLECIAALKEDVLWEDVHVLAHKIAIDGLLKLGILKGDKDEILANRTSVAFFPHGLGHYLGMDTHDTGGNPNYADKDPLFRYLRIRGKVPAGSVVTVEPGIYFCNFIIDPFLKDPKHSKYIDADVLEKYWDVGGVRIEDNLVITKTGAENLTTTLKDPDEVERLISSS

SEQ ID NO: 88 - protein, 460 aa, Myceliophthora heterothallica
MDYDRILQGKYPAKRHAKRVVDYIRTKVPDATGVIYLESRMTKLLEDNDEPEPFRQRRFFYYLTGCPLADSFLVHDMDSAKTTLFIPPIDPDSVIWSGLPVSAQEALEKWDVDDVKYTNEINAELAHIAEQKAKATVFAIPGQVSDQVTFLGFGETNFDVLKQAIEVCRVVKDEFELALMAKANAVSADAHHAVMKQVRHAKNERELEATFLASSISAGAREQAYHGIFASGRAAATLHYVHNDKPLAGKLNLLLDAGAEWNCYASDITRTFPINGKFSKESREIYDIVLKMQLECIAALKEDVLWEDVHVLAHKIAIDGLLKLGILKGDKDEILANRTSVAFFPHGLGHYLGMDTHDTGGNPNYADKDTLFRYLRIRGKVPAGSVVTVEPGIYFCNFIIDPFLKDPKHSKYIDADVLEKYWDVGGVRIEDNLVITKAGAVNLTTTLKDPDEIERLISSS

SEQ ID NO: 89 - protein, 460 aa, Trichoderma reesei
MTAKSHAILPTAGSPDYRNPSAANSHAQQPPHRPNLKKLALGASLALATIGLLYKPAFSHYQRISQHSCSKTLTVEERAHKILSTTPLIDGHVDFPLVLRGPYGNHLTGDDFTGPFENGTLKGHVDLARLRAGRAGGAFWSVYAPCPANGTDFSDDNYADSLQFTLQEIDIMKRLFAAYPDDFAHDIDSSDAIDAFRAGKLISPLGIEGLHQIANLAGNLRMFRDLGVRYATLTHNCHNKFADAALLESPFRKATPVWGGISPLGRQLVHEMNRIGMIVDLAHVSEDTMIDALGGREDWEGSKAPVIFSHSSAYGICPHPRNVKDKVLQLVKETDSLVMVNIAPQFISCIDNGNDNGVPDEDPDNATLERVVDHITYIGSRIGYDHVGIGTDFDGIGSVPKGLEDVSKYPDLIAELLRRGVSDADAAKVVGDNLIRVWKAVDAISAEMKANGAPVMEDKL

SEQ ID NO: 90 - protein, 509 aa, Myceliophthora thermophila
MTSVTELPGPLARMGQKADHSSGQELRARRRSWVARAALYVVLPLFLFAWFVRSAGYSTCFGHGLPFSSTKHRSIEARVKRILRHNPLIDGHNDLAIWIRAYYNNHIYNETFSKPFAEGGLGGHIDIPRLRAGLNGGAFWSAFWPCPANGSDYTDENYHRTVQQTLNQIDLLTRLQQAHSDLFLPPTLTSRQALHRFRHSHGRRLISPLAIEGLHQIGNSAGVLRQLHALGVRYATLTHNCGNRFADAALWEHPAPRKAPPVWGGVSPAGRALVREMNRLGVIVDLSHTSADTMRDVLGGGRDGWEGSLAPPIFSHSSAYALCPHPRNVPDDVLDLVGRRNGVVMVNFNPGFISCVEAPHRDDGLPDFYPQNSTLQQVVRHILHIGRRIGFDHVGLGSDFDGITEVPEGLEDVSKFPALVAELLRQGVSDKDAANIVGGNVLRVWRDVEAVAEKLQRAGEPPLEDDLPRRIAAFDGVDGENFEDATAFAAQQREGEGGRGKKSTESSFF

SEQ ID NO: 91 - protein, 501 aa, Myceliophthora heterothallica
MGQKADHSSGQELRARRRPWAARAARAALYVVLPLVLFAWFVRSAGYYFTCFGHGLPFSSTKHRNSIEARVKRILRHNPLIDGHNDLAIWIRAYYNNHIYNETFSKPFAEGGLGGHVDIPRLRAGLNGGAFWSAFWPCPANGSDYTDENYHRTVQQTLNQIDLLTRLQQAHSDLLLPPTLTSRQALHRFRHSRGRRLISPLAIEGLHQIGNSAGVLRQLHALGVRYATLTHNCGNRFADAALWEHPAPRKAPPVWGGVSPAGRALVREMNRLGVIVDLSHTSADTMRDVLGAGRDGWEGSLAPPIFSHSSAYALCPHPRNVPDDVLDLVGRRNGVVMVNFNPAFISCVEAPHRDDGLPDFYPPNSTLQQVVRHILHIGRRIGFDHVGLGSDFDGIAEVPEGLEDVSRFPALVAELLRQGVSDKDAANIVGGNVLRVWTDVEAVAEKLQRAGEPPLEDDLPRRIAAFDGVDGENFEDATAFAAQQRKGEGGRGEKSTESSFF

SEQ ID NO: 92 - protein, 620 aa, Trichoderma reesei
MGKANTTQKLAKLRALMKEHNVQVYVVPSEDSHSSEYIAACDARREFISGFTGSAGCAVITETAAALATDGRYFNQATQQLDDNWTLLKQGLQDVPTWQEWAAEQAAGGKTVAVDSTLVTASIAKKLAEKIRKSGGSDLVPLDINLVDAVWAEDRPARPQQRITVLSDKFAGKSVQSKLSDVFSELEKKRSPGLFISMLDEVAWLFNLRGSDIPYNPVFFSYAVITPKGAALYVDESKLDEECREHLNKCNVAIKPYESFFRDAELHHQQFVASAQSAEGASPSGSFLMSNKGSWALKRALGGDGAVEEIRSPVGDAKAIKNETEMEGMRACHIRDGAALIEYFAWLEDQLINKKTVLDEVQAADKLEELRSKHEHFVGLSFPTISSTGANAAVIHYGPERGNCAVIDPEAIYLCDSGAQYLDGTTDTTRTLHFGEPSEAEREAYTLVLKGNIALDVAIFPKGTTGFALDALARQHLWKTGLDYRHGTGHGVGSFLNVHEGPIGIGTRIQYTEVPLAPGNVISNEPGYYEDGRFGIRIENIVMVKEVKTKYAFGDKPFLGFEHVTMVPYCRNLINESMLSEAEKTWLNTSNAEILEKTKGFFEGDALTMAWLTRETRPIE

SEQ ID NO: 93 - protein, 617 aa, Myceliophthora thermophila
METVNTTARLARLRSLMKQKGVDVYVVPSEDSHASEYIAACDARRAFISGFTGSAGTAVVTLDKAALATDGRYFNQASKQLDSNWQLLKTGLQDVPTWQEWAAEESAGGKTVGVDPSLISSSVAEKLDESVKKSGGAGLKAVSENLVDAVWGDDRPARPNNPVVLLPEKYAGKAAATKLAELRKELDKKKAAAFVLSMLDEIAWLFNLRGSDIPYNPVFFSYAIVTGDSATLYVDESKLNDECRSYLDQNKVSVKPYGALFEDVKALANAAKAQETSEPPRKYFVSNKGSWALKLALGGDKYVEEVRSPVADAKAVKNETELEGMRQCHIRDGAALIEFFAWLEDQLVNKKAVIDEVAAADQLEAFRKKQKDFVGLSFDTISSTGPNAAIIHYKPEPGTCATIDPDAIYLCDSGAQFLDGTTDVTRTLHFGTPSDEQRKAYTLVLKGNIALDTAVFPKGTTGYAIDCLARQFLWKQGLDYRHGTGHGVGSYLNVHEGPIGIGTRKQYADAALAAGNVLSIEPGFYEDGAYGIRIENLALVREVKTEHSFGDKPFLGFEHVTMVPYCRKLIDETLLTAEEKAWLNRSNEEIREKMKGRFEGEEHALTRAWLERETQPF

SEQ ID NO: 94 - protein, 617 aa, Myceliophthora heterothallica
METVNTTARLARLRSLMKQKGVDVYVVPSEDSHASEYIAACDARRAFISGFTGSAGTAVVTLDKAALATDGRYFNQAGKQLDSNWQLLKTGLQDVPTWQEWAAEESAGGKTVGVDPSLISSSVAEKLDESVRKSGGAGLKAVSENLVDAVWGDDRPARPNNPVVLLPEKYAGKAAAAKLAELRKELDKKKAAAFVLSMLDEIAWLFNLRGSDIPYNPVFFSYAIVTGDSATLYVDESKLNDECRSYLDQNKVSVKPYGALFEDVKALADAAKAQETSEPPRKYFVSNKGSWALKLALGGDKYVEEVRSPVADAKAVKNETELEGMRQCHIRDGAALIEFFAWLEDQLVNKKAVIDEVAAADQLEAFRKKQKDFVGLSFDTISSTGPNAAIIHYKPEPGTCATIDPDAIYLCDSGAQFLDGTTDVTRTLHFGTPSDEQRKAYTLVLKGNIALDTAVFPKGTTGYAIDCLARQFLWKQGLDYRHGTGHGVGSYLNVHEGPIGIGTRKQYADAALAAGNVLSIEPGFYEDGAYGIRIENLALVREVKTEHSFGDKPFLGFEHVTMVPYCRKLIDETLLTAEEKAWLNRSNEEIREKMKGRFEGEEHALTRAWLEKETQPF

SEQ ID NO: 95 - protein, 506 aa, Trichoderma reesei
MDGRPPLDNRACVNCISKLSPSEVCQVNWHVDTDNACRLCAVRDLKPEAFTKPFCDFLRENPTIFHTVEYFKTKLRQSGYVELPARDSWADKIQPGGKYWVTRNGSSLIAFAVGKSYKPGNGVGMIAGHIDALTARLKPVSKKPTAAGYVQLGVAPYAGALNQTWWDRDLSIGGRVIVRDEKTGKTSSKLVQLDWPIAKIPTLAPHFGVGMMGQNNPETQAVPIIGLESSDGSDSELLGSTGSFVNTQPPKLVKLIAKELGITSYDSIVNWELELFDSQPATVFGLDKEFITAGRIDDKLCSWSALLGLLHSTERDSDSYIKLVALFDDEEVGSLLRQGARGNFLPLTIERLVEALNPTTYGPGLIGQTFAKSFLLSADVSHAGHPNFLGNYLPEHIPKLNVGLVVCSDSNAHMTTDAVSSAILHRVADLCGAKLQDFQIRNDSRSGGTVGPMLSSAMGVRCADAGLPQISMHSIRATTGALDPGLGVQIFKGFLDFWEQVDGEWE

SEQ ID NO: 96 - protein, 513 aa, Myceliophthora thermophila
MTRHTPEFLRARSSNLSLRSTALEQSTAMRPDIVIDNTMDAKDLRPEAFTKPYLEFMTENPTVFHAVDYFKQKLLKAGYKELSSRDSWIGKLEPGGKYFTTRNGSSIIAFAVGASYKPGNGVAMIAGHIDALTARLKPTSKKPNTHGYVQLGVAQYAGALNETWWDRDLGIGGRVIVRDPDTGKTTVKLVKLDWPIARIPTLAPHFGIGMTGHNNRETELVPIIGLDNGDLDSAAAPEQPLGAPGSFVSTQPPKLVKLIAKQLSISDPSSILNWELELFDSQPATVGGLDREFIFAGRIDDKLCSWAAFMALLHAKSDPSEGNIKLVALFDDEEIGSLLRQGARGNFLPITIERAVEALASAAKTPFGPGLLGQTYASSFLVSSDVTHAAHPNFPQTNLAGHSPRLNVGVALCVDASAHMTTDSVSMAILDRVADLAGCVNQRHMIRNDSRSGGTVGPMLSAAMGCKAADVGIPQLSMHSIRATTGSLDPGLGVKFYKGFLDHWDAVDREWRN

SEQ ID NO: 97 - protein, 513 aa, Myceliophthora heterothallica
MTRHTPEFLRARSSNLSLRSTALEQSMAMRPDIVIDNTMDAKDLRPEAFTKPYLEFMTENPTVFHAVDYFKQKLLKAGYKELSSRDSWIGKLEPGGKYFTTRNGSSIIAFAVGASYKPGNGVAMIAGHIDALTARLKPTSKKPNTHGYVQLGVAQYAGALNETWWDRDLGIGGRVIVRDPDTGKTTVKLVKLDWPIARIPTLAPHFGIGMTGHNNRETELVPIIGLDNGDLDSAAAPEQPLGAPGSFVSTQPPKLVKLIAKQLSISDPSSILNWELELFDSQPATVGGLDREFIFAGRIDDKLCSWAAFMALLHAKSDPSEGNIKLVALFDDEEIGSLLRQGARGNFLPITIERAVEALASAAKTPFGPGLLGQTYASSFLVSSDVTHAAHPNFPQTNLAGHSPRLNVGVALCVDASAHMTTDSVSMAILDRVADLSGCVNQRHMIRNDSRSGGTVGPMLSAAMGCKAADVGIPQLSMHSIRATTGSLDPGLGVKFYKGFLDHWDAVDREWRN

SEQ ID NO: 98 - protein, 885 aa, Trichoderma reesei
MCRARAGLEGGSAAAPKRELLPANVVPRHYHVTVEPDFDKLTYNGSVVIDLDVAETSSFISLNTLELEIHGAKLSSGGQVVSATPAISYDENSQVTKFDFDGSHTLEKNSKVQLAIDFTGQLNDKMAGFYRSKYKRPDGTEGILASTQMEPTDARRAFPCFDEPALKAKFTVTLVADKNLTCLSNMDVASETEVQSKITGGTRKAVTFNPSPLMSTYLVAFVVGELNYIESRDFRVPVRVYAPPGHDIEHGRFSVDLAAKTLAFYEKAFGIDFPLPKMDQVAIPDFAQGAMENWGLVTYRVVDLMLDEKASGAATKQRVAEVVQHELAHQWFGNLVTMDWWEGLWLNEGFATWASWYSCNIFYPEWRVWQTYVTDDLQSALSLDSLRSSHPIEVPVGRADEINQIFDAISYSKGSCVLRMISTYLGEEKFLEGVRKYLKKYAYGNTQTSDLWDSLAEVSGKPVHEVMTAWTKSVGYPVLTVTEKEGENAIHVKQNRFLRTGDATPEEDKVLYPVFLGLRTKDGVDETLALKEREKDFPVPSLDFFKLNANHTGIYRTLYTPSRLEKLGQAAKEGLLTTEDRAGMIADAAALSSSGYGKTSGFLNLLKGFDAETEFVVWNEIISRLGSIQAAWLFEDQAVRNGIRAFLRELVSAKAHQLGWEFSDSDGHVEQQFKATLFGSAGLSGDETIISASKEMFAKFIAGDKSAIHPNIRKSVFAIALKYGGKEEYESIIKLYHESTNSDERNTCLRSLGRAKDPELIQRTLSLLLNGEVRDQDIYMPASGLRTHPEGIEALFNWMTENWEELYKRHPPNLPMLPAMVSLLTSGFTKPEQLARVEKFFSDKNTNGYDQSLAQSKDSIRSKISWVERDGQDVADWVKTNGYLE

SEQ ID NO: 99 - protein, 874 aa, Myceliophthora thermophila
MAGGVNVQARELLPTNVIPRHYNITLEPDFKKLTFDGTVVIDLDVVEDSKSISLHTLELDIHDAKITSGGQTVSSSPTVSYNEDTQVSTFEFGNAVTKGSKAQLEIKFTGQLNDKMAGFYRSTYKNPDGSEGIMAVTQMEPTDARRSFPCFDEPSLKAEFTVTLVADKKLTCLSNMDVAYEKEVKSEQTGGIKKAVTFNKSPLMSTYLVAFVVGELNYIETNEFRVPVRVYAPPGQDIEHGRFSLNLAAKTLAFYEKVFGIEFPLPKMDQIAIPDFAQGAMENWGLVTYRVVDLLLDEKASGAATKERVAEVVQHELAHQWFGNLVTMDWWDGLWLNEGFATWASWYSCNIFYPEWKVWESYVVDNLQRALSLDSLRSSHPIEVPVKRADEINQIFDAISYSKGSCVLRMISTYLGEETFLEGVRRYLKKHAYGNTQTGDLWASLAEASGKKVEEVMQVWTKNIGFPVVTVTEKDDKTIHLKQNRFLRTGDTKPEEDQVIYPVFLGLRTKDGIDESQTLTKREDTFTVPSTDFFKLNANHTGLYRTAYSPERLKKLGDAAKEGLLSVEDRAGMIADAGALATSGYQRTSGVLSLLKGFNSEPEFVVWNEIIARVSSVQSAWIFEDQADRDALDAFLRDLASPKAHELGWQFSEKDGHILQQFKAMMFGTAGLSGDETIIKAAKDMFKKFMAGDRTAIHPNIRGSVFSMALKYGGTEEYDAVINFYRTSTNSDERNTALRCLGRAKSPELIKRTLDLLFSGEVKDQDIYMPASGLRSHPEGIEALFTWMTENWNELIKKLPPALSMLGTMVTIFTSSFTKKEQLERVEKFFEGKNTNGFDQSLAQSLDAIRSKISWIERDRADVTAWLKENGYRS

SEQ ID NO: 100 - protein, 969 aa, Myceliophthora heterothallica
MFRSSLPSRPLCRGVQQRPVARAGALLQFRARQASRSSTSRYSVLSRLPSSSATASPLNRHRPLSQPQTLRLRSLPVRYCSYRRHNMCRTHAAAEMAGGVNVQARELLPANVIPRHYNITLEPDFKKLTFDGTVVIDLDVVEDSKSISLHTLELDIHDAKITSGGQTVSSSPTVSYNEDTQVSTFEFGNAVTKGSKAQLEIKFTGQLNDKMAGFYRSTYKNPDGSEGIMAVTQMEPTDARRSFPCFDEPSLKAEFTVTLVADKKLTCLSNMDVAYEREVKSEQTGGIKKAVTFNKSPLMSTYLVAFVVGELNYIETNEFRVPVRVYAPPGQDIEHGRFSLNLAAKTLAFYEKVFGIEFPLPKMDQIAIPDFAQGAMENWGLVTYRVVDLLLDEKASGAATKERVAEVVQHELAHQWFGNLVTMDWWDGLWLNEGFATWASWYSCNIFYPEWKVWESYVVDNLQRALSLDSLRSSHPIEVPVKRADEINQIFDAISYSKGSCVLRMISTYLGEDTFLEGVRRYLKKHAYGNTQTGDLWASLAEASGKKVEEVMQVWTKNIGFPVVTVTEKDDKTIHLKQNRFLRTGDTKPEEDQVIYPVFLGLRTKDGIDESQTLTKREDTFTVPSTDFFKLNANHTGLYRTAYSPERLKKLGDAAKQGLLSVEDRAGMIADAGALATSGYQRTSGVLSLLKGFNSEPEFVVWNEIIARASSVQSAWIFEDQADRDALDAFLRDLASPKAHELGWQFSEKDGHILQQFKGMMFGTAGLSGDETIIKAAKDMFKKFMAGDRTAIHPNIRGSVFSMALKYGGTEEYDAVINFYRTSTNSDERNTALRCLGRAKSPELIKRTLDLLFSGEVKDQDIYMPASGLRSHPEGIEALFTWMTENWNELIKKLPPALSMLGTMVTIFTSSFNKKEQLERVEKFFEGKNTNGFDQSLAQSLDAIRSKISWIERDRADVTAWLKENGYRS

SEQ ID NO: 101 - protein, 259 aa, Trichoderma reesei
MAPASQVVSALMLPALALGAAIQPRGADIVGGTAASLGEFPYIVSLQNPNQGGHFCGGVLVNANTVVTAAHCSVVYPASQIRVRAGTLTWNSGGTLVGVSQIIVNPSYNDRTTDFDVAVWHLSSPIRESSTIGYATLPAQGSDPVAGSTVTTAGWGTTSENSNSIPSRLNKVSVPVVARSTCQADYRSQGLSVTNNMFCAGLTQGGKDSCSGDSGGPIVDANGVLQGVVSWGIGCAEAGFPGVYTRIGNFVNYINQNLA

SEQ ID NO: 102 - protein, 950 aa, Trichoderma reesei
MAAVGPLAMHPTVTEHDYRFPRRPDPHRSTDRSFARMDSRDDHVALANGNASAAVTSGQAASSRKNSVLDSALFPSLENAASGSSSSLEQMQSDDPLAAQIWKFFNRTKQQLPNQQRMENLTWRMMALNLRKRQQQQQQQKTHNRSLSQPSQPQNAPSGIAQQLRKTSDSHAEGVDAMNIDDFIFAEEGGASVKDNSPQPPASSLSGPQTAATKTSKSSRPLASAIPIKGRKGSSANQFVAQSVPVAPPHQRGQFDEFHYVPRHDRKTSIDDRRTRKRPANFSPHVSAINPSASGLSLDLEADPDLNGYSLDNTNAMSLQQQMANNQGSAFNLDFMDSDGVLGSAPFHPNFSFSPSTSPMMHNGPFPSVYNNAALQSSLNNPDFYSPTGSGYPSATSTPHPMNEGDGVYFGSQEMQHHPHHRAQGFRQSSGNLNHHLSHQFMYGDSNGGNGSGGGNGAAFPSTGASSEAVASYSAAQNSFGHIDPSQVFQTESQVASPVLPMQQGNMLGFGGESDEDDGTAFLDRGGVGLHGDFTSAVDEAGTFSWDASLPGQFSTQAARFPGGPTRKHVQIGTITTDYVDTSNDWDGHNLSRSHSQTLGMGNDKRQNKLPRNSSTPSHMAAKHSGFEQIAQSLPSSPGPHASGTASGFSSVAPSRPASPPPSSKHGSSTNLHAASGNPGENSAPTTCTNCFTQTTPLWRRNPEGQPLCNACGLFLKLHGVVRPLSLKTDVIKKRNRGSGANLPVGGSRKKNAGNGSNAASRKNSSLSMAAAAAATAAVATANHQAATPPSTTRPSSNKEGDSPASSGHHTAGSTPGSQYGGATIVGGKGVVPIAAAPPKTTPGPGASSSMSRPTTASSKRQRRHSKSEGTSSSAMDLDGPGDSIGRQLGATPSMPSISSTMLSNGSFTMGQRSMMGSSMVSMGGSQGNLVNSAGGSSGPQEWEWLTMSL

SEQ ID NO: 103 - DNA, 3373 bp, Trichoderma reesei
  source 1..3373: mol_type = genomic DNA
atggcagctgtcggaccgcttgcgatgcacccgaccgtcacagaacacgattaccgcttcccgcgaagacccgatcctcatcgaagtaccgaccgcagtttcgcccgcatggattcgcgagacgaccatgtcgcactcgccaacggcaatgcctccgccgccgtgacgagcgggcaagccgcctcgtcgcgcaagaacagcgtcctcgactccgccttgtttccatcgctcgaaaacgccgcctccggctcgtccagcagcctcgagcagatgcagagcgacgatccgctggcggcccagatctggaagttcttcaacaggacgaagcagcagctgccgaaccagcagcgaatggagaacctgacgtggcgcatgatggccctcaacctgcgcaagcgtcagcagcagcaacagcagcagaagacgcacaacaggtattattatagcaaatctcctttgccatccgtctcatgcttcgccatccccatctgattggtctgccgttctttgcatcgatgcccgtctaaaaggctagatacggcttggctttccctttgggggggaggggagcgggttcgcccttcatcacatcatcccgccgctggctgacatgtttttctgatctcgtctttccctgcatcacagatccctctcgcaaccaagtcaaccacaaaacgctcccagcggcatcgcccagcagctgcgaaagacatccgatagccatgctgagggcgtggatgccatgaacattgacgacttcatcttcgccgaggagggaggagcctccgtcaaggacaactcaccgcagccgcccgcatcctctctctctggtccgcagaccgctgccaccaagaccagcaaatcatccaggccgctggcttcggcaatccccatcaagggccgcaagggttcctctgccaatcaattcgtcgcccagtccgtccctgtagcgccacctcaccagcggggacagtttgatgaattccattacgtccctcgacacgatagaaagacgagtatagacgaccgccgggtgagtgcattgatttttgattggaacagaagcccctacccagcggatcaagcctcccaatgccctcccctcctccctcgcagccatcttctcctctattttatacgtttcttgacggcttgtcgtgcatggcacgccacgttccgccttcgcattctgcctgcttatccttcctccttaacttacatgcactccgtccctctgccctttaaataccctagccgtcgttcctgtgtccgaggacatctgcccagttctaatttcgctcttcccgtggactatgtgaggttgctaacaatgtctcccaagacgaggaaacggcctgccaacttctcaccccatgtgagcgccatcaacccgagtgcgtctggtctcagcctcgatctggaagcagacccggatctcaatggatactccctcgacaacacaaacgcgatgtccctgcagcagcaaatggccaacaaccagggctctgctttcaacctggacttcatggacagcgacggcgtccttggctctgcccccttccatccgaacttctccttttctccatcgacgtctccaatgatgcacaatggaccgtttccatccgtgtacaacaacgccgcgctgcaatcttcgctgaacaaccccgacttctactctcccactggctcggggtatccatcggccacatccaccccacatccgatgaacgagggcgacggcgtctatttcggctcccaggaaatgcagcaccatccgcatcatcgtgcacagggcttccgacaaagctccggtaacctcaaccaccacttgagccaccagttcatgtacggcgattccaatggcggcaacggcagcggcggcggcaatggcgctgccttcccctccacaggcgccagctcggaggcggtggcatcgtacagcgctgctcaaaactcttttgggcacatcgacccaagccaagtgttccagacggagtcgcaggtcgcctctcccgtcttaccgatgcagcaaggcaacatgcttggtttcggaggagagtctgacgaagacgacggcaccgccttcttggatcgcggtggcgttggcctccacggtgacttcacatctgccgtcgacgaggcagggacgttcagctgggacgcctctttgccgggacagttcagcacgcaggctgctcgctttcccggcggccccactcgcaagcacgtacagattgggaccatcacgactgattacgtcgacaccagtaacgactgggatggccataacctgagccgatctcactcgcaaacgctcggcatgggcaacgacaagcgacagaacaaacttcctcgcaactcgtcgacaccctcgcacatggccgccaagcacagtggattcgagcaaatcgcccagtctctccccagctcaccgggtccccatgcttcgggaaccgcctccggcttctcgtcagttgcgccaagcagaccagcctcaccgcccccaagctccaagcacggctcctcgaccaacctgcatgcggctagcggaaaccccggagagaacagcgctcccactacctgcaccaactgcttcacccagacgactcctctgtggagacggaaccccgagggccagcccctctgcaatgcctgtggcctcttcctcaagctacacggcgtcgtgcgtcccctcagtctgaagacggacgttatcaagaagagaaacaggggctccggtgccaacctcccggttggaggatcgaggaagaagaacgcaggcaatggctccaacgccgcctcgaggaagaactcatcgctgtccatggctgccgctgccgcggcgacggcagctgttgctaccgcaaaccaccaagccgcgacgccgccatctacaactcgcccaagcagcaataaggagggcgatagccccgccagcagtggccatcacaccgcaggcagcacgcctggcagccaatatggaggagccaccattgtgggcggcaaaggcgtcgtgcccattgcggcggcaccacccaagacaacgcctggacccggagcctcgtcgtccatgtcccgaccgaccacggcgtcctccaaaagacagcgtaggcatagcaagagcgagggcacttcatcgtcggctatggatctcgacggccctggagactctattgggcgacagcttggcgcaactcccagcatgccttccatctctagcactatgctttcgaatggaagcttcaccatgggtcagcgctccatgatgggatcaagcatggtgtccatgggtggttcccagggcaaccttgtgaacagcgccggtggctcgtcaggccctcaggaatgggagtggttgactatgagtctttga

SEQ ID NO: 104 - DNA, 2853 bp, Trichoderma reesei
  source 1..2853: mol_type = other DNA
atggcagctgtcggaccgcttgcgatgcacccgaccgtcacagaacacgattaccgcttcccgcgaagacccgatcctcatcgaagtaccgaccgcagtttcgcccgcatggattcgcgagacgaccatgtcgcactcgccaacggcaatgcctccgccgccgtgacgagcgggcaagccgcctcgtcgcgcaagaacagcgtcctcgactccgccttgtttccatcgctcgaaaacgccgcctccggctcgtccagcagcctcgagcagatgcagagcgacgatccgctggcggcccagatctggaagttcttcaacaggacgaagcagcagctgccgaaccagcagcgaatggagaacctgacgtggcgcatgatggccctcaacctgcgcaagcgtcagcagcagcaacagcagcagaagacgcacaacagatccctctcgcaaccaagtcaaccacaaaacgctcccagcggcatcgcccagcagctgcgaaagacatccgatagccatgctgagggcgtggatgccatgaacattgacgacttcatcttcgccgaggagggaggagcctccgtcaaggacaactcaccgcagccgcccgcatcctctctctctggtccgcagaccgctgccaccaagaccagcaaatcatccaggccgctggcttcggcaatccccatcaagggccgcaagggttcctctgccaatcaattcgtcgcccagtccgtccctgtagcgccacctcaccagcggggacagtttgatgaattccattacgtccctcgacacgatagaaagacgagtatagacgaccgccggacgaggaaacggcctgccaacttctcaccccatgtgagcgccatcaacccgagtgcgtctggtctcagcctcgatctggaagcagacccggatctcaatggatactccctcgacaacacaaacgcgatgtccctgcagcagcaaatggccaacaaccagggctctgctttcaacctggacttcatggacagcgacggcgtccttggctctgcccccttccatccgaacttctccttttctccatcgacgtctccaatgatgcacaatggaccgtttccatccgtgtacaacaacgccgcgctgcaatcttcgctgaacaaccccgacttctactctcccactggctcggggtatccatcggccacatccaccccacatccgatgaacgagggcgacggcgtctatttcggctcccaggaaatgcagcaccatccgcatcatcgtgcacagggcttccgacaaagctccggtaacctcaaccaccacttgagccaccagttcatgtacggcgattccaatggcggcaacggcagcggcggcggcaatggcgctgccttcccctccacaggcgccagctcggaggcggtggcatcgtacagcgctgctcaaaactcttttgggcacatcgacccaagccaagtgttccagacggagtcgcaggtcgcctctcccgtcttaccgatgcagcaaggcaacatgcttggtttcggaggagagtctgacgaagacgacggcaccgccttcttggatcgcggtggcgttggcctccacggtgacttcacatctgccgtcgacgaggcagggacgttcagctgggacgcctctttgccgggacagttcagcacgcaggctgctcgctttcccggcggccccactcgcaagcacgtacagattgggaccatcacgactgattacgtcgacaccagtaacgactgggatggccataacctgagccgatctcactcgcaaacgctcggcatgggcaacgacaagcgacagaacaaacttcctcgcaactcgtcgacaccctcgcacatggccgccaagcacagtggattcgagcaaatcgcccagtctctccccagctcaccgggtccccatgcttcgggaaccgcctccggcttctcgtcagttgcgccaagcagaccagcctcaccgcccccaagctccaagcacggctcctcgaccaacctgcatgcggctagcggaaaccccggagagaacagcgctcccactacctgcaccaactgcttcacccagacgactcctctgtggagacggaaccccgagggccagcccctctgcaatgcctgtggcctcttcctcaagctacacggcgtcgtgcgtcccctcagtctgaagacggacgttatcaagaagagaaacaggggctccggtgccaacctcccggttggaggatcgaggaagaagaacgcaggcaatggctccaacgccgcctcgaggaagaactcatcgctgtccatggctgccgctgccgcggcgacggcagctgttgctaccgcaaaccaccaagccgcgacgccgccatctacaactcgcccaagcagcaataaggagggcgatagccccgccagcagtggccatcacaccgcaggcagcacgcctggcagccaatatggaggagccaccattgtgggcggcaaaggcgtcgtgcccattgcggcggcaccacccaagacaacgcctggacccggagcctcgtcgtccatgtcccgaccgaccacggcgtcctccaaaagacagcgtaggcatagcaagagcgagggcacttcatcgtcggctatggatctcgacggccctggagactctattgggcgacagcttggcgcaactcccagcatgccttccatctctagcactatgctttcgaatggaagcttcaccatgggtcagcgctccatgatgggatcaagcatggtgtccatgggtggttcccagggcaaccttgtgaacagcgccggtggctcgtcaggccctcaggaatgggagtggttgactatgagtctttga

SEQ ID NO: 105 - protein, 1037 aa, Myceliophthora heterothallica
MAATTTDSSAAPGPLAPMHPTTTEHDFRFPRRPPADAATGARKTDRLESASGTKPAPPTSTSTATPRTATAGTTTATAGADGLRPGLLDGRLNLGLAQSGGAAHLDLLRVSAFPPFQQSARREAQSLEEMQREDPLATQIWKFYAKTKQLLPAQERMENLTWRMMHLKLQKTKAANTTKTSRASSSSANTPSGIAQLRKSSEHAPPQLDPMNLDDFIHNDNVGTLASLALTPTPETMRQAEERSAHTTATAIPIKNRKEPSSSSSSSSSSQHLAPQSVPVAAHQRPRDEFGYLPRHPRKTSIDETGQRVSSLTRKRPANFSPHVPAVNSGLSTSGLDADADLQEYSLDHLQHPAAASQPPNQMGVPFPLDTFRLGNDLSISSAGPFQQNFSFSPATSPMAANDPFASLYNSSSMPSGSLGADFYSPPGSAYQSAVSTPHPLGEADGFYFGSMDVRQMRQHPYRNGPAAMANPLGQQFPYGANGSLMFPAASTGPETSSAFTAPSSFSHVDPTRVFQQELPARSPAVGLGQESMFSFGADSDDEDGGAFADRNLPISQDLSPQGSSMDDGAFDSSSLQWDPSLPGNFSMQAARYPAGPTRKQVTIGGTTTDYVDSKGDWEGGSLNRSQSQPFRTTNGRVGKVPRTASTPRLTSKGNPFDRLAQSTPNSPPAEANQNSAFSSVSTSRPSSPPPASRQGSTTNLQAAAGSSQNESSSSGGGGGSSGSTNPNSNASNNNNNNNNNNNNNSSSNTNTPTTCTNCFTQTTPLWRRNPEGQPLCNACGLFLKLHGVVRPLSLKTDVIKKRNRGSGSSLPVGGGGTRSRKNVGSSGGISKRGSTLSVSSSANPPPAQASTPPAASNRTGSANDSESSANGAASTGNGNTPTGYHGNATSSTNVAVGGKGVVPIAAAPAKNLPGPGAASIPRTTAISSSSLSSSKRQRRHSKSVTAAEQPSLNSMDIDSPESSTGSNEGAATSSAGLSKPSLGSSAGLASLQPSASSFSLGGFGSSGGSNGMQRPLAGPGSQRPAPQEWEWLTMSL

SEQ ID NO: 106 - DNA, 3447 bp, Myceliophthora heterothallica
  source 1..3447: mol_type = genomic DNA
atggcagcgaccacgacggattcctctgcggcgcctggtccgctggccccaatgcatcccaccacgacagagcacgactttcgctttccgcgaaggccgccggcagacgccgcgaccggggcgcggaaaaccgaccgactcgaatctgcctccggtaccaagccggcgccgccgacatcaacgtcgacggcgaccccgaggacggcgacggcggggaccacgacggcgacggcgggcgccgacggattgcgtcccggcctgctcgatggccgcctgaacctaggcctggctcagagtggcggcgccgcccaccttgacctgctgcgcgtctccgccttcccgccgttccagcagagtgcccggcgagaggcccagagcctggaggagatgcagagggaagacccgctggccacccagatctggaagttttacgcaaagacaaaacagctcctgcccgcccaggagcgcatggaaaacctgacgtggcggatgatgcatctcaagttacagaagacgaaggcagccaacacgacaaagtaggtgttttttttttctctttctttctttcccgtcttggtgtcccgcggattgtgtggtacttcgccggttggaggactgggagaaagaaagcaaaaaagaaagaaaaaaaaaagagagaaagaaagcttgggagagagccgggtagaaaaagaagaatgcttgctgactcctgtgggcaaacaaagaacaagtcgtgcatccagctcatcagcaaatacccccagtgggattgcgcaattgcgcaagtcgtcggaacatgccccgccacagttggacccgatgaacctggacgatttcattcacaacgacaacgtcggaaccctggccagcctcgccttgacgccgaccccagagacgatgcgccaggcggaggagagatcggctcacaccacggcgacggccattcccatcaagaaccgcaaggagccgtcgtcgtcgtcatcgtcatcgtcgtcgtctcagcatctcgcaccccagtcggtgcccgttgccgcccaccagaggcctcgggacgaatttggctatctgcctcggcacccgcgcaaaactagcattgacgagaccggccaacgtgtaagttctctcgtctttttgctgctgcgtacgggtcgtctttggagtcaattcatcgggcgccccttgaagccgtcttgtattttcgatgacacgagatgccttcctgcttttttttgtgacggcggtctgatgatccattgcctctctctctagacccgcaaacgaccggccaatttctccccccacgtacccgccgtcaacagcggcctgtcgacaagtggactcgatgccgacgctgacctccaggaatactcactcgaccacttacaacatcccgccgccgccagccaaccgcccaatcagatgggggttcccttcccgctcgacaccttccgcttggggaacgacctgagcatcagctctgccgggcccttccagcagaacttctccttctcgccggccacctcgcccatggcggcaaatgacccgttcgccagcctctataatagctcctcgatgccttcgggctccctaggggccgacttctattcccctcccggctcggcctatcagtccgccgtttccaccccgcatcccctgggcgaggccgatggcttctactttggctccatggatgtgcggcagatgcgccaacatccgtataggaatggccctgccgccatggccaacccgctaggccaacaattcccctacggcgcgaatgggagcctgatgttcccggccgcatcgaccggccccgagacgtcctcggcttttaccgcgccgagctctttcagccatgtcgacccgacgcgggtgttccagcaagagctccctgctcgttcccccgccgtcgggctgggccaagagagcatgttctccttcggagccgattcggacgacgaggacggcggcgcctttgccgaccgcaatctgccaatctcgcaggacctctcgccccaaggaagcagtatggatgacggtgcgtttgacagttcgtcactgcagtgggatccatccctcccgggcaacttcagcatgcaggcggccaggtatccggccgggcctactcggaaacaggtgacaataggcggcaccacgaccgactacgtcgactcgaagggggattgggagggcggaagtctgaacaggtcgcagtcgcagcctttccgtacaacgaacggtcgcgtcgggaaggtaccgcggacagcatcgaccccgaggttgacaagcaagggcaatccgttcgacaggttggcacagtcgaccccgaactcgccgccagccgaggccaaccagaactcggccttctcctctgtctctacaagccgcccgtcctcgcctcctccggcttcgaggcagggttccacaaccaacctccaagccgccgcgggaagcagtcagaacgagagcagcagcagcggcggcggcggcggcagcagcggcagcacgaaccccaactccaacgccagcaacaacaacaacaataacaataacaataacaacaacaacagttcctccaacactaacacccccacaacatgtaccaactgcttcacccagacaacgccgctctggcgccgcaatcccgaaggccaacccctctgcaacgcttgcggtcttttcctcaagctgcatggtgtggttcgcccgctgagcttgaagacggacgtcatcaagaagcggaaccgcggctccggttcgagtcttcctgtcggcggcggcggcacccggtcgaggaagaacgtcggctcttctggggggataagcaagaggggctcgactttatccgtctcctccagcgccaaccctcccccagctcaggcgagcaccccgccggcagcctcgaaccggaccggtagcgccaacgacagcgaaagttccgcgaacggcgccgcgtcgaccgggaatggaaacacgccaaccggctaccacggcaatgccacctcctctaccaacgttgccgttggcggcaagggtgtcgttccgatcgccgcggccccggcgaagaaccttcccggcccgggcgccgcctccataccgcgcaccacagccatctcctcttcctcgttgtcttcgtcgaaacgccaacgccgccacagcaagagcgtcacagcggcagagcagccgtcgctgaacagcatggacatcgacagcccggagagctctacgggatccaacgagggcgcggcaacatcgtcggccggcctgtcgaagccgagcctgggttcgagcgcggggctggcttcgttgcagcccagtgctagcagctttagtttgggtggctttggcagtagcggcggcagcaacggcatgcagaggccattggccgggccaggaagccagcggcctgcgccacaggagtgggagtggttgaccatgagtttatag

SEQ ID NO: 107 - DNA, 3114 bp, Myceliophthora heterothallica
  source 1..3114: mol_type = other DNA
atggcagcgaccacgacggattcctctgcggcgcctggtccgctggccccaatgcatcccaccacgacagagcacgactttcgctttccgcgaaggccgccggcagacgccgcgaccggggcgcggaaaaccgaccgactcgaatctgcctccggtaccaagccggcgccgccgacatcaacgtcgacggcgaccccgaggacggcgacggcggggaccacgacggcgacggcgggcgccgacggattgcgtcccggcctgctcgatggccgcctgaacctaggcctggctcagagtggcggcgccgcccaccttgacctgctgcgcgtctccgccttcccgccgttccagcagagtgcccggcgagaggcccagagcctggaggagatgcagagggaagacccgctggccacccagatctggaagttttacgcaaagacaaaacagctcctgcccgcccaggagcgcatggaaaacctgacgtggcggatgatgcatctcaagttacagaagacgaaggcagccaacacgacaaaaacaagtcgtgcatccagctcatcagcaaatacccccagtgggattgcgcaattgcgcaagtcgtcggaacatgccccgccacagttggacccgatgaacctggacgatttcattcacaacgacaacgtcggaaccctggccagcctcgccttgacgccgaccccagagacgatgcgccaggcggaggagagatcggctcacaccacggcgacggccattcccatcaagaaccgcaaggagccgtcgtcgtcgtcatcgtcatcgtcgtcgtctcagcatctcgcaccccagtcggtgcccgttgccgcccaccagaggcctcgggacgaatttggctatctgcctcggcacccgcgcaaaactagcattgacgagaccggccaacgtgtaagttctctcacccgcaaacgaccggccaatttctccccccacgtacccgccgtcaacagcggcctgtcgacaagtggactcgatgccgacgctgacctccaggaatactcactcgaccacttacaacatcccgccgccgccagccaaccgcccaatcagatgggggttcccttcccgctcgacaccttccgcttggggaacgacctgagcatcagctctgccgggcccttccagcagaacttctccttctcgccggccacctcgcccatggcggcaaatgacccgttcgccagcctctataatagctcctcgatgccttcgggctccctaggggccgacttctattcccctcccggctcggcctatcagtccgccgtttccaccccgcatcccctgggcgaggccgatggcttctactttggctccatggatgtgcggcagatgcgccaacatccgtataggaatggccctgccgccatggccaacccgctaggccaacaattcccctacggcgcgaatgggagcctgatgttcccggccgcatcgaccggccccgagacgtcctcggcttttaccgcgccgagctctttcagccatgtcgacccgacgcgggtgttccagcaagagctccctgctcgttcccccgccgtcgggctgggccaagagagcatgttctccttcggagccgattcggacgacgaggacggcggcgcctttgccgaccgcaatctgccaatctcgcaggacctctcgccccaaggaagcagtatggatgacggtgcgtttgacagttcgtcactgcagtgggatccatccctcccgggcaacttcagcatgcaggcggccaggtatccggccgggcctactcggaaacaggtgacaataggcggcaccacgaccgactacgtcgactcgaagggggattgggagggcggaagtctgaacaggtcgcagtcgcagcctttccgtacaacgaacggtcgcgtcgggaaggtaccgcggacagcatcgaccccgaggttgacaagcaagggcaatccgttcgacaggttggcacagtcgaccccgaactcgccgccagccgaggccaaccagaactcggccttctcctctgtctctacaagccgcccgtcctcgcctcctccggcttcgaggcagggttccacaaccaacctccaagccgccgcgggaagcagtcagaacgagagcagcagcagcggcggcggcggcggcagcagcggcagcacgaaccccaactccaacgccagcaacaacaacaacaataacaataacaataacaacaacaacagttcctccaacactaacacccccacaacatgtaccaactgcttcacccagacaacgccgctctggcgccgcaatcccgaaggccaacccctctgcaacgcttgcggtcttttcctcaagctgcatggtgtggttcgcccgctgagcttgaagacggacgtcatcaagaagcggaaccgcggctccggttcgagtcttcctgtcggcggcggcggcacccggtcgaggaagaacgtcggctcttctggggggataagcaagaggggctcgactttatccgtctcctccagcgccaaccctcccccagctcaggcgagcaccccgccggcagcctcgaaccggaccggtagcgccaacgacagcgaaagttccgcgaacggcgccgcgtcgaccgggaatggaaacacgccaaccggctaccacggcaatgccacctcctctaccaacgttgccgttggcggcaagggtgtcgttccgatcgccgcggccccggcgaagaaccttcccggcccgggcgccgcctccataccgcgcaccacagccatctcctcttcctcgttgtcttcgtcgaaacgccaacgccgccacagcaagagcgtcacagcggcagagcagccgtcgctgaacagcatggacatcgacagcccggagagctctacgggatccaacgagggcgcggcaacatcgtcggccggcctgtcgaagccgagcctgggttcgagcgcggggctggcttcgttgcagcccagtgctagcagctttagtttgggtggctttggcagtagcggcggcagcaacggcatgcagaggccattggccgggccaggaagccagcggcctgcgccacaggagtgggagtggttgaccatgagtttatag

SEQ ID NO: 108 - protein, 51 aa, synthetic construct
  REGION 1..51: note = GATA-type zinc finger domain sequence
PTTCTNCFTQTTPLWRRNPEGQPLCNACGLFLKLHGVVRPLSLKTDVIKKR

SEQ ID NO: 109 - skipped

SEQ ID NO: 110 - protein, 1021 aa, Myceliophthora thermophila
MAATTTDSSAAPGPLAPMHPTMTEHDFRFPRRPADAATGARKTDRLESASGTKPVPPTSTSTAASRTATSGTTTAAAGGGGLRPGLLDGRLNLGLAQGGGAAHLDLLRASAFPPFQQSARREAQSLEEMQREDPLATQIWKFYAKTKQLLPAQERMENLTWRMMHLKLQKTKAANTTKTSRASSSSANTPSGIAQLRKSSEHAPPQLDPMNLDDFIHNDNVGTPAGLALTPTPETMRQAEERSAHTTATAIPIKNRKEPSSSSSSSSSQHLAPQSVPVAAHQRPQDEFGYLPRHPRKTSIDETGQRTRKRPANFSPHVPAVNSGLSTSGLDADADLQEYSLDHLQHPAAASQPPNQMGVPFPLDTFRLGNDLSISSAGPFQQNFSFSPATSPMAANDPFASLYNASSMPSGSLGADFYSPPGSAYQSAVSTPHPLGEADGFYFGSMDVRQMRQHPYRNGPAAMANPLGQQFPYGANGGNLMFPAASTGPETSSAFTAPSSFSHVDPTRVFQQDLPARSPAVGLGQESMFSFGADSDDEDGGAFADRNLPISQDLSPQGSSMDDGAFDCSSLQWDPSLPGNFSMQAARYPGGPTRKQVTIGGTTTDYVDSKGDWEGGNLNRSQSQPFRTTNGRVGKVPRTASTPRLTSKGNPFDRLAQSTPNSPPAEANQNSAFSSVSTSRPSSPPPASRQGSTTNLQAAAGSSQNESNSSSTNSNSNATTTTTTTTTTTTNNNNNNNNNNNSSSNTNTPTTCTNCFTQTTPLWRRNPEGQPLCNACGLFLKLHGVVRPLSLKTDVIKKRNRGSGSSLPVGGGGTRKRGSTLSVSSSANPPPAQASTPPAASNRTGNANDSESSANGAASTGNGNTPTGYHGNAISSTNVAVGGKGVVPIAAAPAKNLPGPGAASVPRTTAISSSSLSSSKRQRRHSKSVTAAEQPSLNSMDIDSPESSTGSNEGAATSSAGLSKPSLGSSAGLASLQPSASSFSLGGFGSSGGSSGMQRPLAGPGSQRPAPQEWEWLTMSL

SEQ ID NO: 111 - DNA, 3441 bp, Myceliophthora thermophila
  source 1..3441: mol_type = genomic DNA
atggcagcgaccacgacggattcctctgcggcgcctggtccgctggccccaatgcatcccaccatgacagagcacgactttcgctttccgcgaaggccagcagacgccgcgaccggggcgcggaaaaccgaccgactcgaatctgcctccggtaccaagccggtgccgccgacatcaacgtcgacggcggcctcgaggacggcgacgtcggggaccacgacggcagcggcgggcggcggcggattgcgtcccggcctgctcgatggccgcctgaacctaggcctggctcagggtggtggtgccgcccaccttgacctgctgcgcgcctccgccttcccgcccttccagcagagtgcccggcgagaggcccagagcctggaggagatgcagagggaagacccgctggccacccagatctggaagttttacgcaaagacaaaacagctcctgcccgcccaggagcgcatggaaaacctgacgtggcggatgatgcatctcaagttgcagaagacgaaggcagccaacacgacaaagtaggtttttttctttctttctttctttctttcccgtcttggtgtcccgcggattgtgtggtacttcgccggttggaggactgggagaaagaaagaaagaaagaaaagaaaagaaaagaaagaaagcttgggagagagccgggtagaaaaagaagaatgcttgctgacttctgtgggcaaacaaaggacaagtcgtgcatccagctcatcagcaaatacccccagtgggattgcgcaattgcgcaagtcgtcggaacatgccccgccacagttggacccgatgaacctggacgatttcattcacaacgataacgtcggaaccccggccggcctcgccttgaccccgaccccagaaacgatgcgccaggcggaggagaggtcggctcacaccacggcgacggccattcccatcaagaaccgcaaggagccgtcgtcgtcgtcgtcttcgtcgtcgtctcagcatcttgcaccccagtcggtgcccgttgccgcccaccagaggcctcaggacgaatttggctatctgcctcggcacccgcgcaaaactagcattgacgagaccggccaacgtgtaagttctctcgccttttgctgctgcgtacaggtcgtctttggagtcaaatccatcgcgccccttgaagccgtcttgtattttcgatgacacgagatgccttcctgctttttttgtgatggcggtctgatgatccattgcctctctctctagacccgcaaacgaccggccaatttctccccccacgtacccgccgtcaacagcggcctgtcgacaagtggactcgatgccgacgctgacctccaggagtactcactcgaccacttacaacatcccgccgccgccagccaaccgcccaatcagatgggagttcccttcccgctcgacaccttccgcttggggaacgacctgagcataagctctgccgggcccttccagcagaacttctccttctcgccggccacctcgccaatggcggcgaatgacccgttcgccagcctctataatgcctcctcgatgccttcgggctccctaggggccgacttctattcccctcccggctcggcctatcagtctgccgtttccacaccgcatcccctgggcgaggccgatggcttctactttggctccatggatgtgcggcagatgcgccaacatccgtataggaatggccctgccgccatggccaacccgctaggccaacagttcccctacggcgcgaatggaggaaacctgatgttcccggccgcatcgaccggccccgagacgtcctcggcttttacggcgccgagctctttcagccatgtcgacccgacccgggtgttccagcaagacctccctgctcgttcccccgccgtcgggctgggccaagagagcatgttctccttcggagcggattccgacgacgaggacggcggcgcctttgccgaccgcaatctgccaatctcgcaggacctctcgccccaaggaagcagtatggatgacggtgcgtttgactgttcgtcactgcagtgggatccatctctcccgggcaactttagcatgcaggcggctaggtatcctggcgggcctactcggaaacaggtgacaataggcggcaccacgaccgactacgtcgactcgaagggggattgggagggcggaaatctgaacaggtcgcagtcgcagcctttccgtacaacgaacggtcgcgtcgggaaggtaccgcggacagcatcgaccccgaggttgacaagcaagggcaatccgttcgacaggttggcacagtcgaccccgaactcgccgccagccgaggccaaccagaactcggccttctcctctgtctctacaagccgcccgtcctcgcctcctccagcttcgaggcagggttccacaaccaacctccaagccgccgcgggaagcagtcagaacgagagcaacagcagcagcacgaactccaactccaacgccaccaccaccaccaccaccaccactaccaccaccaccaacaacaacaacaacaacaacaacaacaacaacagttcctccaacactaacacccccacaacatgtaccaactgcttcacccagacaacgccgctctggcgccgcaatcccgaaggccaacccctctgcaatgcttgcggtcttttcctcaagctgcatggtgtggttcgcccgctgagcttgaagacggacgtcatcaagaagcggaaccgcggctccggttcgagtcttcctgtcggcggcggcggcacccggtcgaggaagaacggcggctcctctggggggataagcaagaggggctcgactttatccgtctcctccagcgccaaccctcccccagctcaggcgagcaccccgccggcagcctcgaaccggaccggtaacgccaacgacagcgaaagttccgcgaacggcgccgcgtcgaccgggaatggaaacacgccaaccggctaccacggcaatgccatctcctctaccaatgttgccgttggcggcaagggtgtcgttccgatcgctgcggccccggcgaagaaccttcccggcccgggcgccgcctccgtaccgcgcaccacagccatctcctcttcctcgttgtcttcgtcgaaacgccaacgccgccacagcaagagcgtcacagcggcagagcagccgtcgctgaacagcatggacatcgacagcccggagagctctacgggatccaacgagggcgcggcaacgtcgtcggcgggcctgtcgaagccgagcctgggttcgagcgcggggctggcttcgttgcagcccagtgctagtagctttagtttgggtggctttggcagtagcggcggcagtagcggcatgcagaggccattggccgggccaggaagccagcggcctgcgccacaggagtgggagtggttgaccatgagtttatag

SEQ ID NO: 112 - DNA, 3066 bp, Myceliophthora thermophila
  source 1..3066: mol_type = other DNA
atggcagcgaccacgacggattcctctgcggcgcctggtccgctggccccaatgcatcccaccatgacagagcacgactttcgctttccgcgaaggccagcagacgccgcgaccggggcgcggaaaaccgaccgactcgaatctgcctccggtaccaagccggtgccgccgacatcaacgtcgacggcggcctcgaggacggcgacgtcggggaccacgacggcagcggcgggcggcggcggattgcgtcccggcctgctcgatggccgcctgaacctaggcctggctcagggtggtggtgccgcccaccttgacctgctgcgcgcctccgccttcccgcccttccagcagagtgcccggcgagaggcccagagcctggaggagatgcagagggaagacccgctggccacccagatctggaagttttacgcaaagacaaaacagctcctgcccgcccaggagcgcatggaaaacctgacgtggcggatgatgcatctcaagttgcagaagacgaaggcagccaacacgacaaagacaagtcgtgcatccagctcatcagcaaatacccccagtgggattgcgcaattgcgcaagtcgtcggaacatgccccgccacagttggacccgatgaacctggacgatttcattcacaacgataacgtcggaaccccggccggcctcgccttgaccccgaccccagaaacgatgcgccaggcggaggagaggtcggctcacaccacggcgacggccattcccatcaagaaccgcaaggagccgtcgtcgtcgtcgtcttcgtcgtcgtctcagcatcttgcaccccagtcggtgcccgttgccgcccaccagaggcctcaggacgaatttggctatctgcctcggcacccgcgcaaaactagcattgacgagaccggccaacgtacccgcaaacgaccggccaatttctccccccacgtacccgccgtcaacagcggcctgtcgacaagtggactcgatgccgacgctgacctccaggagtactcactcgaccacttacaacatcccgccgccgccagccaaccgcccaatcagatgggagttcccttcccgctcgacaccttccgcttggggaacgacctgagcataagctctgccgggcccttccagcagaacttctccttctcgccggccacctcgccaatggcggcgaatgacccgttcgccagcctctataatgcctcctcgatgccttcgggctccctaggggccgacttctattcccctcccggctcggcctatcagtctgccgtttccacaccgcatcccctgggcgaggccgatggcttctactttggctccatggatgtgcggcagatgcgccaacatccgtataggaatggccctgccgccatggccaacccgctaggccaacagttcccctacggcgcgaatggaggaaacctgatgttcccggccgcatcgaccggccccgagacgtcctcggcttttacggcgccgagctctttcagccatgtcgacccgacccgggtgttccagcaagacctccctgctcgttcccccgccgtcgggctgggccaagagagcatgttctccttcggagcggattccgacgacgaggacggcggcgcctttgccgaccgcaatctgccaatctcgcaggacctctcgccccaaggaagcagtatggatgacggtgcgtttgactgttcgtcactgcagtgggatccatctctcccgggcaactttagcatgcaggcggctaggtatcctggcgggcctactcggaaacaggtgacaataggcggcaccacgaccgactacgtcgactcgaagggggattgggagggcggaaatctgaacaggtcgcagtcgcagcctttccgtacaacgaacggtcgcgtcgggaaggtaccgcggacagcatcgaccccgaggttgacaagcaagggcaatccgttcgacaggttggcacagtcgaccccgaactcgccgccagccgaggccaaccagaactcggccttctcctctgtctctacaagccgcccgtcctcgcctcctccagcttcgaggcagggttccacaaccaacctccaagccgccgcgggaagcagtcagaacgagagcaacagcagcagcacgaactccaactccaacgccaccaccaccaccaccaccaccactaccaccaccaccaacaacaacaacaacaacaacaacaacaacaacagttcctccaacactaacacccccacaacatgtaccaactgcttcacccagacaacgccgctctggcgccgcaatcccgaaggccaacccctctgcaatgcttgcggtcttttcctcaagctgcatggtgtggttcgcccgctgagcttgaagacggacgtcatcaagaagcggaaccgcggctccggttcgagtcttcctgtcggcggcggcggcacccgcaagaggggctcgactttatccgtctcctccagcgccaaccctcccccagctcaggcgagcaccccgccggcagcctcgaaccggaccggtaacgccaacgacagcgaaagttccgcgaacggcgccgcgtcgaccgggaatggaaacacgccaaccggctaccacggcaatgccatctcctctaccaatgttgccgttggcggcaagggtgtcgttccgatcgctgcggccccggcgaagaaccttcccggcccgggcgccgcctccgtaccgcgcaccacagccatctcctcttcctcgttgtcttcgtcgaaacgccaacgccgccacagcaagagcgtcacagcggcagagcagccgtcgctgaacagcatggacatcgacagcccggagagctctacgggatccaacgagggcgcggcaacgtcgtcggcgggcctgtcgaagccgagcctgggttcgagcgcggggctggcttcgttgcagcccagtgctagtagctttagtttgggtggctttggcagtagcggcggcagtagcggcatgcagaggccattggccgggccaggaagccagcggcctgcgccacaggagtgggagtggttgaccatgagtttatag

SEQ ID NO: 113 - protein, 876 aa, Aspergillus nidulans
MSGLTLGGGSGRVRPTQAAAAFPTSHDFADPDRSSVARNANRPSRRFAAPPQLSDDFSLESPTDSAQVHDNLLQDALFPEWKANSPRGVDSPDEMQKKDPLATQIWKLYSRTKAQLPNQERMENLTWRMMALSLRRQERERAQQQARASSQKSPVPGMSGIAQLRLSDRVSNTPTTTADTVSDAMNLDDFIIPFSPSDHPSPSTTKASEATTGAIPIKARRDQSASEATPVPASFPHPAQDQRRESEFGYVPRRVRKTSIDERQFFNLQIPSRKRPAESSPHVPPVSTSMLAHDPDFSHAVPEYTLDTSHGLSLQNQMNAQQLANAQNHTSPNMAFALDTFNLGDDPILPSAGPYQQQFTFSPSESPMTSGNPFANLYAQTPIASSLNSTDFFSPPPSGYQSTASTPQPAYDGEHSKYFDMPVDARSQRRVVPAYITQRSSNLSASLQPRYMYNQGGSSQDITQQNAHMGAQSSSMQSPGFSIPQHVDPTQVLNPNEFNGNHAAMFSFGADSDVEDDDGNQFSAGGLAMPAEFGDDSISDMNSNMAWETSYPNSFQSLPAFAAQHRKHVTIGSADMMDTPSEWNQGGSLGRTHESAASVSEVRNRDQDPRRQKIARTSSTPNTAQLLRQSMQNQSSHTSPNTPPESGLNSAAPSRPASPGGTKNGEQNGPTTCTNCFTQTTPLWRRNPEGQPLCNACGLFLKLHGVVRPLSLKTDVIKKRNRNSANSLAVGSSRVSKKSARKNSVQQVTPTAPTSSRAQSNTTSESPPAMPGSSGRGSGVVPIAAAPPKSSSAATTSPGTNNGCGAVQVAPKRQRRLEKASDVDMAESPSSTSSGGRSKVVPLAPAMPPAAVNPANHSIAAGQGASQEWEWLTMSL

SEQ ID NO: 114 - DNA, 2692 bp, Aspergillus nidulans
  source 1..2692: mol_type = genomic DNA
atgtctgggttgactctcgggggtggttctggccgtgtgcgaccgacacaagccgctgccgcttttcccacctcgcatgatttcgccgaccctgatcgctcgtccgtcgcccgcaatgccaaccggccctcccgccgtttcgctgcacctccgcagctgtccgacgacttctcgctcgagtcacccactgattccgctcaagttcacgacaacctcctccaggacgctttattccccgaatggaaggccaattctcctcgaggggtcgacagccctgatgagatgcagaagaaggatccgttagcgactcaaatatggaagctctattctagaacgaaagctcagttgcccaaccaagagcgcatggaaaatttgacctggcggatgatggctctgagtctgaggcgtcaggaaagagagcgcgcccagcagcaagcccggtacgtcatcattcccgatcgggggtatttcttttctctttctcttgctgatcttctatagtgcctccagccaaaagagtcctgttcctggcatgagtggcatcgctcagctccgtctgtctgatcgtgtttccaataccccgaccacaactgcagatactgtctcagatgctatgaaccttgacgacttcatcatccctttctctccgtctgaccacccatctccctccacaacgaaagcctccgaagccactactggtgctatacctatcaaagcccgcagggatcagtccgcgtcggaagccaccccagtacctgcatctttccctcacccggcccaggatcagcggagggagagtgagttcggatacgtaccgcgtagagtgcgcaagacgagcatcgatgagcgtcaattcttcaacctgcagattccgtcccgcaagcggccggccgagtcctcgcctcacgtaccgcctgtgtccaccagcatgctagctcatgacccggatttctcacatgctgtaccggagtataccttggatacttcgcatggactgtctctacagaatcagatgaacgctcagcagctcgccaatgctcagaaccacacctcccccaatatggcgttcgctctggatactttcaacctgggcgacgatcctatccttccatctgctggtccttaccagcagcaattcactttctcgcccagcgagtcaccgatgaccagcggtaacccgtttgccaacctctacgctcagactccgattgcctcttcactcaactcaaccgacttcttctctcctccgccgtccggctatcagtcgactgcttcgactcctcagcctgcgtatgatggcgaacactccaagtattttgatatgcccgtcgacgcgcggtcgcaacgccgggttgttcccgcttacattactcagcgatcatcgaatttgtctgcctcgctgcaacctcgatatatgtacaaccagggtggttcgtcccaggatattacacagcagaatgcccatatgggggcccagtcttcatccatgcaatcccctggcttttctatcccgcagcatgttgatccaacgcaagtgttgaatccgaacgagtttaacggtaaccacgccgcaatgttcagtttcggcgccgactcggatgttgaagatgacgatgggaaccaattttcggccggaggactggcaatgccagccgagttcggcgacgacagtatcagtgacatgaatagcaacatggcgtgggagacatcatatcccaactctttccagtcgctgcctgcttttgctgcacagcaccgaaagcatgttaccatcgggtctgcggacatgatggataccccgagcgagtggaaccagggcggaagcctaggtcgaactcacgagtcggctgcgtcagtcagcgaggtgcgcaatcgggatcaagaccctcgacggcagaagatcgctcgcacctcgtccactccaaacacagcccaactactacgccagagcatgcagaaccaatcgtcccacacgtctcccaacactccgcccgagtcgggtctaaacagcgctgctccatcgcggcccgccagtccaggcgggaccaagaacggagagcagaatggtcccaccacctgcacgaactgctttacgcagaccacaccgctgtggcggcgtaaccctgaaggtcagccgctgtgcaacgcctgcggtttgtttttgaagttgcacggcgtggtgcgcccactgtcgctcaagaccgatgtgatcaagaagcgtaaccgcaacagcgccaatagccttgccgttggctcttcacgggtatccaaaaagtcagcccgcaagaactcggttcagcaggtaactccgactgcgcctacctcgagccgggcccaaagcaatacgacatccgagtctcccccagcaatgccgggtagctccggccgaggctctggagtcgttcctattgcggctgccccgcctaaatctagctcagcggcgactacgtctccgggcacaaacaacggttgtggtgcggtacaggtggctcctaaacgtcagcgtaggctggaaaaggcctctgatgttgatatggcggaaagcccgtccagtacctcgtccggtggacgatccaaggttgttccattggcgccggctatgcctccagcggccgtcaacccagccaaccacagtatcgctgctggtcagggcgcaagccaggaatgggagtggttgacgatgagtttgtag

SEQ ID NO: 115 - DNA, 2631 bp, Aspergillus nidulans
  source 1..2631: mol_type = other DNA
atgtctgggttgactctcgggggtggttctggccgtgtgcgaccgacacaagccgctgccgcttttcccacctcgcatgatttcgccgaccctgatcgctcgtccgtcgcccgcaatgccaaccggccctcccgccgtttcgctgcacctccgcagctgtccgacgacttctcgctcgagtcacccactgattccgctcaagttcacgacaacctcctccaggacgctttattccccgaatggaaggccaattctcctcgaggggtcgacagccctgatgagatgcagaagaaggatccgttagcgactcaaatatggaagctctattctagaacgaaagctcagttgcccaaccaagagcgcatggaaaatttgacctggcggatgatggctctgagtctgaggcgtcaggaaagagagcgcgcccagcagcaagcccgtgcctccagccaaaagagtcctgttcctggcatgagtggcatcgctcagctccgtctgtctgatcgtgtttccaataccccgaccacaactgcagatactgtctcagatgctatgaaccttgacgacttcatcatccctttctctccgtctgaccacccatctccctccacaacgaaagcctccgaagccactactggtgctatacctatcaaagcccgcagggatcagtccgcgtcggaagccaccccagtacctgcatctttccctcacccggcccaggatcagcggagggagagtgagttcggatacgtaccgcgtagagtgcgcaagacgagcatcgatgagcgtcaattcttcaacctgcagattccgtcccgcaagcggccggccgagtcctcgcctcacgtaccgcctgtgtccaccagcatgctagctcatgacccggatttctcacatgctgtaccggagtataccttggatacttcgcatggactgtctctacagaatcagatgaacgctcagcagctcgccaatgctcagaaccacacctcccccaatatggcgttcgctctggatactttcaacctgggcgacgatcctatccttccatctgctggtccttaccagcagcaattcactttctcgcccagcgagtcaccgatgaccagcggtaacccgtttgccaacctctacgctcagactccgattgcctcttcactcaactcaaccgacttcttctctcctccgccgtccggctatcagtcgactgcttcgactcctcagcctgcgtatgatggcgaacactccaagtattttgatatgcccgtcgacgcgcggtcgcaacgccgggttgttcccgcttacattactcagcgatcatcgaatttgtctgcctcgctgcaacctcgatatatgtacaaccagggtggttcgtcccaggatattacacagcagaatgcccatatgggggcccagtcttcatccatgcaatcccctggcttttctatcccgcagcatgttgatccaacgcaagtgttgaatccgaacgagtttaacggtaaccacgccgcaatgttcagtttcggcgccgactcggatgttgaagatgacgatgggaaccaattttcggccggaggactggcaatgccagccgagttcggcgacgacagtatcagtgacatgaatagcaacatggcgtgggagacatcatatcccaactctttccagtcgctgcctgcttttgctgcacagcaccgaaagcatgttaccatcgggtctgcggacatgatggataccccgagcgagtggaaccagggcggaagcctaggtcgaactcacgagtcggctgcgtcagtcagcgaggtgcgcaatcgggatcaagaccctcgacggcagaagatcgctcgcacctcgtccactccaaacacagcccaactactacgccagagcatgcagaaccaatcgtcccacacgtctcccaacactccgcccgagtcgggtctaaacagcgctgctccatcgcggcccgccagtccaggcgggaccaagaacggagagcagaatggtcccaccacctgcacgaactgctttacgcagaccacaccgctgtggcggcgtaaccctgaaggtcagccgctgtgcaacgcctgcggtttgtttttgaagttgcacggcgtggtgcgcccactgtcgctcaagaccgatgtgatcaagaagcgtaaccgcaacagcgccaatagccttgccgttggctcttcacgggtatccaaaaagtcagcccgcaagaactcggttcagcaggtaactccgactgcgcctacctcgagccgggcccaaagcaatacgacatccgagtctcccccagcaatgccgggtagctccggccgaggctctggagtcgttcctattgcggctgccccgcctaaatctagctcagcggcgactacgtctccgggcacaaacaacggttgtggtgcggtacaggtggctcctaaacgtcagcgtaggctggaaaaggcctctgatgttgatatggcggaaagcccgtccagtacctcgtccggtggacgatccaaggttgttccattggcgccggctatgcctccagcggccgtcaacccagccaaccacagtatcgctgctggtcagggcgcaagccaggaatgggagtggttgacgatgagtttgtag

SEQ ID NO: 116 - protein, 944 aa, Trichoderma reesei
MAAVGPLAMHPTVTEHDYRFPRRPDPHRSTDRSFARMDSRDDHVALANGNASAAVTSGQAASSRKNSVLDSALFPSLENAASGSSSSLEQMQSDDPLAAQIWKFFNRTKQQLPNQQRMENLTWRMMALNLRKRQQQQQQQKTHNSQPQNAPSGIAQQLRKTSDSHAEGVDAMNIDDFIFAEEGGASVKDNSPQPPASSLSGPQTAATKTSKSSRPLASAIPIKGRKGSSANQFVAQSVPVAPPHQRGQFDEFHYVPRHDRKTSIDDRRTRKRPANFSPHVSAINPSASGLSLDLEADPDLNGYSLDNTNAMSLQQQMANNQGSAFNLDFMDSDGVLGSAPFHPNFSFSPSTSPMMHNGPFPSVYNNAALQSSLNNPDFYSPTGSGYPSATSTPHPMNEGDGVYFGSQEMQHHPHHRAQGFRQSSGNLNHHLSHQFMYGDSNGGNGSGGGNGAAFPSTGASSEAVASYSAAQNSFGHIDPSQVFQTESQVASPVLPMQQGNMLGFGGESDEDDGTAFLDRGGVGLHGDFTSAVDEAGTFSWDASLPGQFSTQAARFPGGPTRKHVQIGTITTDYVDTSNDWDGHNLSRSHSQTLGMGNDKRQNKLPRNSSTPSHMAAKHSGFEQIAQSLPSSPGPHASGTASGFSSVAPSRPASPPPSSKHGSSTNLHAASGNPGENSAPTTCTNCFTQTTPLWRRNPEGQPLCNACGLFLKLHGVVRPLSLKTDVIKKRNRGSGANLPVGGSRKKNAGNGSNAASRKNSSLSMAAAAAATAAVATANHQAATPPSTTRPSSNKEGDSPASSGHHTAGSTPGSQYGGATIVGGKGVVPIAAAPPKTTPGPGASSSMSRPTTASSKRQRRHSKSEGTSSSAMDLDGPGDSIGRQLGATPSMPSISSTMLSNGSFTMGQRSMMGSSMVSMGGSQGNLVNSAGGSSGPQEWEWLTMSL

SEQ ID NO: 117 - protein, 1021 aa, Myceliophthora heterothallica
MAATTTDSSAAPGPLAPMHPTTTEHDFRFPRRPPADAATGARKTDRLESASGTKPAPPTSTSTATPRTATAGTTTATAGADGLRPGLLDGRLNLGLAQSGGAAHLDLLRVSAFPPFQQSARREAQSLEEMQREDPLATQIWKFYAKTKQLLPAQERMENLTWRMMHLKLQKTKAANTTKTSRASSSSANTPSGIAQLRKSSEHAPPQLDPMNLDDFIHNDNVGTLASLALTPTPETMRQAEERSAHTTATAIPIKNRKEPSSSSSSSSSSQHLAPQSVPVAAHQRPRDEFGYLPRHPRKTSIDETGQRTRKRPANFSPHVPAVNSGLSTSGLDADADLQEYSLDHLQHPAAASQPPNQMGVPFPLDTFRLGNDLSISSAGPFQQNFSFSPATSPMAANDPFASLYNSSSMPSGSLGADFYSPPGSAYQSAVSTPHPLGEADGFYFGSMDVRQMRQHPYRNGPAAMANPLGQQFPYGANGSLMFPAASTGPETSSAFTAPSSFSHVDPTRVFQQELPARSPAVGLGQESMFSFGADSDDEDGGAFADRNLPISQDLSPQGSSMDDGAFDSSSLQWDPSLPGNFSMQAARYPAGPTRKQVTIGGTTTDYVDSKGDWEGGSLNRSQSQPFRTTNGRVGKVPRTASTPRLTSKGNPFDRLAQSTPNSPPAEANQNSAFSSVSTSRPSSPPPASRQGSTTNLQAAAGSSQNESSSSGGGGGSSGSTNPNSNASNNNNNNNNNNNNNSSSNTNTPTTCTNCFTQTTPLWRRNPEGQPLCNACGLFLKLHGVVRPLSLKTDVIKKRNRGSGSSLPVGGGGTRKRGSTLSVSSSANPPPAQASTPPAASNRTGSANDSESSANGAASTGNGNTPTGYHGNATSSTNVAVGGKGVVPIAAAPAKNLPGPGAASIPRTTAISSSSLSSSKRQRRHSKSVTAAEQPSLNSMDIDSPESSTGSNEGAATSSAGLSKPSLGSSAGLASLQPSASSFSLGGFGSSGGSNGMQRPLAGPGSQRPAPQEWEWLTMSL

SEQ ID NO: 118 - protein, 119 aa, synthetic construct
  REGION 1..119: note = Synthetic VHH sequence
DVQLVESGGGLVQAGGSLRLSCAASRSIFSINAMDWYRQAPGKQREWVAGITRGGTTKYADSVKGRFTISRDNAKKKVYLQMNSLKPEDTAVYYCNVLRGEQPWTRDYWGQGTQVTVSS

SEQ ID NO: 119 - protein, 119 aa, synthetic construct
  REGION 1..119: note = Synthetic VHH sequence
QVQLVESGGGLVQAGGSLRLSCAASRSIFSINAMDWYRQAPGKQREWVAGITRGGTTKYADSVKGRFTISRDNAKKKVYLQMNSLKPEDTAVYYCNVLRGEQPWTRDYWGQGTQVTVSS

SEQ ID NO: 120 - protein, 10 aa, synthetic construct
  REGION 1..10: note = Synthetic VHH sequence
RSIFSINAMD

SEQ ID NO: 121 - protein, 9 aa, synthetic construct
  REGION 1..9: note = Synthetic VHH sequence
GITRGGTTK

SEQ ID NO: 122 - protein, 11 aa, synthetic construct
  REGION 1..11: note = Synthetic VHH sequence
LRGEQPWTRDY

SEQ ID NO: 123 - protein, 117 aa, synthetic construct
  REGION 1..117: note = Synthetic VHH sequence
QVQLQESGGGLVQAGGSLRLSCAASGTIFRPTAMGWYRQAPGKERELVATITTGGSTKYADSVKGRFTISRGNAKNTVYLQMSSLKPEDTAVYYCNAQWGVRTRDYWGQGTQVTVSS

SEQ ID NO: 124 - protein, 10 aa, synthetic construct
  REGION 1..10: note = Synthetic VHH sequence
GTIFRPTAMG

SEQ ID NO: 125 - protein, 9 aa, synthetic construct
  REGION 1..9: note = Synthetic VHH sequence
TITTGGSTK

SEQ ID NO: 126 - protein, 9 aa, synthetic construct
  REGION 1..9: note = Synthetic VHH sequence
QWGVRTRDY

SEQ ID NO: 127 - protein, 126 aa, synthetic construct
  REGION 1..126: note = Synthetic VHH sequence
QVQLQESGGGLVQAGDSLRLSCAASISDRAFSRHVMGWFRQPPGKEREFVAAIGWTGRRTYYADSVKGRFTISRDNAMNTVYLQMNSLKPEDTAVYYCAASHFYSVSFEINDYDYWGQGTQVTVSS

SEQ ID NO: 128 - protein, 10 aa, synthetic construct
  REGION 1..10: note = Synthetic VHH sequence
ISDRAFSRHV

SEQ ID NO: 129 - protein, 10 aa, synthetic construct
  REGION 1..10: note = Synthetic VHH sequence
AIGWTGRRTY

SEQ ID NO: 130 - protein, 15 aa, synthetic construct
  REGION 1..15: note = Synthetic VHH sequence
SHFYSVSFEINDYDY

SEQ ID NO: 131 - protein, 117 aa, synthetic construct
  REGION 1..117: note = Synthetic VHH sequence
DVQLQESGGGLVQAGGSLRLSCAASGTIFRPTAMGWYRQAPGKERELVATITTGGSTKYADSVKGRFTISRGNAKNTVYLQMSSLKPEDTAVYYCNAQWGVRTRDYWGQGTQVTVSS

SEQ ID NO: 132 - protein, 126 aa, synthetic construct
  REGION 1..126: note = Synthetic VHH sequence
DVQLQESGGGLVQAGDSLRLSCAASISDRAFSRHVMGWFRQPPGKEREFVAAIGWTGRRTYYADSVKGRFTISRDNAMNTVYLQMNSLKPEDTAVYYCAASHFYSVSFEINDYDYWGQGTQVTVSS

SEQ ID NO: 133 - protein, 286 aa, Trichoderma reesei
MARFILQTLLAIAVATSALPTDNSTNFVGPENGNLVIVGGGSLDDSILQKVIDLAGGNDSSIVVIPTAQGDPSYDQNAANADDFRRLGSKSVTVLHTYDPEVANTEEFVQPLVNATGVWFGGGRQWRLVDAYAGTLTEQKIRAVLDAGGVIGGSSAGASIQGDFLARGDTKSNSLIIGDHQQGFAYVKNVAIDQHVLVRNRHFDMLNVLKYKPGILGIAINENTAVHVSQNTAEVFGASYAIIYDGTYWSRDGDDANQIPDPSGLFYFLRQGDIYDLGLRKVVHAS

SEQ ID NO: 134 - protein, 610 aa, Trichoderma reesei
MLSAAVLLGGLSSVALAQFPPKPEGVTVLRSKFHENVTISFKEPGICETTPGVKSYAGHVHLPPRLLEDADGEPQNYPVNTFFWFFEARENPSTAPLAIWLNGGPGSSSMLGLLMENGPCFVNEDSKSTVLNPWSWNNEVNMLYIDEPNQVGFSYDTPTNCTANLSHDGFILNPADFSAQLPELNLTSMVGTFSSQKPTHTTNSTAQAAHALWHFAQAWFSEFPHYKPSDDRISIWAESYGGHYGPGFMRFFQEQNEKIANGTIEDEHAHYLHLDTLGIVNGWLDSVIQEEAYIDFPYNNTYGIQVFNQSIYEELKHNFTKPNGCRDQIKRCQQSLSLFDVATINAQRVNFVDLCDMESWCMSPAVSTYFTLNSGWFDISHPIRDPFPPSHAYGYLTEESVLAALGSPVNFTATSAAVSSNFASSYDEFHGGFVDAVGYLLDHGVKVHMMYGDRDYACNWIGGERASLVVPYSRAADFARAGYAPLLTDRGVAGLTRQHGNYSFSRVFQAGHMVPSYQPEAAYDIFMRATFNRDIPTGLQPVRDDLSTEGPSDTWHVRHEPPEAPAPRCYILSPQSCVPELWDKVVAGTALVRDWFVDGEAEEGLFSDEL

SEQ ID NO: 135 - protein, 641 aa, Myceliophthora heterothallica
MTMKGSTLLALALGFGAHAQFPPKREGITVIESKFYKNVSISFKEPGICETTPGVKSYSGYVHLPPNLIEGADQDYPINTFFWFFEARKDPANAPLAIWLNGGPGGSSMMGLLEENGPCFVGPDSKTTYLNRWSWNNEANMLYIDQPVQTGFSYDVLTNVTVQLDVDDPSEPIITPTNFTDGHIPRTNNTFRIGTVGSQKASQVTNSTELSAHAMWHFLQTWLFEFPHYRSDDGRISLWAESYGGTYGPAFFRFFQQQNERIADGQLEGRYLHLDTLGIINGAVDWPILAESLIDYPYNNSYGIQFYNDTFHAALKHNWTRPSGWREQMQACTESLASSSSSSSPPAAGCEAVRSVLDDVLAAAFPRQSGRAPFDLAHPRADPFPPPHPHGFLARADVQAALGVPVNHTAVSLPVNRAFDATFDPLRGGQLDALAGLLDRRAGGGVKVHLVYGDRDPSCNWAGGEKVSLAVPWSRRDVFAAAGYAPLVVVSGKGGGDGGNTGGGNTGGGEEEVVVVRGLTRQVGRFSFTRVFQAGHEVPSYQPQAGYEIFRRAMAGLDLPTGRVRAGDDFVTAGLRDAWAVKNAAPDMVEPRCYVLKPESCEPEVWKTVVDGTAIVKDWFVVGSTGGEGRGVEGGIDGDEL

SEQ ID NO: 136 - protein, 554 aa, Myceliophthora heterothallica; identical to SEQ ID NO: 82

SEQ ID NO: 137 - protein, 442 aa, Trichoderma reesei
MQLLTTLFLLAQVVASRAALGDSPPGPRSEKVSYDGYHVYSVTTTDRSQAEFLATNLAAYPTEFTSRGFEVAIPPNEIQSFNKLGLAAQLLSDDLGKLIRRERKAPTYNRGHHKAGELPDLTWYDTYHDYDDHLQYWDDLVAAFPRNSEKYDIGTSYENRTIYAFHLFGDKGETANKPIILWHATVHAREWITTLVIEYWAWQLINGYRIKDKEITRFLDYYDFWLVPFHNPDGFSYTQTTDRMWRKNRLPRGNTTCVGTDLNRNWRFEWGGEPGTGAASTDPCDSTFQGLSPGDTPENAAVSGLSDKLGKSPRGIRSYIDLHSYGQKILTPPGWTCNTTQYPATLPRMLEVGGGFANAVQAYDSRNETYQYGTGCDIEYYSAGNGRDHHYGAYGADHSWTLELDPVTSGQGGFVLPPEDIWPVVKEQWAGVSWLLNNVWHN

SEQ ID NO: 138 - protein, 535 aa, Myceliophthora heterothallica
MRLLVRSLAALPIALSLVFPLPTAGAGLQPSPYQRLDRSESTSHVFPFLKWLRDSAVEAVFGRPPTKTKDQRPGPQLQSRYRNDVVVRFNVTNPEEEGAIAQAVSQMFLDVWAFAPDFVDVRLAKDDLSALLTLLPGSLEPSILIPDVAAAVWATYPSSSAGASRLQWSTADAAKMRTSLDGVGNIFFQDYQPLAVITNWMRLLEAMFPSVASMSSIGKSYEGRDLYALRVGAPSDDSEASGPRKTILITGGLHGREWISTSSVNYLLWSAVTSYGTDSMMTKLLKHFDIVFIPVLNPDGYEYTWKVDRLWRKSRQPTKMRFCRGLDLDHAFGHSWDAGRHTSDPCSESYGGEQPFQAVEAAALAQWALNETENGLNFVAFLDLHSYSQQVLFPYAYTCSLEPPNLENLEELAVGLAKAIRISSGEPYTVSSACEGAVAGAYSASDSLRRIEARGGAAIDWFYHELHARYSYQIKLRDTGSYGFLVPSDNIVPTGEEMLNALKYLGDYLLGNNGIEKLLAQQPLDHWKDLKRRRR

SEQ ID NO: 139 - protein, 534 aa, Myceliophthora heterothallica
MRLLVRTLAALPIALSLVFPLPTAGAGLQPSPYQRLDRSESTSHVFPFLKWLRDSAVEAVFGRPPTKTKDQRPGPQLQSRYRNDVVVRFNVTNPEEERAIAQAVSQMFLDVWAFAPDFVDVRLAKDDVSALLTLLPGSLEPSILIPDVAAAVWATYPSSSAGASRLQWSTADAAKMRTSLDGVGNIFFQDYQPLAVITNWMRLLEAMFPSVASLSSIGKSYEGRDLYALRVGAPSDDSEASGPRKTILITGGLHGREWISISSVNYLLWSAVTSYGTDSMMTKLLKHFDIVFIPVLNPDGYEYTWKVDRLWRKSRQPTKMRFCRGLDLDHAFGHGWDAGRHTSDPCSESYGGEQPFQAVEAAALAQWAFNETENGLNFVAFLDLHSYSQQVLFPYAYTCSLEPPNLENLEELAVGLAKAIRISSGEPYTVSSACEGAVAGAYSASDSRRRIEAGDGAAIDWFYHELHARFSYHIKLRDTGSYGFLVPSDNIVPTGEEMLNALKYLGDYLLGNNGIEKLLAKPLDHWKDLKRRRR

(19) United States
(12) Patent Application Publication
(10) Pub. No.: US 2025/0137001 A1
(43) Pub. Date: May 1, 2025

(54) MODIFIED MICROBIAL CELLS AND USES THEREOF

(71) Applicant: Biotalys NV, Sint-Denijs-Westrem (BE)

(72) Inventors: Lucia Nancy COCONI LINARES, Sint-Denijs-Westrem (BE); Benjamin S BOWER, Sint-Denijs-Westrem (BE)

(21) Appl. No.: 18/834,089

(22) PCT Filed: Feb. 2, 2023
(86) PCT No.: PCT/EP2023/052615

§ 371 Date: Jul. 29, 2024

Related U.S. Application Data
(60) Provisional application No. 63/305,869, filed on Feb. 2, 2022.

(51) Int. Cl. C12N 15/80; C07K 16/00

(52) U.S. Cl. CPC C12N 15/80; C07K 16/00; C07K 2317/14; C07K 2317/569

(57) ABSTRACT

The present invention relates to modified microbial host cells, the modification modulates protease activity of at least one endogenous protease if compared with a parent microbial host cell which lacks the modification and measured under the same or substantially the same conditions. The present invention further relates to a method of producing a compound of interest. The present invention further provides a method of increasing production of a compound of interest. The present invention further relates to a modified microbial host cell that has both a modification modulating the protease activity of at least one endogenous protease and a further modification in a regulator of transcription that regulates the transcription of one or more protease genes, resulting in a further increase in production of a compound of interest. The present invention also relates to a method of producing the modified microbial host cells of the invention. The present invention provides nucleic acids, genetic constructs, host cells and kits for use in the method of the invention as well as polypeptides obtained by the method of the invention.

### FIELD OF THE INVENTION

The present invention relates to modified microbial host cells, such as modified filamentous fungal cells. More specifically, the present invention relates to the modified microbial host cells wherein the modification modulates protease activity of at least one endogenous protease if compared with a parent microbial cell which has not been modified and measured under the same or substantially the same conditions. The present invention further relates to a method of producing a compound of interest. The present invention further provides a method of increasing production of a compound of interest. The present invention also relates to a method of producing the microbial host cells of the invention. The present invention provides nucleic acids, genetic constructs, host cells and kits for use in the method of the invention as well as polypeptides obtained by the method of the invention.

Other aspects, embodiments, advantages and applications of the invention will become clear from the further description herein.

### BACKGROUND

The efficient and cost-effective production of recombinant proteins is very important in the field of pharmacology but even more so in the field of agriculture where greater amounts of active protein may be required. This puts high demands on the production process and development of biological products. Different species of filamentous fungi have historically been used in fermentations and were selected by centuries of use. In more recent times, filamentous fungi are being used for their properties to produce extracellular plant biomass degrading enzymes. This interesting aspect was mainly exploited with the production of biofuels as a goal. The key producers of extracellular (hemi)-cellulases are Aspergillus, Trichoderma, Penicillium and Neurospora species and over the past decades these strains have been improved using random mutagenesis, selection and genetic engineering with some species and strains now reported to produce up to 100 g/I of extra-cellular (hemi)cellulases (Cherry J R, Fidantsef A L, Opin. Biotechnol. 14(4), 438-443). Such protein production levels have spurred researchers to try and utilize filamentous fungi for the production of recombinant proteins by using strong endogenous promoters, signal peptides, and carrier (hemi)cellulolytic genes fused to the target genes. Very often however, these attempts did not produce the desired or hoped for expression levels of recombinant proteins. For example, during the production of a biological product, such as conventional monoclonal antibodies, unsatisfactory yields were reported ranging from 0.15 g/I in T. reesei to 0.9 g/I in A. niger. Such low amounts of biological product are insufficient for profitable production of proteins in industrial biotechnology, pharmacological and agricultural applications (Nyyssönen et al, 1993, Biotechnology. 11; Ward et al 2004, Appl. Environ. Microbiol. 70).

Many efforts have been undertaken to increase expression levels from filamentous fungi, such as searching for new promoters, deleting regulators such as catabolite repression modulators, introduction of chaperones, and so forth (Nevalainen, 2004, Handbook of fungal biotechnology). But despite all these previous and ongoing efforts, no substantial progress has yet been reported in the yields of recombinant protein production in fungal hosts (Nevalainen et al 2014, Front. Microbiol. 5:75). This is due to the industry's focus on mammalian cell culture technology for such a long time, the fungal cell expression systems such as Trichoderma are not as well established as mammalian cell culture and therefore suffer from drawbacks when expressing a compound of interest. Many problems particularly with the rapid proteolytic degradation of the compound of interest remain.

While others have created Trichoderma fungal cells with one or more proteases inactivated, they have not provided guidance as to which proteases are most relevant to increasing the expression and stability of specific types of proteins, such as heavy chain antibodies or VHH.

For example, WO2011/075677, WO2013/102674 and WO2015/004241 discloses certain proteases that can be knocked out in Trichoderma and even discloses Trichoderma fungal cells that are deficient in multiple proteases. However, WO2011/075677, WO2013/102674 and WO2015/004241 do not provide any guidance regarding which of the proteases have an adverse impact on the expression and stability compounds of interest, such as heavy chain antibodies or VHH, as no examples of expression of any heavy chain antibodies or VHH are described therein. Moreover, WO2011/075677 only discloses heterologous expression of a single fungal protein in each of three different fungal strains deficient in a single protease. Thus, one of skill in the art would likely read WO2011/075677 as teaching that inactivating each single protease would be sufficient for heterologous protein production. Yoon et al (2009, Appl. Microbial Biotechnol 82: 691-701, 2010: Appl. Microbial Biotechnol DOI 10.1007/s00253-010-2937-0) reported the construction of quintuple and tenfold protease gene disruptants for heterologous protein production in Aspergillus oryzae. The 10 protease disruptant cells improve the production yield of chymosin by only 3.8 fold, despite the high number of disrupted protease genes. Van den Hombergh et al reported a triple protease gene disruptant of Aspergillus niger. While the data show a reduction in protease activity, there is no example of any heavy chain antibody or VHH production described herein. WO2002068623 further report Aspergillus niger amp1, sep1 and pep9 proteases, and WO2012048334 reports Myceliophthora thermophila amp2, sep1 and pep9 proteases. Other reports describe the cloning and characterization of sep1 protease in filamentous fungal strains (WO2011077359, WO2009144269, WO200762936 and WO2002045524). The cloning and characterization of pep9 has also been described in WO2012032472, and WO2006110677.

Thus, a need remains in the art for improved filamentous fungal cells, such as Trichoderma fungus cells, that can stably produce heterologous proteins, such as VHH, preferably at high levels of expression.

### SUMMARY OF THE INVENTION

Applicants have surprisingly shown that multiple proteases are relevant to reduction of total protease activity, increasing production of heterologous proteins and stabilizing the heterologous proteins after expression, in filamentous fungal cells, such as Trichoderma fungal cells. In particular, the inventors have identified proteases that are actually expressed in Trichoderma fungal cells (as opposed to merely being coded for in the genome) by performing detailed mass spectrometry analysis on fermentation broths obtained under conditions relevant to the industrial production of compounds of interest such as heavy chain antibodies or VHH. In so doing, previously unrecognized proteases and/or previously unrecognized combination of proteases present in the fermentation broths were identified. Additionally, the inventors confirmed that deleting the genes responsible for the particular protease activities achieved a substantial reduction in total protease activity, which correlates to an increase in production of a compound of interest in filamentous fungal cells containing such deletions.

Applicants have further shown that modifying a regulator of transcription that regulates the transcription of one or more protease genes (for example a GATA-type regulator of transcription such as the AreA or Are1 transcription factor found in filamentous fungal cells), together with deleting or inactivating the genes of the particular proteases as described above, achieved a substantial reduction in total protease activity, which correlates to an increase in production of a compound of interest in filamentous fungal cells containing such deletions. Surprisingly, the increase in the production and/or stability of a compound of interest was shown to be increased to a higher extent than what might be expected when assessing the separate modifications (i.e. modifications of the regulator of transcription or of the particular (set of) protease(s)) in isolation. In other words, combining the modification that leads to reduced or no protease activity of at least one endogenous protease (e.g., deletion of endogenous protease genes) with a further modification in a regulator of transcription (e.g., deletion of a transcription factor that regulates the transcription of one or more proteases) resulted in a synergistic increase in production/stability of the compound of interest.

The present invention provides modified microbial host cells, which may be suitable for the production of compounds of interest, in particular recombinant proteins.

In a first aspect of the invention there is provided a microbial host cell which is characterized by
a. having a modification that leads to a reduced or no protease activity of at least one endogenous protease and,
b. comprising a recombinant polynucleotide encoding a compound of interest;
wherein the at least one endogenous protease is selected from the proteases comprised in the family of serine proteases, metalloproteases, aspartyl proteases and glutamic proteases, and wherein production of the compound of interest from the modified microbial cell is increased compared to the production of the same compound of interest from a parent microbial host cell lacking the modification that leads to a reduced or no protease activity of the at least one endogenous protease.

In a second aspect of the invention there is provided a microbial host cell which is characterized by
a. having a modification that leads to a reduced or no protease activity of at least one endogenous protease and a further modification that affects the production, stability and/or function of a regulator of transcription; and,
b. comprising a recombinant polynucleotide encoding a compound of interest;
wherein the at least one endogenous protease is selected from the proteases comprised in the family of serine proteases, metalloproteases, aspartyl proteases and glutamic proteases, and
wherein production of the compound of interest from the modified microbial cell is increased compared to the production of the same compound of interest from a parent microbial host cell lacking the modification that leads to a reduced or no protease activity of the at least one endogenous protease and lacking the further modification that affects the production, stability and/or function of the regulator of transcription. In some embodiments, production of the compound of interest from the modified microbial cell is increased compared to the production of the same compound of interest from a parent microbial host cell lacking the modification that leads to a reduced or no protease activity of the at least one endogenous protease and/or lacking the further modification that affects the production, stability and/or function of the regulator of transcription.

In a third aspect of the invention there is provided a use of the microbial host in a method for producing a compound of interest.

In a fourth aspect of the invention there is provided method for the production of a compound of interest comprising the steps of: providing the microbial host cell of the invention; culturing the cell such that the compound of interest is produced; optionally isolating the compound of interest.

In a fifth aspect of the invention there is provided a compound of interest produced by the use of the microbial cell of the invention in a method for the production of a compound of interest.

In a sixth aspect of the invention there is provided a method of increasing the production of a compound of interest from a microbial cell comprising the steps of: providing a microbial host cell comprising a recombinant polynucleotide encoding a compound of interest; modifying the microbial host cell such that the modification leads to a reduced or no protease activity of at least one endogenous protease, wherein the at least one endogenous protease is selected from the proteases comprised in the family of serine proteases, metalloproteases, aspartyl proteases and glutamic proteases, and wherein production of the compound of interest from the modified microbial cell is increased compared to the production of the same compound of interest from a parent microbial host cell lacking the modification that leads to a reduced or no protease activity of the at least one endogenous protease.

In a seventh aspect of the invention there is provided a method of increasing the production of a compound of interest from a microbial cell comprising the steps of: providing a microbial host cell comprising a recombinant polynucleotide encoding a compound of interest; modifying the microbial host cell such that the modification leads to a reduced or no protease activity of at least one endogenous protease, and wherein the at least one endogenous protease is selected from the proteases comprised in the family of serine proteases, metalloproteases, aspartyl proteases and glutamic proteases, and further modifying the microbial host cell such that the further modification affects the production, stability and/or function of a regulator of transcription and wherein production of the compound of interest from the modified microbial cell is increased compared to the production of the same compound of interest from a parent microbial host cell lacking the modification that leads to a reduced or no protease activity of the at least one endogenous protease and lacking the further modification that affects the production, stability and/or function of the regulator of transcription. In some embodiments, production of the compound of interest from the modified microbial cell is increased compared to the production of the same compound of interest from a parent microbial host cell lacking the modification that leads to a reduced or no protease activity of the at least one endogenous protease and/or lacking the further modification that affects the production, stability and/or function of the regulator of transcription.

In an eight aspect of the invention, there is provided a method of producing a modified microbial host cell of the invention comprising the steps of: providing a microbial host cell; and modifying the parent microbial host cell, to yield a modified microbial host cell having at least one endogenous protease having reduced or no protease activity.

In a ninth aspect of the invention, there is provided a method of producing a modified microbial host cell of the invention comprising the steps of: providing a microbial host cell; and modifying the parent microbial host cell, to yield a modified microbial host cell having at least one endogenous protease having reduced or no protease activity; and further modifying the microbial host cell such that the further modification affects the production, stability and/or function of a regulator of transcription.

In a tenth aspect of the invention, there is provided a method of producing a modified microbial host cell of the invention comprising the steps of: providing a microbial host cell; and modifying the microbial host cell such that the modification leads to a reduced or no protease activity of at least one endogenous protease, thereby obtaining the modified host cell and where the at least one protease is selected from the family of serine proteases, metalloproteases, aspartyl proteases and glutamic proteases and wherein production of a compound of interest expressed by the modified microbial cell is increased compared to the production of the same compound of interest from a parent microbial host cell lacking the modification that leads to a reduced or no protease activity of the at least one endogenous protease.

In an eleventh aspect of the invention, there is provided a method of producing a modified microbial host cell of the invention comprising the steps of: providing a microbial host cell; and modifying the microbial host cell such that the modification leads to a reduced or no protease activity of at least one endogenous protease, and further modifying the microbial host cell such that the further modification affects the production, stability and/or function of a regulator of transcription, thereby obtaining the modified host cell and where the at least one protease is selected from the family of serine proteases, metalloproteases, aspartyl proteases and glutamic proteases and wherein production of a compound of interest expressed by the modified microbial cell is increased compared to the production of the same compound of interest from a parent microbial host cell lacking the modification that leads to a reduced or no protease activity of the at least one endogenous protease and lacking the further modification that affects the production, stability and/or function of the regulator of transcription. In some embodiments, production of the compound of interest from the modified microbial cell is increased compared to the production of the same compound of interest from a parent microbial host cell lacking the modification that leads to a reduced or no protease activity of the at least one endogenous protease and/or lacking the further modification that affects the production, stability and/or function of the regulator of transcription,

In a still further aspect of the invention, there is provided a kit of parts, wherein the kit comprises one or more of a vector or donor DNA construct for homologous recombination, for example for effecting a full or partial deletion of a gene encoding at least one endogenous protease in the microbial cell and/or for effecting a modification that affects the production, stability and/or function of a gene encoding a regulator of transcription in the microbial cell; and optionally further comprising a microbial host cell and/or a vector encoding a compound of interest.

### BRIEF DESCRIPTION OF THE FIGURES

FIG. 1: Table depicting the presence of protease genes in fermentation broths of Trichoderma. Proteases are identified by their given name, UniProt ID and JGI Genome Portal ID respectively.

FIG. 2: Results of an experiment comparing the stability over the indicated time of a VHH molecule spiked in the fermentation broth of wild-type Trichoderma reesei strain, a Trichoderma reesei strain having a deletion in the are1 gene, a Trichoderma reesei strain having a deletion in 8 different proteases (Δ8x) and a Trichoderma reesei strain having a deletion in the are1 gene and 8 different proteases (Δ8x).

FIG. 3: Results of an experiment comparing the expression of a VHH after 4 days post induction, from a T. reesei wild-type strain WT, T. reesei strain having a deletion in the are1 gene, a T. reesei strain having a deletion in 9 different proteases (Δ9x), a T. reesei strain having deletion in are1 and 9 different proteases (Δ9x), a T. reesei strain having a deletion in 12 different proteases (Δ12x) and a T. reesei strain having a deletion in are1 and 12 different proteases (Δ12x).

### BRIEF DESCRIPTION OF THE SEQUENCE INFORMATION

SEQ ID NO: 1 sets out the amino acid sequence of the protease pep1 from Trichoderma reesei (strain QM6a).

SEQ ID NO: 2 sets out the amino acid sequence of the protease pep1 from Myceliophthora thermophila (strain ATCC 42464).

SEQ ID NO: 3 sets out the amino acid sequence of the protease pep1 from Myceliophthora heterothallica.

SEQ ID NO: 4 sets out the amino acid sequence of the protease pep2 from Trichoderma reesei (strain QM6a).

SEQ ID NO: 5 sets out the amino acid sequence of the protease pep2 from Myceliophthora thermophila (strain ATCC 42464).

SEQ ID NO: 6 sets out the amino acid sequence of the protease pep2 from Myceliophthora heterothallica.

SEQ ID NO: 7 sets out the amino acid sequence of the protease pep3 from Trichoderma reesei (strain QM6a).

SEQ ID NO: 8 sets out the amino acid sequence of the protease pep3 from Myceliophthora thermophila (strain ATCC 42464).

SEQ ID NO: 9 sets out the amino acid sequence of the protease pep3 from Myceliophthora heterothallica.

SEQ ID NO: 10 sets out the amino acid sequence of the protease pep4 from Trichoderma reesei (strain QM6a).

SEQ ID NO: 11 sets out the amino acid sequence of the protease pep4 from Myceliophthora thermophila (strain ATCC 42464).

SEQ ID NO: 12 sets out the amino acid sequence of the protease pep4 from Myceliophthora heterothallica.

SEQ ID NO: 13 sets out the amino acid sequence of the protease pep5 from Trichoderma reesei (strain QM6a).

SEQ ID NO: 14 sets out the amino acid sequence of the protease pep5 from Myceliophthora heterothallica.

SEQ ID NO: 15 sets out the amino acid sequence of the protease pep8 from Trichoderma reesei (strain QM6a).

SEQ ID NO: 16 sets out the amino acid sequence of the protease pep8 from Myceliophthora thermophila (strain ATCC 42464).

SEQ ID NO: 17 sets out the amino acid sequence of the protease pep8 from Myceliophthora heterothallica.

SEQ ID NO: 18 sets out the amino acid sequence of the protease pep9 from Trichoderma reesei (strain QM6a).

SEQ ID NO: 19 sets out the amino acid sequence of the protease pep9 from Myceliophthora thermophila (strain ATCC 42464).

SEQ ID NO: 20 sets out the amino acid sequence of the protease pep9 from Myceliophthora heterothallica.

SEQ ID NO: 21 sets out the amino acid sequence of the protease pep11 from Trichoderma reesei (strain QM6a).

SEQ ID NO: 22 sets out the amino acid sequence of the protease pep11 from Myceliophthora thermophila (strain ATCC 42464).

SEQ ID NO: 23 sets out the amino acid sequence of the protease pep11 from Myceliophthora heterothallica.

SEQ ID NO: 24 sets out the amino acid sequence of the protease pep12 from Trichoderma reesei (strain QM6a).

SEQ ID NO: 25 sets out the amino acid sequence of the protease pep12 from Myceliophthora thermophila (strain ATCC 42464).

SEQ ID NO: 26 sets out the amino acid sequence of the protease pep12 from Myceliophthora heterothallica.

SEQ ID NO: 27 sets out the amino acid sequence of the protease aspX-7 from Trichoderma reesei (strain QM6a).

SEQ ID NO: 28 sets out the amino acid sequence of the protease aspX-7 from Myceliophthora thermophila (strain ATCC 42464).

SEQ ID NO: 29 sets out the amino acid sequence of the protease aspX-7 from Myceliophthora heterothallica.

SEQ ID NO: 30 sets out the amino acid sequence of the protease aspX-11 from Trichoderma reesei (strain QM6a).

SEQ ID NO: 31 sets out the amino acid sequence of the protease aspX-11 from Myceliophthora thermophila (strain ATCC 42464).

SEQ ID NO: 32 sets out the amino acid sequence of the protease aspX-11 from Myceliophthora heterothallica.

SEQ ID NO: 33 sets out the amino acid sequence of the protease gap1 from Trichoderma reesei (strain QM6a).

SEQ ID NO: 34 sets out the amino acid sequence of the protease gap1 from Myceliophthora thermophila (strain ATCC 42464).

SEQ ID NO: 35 sets out the amino acid sequence of the protease gap1 from Myceliophthora heterothallica.

SEQ ID NO: 36 sets out the amino acid sequence of the protease gap2 from Trichoderma reesei (strain QM6a).

SEQ ID NO: 37 sets out the amino acid sequence of the protease slp2 from Trichoderma reesei (strain QM6a).

SEQ ID NO: 38 sets out the amino acid sequence of the protease slp2 from Myceliophthora thermophila (strain ATCC 42464).

SEQ ID NO: 39 sets out the amino acid sequence of the protease slp2 from Myceliophthora heterothallica.

SEQ ID NO: 40 sets out the amino acid sequence of the protease slp6 from Trichoderma reesei (strain QM6a).

SEQ ID NO: 41 sets out the amino acid sequence of the protease slp6 from Myceliophthora thermophila (strain ATCC 42464).

SEQ ID NO: 42 sets out the amino acid sequence of the protease slp6 from Myceliophthora heterothallica.

SEQ ID NO: 43 sets out the amino acid sequence of the protease serX-3 from Trichoderma reesei (strain QM6a).

SEQ ID NO: 44 sets out the amino acid sequence of the protease serX-3 from Myceliophthora thermophila (strain ATCC 42464).

SEQ ID NO: 45 sets out the amino acid sequence of the protease serX-3 from Myceliophthora heterothallica.

SEQ ID NO: 46 sets out the amino acid sequence of the protease serX-9 from Trichoderma reesei (strain QM6a).

SEQ ID NO: 47 sets out the amino acid sequence of the protease serX-9 from Myceliophthora thermophila (strain ATCC 42464).

SEQ ID NO: 48 sets out the amino acid sequence of the protease serX-9 from Myceliophthora heterothallica.

SEQ ID NO: 49 sets out the amino acid sequence of the protease tpp-1 from Trichoderma reesei (strain QM6a).

SEQ ID NO: 50 sets out the amino acid sequence of the protease tpp-1 from Myceliophthora thermophila (strain ATCC 42464).

SEQ ID NO: 51 sets out the amino acid sequence of the protease tpp-1 from Myceliophthora heterothallica.

SEQ ID NO: 52 sets out the amino acid sequence of the protease ser1-4 from Trichoderma reesei (strain QM6a).

SEQ ID NO: 53 sets out the amino acid sequence of the protease ser1-4 from Myceliophthora thermophila (strain ATCC 42464).

SEQ ID NO: 54 sets out the amino acid sequence of the protease ser1-4 from Myceliophthora heterothallica.

SEQ ID NO: 55 sets out the amino acid sequence of the protease ser1-5 from Trichoderma reesei (strain QM6a).

SEQ ID NO: 56 sets out the amino acid sequence of the protease ser1-5 from Myceliophthora thermophila (strain ATCC 42464).

SEQ ID NO: 57 sets out the amino acid sequence of the protease ser1-5 from Myceliophthora heterothallica.

SEQ ID NO: 58 sets out the amino acid sequence of the protease serX-10 from Trichoderma reesei (strain QM6a).

SEQ ID NO: 59 sets out the amino acid sequence of the protease serX-10 from Myceliophthora thermophila (strain ATCC 42464).

SEQ ID NO: 60 sets out the amino acid sequence of the protease serX-10 from Myceliophthora heterothallica.

SEQ ID NO: 61 sets out the amino acid sequence of the protease sep1 from Trichoderma reesei (strain QM6a).

SEQ ID NO: 62 sets out the amino acid sequence of the protease sep1 from Myceliophthora thermophila (strain ATCC 42464).

SEQ ID NO: 63 sets out the amino acid sequence of the protease sep1 from Myceliophthora heterothallica.

SEQ ID NO: 64 sets out the amino acid sequence of the protease slp1 from Trichoderma reesei (strain QM6a).

SEQ ID NO: 65 sets out the amino acid sequence of the protease slp1 from Myceliophthora thermophila (strain ATCC 42464).

SEQ ID NO: 66 sets out the amino acid sequence of the protease slp1 from Myceliophthora heterothallica.

SEQ ID NO: 67 sets out the amino acid sequence of the protease metX-11 from Trichoderma reesei (strain QM6a).

SEQ ID NO: 68 sets out the amino acid sequence of the protease metX-11 from Myceliophthora thermophila (strain ATCC 42464).

SEQ ID NO: 69 sets out the amino acid sequence of the protease metX-11 from Myceliophthora heterothallica.

SEQ ID NO: 70 sets out the amino acid sequence of the protease amp1 from Trichoderma reesei (strain QM6a).

SEQ ID NO: 71 sets out the amino acid sequence of the protease amp1 from Myceliophthora thermophila (strain ATCC 42464).

SEQ ID NO: 72 sets out the amino acid sequence of the protease amp1 from Myceliophthora heterothallica.

SEQ ID NO: 73 sets out the amino acid sequence of the protease amp2 from Trichoderma reesei (strain QM6a).

SEQ ID NO: 74 sets out the amino acid sequence of the protease amp2 from Myceliophthora thermophila (strain ATCC 42464).

SEQ ID NO: 75 sets out the amino acid sequence of the protease amp2 from Myceliophthora heterothallica.

SEQ ID NO: 76 sets out the amino acid sequence of the protease cpa2 from Trichoderma reesei (strain QM6a).

SEQ ID NO: 77 sets out the amino acid sequence of the protease cpa2 from Myceliophthora thermophila (strain ATCC 42464).

SEQ ID NO: 78 sets out the amino acid sequence of the protease cpa2 from Myceliophthora heterothallica.

SEQ ID NO: 79 sets out the amino acid sequence of the protease cpa3 from Trichoderma reesei (strain QM6a).

SEQ ID NO: 80 sets out the amino acid sequence of the protease cpa5 from Trichoderma reesei (strain QM6a).

SEQ ID NO: 81 sets out the amino acid sequence of the protease cpa5 from Myceliophthora thermophila (strain ATCC 42464).

SEQ ID NO: 82 sets out the amino acid sequence of the protease cpa5 from Myceliophthora heterothallica.

SEQ ID NO: 83 sets out the amino acid sequence of the protease met1-1 from Trichoderma reesei (strain QM6a).

SEQ ID NO: 84 sets out the amino acid sequence of the protease met1-1 from Myceliophthora thermophila (strain ATCC 42464).

SEQ ID NO: 85 sets out the amino acid sequence of the protease met1-1 from Myceliophthora heterothallica.

SEQ ID NO: 86 sets out the amino acid sequence of the protease met1-2 from Trichoderma reesei (strain QM6a).

SEQ ID NO: 87 sets out the amino acid sequence of the protease met1-2 from Myceliophthora thermophila (strain ATCC 42464).

SEQ ID NO: 88 sets out the amino acid sequence of the protease met1-2 from Myceliophthora heterothallica.

SEQ ID NO: 89 sets out the amino acid sequence of the protease met1-3 from Trichoderma reesei (strain QM6a).

SEQ ID NO: 90 sets out the amino acid sequence of the protease met1-3 from Myceliophthora thermophila (strain ATCC 42464).

SEQ ID NO: 91 sets out the amino acid sequence of the protease met1-3 from Myceliophthora heterothallica.

SEQ ID NO: 92 sets out the amino acid sequence of the protease met1-6 from Trichoderma reesei (strain QM6a).

SEQ ID NO: 93 sets out the amino acid sequence of the protease met1-6 from Myceliophthora thermophila (strain ATCC 42464).

SEQ ID NO: 94 sets out the amino acid sequence of the protease met1-6 from Myceliophthora heterothallica.

SEQ ID NO: 95 sets out the amino acid sequence of the protease met1-7 from Trichoderma reesei (strain QM6a).

SEQ ID NO: 96 sets out the amino acid sequence of the protease met1-7 from Myceliophthora thermophila (strain ATCC 42464).

SEQ ID NO: 97 sets out the amino acid sequence of the protease met1-7 from Myceliophthora heterothallica.

SEQ ID NO: 98 sets out the amino acid sequence of the protease vacX-1 from Trichoderma reesei (strain QM6a).

SEQ ID NO: 99 sets out the amino acid sequence of the protease vacX-1 from Myceliophthora thermophila (strain ATCC 42464).

SEQ ID NO: 100 sets out the amino acid sequence of the protease vacX-1 from Myceliophthora heterothallica.

SEQ ID NO: 101 sets out the amino acid sequence of the protease tsp-1 from Trichoderma reesei (strain QM6a).

SEQ ID NO: 102 sets out the amino acid sequence of a target transcription factor of the invention (i.e. a transcription factor which is modified to affect its production, stability and/or function). This example is the sequence of Are1 from Trichoderma reesei.

SEQ ID NO: 103 sets out the genomic nucleotide sequence encoding a target transcription factor of the invention.

SEQ ID NO: 104 sets out a nucleotide sequence encoding a target transcription factor of the invention.

SEQ ID NO: 105 sets out the amino acid sequence of a further target transcription factor of the invention (i.e. a further transcription factor which is modified to affect its production, stability and/or function). This example is the sequence of AreA from Myceliophthora heterothallica.

SEQ ID NO: 106 sets out the genomic nucleotide sequence encoding the further target transcription factor of SEQ ID NO: 105.

SEQ ID NO: 107 sets out a nucleotide sequence encoding the further target transcription factor of SEQ ID NO: 105.

SEQ ID NO: 108 sets out the sequence of a GATA-type zinc finger domain present in target polypeptides of the invention.

SEQ ID NO: 109 is the DNA sequence bound by GATA-type zinc fingers.

SEQ ID NO: 110 sets out the amino acid sequence of a further target polypeptide of the invention (i.e. a further polypeptide which is modified to affect its production, stability and/or function). This example is the amino acid sequence of AreA from Myceliophthora thermophila.

SEQ ID NO: 111 sets out the genomic nucleotide sequence encoding the further target transcription factor of SEQ ID NO: 110.

SEQ ID NO: 112 sets out the genomic nucleotide sequence encoding the further target transcription factor of SEQ ID NO: 110.

SEQ ID NO: 113 sets out the amino acid sequence of a further target transcription factor of the invention (i.e. a further transcription factor which is modified to affect its production, stability and/or function). This example is the amino acid sequence the amino acid sequence of AreA from Aspergillus nidulans SEQ ID NO: 114 sets out the genomic nucleotide sequence encoding the further transcription factor of SEQ ID NO: 113.

SEQ ID NO: 115 sets out the genomic nucleotide sequence encoding the further target transcription factor of SEQ ID NO: 113.

SEQ ID NO: 116 is an alternative polypeptide sequence of Are1 from Trichoderma reesei.

SEQ ID NO: 117 is an alternative polypeptide sequence of AreA from Myceliophthora heterothallica.

SEQ ID NOs: 118 to 122 are the sequence of VHH-1, where SEQ ID NO: 118 is the full length sequence of VHH-1, SEQ ID NO: 119 is the full length sequence of VHH-1 but in which the first residue is changed to a Q residue, SEQ ID NO: 120 is the CDR1 of VHH-1, SEQ ID NO: 121 is the CDR2 of VHH-1 and SEQ ID NO: 122 is the CDR3 of VHH-1.

SEQ ID NOs: 123 to 126 and 131 are the sequences of VHH-2, where SEQ ID NO: 123 is the full length sequence of VHH-1, SEQ ID NO: 131 is the full length sequence of VHH-2 but in which the first residue is changed to a D residue, SEQ ID NO: 124 is the CDR1 of VHH-2, SEQ ID NO: 125 is the CDR2 of VHH-2 and SEQ ID NO: 126 is the CDR3 of VHH-2.

SEQ ID NOs: 127 to 130 and 132 are the sequences of VHH-3, where SEQ ID NO: 127 is the full length sequence of VHH-1, SEQ ID NO: 132 is the full length sequence of VHH-3 but in which the first residue is changed to a D residue, SEQ ID NO: 128 is the CDR1 of VHH-3, SEQ ID NO: 129 is the CDR2 of VHH-3 and SEQ ID NO: 130 is the CDR3 of VHH-3.

SEQ ID NO: 133 sets out the amino acid sequence of the protease serX-4 from Trichoderma reesei (strain QM6a).

SEQ ID NO: 134 sets out the amino acid sequence of the protease serX-5 from Trichoderma reesei (strain QM6a).

SEQ ID NO: 135 sets out the amino acid sequence of the protease serX-5 from Myceliophthora thermophila (strain ATCC 42464).

SEQ ID NO: 136 sets out the amino acid sequence of the protease serX-5 from Myceliophthora heterothallica.

SEQ ID NO: 137 sets out the amino acid sequence of the protease metX-12 from Trichoderma reesei (strain QM6a).

SEQ ID NO: 138 sets out the amino acid sequence of the protease metX-12 from Myceliophthora thermophila (strain ATCC 42464).

SEQ ID NO: 139 sets out the amino acid sequence of the protease metX-12 from Myceliophthora heterothallica.

The sequence identity (percentages) between SEQ ID NOs: 102, 105, 110, 113, 116 and 117 is shown below:

| | SEQ ID | SEQ ID | SEQ ID | SEQ ID | SEQ ID | SEQ ID |
|---|---|---|---|---|---|---|
| | NO: 102 | NO: 105 | NO: 110 | NO: 113 | NO: 116 | NO: 117 |
| T. reesei Are1 (SEQ ID 102) | 100 | 43.5 | 43 | 36.7 | 99.4 | 43.4 |
| M. heterothallica AreA (SEQ ID | 42.3 | 100 | 93.5 | 37.2 | 42.4 | 98.5 |
| 105) | | | | | | |
| M. thermophilla AreA (SEQ ID | 43.2 | 94 | 100 | 36.7 | 43.2 | 94.4 |
| 110) | | | | | | |
| A. nidulans AreA (SEQ ID 113) | 37.3 | 36.4 | 36.1 | 100 | 37.3 | 36.7 |
| T. reesei Are1 (SEQ ID 116) | 99.4 | 43.6 | 43 | 36.7 | 100 | 43.5 |
| M. heterothallica AreA (SEQ ID | 48.1 | 98.5 | 94.9 | 37.1 | 48.1 | 100 |
| 117) | | | | | | |

### DETAILED DESCRIPTION OF THE INVENTION

Reference to any prior art in this specification is not, and should not be taken as, an acknowledgment or any form of suggestion that this prior art forms part of the common general knowledge in any country.

All documents cited in the present specification are hereby incorporated by reference in their entirety. Unless otherwise defined, all terms used in disclosing the invention, including technical and scientific terms, have the meaning as commonly understood by one of ordinary skill in the art to which this invention belongs.

The present invention will be described with respect to particular embodiments but the invention is not limited thereto but only by the claims. Any reference signs in the claims shall not be construed as limiting the scope.

Where the term “comprising” is used in the present description and claims, it does not exclude other elements or steps.

Where an indefinite or definite article is used when referring to a singular noun e.g. “a” or “an”, “the”, this includes a plural of that noun unless something else is specifically stated.

The term “about” as used herein when referring to a measurable value such as a parameter, an amount, a temporal duration, and the like, is meant to encompass variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier ‘about’ refers is itself also specifically, and preferably, disclosed.

The following terms or definitions are provided solely to aid in the understanding of the invention. Unless specifically defined herein, all terms used herein have the same meaning as they would to one skilled in the art of the present invention. Practitioners are particularly directed to Sambrook et al., Molecular Cloning: A Laboratory Manual, 2^{nd} ed., Cold Spring Harbor Press, Plainsview, New York (1989); and Ausubel et al., Current Protocols in Molecular Biology (Supplement 47), John Wiley & Sons, New York (1999), for definitions and terms of the art. The definitions provided herein should not be construed to have a scope less than understood by a person of ordinary skill in the art.

Unless indicated otherwise, all methods, steps, techniques and manipulations that are not specifically described in detail can be performed and have been performed in a manner known per se, as will be clear to the skilled person. Reference is for example again made to the standard handbooks, to the general background art referred to above and to the further references cited therein.

The following invention relates to a microbial cell, such as a fungal host cell, which has been modified, and where this modification affects the production, stability and/or function of at least one endogenous protease, and where the at least one endogenous protease is selected from the family of serine proteases, metalloproteases, aspartyl proteases and glutamic proteases. In preferred embodiments this modification leads to a reduced or no protease activity of at least one endogenous protease selected from the family of serine proteases, metalloproteases, aspartyl proteases and glutamic proteases. Hence, the modified microbial host cell of the invention has a decrease in protease activity if compared with a parent microbial host cell lacking the modification and when measured under the same or substantially the same conditions. A reduction or deficiency in protease activity may be particularly suited for embodiments relating to the production of a compound of interest, in particular a proteinaceous compound of interest and where the reduced or no protease activity in the at least one protease leads to an increase in production of a compound of interest.

For example, it has been surprisingly found that when the modified microbial host cell according to the invention, and which is further capable of expressing a compound of interest, is used in a method to produce a compound of interest, for example a polypeptide, an improved yield of said compound is obtained if compared to a method in which a parent host cell is used and measured under the same or substantially the same conditions.

In addition, it has been found that when the microbial host cell according to the invention which has been modified and where this modification affects the production, stability and/or function of at least one endogenous protease, and which is capable of expressing a compound of interest, is used in a method to produce a compound of interest, the fermentation broth or cell culture medium comprising the microbial host cell and/or the intracellular environment of the microbial host cell may demonstrate a reduction in protease activity compared to a method in which a parent host cell is used and measured under the same or substantially the same conditions.

Surprisingly, the reduction in protease activity in the fermentation broth (or cell culture medium) or intracellular environment of the modified microbial host cell according to this invention and capable of expressing a compound of interest, increases in the yield of the compound of interest, such as a polypeptide produced by the modified microbial host cell. Thus, the production level of the compound of interest from the modified microbial cell is increased compared to the production level of the same compound of interest as produced from the parent microbial host cell lacking the modification. Additionally, the reduced production and/or activity of proteases from the modified microbial host cell according to this invention can lead to increased stability of the compound of interest over time, due to a reduction in proteolytic degradation of the compound of interest. In this regard, the reduced production and/or activity of proteases from the modified microbial host cell according to this invention may lead to an increased shelf-life and storage stability of the compound of interest produced by the microbial host cell according to this invention.

The microbial host cell according to this invention and the production of a compound of interest can be useful for the industrial production of compounds of interest such as heterologous polypeptides. Heterologous polypeptides may be useful in the preparation of agrochemical or pharmaceutical compositions.

Microbial Host Cells and Methods for Making them

The present invention provides modified microbial cells, specifically microbial host cells. This modification affects the production, stability and/or function of one or more endogenous proteases.

A “microbial host cell which has been modified” or a “modified microbial host cell” is herewith defined as a microbial host cell which has been modified, to obtain a different genotype and/or a different phenotype if compared to an unmodified parent host cell. The modified microbial host cell may have one or multiple alterations (e.g., genetic alterations), relative to the unmodified parent host cell, that result in the different genotype and/or different phenotype. Thus, the term “modification”, as used herein, can encompass more than one alteration to the host cell. The modification can either be affected by, for example:
a. subjecting the parent microbial host cell to recombinant genetic manipulation techniques;
b. subjecting the parent microbial host cell to (classical) mutagenesis; and/or
c. subjecting the parent microbial host cell to an inhibiting compound or composition.

In preferred embodiments, the modification may be a genetic modification.

A “modification that leads to a reduced or no protease activity of at least one endogenous protease” means that at least one endogenous protease is reduced in its activity and/or its intracellular and/or extracellular concentration, when compared to the parent host cell and measured under the same or substantially the same conditions. Similarly, a “modification that affects the production, stability and/or function of a regulator of transcription” means that the regulator of transcription is modulated in its activity and/or its intracellular and/or extracellular concentration is modulated when compared to the parent host cell and measured under the same or substantially the same conditions.

“Production” of a polypeptide or a compound of interest refers to production of a polypeptide or a compound of interest by the microbial host cell. In the context of the present invention, “production” refers to the quantity of the intact compound or polypeptide produced, i.e., not including degraded fragments, e.g., proteolytic fragments, of the compound or polypeptide. The level of production may be assessed by quantifying the amount of compound or polypeptide present in a cell culture broth during or after culturing the modified microbial host cell, for example. “Stability and/or function” of a polypeptide or a compound of interest refers to the stability and/or function of a polypeptide or a compound of interest inside or outside the microbial cell. In this regard, an increase in “stability” of a compound of interest may refer to a decreased rate of degradation of the compound over time. For example, a purified or partially purified compound of interest may have increased “stability” if degradation of the compound is reduced due to a reduction in activity of residual endogenous proteases (e.g., due to reduction in the amount of the proteases present).

As used herein, a modification, modified or a similar term in the context of polynucleotides may refer to modification in a coding or non-coding region of the polynucleotide, such as a regulatory sequence, 5′ untranslated region, 3′ untranslated region, up-regulating genetic element, down-regulating genetic element, enhancer, suppressor, promoter, exon and/or intron region. Modifications may be made to a polynucleotide coding for the at least one endogenous protease in the microbial host cell to achieve modification of the at least one polypeptide. As used herein a modification, modified or a similar term in the context of polypeptides, in particular a modification that affects the production, stability and/or function of a polypeptide, may refer to a modification of a polynucleotide coding for the at least one endogenous protease. The polynucleotides that are modified in the present invention are polynucleotides that are present in the genome of the parental or wild-type microbial host cell. The modification of these polynucleotides in turn leads to modification of the at least one endogenous proteases encoded by those polynucleotides.

A modification, modified or a similar term can be a genetic modification, for example a genetic modification in the gene(s) or polynucleotide(s) encoding the at least one endogenous protease. Preferably, the genetic modification is a partial or full deletion, that is a partial or full deletion of the gene(s) or polynucleotide(s) encoding the at least one endogenous protease. Methods for deleting or partially deleting genes in microbial host cells are well known in the art and include the methods described in Example 5. In such deletions, the genomic DNA containing the genetic information for the production of the at least one polypeptide of a microbial host cell is removed in its entirety or where at least one nucleotide is removed leading to the modified microbial host cell to produce less of the at least one endogenous protease or produces substantially no polypeptide and/or produces a polypeptide having a decreased activity or decreased specific activity or a polypeptide having no activity or no specific activity. The at least one endogenous protease is therefore coded for by the gene(s) or polynucleotide(s) in the parental microbial host cell genome. The gene(s) or polynucleotide(s) encoding the at least one endogenous protease may be absent from the genome of the modified microbial host cell (for example in the case of a full deletion) or the gene(s) or polynucleotide(s) may simply be modified to alter its production, stability and/or function.

A modification, modified or a similar term can also be one or more mutations performed by specific or random mutagenesis, nucleotide insertion and/or nucleotide substitution and/or nucleotide deletion. Preferably, the one or more mutations are in the gene(s) or polynucleotide(s) encoding the at least one endogenous protease. Such modifications lead to the modified microbial host cell to produce less of the at least one endogenous protease or produce substantially no polypeptide and/or produces a polypeptide having a decreased activity or decreased specific activity or a polypeptide having no activity or no specific activity.

A modification, modified or a similar term can also involve targeting the at least one endogenous protease, its corresponding chromosomal gene and/or its corresponding mRNA by techniques well known in the art such as anti-sense techniques, RNAi techniques, CRISPR techniques, ADAR techniques, Zinc-finger nuclease (ZFN) techniques, transcription activator-like effector nuclease (TALEN) techniques, a small molecule inhibitor, antibody, antibody fragment or a combination thereof leading to the modified microbial host cell to produces less of the at least one endogenous protease or produces substantially no polypeptide and/or produces a polypeptide having a decreased activity or decreased specific activity or a polypeptide having no activity or no specific activity and/or where an interaction with the at least one endogenous protease by specific or non-specific binding leads to degradation, precipitation of the at least one endogenous protease, or where this interaction leads to the at least one endogenous protease having decreased activity or decreased specific activity or a having no activity or no specific activity.

A microbial host cell, be it a microbial host cell which has been modified or a parent host cell, is defined here as a single cellular organism used during a fermentation process or during cell culture to produce a compound of interest. Preferably, a microbial host cell is selected from the kingdom Fungi. In particular, the fungus may be a filamentous fungus.

In some embodiments the microbial host cell may preferably be from the division Ascomycota, subdivision Pezizomycotina. In some embodiments, the fungi may preferably from the Class Sordariomycetes, optionally the Subclass Hypocreomycetidae. In some embodiments, the fungi may be from an Order selected from the group consisting of Hypocreales, Microascales, Eurotiales, Onygenales and Sordariales. In some embodiments, the fungi may be from a Family selected from the group consisting of Hypocreaceae, Nectriaceae, Clavicipitaceae and Microascaceae. In some more specific embodiments, the fungus may be from a Genus selected from the group consisting of Trichoderma (anamorph of Hypocrea), Myceliophthora, Fusarium, Gibberella, Nectria, Stachybotrys, Claviceps, Metarhizium, Villosiclava, Ophiocordyceps, Cephalosporium, Neurospora, Rasamsonia and Scedosporium. In some further and more specific embodiments, the fungi may be selected from the group consisting of Trichoderma reesei (Hypocrea jecorina), T. citrinoviridae, T. longibrachiatum, T. virens, T. harzianum, T. asperellum, T. atroviridae, T. parareesei, Fusarium oxysporum, F. gramineanum, F. pseudograminearum, F. venenatum, Gibberella fujikuroi, G. moniliformis, G. zeaea, Nectria (Haematonectria) haematococca, Stachybotrys chartarum, S. chlorohalonata, Claviceps purpurea, Metarhizium acridum, M. anisopliae, Villosiclava virens, Ophiocordyceps sinensis, Neurospora crassa, Rasamsonia emersoniim, Acremonium (Cephalosporium) chrysogenum, Scedosporium apiospermum, Aspergillus niger, A. awamori, A. oryzae, A. nidulans, Chrysosporium lucknowense, Thermothelomyces thermophilus, Myceliophthora thermophila, Myceliophthora heterothallica, Thermothelomyces heterothallica, Humicola insolens, and Humicola grisea, most preferably Trichoderma reesei or Myceliophthora heterothallica. If the host cell is a Trichoderma reesei cell, it may be selected from the following group of Trichoderma reesei strains obtainable from public collections: QM6a, ATCC13631; RutC-30, ATCC56765; QM9414, ATCC26921, RL-P37 and derivatives thereof. If the host cell is a Myceliophthora heterothallica, it may be selected from the following group of Myceliophthora heterothallica or Thermothelomyces thermophilus strains: CBS 131.65, CBS 203.75, CBS 202.75, CBS 375.69, CBS 663.74 and derivatives thereof. If the host cell is a Myceliophthora thermophila it may be selected from the following group of Myceliophthora thermophila strains ATCC42464, ATCC26915, ATCC48104, ATCC34628, Thermothelomyces heterothallica C1, Thermothelomyces thermophilus M77 and derivatives thereof. If the host cell is an Aspergillus nidulans it may be selected from the following group of Aspergillus nidulans strains: FGSC A4 (Glasgow wild-type), GR5 (FGSC A773), TNO2A3 (FGSC A1149), TNO2A25, (FGSC A1147), ATCC 38163, ATCC 10074 and derivatives thereof. Within the context of the present invention “measured under the same conditions” or “measured under substantially the same conditions” means that the microbial host cell which has been modified and the parent microbial host cell are cultured under the same conditions and a certain aspect related to the microbial host cell is measured in the microbial host cell which has been modified, and in the parent host cell, respectively, using the same conditions, preferably by using the same assay and/or methodology, more preferably within the same experiment. The same conditions refers to the culture conditions (e.g., temperature, pH, dissolved oxygen concentration, inoculation density etc) used to culture the parent and modified microbial host cell. The same conditions may also refer to the use of the same assay to determine protease activity or production of a compound of interest in a cultured parent microbial host cell and a cultured modified microbial host cell.

For example, in some embodiments, the method for measuring protease activity comprises providing a microbial cell whose protease activity is to be measured, culturing the microbial host cell in a cell culture medium, and measuring the level of protease activity in the culture broth, for example either by obtaining a sample of the culture broth and determining its protease activity by measuring the ability of the broth sample to degrade a test protein, or spiking the culture broth with a test protein (i.e. adding a quantity of test protein to the cell culture medium) and measuring the extent of the degradation of the test protein in the culture broth over time (e.g., by SDS-PAGE as described in Example 3), or by identifying and/or quantifying the proteases present in the broth sample by mass spectrometry techniques for example by liquid chromatography-tandem mass spectrometry (LC-MS/MS), e.g., as described in Example 1.

In some embodiments, the method for measuring protease activity comprises providing a microbial cell whose protease activity is to be measured, culturing the microbial cell in a liquid cell culture medium at 28° C. for 48 hours, followed by adding a test protein to the liquid cell culture medium (for example 500 μL of monoclonal antibody solution having a concentration of 30 mg/mL), obtaining one or more samples of the liquid cell culture medium at periodic intervals and measuring the level of intact test protein (and/or measuring the extent of degradation of the test protein) in each sample to determine the protease activity of the microbial host cell. Alternatively, the test protein may be casein and the level of protease activity is estimated by measuring the level of free tyrosine released during proteolytic degradation of casein in one or more samples of the liquid cell culture. Alternatively, the casein is fluorescein-labeled casein, allowing for a fluorometric readout of the protease activity present in one or more samples of the liquid cell culture. The method may further comprise carrying out the same method on a test microbial host cell that has not been modified (i.e. a parent microbial host cell) and comparing the rate and/or extent of degradation of the test protein with the modified microbial host cell to quantify the change in protease activity caused by the modification to the microbial host cell.

Other methods of determining a change in protease activity as a result of one or more modifications to the microbial host cell leading to a reduced or no protease activity in at least on endogenous protease will be apparent to the skilled person. For example, when the microbial host cell comprises a nucleotide sequence coding for a compound of interest (i.e. a heterologous nucleotide sequence coding for a compound of interest), the method may comprise culturing the microbial host cell in a cell culture medium under conditions to cause production of the compound of interest by the microbial host cell, obtaining one or more samples of the liquid cell culture medium at periodic intervals and measuring the concentration of the compound of interest in each sample to determine the protease activity of the microbial host cell, whereby an increase in the concentration of the compound of interest, in this example, is indicative of a reduction in protease activity. The method may further comprise carrying out the same method on a test microbial host cell that has not been modified (i.e. a parent microbial host cell comprising the nucleotide sequence coding for the compound of interest) and comparing the concentration of the compound of interest in the cell culture medium with the concentration of the compound of interest in the cell culture medium for the modified microbial host cell to quantify a change in protease activity caused by the modification to the microbial host cell. Similar methods can be used to determine the level of production (i.e., yield) of the compound of interest, for example culturing the microbial host cell in a cell culture medium under conditions to cause production of the compound of interest by the microbial host cell, obtaining one or more samples of the liquid cell culture medium at periodic intervals and measuring the concentration of the compound of interest in each sample to determine the level of production (i.e., yield) of the compound of interest. The method may further comprise carrying out the same method on a test microbial host cell that has not been modified (i.e. a parent microbial host cell comprising the nucleotide sequence coding for the compound of interest) and comparing the concentration of the compound of interest in the cell culture medium with the concentration of the compound of interest in the cell culture medium for the modified microbial host cell to quantify a change in level of production (i.e., yield) of the compound of interest caused by the modification to the microbial host cell.

In some embodiments obtaining a sample of the culture broth can include the step of removing the microbial host cell before obtaining a sample, or a sample of the culture broth can contain both the culture broth as the microbial host cell, or the microbial host cell can be lysed prior to taking a sample of the culture broth. In other embodiments the compound of interest can be further isolated or purified using techniques well known in the art such as filtration of chromatography before one or more samples are obtained. In some embodiments the compound of interest can be formulated in an agrochemically or pharmaceutically suitable formulation before one or more samples are obtained. The skilled person will be aware that proteases can be carried over in a purified and/or formulated end product or intermediate product and that differences in stability and/or shelf life may exist between a compound of interest produced by the modified microbial host cell of the invention and the same compound produced by the parent microbial host cell and where the difference can be measured by for example the methods as set out here.

As the activity of a protease occurs over a period of time, when making comparisons in the protease activity between modified and parental microbial host cells, the skilled person will be aware the comparison may be made using protease activity measurement determined after the same culture time (i.e. after the modified and parental microbial host cells have been cultured for the same length of time). In addition, or as an alternative, the skilled person will be aware that the comparisons may be made using protease activity measurements from cultures that contain a similar amount of the microbial host cells. The skilled person will be aware that the comparison may be made using protease activity measurements starting from samples containing similar amounts of the microbial host cell (i.e. by making appropriate dilutions or concentrating samples before measurements). Similarly, when making comparisons in the yield of the compound of interest produced by modified and parental microbial host cells, the skilled person will be aware the comparison may be made using compound yield determined after the same culture time and/or starting from samples with the same amount of microbial host cell. This is simply an extension of the concept of measuring the protease activity and/or the compound yield under the same or substantially the same conditions for both the modified microbial host cell and the parental microbial host cell, and the skilled person would understand how to compare the protease activity between the modified and parental microbial host cells in this way.

A “parent microbial host cell” or “parental microbial host cell” is defined as a microbial host cell that has not been modified to affect the production, stability and/or function of the at least one endogenous protease (and hence may be referred to as an unmodified microbial host cell). The parent microbial host cell therefore lacks one or more genetic modifications that affect the production, stability and/or function of the at least one endogenous protease, and/or the parent microbial host cell is not subjected to an inhibiting compound or composition, wherein the inhibiting compound or composition affects the production, stability and/or function of the at least one endogenous protease. The parent microbial host cell does need not be the exact cell from which the modified host cell was obtained. However, the parent microbial host cell will generally be genetically identical to the modified microbial host cell, with the exception of the modification (if the modification is a genetic modification) that leads to a reduced or no protease activity of at least one endogenous protease. Thus, the parent microbial host cell comprises a recombinant polynucleotide encoding the same compound of interest as the modified microbial host cell. The parent microbial host cell may therefore be considered a wild-type host cell (and can be referred to herein as such), since the host cell has not been modified to affect the production, stability and/or function of the at least one endogenous protease. Generally therefore, a parent microbial host cell will not have been modified to cause a reduction or deficiency in protease activity.

In some embodiments, the parent host cell has not been modified in the same way as the modified host cell. In other words, the parent host cell may have undergone modification, but it has not undergone the modification to affect the production, stability and/or function of the at least one endogenous protease. Thus, the parent host cell may not have a reduction in protease activity.

In specific embodiments the parent microbial host cell may already have been modified to reduce protease activity and the modified microbial host cell is characterized by having an additional modification over the parent microbial host cell. In other words, the modified microbial host cell has one or more additional modifications leading to a reduced or no protease activity in at least one endogenous protease and where these additional modifications lead to an even further reduction in protease activity. For example, the parent microbial cell may have a deletion of a gene (e.g., Are1) which results in a decrease in production and/or secretion of proteases by the host cell. In this context, the modified microbial host cell would have the same deletion and a further modification leading to a reduced or no protease activity of at least one endogenous protease.

With a reduction or deficiency in protease activity it is meant that in the cell culture media or fermentation broth and/or the intracellular environment of a modified microbial host cell, at least one endogenous protease is reduced in activity and/or abundance when compared with a parent microbial host cell and measured under the same conditions. So, when the modified microbial cells are cultured in a culture medium or fermentation broth, the protease activity of the at least one endogenous protease is reduced relative to the parent microbial cell when cultured in the same culture medium or fermentation broth under the same culture conditions (e.g., temperature, pH etc). The reduction in protease activity of the at least one endogenous protease may result in a decrease in the total protease activity (i.e., the overall protease activity of all proteases present).

A reduction in protease activity of the at least one endogenous protease may be at least about 1% less protease activity if compared with the parent host cell and measured under the same conditions, at least about 5% less, at least about 10% less, at least about 20% less, at least about 30% less, at least about 40% less, at least about 50% less, at least about 60% less, at least about 70% less, at least about 80% less, at least about 90% less, at least about 91% less, at least about 92% less, at least about 93% less, at least about 94% less at least about 95% less, at least about 96% less, at least about 97% less, at least about 98% less, at least about 99% less, or at least about 99.9% less, or the modified microbial host cell has substantially no protease activity of the at least one endogenous protease if compared with the parent host cell and measured under the same conditions. In preferred embodiments, the modified microbial host cell may have at least about a 40% reduction in protease activity of the at least one endogenous protease compared to the parent microbial host cell. More preferably, the modified microbial host cell may have at least about a 90% reduction in protease activity of the at least one endogenous protease, or no protease activity of the at least one endogenous protease, when compared to the parent microbial host cell.

A reduction in total protease activity may be at least about 1% less protease activity if compared with the parent host cell and measured under the same conditions, at least about 5% less, at least about 10% less, at least about 20% less, at least about 30% less, at least about 40% less, at least about 50% less, at least about 60% less, at least about 70% less, at least about 80% less, at least about 90% less, at least about 91% less, at least about 92% less, at least about 93% less, at least about 94% less at least about 95% less, at least about 96% less, at least about 97% less, at least about 98% less, at least about 99% less, or at least about 99.9% less, or the modified microbial host cell has substantially no protease activity if compared with the parent host cell and measured under the same conditions. In preferred embodiments, the total protease activity is at least about 1% less, preferably about 10% or less, more preferably about 40% less than the total protease activity of the parent microbial host cell.

Total protease activity may be quantified by measuring the protease activity present in the culture media or fermentation broth containing the modified microbial host cell. In one embodiment the culture media or fermentation broth containing the microbial host cell that has been modified contains a protease activity which is reduced by at least about 1% if compared with the culture media or fermentation broth of the parent host cell and measured under the same conditions, for example at least about 5% less, at least about 10% less, at least about 20% less, at least about 30% less, at least about 40% less, at least about 50% less, at least about 60% less, at least about 70% less, at least about 80% less, at least about 90% less, at least about 91% less, at least about 92% less, at least about 93% less, at least about 94% less at least about 95% less, at least about 96% less, at least about 97% less, at least about 98% less, at least about 99% less, or at least about 99.9% less, or the culture media or fermentation broth contains substantially no protease activity if compared with the parent microbial host cell and measured under the same conditions. In preferred embodiments the culture media or fermentation broth containing microbial host cells that have been modified has a protease activity which is reduced by at least 40% compared to culture media or fermentation broth containing microbial host cells that have not been modified (i.e. a parental microbial host cells). “Containing” in this context refers to the culture media or fermentation broth that has been used to culture or ferment either the modified microbial host cell, or a parental microbial host cell.

In another embodiment the intracellular environment of the microbial host that has been modified contains a protease activity which is reduced by at least about 1% if compared with the culture media or fermentation broth of the parent microbial host cell and measured under the same conditions, for example at least about 5% less, at least about 10% less, at least about 20% less, at least about 30% less, at least about 40% less, at least about 50% less, at least about 60% less, at least about 70% less, at least about 80% less, at least about 90% less, at least about 91% less, at least about 92% less, at least about 93% less, at least about 94% less at least about 95% less, at least about 96% less, at least about 97% less, at least about 98% less, at least about 99% less, or at least about 99.9% less, or the culture media or fermentation broth contains no substantially no protease activity if compared with the parent microbial host cell and measured under the same conditions. In preferred embodiments the intracellular environment of the microbial host that has been modified contains a protease activity which is reduced by at least 40% compared to a microbial host cell that has not been modified (i.e. a parental microbial host cell).

A reduction in intracellular protease activity may be a result of a reduction in the production, stability and/or function of the protease. A reduction in extracellular protease activity (i.e. activity of protease in the culture media or fermentation broth) may be a result of a reduction in the production, stability and/or function of the protease. Alternatively or additionally, the reduction may be a result of a reduction in the secretion of the protease into the culture media or fermentation broth. Accordingly, in some embodiments, the modified microbial host cell may have a reduction in the secretion of one or more proteases compared with a parent microbial host cell which has not been modified.

In embodiments in which the filamentous microbial host cell comprises a recombinant polynucleotide encoding a heterologous polypeptide, the reduction in protease activity (for example at least about a 40% reduction) may be present during conditions suitable for or conducive to the production of the compound of interest by the filamentous fungal cell. In this way, the filamentous fungal cell can produce higher yields of the compound of interest.

With a “heterologous polypeptide” it is meant any recombinant protein such as an antibody or a functional fragment thereof, a carbohydrate binding domain, a heavy chain antibody or a functional fragment thereof, a single domain antibody, a heavy chain variable domain of an antibody or a functional fragment thereof, a heavy chain variable domain of a heavy chain antibody or a functional fragment thereof, a variable domain of camelid heavy chain antibody (VHH) or a functional fragment thereof, a variable domain of a new antigen receptor a variable domain of shark new antigen receptor (vNAR) or a functional fragment thereof, a minibody, a nanobody, a nanoantibody, an affibody, an alphabody, a designed ankyrin-repeat domain, an anticalins, a knottins or an engineered CH2 domain. In some embodiments, the heterologous polypeptide is an antibody, for example a VHH.

In some embodiments, the heterologous polypeptide is a therapeutic protein, biosimilar, multi-domain protein, peptide hormone, antimicrobial peptide, peptide, carbohydrate binding module, enzyme, cellulase, protease, protease inhibitor, aminopeptidase, amylase, carbohydrase, carboxypeptidase, catalase, chitinase, cutinase, deoxyribonuclease, esterase, alpha-galactosidase, beta-galactosidase, glucoamylase, alpha-glucosidase, beta-glucosidase, invertase, laccase, lipase, mannanase, mutanase, oxidase, pectinolytic enzyme, peroxidase, phospholipase, phytase, phosphatase, polyphenoloxidase, redox enzyme, proteolytic enzyme, ribonuclease, transglutaminase or xylanase.

As used herein, the terms “nucleic acid molecule”, “polynucleotide”, “polynucleic acid”, “nucleic acid” are used interchangeably and refer to a polymeric form of nucleotides of any length, either deoxyribonucleotides or ribonucleotides, or analogs thereof. Polynucleotides may have any three-dimensional structure, and may perform any function, known or unknown. Non-limiting examples of polynucleotides include a gene, a gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, control regions, promotor regions, isolated RNA of any sequence, nucleic acid probes, and primers. The nucleic acid molecule may be linear or circular.

The term “recombinant polynucleotide” refers to a nucleic acid molecule that was introduced in the filamentous fungus cell by means of recombinant DNA technology as is well known in the art and described in for example Molecular Cloning: A Laboratory Manual, 3rd ed., Vols 1,2 and 3 J. F. Sambrook and D. W. Russell, ed., Cold Spring Harbor Laboratory Press, 2001, 2100 pp. Recombinant DNA molecules can have its origin in a species other than the filamentous fungal cell or can be a polynucleotide native to the filamentous fungal cell.

“Proteases” are enzymes that catalyze a proteolysis reaction which is the breakdown of proteins into smaller fragments or into individual amino acids and where the proteolysis reaction occurs either at specific recognition sites or at random sites. Proteases can be classified, without limitation, into aspartic proteases, serine proteases (which include trypsin-like serine proteases and subtilisin proteases), glutamic proteases, metalloproteases and sedolisin proteases. Such proteases may be identified and isolated from filamentous fungal cells and tested to determine whether reduction in their activity affects the production of a recombinant polypeptide from the filamentous fungal cell. Methods for identifying and isolating proteases are well known in the art, and include, without limitation, affinity chromatography, zymogram assays, and gel electrophoresis. The term “endogenous protease” refers to a protease native to the microbial host cell.

The at least one endogenous protease having a reduced or no protease activity is a protease expressed by a gene that is contained within the genome of a parental or wild-type microbial host cell. In other words, the at least one endogenous protease is not a heterologous polypeptide. Instead, it is a protease that is coded for by the genome of a parental or wild-type microbial host cell. After modification, the microbial host cell might no longer contain the gene that codes for or expresses the protease, for example in the embodiments in which partial or full deletion of the gene occurs to adversely affect its production, stability and/or function. However, in some embodiments, the microbial host cell might still contain a full copy of the gene that codes for or expresses the at least one endogenous protease, for example in the embodiments in which the modification is one cause by administration of an inhibitor compounds (such as an RNAi or siRNA molecule that targets the gene encoding the at least one endogenous protease).

### Aspartic Proteases

Aspartic or aspartyl proteases are enzymes that use an aspartate residue for hydrolysis of the peptide bonds in polypeptides and proteins. Typically, aspartic proteases contain two highly-conserved aspartate residues in their active site which are optimally active at acidic pH. Aspartic proteases from eukaryotic organisms such as Trichoderma fungi include pepsins, cathepsins, and renins Such aspartic proteases have a two-domain structure, which is thought to arise from an ancestral gene duplication. Consistent with such a duplication event, the overall fold of each domain is similar, though the sequences of the two domains have begun to diverge. Each domain contributes one of the catalytic aspartate residues. The active site is in a cleft formed by the two domains of the aspartic proteases. Eukaryotic aspartic proteases further include conserved disulfide bridges, which can assist in identification of the polypeptides as being aspartic acid proteases.

pep1

Examples of suitable pep1 genes include, without limitation, Trichoderma reesei pep1 with Uniprot ID G0R8T0 (SEQ ID NO: 1), Myceliophthora thermophila pep1 with Uniprot ID G2QIG8 (SEQ ID NO: 2), Myceliophthora heterothallica pep1 (SEQ ID NO: 3) and homologs thereof. Accordingly, in certain embodiments, a protease of the present disclosure, typically a pep1 protease, has an amino acid sequence having 50% or more identity (e.g. 60%, 65%, 70%, 75%, 80%, 85%, 90%, 91%, 92%, 93%, 94%, 95%, 96%, 97%, 98%, 99%, 99.5% or more to an amino acid sequence selected from SEQ ID NOs: 1-3. In some embodiments, the protease has 100% identity to an amino acid sequence selected from SEQ ID NOs: 1-3. In some embodiments, pep1 is T. reesei pep1. The amino acid sequence encoded by T. reesei pep1 is set forth in SEQ ID NO: 1. In other embodiments, a protease of the present disclosure has an amino acid sequence having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 1. In further embodiments, the protease has 100% identity to SEQ ID NO: 1.

### Pep2

Examples of suitable pep2 genes include, without limitation, Trichoderma reesei pep2 with Uniprot ID G0R9K1 (SEQ ID NO: 4), Myceliophthora thermophila pep2 with Uniprot ID G2Q6W1 (SEQ ID NO: 5), Myceliophthora heterothallica pep2 (SEQ ID NO: 6) and homologs thereof. Accordingly, in certain embodiments, a protease of the present disclosure, typically a pep2 protease, has an amino acid sequence having 50% or more identity (e.g. 60%, 65%, 70%, 75%, 80%, 85%, 90%, 91%, 92%, 93%, 94%, 95%, 96%, 97%, 98%, 99%, 99.5% or more to an amino acid sequence selected from SEQ ID NOs: 4-6. In some embodiments, the protease has 100% identity to an amino acid sequence selected from SEQ ID NOs: 4-6. In some embodiments, pep2 is T. reesei pep2. The amino acid sequence encoded by T. reesei pep2 is set forth in SEQ ID NO: 4. In other embodiments, a protease of the present disclosure has an amino acid sequence having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 4. In further embodiments, the protease has 100% identity to SEQ ID NO: 4.

### Pep3

Examples of suitable pep3 genes include, without limitation, Trichoderma reesei pep3 with Uniprot ID G0RG34 (SEQ ID NO: 7), Myceliophthora thermophila pep3 with Uniprot ID G2Q837 (SEQ ID NO: 8), Myceliophthora heterothallica pep3 (SEQ ID NO: 9) and homologs thereof. Accordingly, in certain embodiments, a protease of the present disclosure, typically a pep3 protease, has an amino acid sequence having 50% or more identity (e.g. 60%, 65%, 70%, 75%, 80%, 85%, 90%, 91%, 92%, 93%, 94%, 95%, 96%, 97%, 98%, 99%, 99.5% or more to an amino acid sequence selected from SEQ ID NOs: 7-9. In some embodiments, the protease has 100% identity to an amino acid sequence selected from SEQ ID NOs: 7-9. In some embodiments, pep3 is T. reesei pep3. The amino acid sequence encoded by T. reesei pep3 is set forth in SEQ ID NO: 7. In other embodiments, a protease of the present disclosure has an amino acid sequence having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 7. In further embodiments, the protease has 100% identity to SEQ ID NO: 7.

### Pep4

Examples of suitable pep4 genes include, without limitation, Trichoderma reesei pep4 with Uniprot ID G0RIW3 (SEQ ID NO: 10), Myceliophthora thermophila pep4 with Uniprot ID G2QK78 (SEQ ID NO: 11), Myceliophthora heterothallica pep4 (SEQ ID NO: 12) and homologs thereof. Accordingly, in certain embodiments, a protease of the present disclosure, typically a pep4 protease, has an amino acid sequence having 50% or more identity (e.g. 60%, 65%, 70%, 75%, 80%, 85%, 90%, 91%, 92%, 93%, 94%, 95%, 96%, 97%, 98%, 99%, 99.5% or more to an amino acid sequence selected from SEQ ID NOs: 10-12. In some embodiments, the protease has 100% identity to an amino acid sequence selected from SEQ ID NOs: 10-12. In some embodiments, pep4 is T. reesei pep4. The amino acid sequence encoded by T. reesei pep4 is set forth in SEQ ID NO: 10. In other embodiments, a protease of the present disclosure has an amino acid sequence having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 10. In further embodiments, the protease has 100% identity to SEQ ID NO: 10.

pep5

Examples of suitable pep5 genes include, without limitation, Trichoderma reesei pep5 with Uniprot ID G0RSP8 (SEQ ID NO: 13), Myceliophthora heterothallica pep5 (SEQ ID NO: 14) and homologs thereof. Accordingly, in certain embodiments, a protease of the present disclosure, typically a pep5 protease, has an amino acid sequence having 50% or more identity (e.g. 60%, 65%, 70%, 75%, 80%, 85%, 90%, 91%, 92%, 93%, 94%, 95%, 96%, 97%, 98%, 99%, 99.5% or more to an amino acid sequence selected from SEQ ID NOs: 13-14. In some embodiments, the protease has 100% identity to an amino acid sequence selected from SEQ ID NOs: 13-14. In some embodiments, pep5 is T. reesei pep5. The amino acid sequence encoded by T. reesei pep5 is set forth in SEQ ID NO: 13. In other embodiments, a protease of the present disclosure has an amino acid sequence having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 13. In further embodiments, the protease has 100% identity to SEQ ID NO: 13.

pep8

Examples of suitable pep8 genes include, without limitation, Trichoderma reesei pep8 with Uniprot ID G0RKE2 (SEQ ID NO: 15), Myceliophthora thermophila pep8 with Uniprot ID G2Q4F9 (SEQ ID NO: 16), Myceliophthora heterothallica pep8 (SEQ ID NO: 17) and homologs thereof. Accordingly, in certain embodiments, a protease of the present disclosure, typically a pep8 protease, has an amino acid sequence having 50% or more identity (e.g. 60%, 65%, 70%, 75%, 80%, 85%, 90%, 91%, 92%, 93%, 94%, 95%, 96%, 97%, 98%, 99%, 99.5% or more to an amino acid sequence selected from SEQ ID NOs: 15-17. In some embodiments, the protease has 100% identity to an amino acid sequence selected from SEQ ID NOs: 15-17. In some embodiments, pep8 is T. reesei pep8. The amino acid sequence encoded by T. reesei pep8 is set forth in SEQ ID NO: 15. In other embodiments, a protease of the present disclosure has an amino acid sequence having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 15. In further embodiments, the protease has 100% identity to SEQ ID NO: 15.

pep9

Examples of suitable pep9 genes include, without limitation, Trichoderma reesei pep9 with Uniprot ID A0A024S610 (SEQ ID NO: 18), Myceliophthora thermophila pep9 with Uniprot ID G2QN49 (SEQ ID NO: 19), Myceliophthora heterothallica pep9 (SEQ ID NO: 20) and homologs thereof. Accordingly, in certain embodiments, a protease of the present disclosure, typically a pep9 protease, has an amino acid sequence having 50% or more identity (e.g. 60%, 65%, 70%, 75%, 80%, 85%, 90%, 91%, 92%, 93%, 94%, 95%, 96%, 97%, 98%, 99%, 99.5% or more to an amino acid sequence selected from SEQ ID NOs: 18-20. In some embodiments, the protease has 100% identity to an amino acid sequence selected from SEQ ID NOs: 18-20. In some embodiments, pep9 is T. reesei pep9. The amino acid sequence encoded by T. reesei pep9 is set forth in SEQ ID NO: 18. In other embodiments, a protease of the present disclosure has an amino acid sequence having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 18. In further embodiments, the protease has 100% identity to SEQ ID NO: 18.

pep11

Examples of suitable pep11 genes include, without limitation, Trichoderma reesei pep11 with Uniprot ID G0RHF7 (SEQ ID NO: 21), Myceliophthora thermophila pep11 with Uniprot ID G2QNW6 (SEQ ID NO: 22), Myceliophthora heterothallica pep11 (SEQ ID NO: 23) and homologs thereof. Accordingly, in certain embodiments, a protease of the present disclosure, typically a pep11 protease, has an amino acid sequence having 50% or more identity (e.g. 60%, 65%, 70%, 75%, 80%, 85%, 90%, 91%, 92%, 93%, 94%, 95%, 96%, 97%, 98%, 99%, 99.5% or more to an amino acid sequence selected from SEQ ID NOs: 21-23. In some embodiments, the protease has 100% identity to an amino acid sequence selected from SEQ ID NOs: 21-23. In some embodiments, pep11 is T. reesei pep11. The amino acid sequence encoded by T. reesei pep11 is set forth in SEQ ID NO: 21. In other embodiments, a protease of the present disclosure has an amino acid sequence having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 21. In further embodiments, the protease has 100% identity to SEQ ID NO: 21.

pep12

Examples of suitable pep12 genes include, without limitation, Trichoderma reesei pep12 with Uniprot ID G0R6X8 (SEQ ID NO: 24), Myceliophthora thermophila pep12 with Uniprot ID G2QBW3 (SEQ ID NO: 25), Myceliophthora heterothallica pep12 (SEQ ID NO: 26) and homologs thereof. Accordingly, in certain embodiments, a protease of the present disclosure, typically a pep12 protease, has an amino acid sequence having 50% or more identity (e.g. 60%, 65%, 70%, 75%, 80%, 85%, 90%, 91%, 92%, 93%, 94%, 95%, 96%, 97%, 98%, 99%, 99.5% or more to an amino acid sequence selected from SEQ ID NOs: 24-26. In some embodiments, the protease has 100% identity to an amino acid sequence selected from SEQ ID NOs: 24-26. In some embodiments, pep12 is T. reesei pep12. The amino acid sequence encoded by T. reesei pep12 is set forth in SEQ ID NO: 24. In other embodiments, a protease of the present disclosure has an amino acid sequence having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 24. In further embodiments, the protease has 100% identity to SEQ ID NO: 24.

aspX-7

Examples of suitable aspX-7 genes include, without limitation, Trichoderma reesei aspX-7 with Uniprot ID G0RGD6 (SEQ ID NO: 27), Myceliophthora thermophila aspX-7 with Uniprot ID G2QH17 (SEQ ID NO: 28), Myceliophthora heterothallica aspX-7 (SEQ ID NO: 29) and homologs thereof. Accordingly, in certain embodiments, a protease of the present disclosure, typically a aspX-7 protease, has an amino acid sequence having 50% or more identity (e.g. 60%, 65%, 70%, 75%, 80%, 85%, 90%, 91%, 92%, 93%, 94%, 95%, 96%, 97%, 98%, 99%, 99.5% or more to an amino acid sequence selected from SEQ ID NOs: 27-29. In some embodiments, the protease has 100% identity to an amino acid sequence selected from SEQ ID NOs: 27-29. In some embodiments, aspX-7 is T. reesei aspX-7. The amino acid sequence encoded by T. reesei aspX-7 is set forth in SEQ ID NO: 27. In other embodiments, a protease of the present disclosure has an amino acid sequence having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 27. In further embodiments, the protease has 100% identity to SEQ ID NO: 27.

aspX-11

Examples of suitable aspX-11 genes include, without limitation, Trichoderma reesei aspX-11 with Uniprot ID G0RVH9 (SEQ ID NO: 30), Myceliophthora thermophila aspX-11 with Uniprot ID G2Q6G1 (SEQ ID NO: 31), Myceliophthora heterothallica aspX-11 (SEQ ID NO: 32) and homologs thereof. Accordingly, in certain embodiments, a protease of the present disclosure, typically a aspX-11 protease, has an amino acid sequence having 50% or more identity (e.g. 60%, 65%, 70%, 75%, 80%, 85%, 90%, 91%, 92%, 93%, 94%, 95%, 96%, 97%, 98%, 99%, 99.5% or more to an amino acid sequence selected from SEQ ID NOs: 30-32. In some embodiments, the protease has 100% identity to an amino acid sequence selected from SEQ ID NOs: 30-32. In some embodiments, aspX-11 is T. reesei aspX-11. The amino acid sequence encoded by T. reesei aspX-11 is set forth in SEQ ID NO: 30. In other embodiments, a protease of the present disclosure has an amino acid sequence having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 30. In further embodiments, the protease has 100% identity to SEQ ID NO: 30.

### Glutamic Proteases

Glutamic proteases are enzymes that hydrolyze the peptide bonds in polypeptides and proteins. Glutamic proteases are insensitive to pepstatin A, and so are sometimes referred to as pepstatin insensitive acid proteases. While glutamic proteases were previously grouped with the aspartic proteases and often jointly referred to as acid proteases, it has been recently found that glutamic proteases have very different active site residues than aspartic proteases.

gap1

Examples of suitable gap1 genes include, without limitation, Trichoderma reesei gap1 with Uniprot ID G0RVK0 (SEQ ID NO: 33), Myceliophthora thermophila gap1 with Uniprot ID G2QCB6 (SEQ ID NO: 34), Myceliophthora heterothallica gap1 (SEQ ID NO: 35) and homologs thereof. Accordingly, in certain embodiments, a protease of the present disclosure, typically a gap1 protease, has an amino acid sequence having 50% or more identity (e.g. 60%, 65%, 70%, 75%, 80%, 85%, 90%, 91%, 92%, 93%, 94%, 95%, 96%, 97%, 98%, 99%, 99.5% or more to an amino acid sequence selected from SEQ ID NOs: 33-35. In some embodiments, the protease has 100% identity to an amino acid sequence selected from SEQ ID NOs: 33-35. In some embodiments, gap1 is T. reesei gap1. The amino acid sequence encoded by T. reesei gap1 is set forth in SEQ ID NO: 33. In other embodiments, a protease of the present disclosure has an amino acid sequence having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 33. In further embodiments, the protease has 100% identity to SEQ ID NO: 33.

gap2

Examples of suitable gap2 genes include, without limitation, Trichoderma reesei gap2 with Uniprot ID G0RH05 (SEQ ID NO: 36) and homologs thereof. Accordingly, in certain embodiments, a protease of the present disclosure, typically a gap2 protease, has an amino acid sequence having 50% or more identity (e.g. 60%, 65%, 70%, 75%, 80%, 85%, 90%, 91%, 92%, 93%, 94%, 95%, 96%, 97%, 98%, 99%, 99.5% or more to an amino acid sequence selected from SEQ ID NOs: 36. In some embodiments, the protease has 100% identity to an amino acid sequence selected from SEQ ID NOs: 36. In some embodiments, gap2 is T. reesei gap2. The amino acid sequence encoded by T. reesei gap2 is set forth in SEQ ID NO: 36. In other embodiments, a protease of the present disclosure has an amino acid sequence having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 36. In further embodiments, the protease has 100% identity to SEQ ID NO: 36.

### Serine Proteases

Serine proteases are enzymes with substrate specificity similar to that of trypsin. Serine proteases use a serine residue for hydrolysis of the peptide bonds in polypeptides and proteins serine proteases generally contain a catalytic triad of three amino acid residues (such as histidine, aspartate, and serine) that form a charge relay that serves to make the active site serine nucleophilic. Serine proteases fall into two broad categories based on their structure: chymotrypsin-like (trypsin-like) or subtilisin-like. Trypsin-like serine proteases are enzymes with substrate specificity similar to that of trypsin. Trypsin-like serine proteases use a serine residue for hydrolysis of the peptide bonds in polypeptides and proteins. Typically, trypsin-like serine proteases cleave peptide bonds following a positively-charged amino acid residue. Trypsin-like serine proteases from eukaryotic organisms such as Trichoderma fungi include trypsin 1, trypsin 2, and mesotrypsin. Such trypsin-like serine proteases generally contain a catalytic triad of three amino acid residues (such as histidine, aspartate, and serine) that form a charge relay that serves to make the active site serine nucleophilic. Eukaryotic trypsin-like serine proteases further include an “oxyanion hole” formed by the backbone amide hydrogen atoms of glycine and serine, which can assist in identification of the polypeptides as being trypsin-like serine proteases. Subtilisin proteases are enzymes with substrate specificity similar to that of subtilisin. Subtilisin proteases use a serine residue for hydrolysis of the peptide bonds in polypeptides and proteins. Generally, subtilisin proteases are serine proteases that contain a catalytic triad of the three amino acids aspartate, histidine, and serine. The arrangement of these catalytic residues is shared with the prototypical subtilisin from Bacillus licheniformis. Subtilisin proteases from eukaryotic organisms such as Trichoderma fungi include furin, MBTPSI, and TPP2. Eukaryotic trypsin-like serine proteases further include an aspartic acid residue in the oxyanion hole. Subtilisin protease slp7 resembles also sedolisin protease tppI.

Sep proteases are serine proteases belonging to the S28 subtype. They have a catalytic triad of serine, aspartate, and histidine: serine acts as a nucleophile, aspartate as an electrophile, and histidine as a base. These serine proteases include several eukaryotic enzymes such as lysosomal Pro-X carboxypeptidase, dipeptidyl-peptidase 1l, and thymus-specific serine peptidase.

Sedolisin proteases are enzymes that use a serine residue for hydrolysis of the peptide bonds in polypeptides and proteins. Sedolisin proteases generally contain a unique catalytic triad of serine, glutamate, and aspartate. Sedolisin proteases also contain an aspartate residue in the oxyanion hole. Sedolisin proteases from eukaryotic orgamsms such as Trichoderma fungi include tripeptidyl peptidase.

slp2

Examples of suitable slp2 genes include, without limitation, Trichoderma reesei slp2 with Uniprot ID G0RRK1 (SEQ ID NO: 37), Myceliophthora thermophila slp2 with Uniprot ID G2Q6Z6 (SEQ ID NO: 38), Myceliophthora heterothallica slp2 (SEQ ID NO: 39) and homologs thereof. Accordingly, in certain embodiments, a protease of the present disclosure, typically a slp2 protease, has an amino acid sequence having 50% or more identity (e.g. 60%, 65%, 70%, 75%, 80%, 85%, 90%, 91%, 92%, 93%, 94%, 95%, 96%, 97%, 98%, 99%, 99.5% or more to an amino acid sequence selected from SEQ ID NOs: 37-39. In some embodiments, the protease has 100% identity to an amino acid sequence selected from SEQ ID NOs: 37-39. In some embodiments, slp2 is T. reesei slp2. The amino acid sequence encoded by T. reesei slp2 is set forth in SEQ ID NO: 37. In other embodiments, a protease of the present disclosure has an amino acid sequence having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 37. In further embodiments, the protease has 100% identity to SEQ ID NO: 37.

slp6

Examples of suitable slp6 genes include, without limitation, Trichoderma reesei slp6 with Uniprot ID G0RHA8 (SEQ ID NO: 40), Myceliophthora thermophila slp6 with Uniprot ID G2Q925 (SEQ ID NO: 41), Myceliophthora heterothallica slp6 (SEQ ID NO: 42) and homologs thereof. Accordingly, in certain embodiments, a protease of the present disclosure, typically a slp6 protease, has an amino acid sequence having 50% or more identity (e.g. 60%, 65%, 70%, 75%, 80%, 85%, 90%, 91%, 92%, 93%, 94%, 95%, 96%, 97%, 98%, 99%, 99.5% or more to an amino acid sequence selected from SEQ ID NOs: 40-42. In some embodiments, the protease has 100% identity to an amino acid sequence selected from SEQ ID NOs: 40-42. In some embodiments, slp6 is T. reesei slp6. The amino acid sequence encoded by T. reesei slp6 is set forth in SEQ ID NO: 40. In other embodiments, a protease of the present disclosure has an amino acid sequence having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 40. In further embodiments, the protease has 100% identity to SEQ ID NO: 40.

serX-3

Examples of suitable serX-3 genes include, without limitation, Trichoderma reesei serX-3 with Uniprot ID G0RTY5 (SEQ ID NO: 43), Myceliophthora thermophila serX-3 with Uniprot ID G2QDE2 (SEQ ID NO: 44), Myceliophthora heterothallica serX-3 (SEQ ID NO: 45) and homologs thereof. Accordingly, in certain embodiments, a protease of the present disclosure, typically a serX-3 protease, has an amino acid sequence having 50% or more identity (e.g. 60%, 65%, 70%, 75%, 80%, 85%, 90%, 91%, 92%, 93%, 94%, 95%, 96%, 97%, 98%, 99%, 99.5% or more to an amino acid sequence selected from SEQ ID NOs: 43-45. In some embodiments, the protease has 100% identity to an amino acid sequence selected from SEQ ID NOs: 43-45. In some embodiments, serX-3 is T. reesei serX-3. The amino acid sequence encoded by T. reesei serX-3 is set forth in SEQ ID NO: 43. In other embodiments, a protease of the present disclosure has an amino acid sequence having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 43. In further embodiments, the protease has 100% identity to SEQ ID NO: 43.

serX-4

Examples of suitable serX-4 genes include, without limitation, Trichoderma reesei serX-4 with Uniprot ID G0R938 (SEQ ID NO: 133) and homologs thereof. Accordingly, in certain embodiments, a protease of the present disclosure, typically a serX-4 protease, has an amino acid sequence having 50% or more identity (e.g. 60%, 65%, 70%, 75%, 80%, 85%, 90%, 91%, 92%, 93%, 94%, 95%, 96%, 97%, 98%, 99%, 99.5% or more to an amino acid sequence selected from SEQ ID NOs: 133. In some embodiments, the protease has 100% identity to an amino acid sequence selected from SEQ ID NOs: 133. In some embodiments, serX-4 is T. reesei serX-4. The amino acid sequence encoded by T. reesei serX-4 is set forth in SEQ ID NO: 133. In other embodiments, a protease of the present disclosure has an amino acid sequence having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 133. In further embodiments, the protease has 100% identity to SEQ ID NO: 133.

serX-5

Examples of suitable serX-5 genes include, without limitation, Trichoderma Reesei serX-5 with Uniprot ID G0RVE6 (SEQ ID NO: 134), Myceliophthora thermophila serX-5 with Uniprot ID G2QK57 (SEQ ID NO: 135), Myceliophthora heterothallica serX-5 (SEQ ID NO: 136) and homologs thereof. Accordingly, in certain embodiments, a protease of the present disclosure, typically a serX-5 protease, has an amino acid sequence having 50% or more identity (e.g. 60%, 65%, 70%, 75%, 80%, 85%, 90%, 91%, 92%, 93%, 94%, 95%, 96%, 97%, 98%, 99%, 99.5% or more to an amino acid sequence selected from SEQ ID NOs: 134-136. In some embodiments, the protease has 100% identity to an amino acid sequence selected from SEQ ID NOs: 134-136. In some embodiments, serX-5 is T. reesei serX-5. The amino acid sequence encoded by T. reesei serX-5 is set forth in SEQ ID NO: 134. In other embodiments, a protease of the present disclosure has an amino acid sequence having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 134. In further embodiments, the protease has 100% identity to SEQ ID NO: 134.

serX-9

Examples of suitable serX-9 genes include, without limitation, Trichoderma reesei serX-9 with Uniprot ID G0RIL6 (SEQ ID NO: 46), Myceliophthora thermophila serX-9 with Uniprot ID G2Q6L2 (SEQ ID NO: 47), Myceliophthora heterothallica serX-9 (SEQ ID NO: 48) and homologs thereof. Accordingly, in certain embodiments, a protease of the present disclosure, typically a serX-9 protease, has an amino acid sequence having 50% or more identity (e.g. 60%, 65%, 70%, 75%, 80%, 85%, 90%, 91%, 92%, 93%, 94%, 95%, 96%, 97%, 98%, 99%, 99.5% or more to an amino acid sequence selected from SEQ ID NOs: 46-48. In some embodiments, the protease has 100% identity to an amino acid sequence selected from SEQ ID NOs: 46-48. In some embodiments, serX-9 is T. reesei serX-9. The amino acid sequence encoded by T. reesei serX-9 is set forth in SEQ ID NO: 46. In other embodiments, a protease of the present disclosure has an amino acid sequence having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 46. In further embodiments, the protease has 100% identity to SEQ ID NO: 46.

tpp1

Examples of suitable tpp1 genes include, without limitation, Trichoderma reesei tpp1 with Uniprot ID G0RXE9 (SEQ ID NO: 49), Myceliophthora thermophila tpp1 with Uniprot ID G2Q9P8 (SEQ ID NO: 50), Myceliophthora heterothallica tpp1 (SEQ ID NO: 51) and homologs thereof. Accordingly, in certain embodiments, a protease of the present disclosure, typically a tpp1 protease, has an amino acid sequence having 50% or more identity (e.g. 60%, 65%, 70%, 75%, 80%, 85%, 90%, 91%, 92%, 93%, 94%, 95%, 96%, 97%, 98%, 99%, 99.5% or more to an amino acid sequence selected from SEQ ID NOs: 49-51. In some embodiments, the protease has 100% identity to an amino acid sequence selected from SEQ ID NOs: 49-51. In some embodiments, tpp1 is T. reesei tpp1. The amino acid sequence encoded by T. reesei tpp1 is set forth in SEQ ID NO: 49. In other embodiments, a protease of the present disclosure has an amino acid sequence having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 49. In further embodiments, the protease has 100% identity to SEQ ID NO: 49.

ser1-4

Examples of suitable ser1-4 genes include, without limitation, Trichoderma reesei ser1-4 with Uniprot ID G0RVZ5 (SEQ ID NO: 52), Myceliophthora thermophila ser1-4 (SEQ ID NO: 53), Myceliophthora heterothallica ser1-4 (SEQ ID NO: 54) and homologs thereof. Accordingly, in certain embodiments, a protease of the present disclosure, typically a ser1-4 protease, has an amino acid sequence having 50% or more identity (e.g. 60%, 65%, 70%, 75%, 80%, 85%, 90%, 91%, 92%, 93%, 94%, 95%, 96%, 97%, 98%, 99%, 99.5% or more to an amino acid sequence selected from SEQ ID NOs: 52-54. In some embodiments, the protease has 100% identity to an amino acid sequence selected from SEQ ID NOs: 52-54. In some embodiments, ser1-4 is T. reesei ser1-4. The amino acid sequence encoded by T. reesei ser1-4 is set forth in SEQ ID NO: 52. In other embodiments, a protease of the present disclosure has an amino acid sequence having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 52. In further embodiments, the protease has 100% identity to SEQ ID NO: 52.

ser1-5

Examples of suitable ser1-5 genes include, without limitation, Trichoderma reesei ser1-5 with Uniprot ID G0RRG3 (SEQ ID NO: 55), Myceliophthora thermophila ser1-5 with Uniprot ID G2Q5V6 (SEQ ID NO: 56), Myceliophthora heterothallica ser1-5 (SEQ ID NO: 57) and homologs thereof. Accordingly, in certain embodiments, a protease of the present disclosure, typically a ser1-5 protease, has an amino acid sequence having 50% or more identity (e.g. 60%, 65%, 70%, 75%, 80%, 85%, 90%, 91%, 92%, 93%, 94%, 95%, 96%, 97%, 98%, 99%, 99.5% or more to an amino acid sequence selected from SEQ ID NOs: 55-57. In some embodiments, the protease has 100% identity to an amino acid sequence selected from SEQ ID NOs: 55-57. In some embodiments, ser1-5 is T. reesei ser1-5. The amino acid sequence encoded by T. reesei ser1-5 is set forth in SEQ ID NO: 55. In other embodiments, a protease of the present disclosure has an amino acid sequence having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 55. In further embodiments, the protease has 100% identity to SEQ ID NO: 55.

serX-10

Examples of suitable serX-10 genes include, without limitation, Trichoderma reesei serX-10 with Uniprot ID G0RCP2 (SEQ ID NO: 58), Myceliophthora thermophila serX-10 with Uniprot ID G2Q0R5 (SEQ ID NO: 59), Myceliophthora heterothallica serX-10 (SEQ ID NO: 60) and homologs thereof. Accordingly, in certain embodiments, a protease of the present disclosure, typically a serX-10 protease, has an amino acid sequence having 50% or more identity (e.g. 60%, 65%, 70%, 75%, 80%, 85%, 90%, 91%, 92%, 93%, 94%, 95%, 96%, 97%, 98%, 99%, 99.5% or more to an amino acid sequence selected from SEQ ID NOs: 58-60. In some embodiments, the protease has 100% identity to an amino acid sequence selected from SEQ ID NOs: 58-60. In some embodiments, serX-10 is T. reesei serX-10. The amino acid sequence encoded by T. reesei serX-10 is set forth in SEQ ID NO: 58. In other embodiments, a protease of the present disclosure has an amino acid sequence having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 58. In further embodiments, the protease has 100% identity to SEQ ID NO: 58.

sep1

Examples of suitable sep1 genes include, without limitation, Trichoderma reesei sep1 with Uniprot ID G0RW36 (SEQ ID NO: 61), Myceliophthora thermophila sep1 with Uniprot ID G2QG96 (SEQ ID NO: 62), Myceliophthora heterothallica sep1 (SEQ ID NO: 63) and homologs thereof. Accordingly, in certain embodiments, a protease of the present disclosure, typically a sep1 protease, has an amino acid sequence having 50% or more identity (e.g. 60%, 65%, 70%, 75%, 80%, 85%, 90%, 91%, 92%, 93%, 94%, 95%, 96%, 97%, 98%, 99%, 99.5% or more to an amino acid sequence selected from SEQ ID NOs: 61-63. In some embodiments, the protease has 100% identity to an amino acid sequence selected from SEQ ID NOs: 61-63. In some embodiments, sep1 is T. reesei sep1. The amino acid sequence encoded by T. reesei sep1 is set forth in SEQ ID NO: 61. In other embodiments, a protease of the present disclosure has an amino acid sequence having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 61. In further embodiments, the protease has 100% identity to SEQ ID NO: 61.

slp1

Examples of suitable slp1 genes include, without limitation, Trichoderma reesei slp1 with Uniprot ID G0RT14 (SEQ ID NO: 64), Myceliophthora thermophila slp1 with Uniprot ID G2QGG5 (SEQ ID NO: 65), Myceliophthora heterothallica slp1 (SEQ ID NO: 66) and homologs thereof. Accordingly, in certain embodiments, a protease of the present disclosure, typically a slp1 protease, has an amino acid sequence having 50% or more identity (e.g. 60%, 65%, 70%, 75%, 80%, 85%, 90%, 91%, 92%, 93%, 94%, 95%, 96%, 97%, 98%, 99%, 99.5% or more to an amino acid sequence selected from SEQ ID NOs: 64-66. In some embodiments, the protease has 100% identity to an amino acid sequence selected from SEQ ID NOs: 64-66. In some embodiments, slp1 is T. reesei slp1. The amino acid sequence encoded by T. reesei slp1 is set forth in SEQ ID NO: 64. In other embodiments, a protease of the present disclosure has an amino acid sequence having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 64. In further embodiments, the protease has 100% identity to SEQ ID NO: 64.

tsp1

Examples of suitable tsp1 genes include, without limitation, Trichoderma reesei tsp1 with Uniprot ID G0R816 (SEQ ID NO: 101), and homologs thereof. Accordingly, in certain embodiments, a protease of the present disclosure, typically a tsp1 protease, has an amino acid sequence having 50% or more identity (e.g. 60%, 65%, 70%, 75%, 80%, 85%, 90%, 91%, 92%, 93%, 94%, 95%, 96%, 97%, 98%, 99%, 99.5% or more to an amino acid sequence selected from SEQ ID NO: 101. In some embodiments, the protease has 100% identity to an amino acid sequence selected from SEQ ID NO: 101. In some embodiments, tsp1 is T. reesei tsp1. The amino acid sequence encoded by T. reesei tsp1 is set forth in SEQ ID NO: 101. In other embodiments, a protease of the present disclosure has an amino acid sequence having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 101. In further embodiments, the protease has 100% identity to SEQ ID NO: 101.

### Metalloprotease and Aminopeptidase Proteases

Aminopeptidases catalyze the cleavage of amino acids from the amino terminus of protein or peptide substrates. They are widely distributed throughout the animal and plant kingdoms and are found in many subcellular organelles, in cytoplasm, and as membrane components. Many, but not all, of these peptidases are zinc metalloenzymes. Amp2 is a bifunctional enzyme. It is a leukotriene A4 hydrolase with aminopeptidase activity (EC 3.3.2.6). A metalloprotease, such as a zinc metalloprotease is any protease enzyme whose catalytic mechanism involves a metal.

metX-11

Examples of suitable metX-11 genes include, without limitation, Trichoderma reesei metX-11 with Uniprot ID G0RC20 (SEQ ID NO: 67), Myceliophthora thermophila metX-11 with Uniprot ID G2QLU9 (SEQ ID NO: 68), Myceliophthora heterothallica metX-11 (SEQ ID NO: 69) and homologs thereof. Accordingly, in certain embodiments, a protease of the present disclosure, typically a metX-11 protease, has an amino acid sequence having 50% or more identity (e.g. 60%, 65%, 70%, 75%, 80%, 85%, 90%, 91%, 92%, 93%, 94%, 95%, 96%, 97%, 98%, 99%, 99.5% or more to an amino acid sequence selected from SEQ ID NOs: 67-69. In some embodiments, the protease has 100% identity to an amino acid sequence selected from SEQ ID NOs: 67-69. In some embodiments, metX-11 is T. reesei metX-11. The amino acid sequence encoded by T. reesei metX-11 is set forth in SEQ ID NO: 67. In other embodiments, a protease of the present disclosure has an amino acid sequence having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 67. In further embodiments, the protease has 100% identity to SEQ ID NO: 67.

metX-12

Examples of suitable metX-12 genes include, without limitation, Trichoderma reesei metX-12 with Uniprot ID G0RT39 (SEQ ID NO: 137), Myceliophthora thermophila metX-12 with Uniprot ID G2Q970 (SEQ ID NO: 138), Myceliophthora heterothallica metX-12 (SEQ ID NO: 139) and homologs thereof. Accordingly, in certain embodiments, a protease of the present disclosure, typically a metX-12 protease, has an amino acid sequence having 50% or more identity (e.g. 60%, 65%, 70%, 75%, 80%, 85%, 90%, 91%, 92%, 93%, 94%, 95%, 96%, 97%, 98%, 99%, 99.5% or more to an amino acid sequence selected from SEQ ID Nos: 137-139. In some embodiments, the protease has 100% identity to an amino acid sequence selected from SEQ ID Nos: 137-139. In some embodiments, metX-12 is T. reesei metX-12. The amino acid sequence encoded by T. reesei metX-12 is set forth in SEQ ID NO: 137. In other embodiments, a protease of the present disclosure has an amino acid sequence having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 137. In further embodiments, the protease has 100% identity to SEQ ID NO: 137.

amp1

Examples of suitable amp1 genes include, without limitation, Trichoderma reesei amp1 with Uniprot ID G0RSV5 (SEQ ID NO: 70), Myceliophthora thermophila amp1 with Uniprot ID G2QNT3 (SEQ ID NO: 71), Myceliophthora heterothallica amp1 (SEQ ID NO: 72) and homologs thereof. Accordingly, in certain embodiments, a protease of the present disclosure, typically a amp1 protease, has an amino acid sequence having 50% or more identity (e.g. 60%, 65%, 70%, 75%, 80%, 85%, 90%, 91%, 92%, 93%, 94%, 95%, 96%, 97%, 98%, 99%, 99.5% or more to an amino acid sequence selected from SEQ ID NOs: 70-72. In some embodiments, the protease has 100% identity to an amino acid sequence selected from SEQ ID NOs: 70-72. In some embodiments, amp1 is T. reesei amp1. The amino acid sequence encoded by T. reesei amp1 is set forth in SEQ ID NO: 70. In other embodiments, a protease of the present disclosure has an amino acid sequence having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 70. In further embodiments, the protease has 100% identity to SEQ ID NO: 70.

amp2

Examples of suitable amp2 genes include, without limitation, Trichoderma reesei amp2 with Uniprot ID G0RM29 (SEQ ID NO: 73), Myceliophthora thermophila amp2 with Uniprot ID G2Q7T0 (SEQ ID NO: 74), Myceliophthora heterothallica amp2 (SEQ ID NO: 75) and homologs thereof. Accordingly, in certain embodiments, a protease of the present disclosure, typically a amp2 protease, has an amino acid sequence having 50% or more identity (e.g. 60%, 65%, 70%, 75%, 80%, 85%, 90%, 91%, 92%, 93%, 94%, 95%, 96%, 97%, 98%, 99%, 99.5% or more to an amino acid sequence selected from SEQ ID NOs: 73-75. In some embodiments, the protease has 100% identity to an amino acid sequence selected from SEQ ID NOs: 73-75. In some embodiments, amp2 is T. reesei amp2. The amino acid sequence encoded by T. reesei amp2 is set forth in SEQ ID NO: 73. In other embodiments, a protease of the present disclosure has an amino acid sequence having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 73. In further embodiments, the protease has 100% identity to SEQ ID NO: 73.

cpa2

Examples of suitable cpa2 genes include, without limitation, Trichoderma reesei cpa2 with Uniprot ID G0RIK1 (SEQ ID NO: 76), Myceliophthora thermophila cpa2 with Uniprot ID G2Q9Z6 (SEQ ID NO: 77), Myceliophthora heterothallica cpa2 (SEQ ID NO: 78) and homologs thereof. Accordingly, in certain embodiments, a protease of the present disclosure, typically a cpa2 protease, has an amino acid sequence having 50% or more identity (e.g. 60%, 65%, 70%, 75%, 80%, 85%, 90%, 91%, 92%, 93%, 94%, 95%, 96%, 97%, 98%, 99%, 99.5% or more to an amino acid sequence selected from SEQ ID NOs: 76-78. In some embodiments, the protease has 100% identity to an amino acid sequence selected from SEQ ID NOs: 76-78. In some embodiments, cpa2 is T. reesei cpa2. The amino acid sequence encoded by T. reesei cpa2 is set forth in SEQ ID NO: 76. In other embodiments, a protease of the present disclosure has an amino acid sequence having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 76. In further embodiments, the protease has 100% identity to SEQ ID NO: 76.

cpa3

Examples of suitable cpa3 genes include, without limitation, Trichoderma reesei cpa3 with Uniprot ID G0RKF5 (SEQ ID NO: 79) and homologs thereof. Accordingly, in certain embodiments, a protease of the present disclosure, typically a cpa3 protease, has an amino acid sequence having 50% or more identity (e.g. 60%, 65%, 70%, 75%, 80%, 85%, 90%, 91%, 92%, 93%, 94%, 95%, 96%, 97%, 98%, 99%, 99.5% or more to an amino acid sequence selected from SEQ ID NOs: 79. In some embodiments, the protease has 100% identity to an amino acid sequence selected from SEQ ID NOs: 79. In some embodiments, cpa3 is T. reesei cpa3. The amino acid sequence encoded by T. reesei cpa3 is set forth in SEQ ID NO: 79. In other embodiments, a protease of the present disclosure has an amino acid sequence having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 79. In further embodiments, the protease has 100% identity to SEQ ID NO: 79.

cpa5

Examples of suitable cpa5 genes include, without limitation, Trichoderma reesei cpa5 with Uniprot ID G0RF39 (SEQ ID NO: 80), Myceliophthora thermophila cpa5 with Uniprot ID G2QBI0 (SEQ ID NO: 81), Myceliophthora heterothallica cpa5 (SEQ ID NO: 82) and homologs thereof. Accordingly, in certain embodiments, a protease of the present disclosure, typically a cpa5 protease, has an amino acid sequence having 50% or more identity (e.g. 60%, 65%, 70%, 75%, 80%, 85%, 90%, 91%, 92%, 93%, 94%, 95%, 96%, 97%, 98%, 99%, 99.5% or more to an amino acid sequence selected from SEQ ID NOs: 80-82. In some embodiments, the protease has 100% identity to an amino acid sequence selected from SEQ ID NOs: 80-82. In some embodiments, cpa5 is T. reesei cpa5. The amino acid sequence encoded by T. reesei cpa5 is set forth in SEQ ID NO: 80. In other embodiments, a protease of the present disclosure has an amino acid sequence having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 80. In further embodiments, the protease has 100% identity to SEQ ID NO: 80.

met1-1

Examples of suitable met1-1 genes include, without limitation, Trichoderma reesei met1-1 with Uniprot ID G0RCI8 (SEQ ID NO: 83), Myceliophthora thermophila met1-1 with Uniprot ID G2Q2U1 (SEQ ID NO: 84), Myceliophthora heterothallica met1-1 (SEQ ID NO: 85) and homologs thereof. Accordingly, in certain embodiments, a protease of the present disclosure, typically a met1-1 protease, has an amino acid sequence having 50% or more identity (e.g. 60%, 65%, 70%, 75%, 80%, 85%, 90%, 91%, 92%, 93%, 94%, 95%, 96%, 97%, 98%, 99%, 99.5% or more to an amino acid sequence selected from SEQ ID NOs: 83-85. In some embodiments, the protease has 100% identity to an amino acid sequence selected from SEQ ID NOs: 83-85. In some embodiments, met1-1 is T. reesei met1-1. The amino acid sequence encoded by T. reesei met1-1 is set forth in SEQ ID NO: 83. In other embodiments, a protease of the present disclosure has an amino acid sequence having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 83. In further embodiments, the protease has 100% identity to SEQ ID NO: 83.

met1-2

Examples of suitable met1-2 genes include, without limitation, Trichoderma reesei met1-2 with Uniprot ID G0RT10 (SEQ ID NO: 86), Myceliophthora thermophila met1-2 with Uniprot ID G2QGX6 (SEQ ID NO: 87), Myceliophthora heterothallica met1-2 (SEQ ID NO: 88) and homologs thereof. Accordingly, in certain embodiments, a protease of the present disclosure, typically a met1-2 protease, has an amino acid sequence having 50% or more identity (e.g. 60%, 65%, 70%, 75%, 80%, 85%, 90%, 91%, 92%, 93%, 94%, 95%, 96%, 97%, 98%, 99%, 99.5% or more to an amino acid sequence selected from SEQ ID NOs: 86-88. In some embodiments, the protease has 100% identity to an amino acid sequence selected from SEQ ID NOs: 86-88. In some embodiments, met1-2 is T. reesei met1-2. The amino acid sequence encoded by T. reesei met1-2 is set forth in SEQ ID NO: 86. In other embodiments, a protease of the present disclosure has an amino acid sequence having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 86. In further embodiments, the protease has 100% identity to SEQ ID NO: 86.

met1-3

Examples of suitable met1-3 genes include, without limitation, Trichoderma reesei met1-3 with Uniprot ID G0RV68 (SEQ ID NO: 89), Myceliophthora thermophila met1-3 with Uniprot ID G2Q928 (SEQ ID NO: 90), Myceliophthora heterothallica met1-3 (SEQ ID NO: 91) and homologs thereof. Accordingly, in certain embodiments, a protease of the present disclosure, typically a met1-3 protease, has an amino acid sequence having 50% or more identity (e.g. 60%, 65%, 70%, 75%, 80%, 85%, 90%, 91%, 92%, 93%, 94%, 95%, 96%, 97%, 98%, 99%, 99.5% or more to an amino acid sequence selected from SEQ ID NOs: 89-91. In some embodiments, the protease has 100% identity to an amino acid sequence selected from SEQ ID NOs: 89-91. In some embodiments, met1-3 is T. reesei met1-3. The amino acid sequence encoded by T. reesei met1-3 is set forth in SEQ ID NO: 89 In other embodiments, a protease of the present disclosure has an amino acid sequence having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 89. In further embodiments, the protease has 100% identity to SEQ ID NO: 89.

met1-6

Examples of suitable met1-6 genes include, without limitation, Trichoderma reesei met1-6 with Uniprot ID G0RWW0 (SEQ ID NO: 92), Myceliophthora thermophila met1-6 with Uniprot ID G2QCF2 (SEQ ID NO: 93), Myceliophthora heterothallica met1-6 (SEQ ID NO: 94) and homologs thereof. Accordingly, in certain embodiments, a protease of the present disclosure, typically a met1-6 protease, has an amino acid sequence having 50% or more identity (e.g. 60%, 65%, 70%, 75%, 80%, 85%, 90%, 91%, 92%, 93%, 94%, 95%, 96%, 97%, 98%, 99%, 99.5% or more to an amino acid sequence selected from SEQ ID NOs: 92-94. In some embodiments, the protease has 100% identity to an amino acid sequence selected from SEQ ID NOs: 92-94. In some embodiments, met1-6 is T. reesei met1-6. The amino acid sequence encoded by T. reesei met1-6 is set forth in SEQ ID NO: 92 In other embodiments, a protease of the present disclosure has an amino acid sequence having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 92. In further embodiments, the protease has 100% identity to SEQ ID NO: 92.

met1-7

Examples of suitable met1-7 genes include, without limitation, Trichoderma reesei met1-7 with Uniprot ID G0RHD6 (SEQ ID NO: 95), Myceliophthora thermophila met1-7 with Uniprot ID G2Q8S9 (SEQ ID NO: 96), Myceliophthora heterothallica met1-7 (SEQ ID NO: 97) and homologs thereof. Accordingly, in certain embodiments, a protease of the present disclosure, typically a met1-7 protease, has an amino acid sequence having 50% or more identity (e.g. 60%, 65%, 70%, 75%, 80%, 85%, 90%, 91%, 92%, 93%, 94%, 95%, 96%, 97%, 98%, 99%, 99.5% or more to an amino acid sequence selected from SEQ ID NOs: 95-97. In some embodiments, the protease has 100% identity to an amino acid sequence selected from SEQ ID NOs: 95-97. In some embodiments, met1-7 is T. reesei met1-7. The amino acid sequence encoded by T. reesei met1-7 is set forth in SEQ ID NO: 95 In other embodiments, a protease of the present disclosure has an amino acid sequence having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 95. In further embodiments, the protease has 100% identity to SEQ ID NO: 95.

vacX-1

Examples of suitable vacX-1 genes include, without limitation, Trichoderma reesei vacX-1 with Uniprot ID G0RBH0 (SEQ ID NO: 98), Myceliophthora thermophila vacX-1 with Uniprot ID G2PZW6 (SEQ ID NO: 99), Myceliophthora heterothallica vacX-1 (SEQ ID NO: 100) and homologs thereof. Accordingly, in certain embodiments, a protease of the present disclosure, typically a vacX-1 protease, has an amino acid sequence having 50% or more identity (e.g. 60%, 65%, 70%, 75%, 80%, 85%, 90%, 91%, 92%, 93%, 94%, 95%, 96%, 97%, 98%, 99%, 99.5% or more to an amino acid sequence selected from SEQ ID NOs: 98-100. In some embodiments, the protease has 100% identity to an amino acid sequence selected from SEQ ID NOs: 98-100. In some embodiments, vacX-1 is T. reesei vacX-1. The amino acid sequence encoded by T. reesei vacX-1 is set forth in SEQ ID NO: 98 In other embodiments, a protease of the present disclosure has an amino acid sequence having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 98. In further embodiments, the protease has 100% identity to SEQ ID NO: 98.

### Modified Microbial Host Cells of the Invention

The modified microbial host cell of the invention is characterized by having been modified in a way that leads to a reduced or no protease activity in at least one endogenous protease.

In some embodiments, the at least one endogenous protease may be a protease selected from the family of serine proteases. For example, the at least one endogenous protease may be selected from the proteases slp2, slp6, serX-3, serX-4, serX-5, serX-9, tpp1, ser1-4, ser1-5, serX-10, sep1, slp1 and tsp1. The at least one endogenous protease may be selected from the proteases slp2, slp6, serX-3, serX-9, tpp1, ser1-4, ser1-5, serX-10, sep1, slp1 and tsp1.

In some embodiments, the at least one endogenous protease may be a protease selected from the family of metalloproteases. For example, the at least one endogenous protease may be selected from the proteases metX-11, metX-12, amp1, amp2, cpa2, cpa3, cpa5, met1-1, met1-2, met1-3, met1-6, met1-7 and vacX-1. The at least one endogenous protease may be selected from the proteases metX-11, amp1, amp2, cpa2, cpa3, cpa5, met1-1, met1-2, met1-3, met1-6, met1-7 and vacX-1.

In some embodiments, the at least one endogenous protease may be an endogenous protease selected from the family of Aspartyl proteases. For example, the at least one endogenous protease may be selected from the proteases pep1, pep2, pep3, pep4, pep5, pep8, pep9, pep11, pep12, aspX-7, and aspX-11.

In some embodiments, the at least one endogenous protease may be an endogenous protease selected from the family of glutamic proteases. In a more preferred embodiment, the at least one endogenous protease may be a protease selected from the proteases gap1 and gap2.

In a more specific embodiment, the microbial host cell has reduced or no detectable protease activity in at least three proteases including either:
1. pep2, pep3 and pep4; or
2. slp1, pep1 and gap1.

In a more specific embodiment, the microbial host cell has reduced or no detectable protease activity in at least five proteases including:
1. pep2, pep3, pep4, pep5 and sep1.

In a more specific embodiment, the microbial host cell has reduced or no detectable protease activity in at least six proteases including:
1. pep2, pep3, pep4, pep5, gap2 and sep1;
2. slp1, pep1, gap1, pep5, gap2 and sep1; or
3. slp1, pep1, gap1, pep2, pep5 and sep1.

In a more specific embodiment, the microbial host cell has reduced or no detectable protease activity in at least seven proteases including:
1. pep2, pep3, pep4, pep5, gap2, sep1 and pep1;
2. slp1, pep1, gap1, pep5, gap2, sep1 and pep2;
3. slp1, pep1, gap1, pep2, pep5, sep1 and gap1; or
4. slp1, pep1, gap1, pep2, pep5, sep1 and gap2.

In a more specific embodiment, the microbial host cell has reduced or no detectable protease activity in at least eight proteases including:
1. pep2, pep3, pep4, pep5, gap2, sep1, pep1 and gap1; or
2. pep2, pep3, pep4, pep5, gap2, sep1, slp1 and gap1.

In a more specific embodiment, the microbial host cell has reduced or no detectable protease activity in at least nine proteases including:
1. pep2, pep3, pep4, pep5, gap2, sep1, slp1, pep1 and gap1.

In a more specific embodiment, the microbial host cell has reduced or no detectable protease activity in at least 10 proteases including:
1. pep2, pep3, pep4, pep5, gap2, sep1, slp1, pep1, gap1 and serX-4.
2. pep2, pep3, pep4, pep5, gap2, sep1, slp1, pep1, gap1 and serX-5.
3. pep2, pep3, pep4, pep5, gap2, sep1, slp1, pep1, gap1 and metX-12.

In a more specific embodiment, the microbial host cell has reduced or no detectable protease activity in at least eleven proteases including:
1. pep2, pep3, pep4, pep5, gap2, sep1, slp1, pep1, gap1, serX-4 and serX-5.
2. pep2, pep3, pep4, pep5, gap2, sep1, slp1, pep1, gap1, serX-4 and metX-12.
3. pep2, pep3, pep4, pep5, gap2, sep1, slp1, pep1, gap1, serX-5 and metX-12.

In a more specific embodiment, the microbial host cell has reduced or no detectable protease activity in at least twelve proteases including:
1. pep2, pep3, pep4, pep5, gap2, sep1, slp1, pep1, gap1, serX-4, serX-5 and metX-12.

The modified microbial host cell of the invention as described above is further characterized by comprising a recombinant polynucleotide encoding a compound of interest and wherein production of the compound of interest from the modified microbial cell is increased compared to the production of the same compound of interest from a parent microbial host cell lacking the modification that leads to a reduced or no protease activity of at least one endogenous protease, when measured under the same or substantially the same conditions.

The skilled person will understand that the optimal set of proteases may depend on the nature of the compound of interest. Proteases may need to recognize specific sites or a specific amino acid sequence in the compound of interest prior to being able to hydrolyze a certain peptide bond in the compound of interest. So, the optimal set of proteases that need to be modified to have reduced or no protease activity may depend on the primary structure or amino acid sequence of the compound of interest. However the skilled person will also understand that the tertiary or quaternary structure of the compound of interest is also important and may influence the optimal set of proteases for the production of a compound of interest. Indeed, specific sites recognized by specific proteases might be shielded due to steric hindrance inherently present in a folded protein, leading to the protease not being capable to reaching the target at which it can hydrolyze the peptide bond. The skilled person would thus understand that an optimal combination of modified proteases exists where the production level of the compound of interest from the modified microbial cell is increased compared to the production level of the same compound of interest as produced from the parent microbial host cell.

From the above it follows that the reduction in protease activity can be tailored to the compound of interest in that it can be more or less sensitive to degradation by specific proteases, for example depending on amino acid sequence. In some embodiments the modified microbial host cell can be altered in the proteases to which the compound of interest is more sensitive. For example, it can be determined by in silico methods what classes of proteases are likely to degrade the compound of interest. Such in silico methods as for example described in F. Li, Y. Wang, C. Li, T. T. Marquez-Lago, A. Leier, N. D. Rawlings, et al. Twenty years of bioinformatics research for protease-specific substrate and cleavage site prediction: a comprehensive revisit and benchmarking of existing methods Brief Bioinform, 20 (2018), pp. 2150-2166. Alternatively, stability of the compound of interest can be tested in an experimental setting where a fermentation broth is supplemented with specific protease inhibitors targeting a certain family of proteases and assessing compound of interest stability. Examples of such protease inhibitors are Pepstatin A inhibiting aspartic proteases or phenylmethylsulfonyl fluoride, Aprotinin and AEBSF inhibiting serine proteases, or EDTA for inhibiting metalloproteases. It thus follows that in some embodiments the microbial host cell is chosen so to have reduced or no reduction in protease either serine proteases, metalloproteases or aspartyl proteases or any combination thereof, depending on the outcome of in silico or in vitro assays. In specific embodiments the modified microbial host cell may be modified to optimize the production of an antibody. In a more specific embodiment, the modified microbial host cell might be optimized for the production of a VHH.

### Regulator of Transcription

In addition to the modification that leads to a reduced or no protease activity of at least one endogenous protease, the microbial host cell of the invention may have a further modification that affects the production, stability and/or function of the regulator of transcription.

As used herein, a “regulator of transcription” is a protein that regulates transcription, i.e. a regulator of transcription that causes, promotes, initiates, interrupts, represses or halts the transcription (and hence expression) of one or more genes (for example one or more genes coding for one or more proteases encoded by the microbial host cell genome). Generally, this is achieved by binding of the regulator of transcription to a specific DNA region (through use of a DNA binding domain), usually on or in the vicinity of one or more promoters, but essentially having its effect on the activity of said promoter or promoters, the genes being under the control of the promoters. For example, the proteases whose expression and/or activity is modulated may be under the control of the regulator of transcription. “Under the control” of the regulator of transcription means the regulator of transcription may directly control the rate of transcription of the relevant gene or genes (the one or more protease genes). Alternatively or additionally, the regulator of transcription may indirectly control the rate of transcription of the relevant gene or genes (the one or more protease genes). For example, the regulator of transcription may directly control the rate of transcription of one or more genes that in turn directly or indirectly control the transcription of the relevant gene or genes (the one or more protease genes). The number of stages in the pathway may differ, but importantly, the regulator of transcription should preferably (directly or indirectly) affect the protease activity of the microbial host cell.

By modifying the production, stability and/or function of a regulator of transcription, for example a regulator of transcription that controls the activity of one or more protease genes, one can affect the protease activity of the modified microbial host cell. The protease activity of the microbial host cell may be considered the cumulative activity of one or more proteases expressed by the microbial host cell, for example during culture or fermentation. In some embodiments, the modified protease activity of the modified microbial host cell may be the protease activity of one or more protease genes under the control of the regulator of transcription whose production, stability and/or function has been modified.

The regulator of transcription may be a “promoter of transcription” or a “repressor of transcription”. A “promoter of transcription” is a protein that causes, promotes or initiates the transcription (and hence expression) of one or more genes (e.g., endogenous protease genes). A promoter of transcription may be considered an enhancer of transcription and the terms “promoter of transcription” and “enhancer of transcription” may be used interchangeably”. A “repressor of transcription” is a protein that interrupts, represses or halts the transcription (and hence expression) of one or more genes. The regulator of transcription may be modified to adversely affect the production, stability and/or function of the said regulator of transcription, i.e. modified to diminish or reduce in some way the production, stability and/or function of the said regulator of transcription, or the regulator of transcription may be modified to positively affect the production, stability and/or function of the said regulator of transcription, i.e. modified to enhance or increase in some way the production, stability and/or function of the said regulator of transcription. The choice of an adverse modification or a positive modification may depend on the type of regulator of transcription that may be modified. For example, if the regulator of transcription is a promoter of transcription, the promoter of transcription may be modified to adversely affect the production, stability and/or function of the said promoter of transcription, i.e. modified to diminish or reduce in some way the production, stability and/or function of the said promoter of transcription, to reduce the level of expression of the gene or genes under control of the promoter of transcription. Alternatively, if the regulator of transcription is a repressor of transcription, the repressor of transcription may be modified to positively affect the production, stability and/or function of the said promoter of transcription, i.e. modified to enhance or increase in some way the production, stability and/or function of the said repressor of transcription, to reduce the level of expression of the gene or genes under control of the promoter of transcription. Preferably, the regulator of transcription is a promoter of transcription of at least one endogenous protease and the modification that affects the production, stability and/or function of the regulator of transcription is an adverse modification that reduces the production, stability and/or function of the regulator of transcription.

Suitable modifications to microbial host cells are described hereinabove in relation to endogenous protease genes. It is to be understood that these types of modifications can also apply, mutatis mutandis, to the modification that affects the production, stability and/or function of the regulator of transcription. For example, preferably, the modification that affects the production, stability and/or function of the regulator of transcription may be a genetic modification. The genetic modification may be in the polynucleotide encoding the regulator of transcription. For example, the genetic modification may be a partial or full deletion of the polynucleotide encoding the regulator of transcription.

In some embodiments, the regulator of transcription is a regulator of transcription having (comprising or consisting of) a sequence selected from the group consisting of SEQ ID NOs: 102, 105, 110, 113, 116 and 117. In some embodiments, the regulator of transcription may have an amino acid sequence that is at least about 80%, at least about 85%, at least about 90%, at least about 95%, or at least about 98% identical to a sequence selected from the group consisting of SEQ ID NOs: 102, 105, 110, 113, 116 and 117. In some embodiments, the regulator of transcription whose production, structure and/or function is being modulated is a regulator of transcription that is an ortholog of the regulator of transcription having (comprising or consisting of) a sequence selected from the group consisting of SEQ ID NOs: 102, 105, 110, 113, 116 and 117.

In some embodiments, the regulator of transcription whose production, structure and/or function is being modulated is a regulator of transcription encoded by a genomic nucleotide sequence having (comprising or consisting of) a sequence selected from the group consisting of SEQ ID NOs: 103, 106, 111 and 114. In some embodiments, the nucleotide sequence may have a sequence that is at least about 80%, at least about 85%, at least about 90%, at least about 95%, or at least about 98% identical to a sequence selected from the group consisting of SEQ ID NOs: 103, 106, 111 and 114. In some embodiments, the regulator of transcription whose production, structure and/or function is being modulated is a regulator of transcription that is an ortholog of the regulator of transcription encoded by a genomic nucleotide sequence having (comprising or consisting of) a sequence selected from the group consisting of SEQ ID NOs: 103, 106, 111 and 114.

In some embodiments, the regulator of transcription whose production, structure and/or function is being modulated is a regulator of transcription encoded by a nucleotide sequence having (comprising or consisting of) a sequence according to a sequence selected from the group consisting of SEQ ID NOs: 104, 107, 112 and 115. In some embodiments, the nucleotide sequence may have a sequence that is at least about 80%, at least about 85%, at least about 90%, at least about 95% identical, or at least about 98% identical to a sequence selected from the group consisting of SEQ ID NOs: 104, 107, 112 and 115. In some embodiments, the regulator of transcription whose production, structure and/or function is being modulated is a regulator of transcription that is an ortholog of the regulator of transcription encoded by a nucleotide sequence having (comprising or consisting of) a sequence according to a sequence selected from the group consisting of SEQ ID NOs: 104, 107, 112 and 115.

In some embodiments, the regulator of transcription whose production, structure and/or function is being modulated is a regulator of transcription having (comprising or consisting of) a sequence according to SEQ ID NO: 102. In some embodiments, the regulator of transcription may have an amino acid sequence that is at least about 80%, at least about 85%, at least about 90%, at least about 95%, or at least about 98% identical to SEQ ID NO: 102. In some embodiments, the regulator of transcription whose production, structure and/or function is being modulated is a regulator of transcription that is an ortholog of the regulator of transcription having (comprising or consisting of) a sequence according to SEQ ID NO: 102.

In some embodiments, the regulator of transcription whose production, structure and/or function is being modulated is a regulator of transcription encoded by a genomic nucleotide sequence having (comprising or consisting of) a sequence according to SEQ ID NO: 103. In some embodiments, the nucleotide sequence may have a sequence that is at least about 80%, at least about 85%, at least about 90%, at least about 95%, or at least about 98% identical to SEQ ID NO: 103. In some embodiments, the regulator of transcription whose production, structure and/or function is being modulated is a regulator of transcription that is an ortholog of the regulator of transcription encoded by a genomic nucleotide sequence having (comprising or consisting of) a sequence according to SEQ ID NO: 103.

In some embodiments, the regulator of transcription whose production, structure and/or function is being modulated is a regulator of transcription encoded by a nucleotide sequence having (comprising or consisting of) a sequence according to SEQ ID NO: 104. In some embodiments, the nucleotide sequence may have a sequence that is at least about 80%, at least about 85%, at least about 90%, at least about 95% identical, or at least about 98% identical to SEQ ID NO: 104. In some embodiments, the regulator of transcription whose production, structure and/or function is being modulated is a regulator of transcription that is an ortholog of the regulator of transcription encoded by a nucleotide sequence having (comprising or consisting of) a sequence according to SEQ ID NO: 104.

In some embodiments, the regulator of transcription whose production, structure and/or function is being modulated is a regulator of transcription having (comprising or consisting of) a sequence according to SEQ ID NO: 105. In some embodiments, the regulator of transcription may have an amino acid sequence that is at least about 80%, at least about 85%, at least about 90%, at least about 95%, or at least about 98% identical to SEQ ID NO: 105. In some embodiments, the regulator of transcription whose production, structure and/or function is being modulated is a regulator of transcription that is an ortholog of the regulator of transcription having (comprising or consisting of) a sequence according to SEQ ID NO: 105.

In some embodiments, the regulator of transcription whose production, structure and/or function is being modulated is a regulator of transcription encoded by a genomic nucleotide sequence having (comprising or consisting of) a sequence according to SEQ ID NO: 106. In some embodiments, the nucleotide sequence may have a sequence that is at least about 80%, at least about 85%, at least about 90%, at least about 95%, or at least about 98% identical to SEQ ID NO: 106. In some embodiments, the regulator of transcription whose production, structure and/or function is being modulated is a regulator of transcription that is an ortholog of the regulator of transcription encoded by a genomic nucleotide sequence having (comprising or consisting of) a sequence according to SEQ ID NO: 106.

In some embodiments, the regulator of transcription whose production, structure and/or function is being modulated is a regulator of transcription encoded by a nucleotide sequence having (comprising or consisting of) a sequence according to SEQ ID NO: 107. In some embodiments, the nucleotide sequence may have a sequence that is at least about 80%, at least about 85%, at least about 90%, at least about 95% identical, or at least about 98% identical to SEQ ID NO: 107. In some embodiments, the regulator of transcription whose production, structure and/or function is being modulated is a regulator of transcription that is an ortholog of the regulator of transcription encoded by a nucleotide sequence having (comprising or consisting of) a sequence according to SEQ ID NO: 107.

In some embodiments, the regulator of transcription whose production, structure and/or function is being modulated is a regulator of transcription having (comprising or consisting of) a sequence according to SEQ ID NO: 110. In some embodiments, the regulator of transcription may have an amino acid sequence that is at least about 80%, at least about 85%, at least about 90%, at least about 95%, or at least about 98% identical to SEQ ID NO: 110. In some embodiments, the regulator of transcription whose production, structure and/or function is being modulated is a regulator of transcription that is an ortholog of the regulator of transcription having (comprising or consisting of) a sequence according to SEQ ID NO: 110.

In some embodiments, the regulator of transcription whose production, structure and/or function is being modulated is a regulator of transcription encoded by a genomic nucleotide sequence having (comprising or consisting of) a sequence according to SEQ ID NO: 111. In some embodiments, the nucleotide sequence may have a sequence that is at least about 80%, at least about 85%, at least about 90%, at least about 95%, or at least about 98% identical to SEQ ID NO: 111. In some embodiments, the regulator of transcription whose production, structure and/or function is being modulated is a regulator of transcription that is an ortholog of the regulator of transcription encoded by a genomic nucleotide sequence having (comprising or consisting of) a sequence according to SEQ ID NO: 111.

In some embodiments, the regulator of transcription whose production, structure and/or function is being modulated is a regulator of transcription encoded by a nucleotide sequence having (comprising or consisting of) a sequence according to SEQ ID NO: 112. In some embodiments, the nucleotide sequence may have a sequence that is at least about 80%, at least about 85%, at least about 90%, at least about 95% identical, or at least about 98% identical to SEQ ID NO: 112. In some embodiments, the regulator of transcription whose production, structure and/or function is being modulated is a regulator of transcription that is an ortholog of the regulator of transcription encoded by a nucleotide sequence having (comprising or consisting of) a sequence according to SEQ ID NO: 112.

In some embodiments, the regulator of transcription whose production, structure and/or function is being modulated is a regulator of transcription having (comprising or consisting of) a sequence according to SEQ ID NO: 113. In some embodiments, the regulator of transcription may have an amino acid sequence that is at least about 80%, at least about 85%, at least about 90%, at least about 95%, or at least about 98% identical to SEQ ID NO: 113. In some embodiments, the regulator of transcription whose production, structure and/or function is being modulated is a regulator of transcription that is an ortholog of the regulator of transcription having (comprising or consisting of) a sequence according to SEQ ID NO: 113.

In some embodiments, the regulator of transcription whose production, structure and/or function is being modulated is a regulator of transcription encoded by a genomic nucleotide sequence having (comprising or consisting of) a sequence according to SEQ ID NO: 114. In some embodiments, the nucleotide sequence may have a sequence that is at least about 80%, at least about 85%, at least about 90%, at least about 95%, or at least about 98% identical to SEQ ID NO: 114. In some embodiments, the regulator of transcription whose production, structure and/or function is being modulated is a regulator of transcription that is an ortholog of the regulator of transcription encoded by a genomic nucleotide sequence having (comprising or consisting of) a sequence according to SEQ ID NO: 114.

In some embodiments, the regulator of transcription whose production, structure and/or function is being modulated is a regulator of transcription encoded by a nucleotide sequence having (comprising or consisting of) a sequence according to SEQ ID NO: 115. In some embodiments, the nucleotide sequence may have a sequence that is at least about 80%, at least about 85%, at least about 90%, at least about 95% identical, or at least about 98% identical to SEQ ID NO: 115. In some embodiments, the regulator of transcription whose production, structure and/or function is being modulated is a regulator of transcription that is an ortholog of the regulator of transcription encoded by a nucleotide sequence having (comprising or consisting of) a sequence according to SEQ ID NO: 115.

In some embodiments, the regulator of transcription whose production, structure and/or function is being modulated is a regulator of transcription having (comprising or consisting of) a sequence according to SEQ ID NO: 116. In some embodiments, the regulator of transcription may have an amino acid sequence that is at least about 80%, at least about 85%, at least about 90%, at least about 95%, or at least about 98% identical to SEQ ID NO: 116. In some embodiments, the regulator of transcription whose production, structure and/or function is being modulated is a regulator of transcription that is an ortholog of the regulator of transcription having (comprising or consisting of) a sequence according to SEQ ID NO: 116.

In some embodiments, the regulator of transcription whose production, structure and/or function is being modulated is a regulator of transcription having (comprising or consisting of) a sequence according to SEQ ID NO: 117. In some embodiments, the regulator of transcription may have an amino acid sequence that is at least about 80%, at least about 85%, at least about 90%, at least about 95%, or at least about 98% identical to SEQ ID NO: 117. In some embodiments, the regulator of transcription whose production, structure and/or function is being modulated is a regulator of transcription that is an ortholog of the regulator of transcription having (comprising or consisting of) a sequence according to SEQ ID NO: 117.

In some embodiments, the host cell may be modified to affect the production, stability and/or function of at least one regulator of transcription comprising a sequence selected from the group consisting of SEQ ID NOs: 102, 105, 110, 113, 116 and 117 or a regulator of transcription at least about 80%, at least about 85%, at least about 90%, at least about 95%, or at least about 98% identical thereto, or an ortholog thereof, and wherein the microbial host cell has been further modified to affect the production, stability and/or function of one or more additional regulator of transcriptions. In such embodiments, the modification of a plurality of regulator of transcriptions may have beneficial effects, such as an increase in yields in embodiments related to the production of a compound of interest. The additional regulator of transcriptions that may be modified in such approaches may have any of the preferred or more specific features of the modified regulator of transcriptions as described herein.

When amino acid or nucleotide sequences are used having a defined percent identity, they will generally still retain the function of the full-length reference sequence.

For example, the host cell may be modified to affect the production, stability and/or function of at least one regulator of transcription comprising a sequence selected from the group consisting of SEQ ID NOs: 102, 105, 110, 113, 116 and 117 or a functional variant thereof, wherein a functional variant is a variant having at least about 80%, at least about 85%, at least about 90%, at least about 95%, or at least about 98% identity to a sequence selected from the group consisting of SEQ ID NOs: 102, 105, 110, 113, 116 and 117, wherein the functional variant retains the same function as the regulator of transcription having an amino acid sequence that is 100% identical to a sequence selected from the group consisting of SEQ ID NOs: 102, 105, 110, 113, 116 and 117.

In some embodiments, functional variants (i.e. those having a certain percent identity relative to a sequence selected from the group consisting of SEQ ID NOs: 102, 105, 110, 113, 116 and 117) may retain the regulation of transcription function. More specifically, the functional variants may retain the ability to control (directly or indirectly) the same one or more genes (such as protease genes) whose transcription is/are controlled by a regulator of transcription having a sequence selected from the group consisting of SEQ ID NOs: 102, 105, 110, 113, 116 and 117.

When the regulator of transcription is a promoter of transcription, the functional variants of the promoter of transcription may retain the promoter of transcription function. More specifically, the functional variants may retain the ability to cause, promote or initiate (directly or indirectly) the same one or more genes (such as protease genes) whose transcription is/are caused, promoted or initiated by a promoter of transcription having a sequence selected from the group consisting of SEQ ID NOs: 102, 105, 110, 113, 116 and 117.

When the regulator of transcription is a repressor of transcription, the functional variants of the repressor of transcription may retain the repressor of transcription function. More specifically, the functional variants may retain the ability to interrupt, repress or halt (directly or indirectly) the same one or more genes (such as protease genes) whose transcription is/are interrupted, repressed or halted by a repressor of transcription having a sequence selected from the group consisting of SEQ ID NOs: 102, 105, 110, 113, 116 and 117.

The functional variants of regulators of transcription encoded by a sequence selected from the group consisting of SEQ ID NOs: 103, 104, 106, 107, 111, 112, 114 and 115 may retain their function in the same way as described above for variants of regulators of transcription having a sequence selected from the group consisting of SEQ ID NOs: 102, 105, 110 and 113.

In some embodiments, the regulator of transcription whose production, structure and/or function is being modulated is an ortholog of the target regulator of transcription, i.e. an ortholog of the regulator of transcription having an amino acid sequence selected from the group consisting of SEQ ID NOs: 102, 105, 110, 113, 116 and 117 (or an ortholog of a regulator of transcription encoded by a genomic nucleotide sequence comprising a sequence selected from the group consisting of SEQ ID NOs: 103, 106, 111 and 114 or a nucleotide sequence comprising a sequence selected from the group consisting of SEQ ID NO: 104, 107, 112 and 115).

An ortholog refers to any of two or more homologous gene or protein sequences found in different species related by linear descent. The orthologs serve the same or similar function in a different species.

In some embodiments, the ortholog may be from a different genus. In some embodiments, the ortholog may be from the same genus. In some embodiments, the orthologs may be from the same species, but a different strain.

Preferably, the ortholog performs the same or similar function as the reference regulator of transcription. More specifically, the ortholog may retain the ability to control (directly or indirectly) the same (or similar) one or more genes (such as protease genes, or orthologs thereof) whose transcription is/are controlled by the reference regulator of transcription having a sequence selected from the group consisting of SEQ ID NOs: 102, 105, 110, 113, 116 and 117. When the reference regulator of transcription is a promoter of transcription, the ortholog may also be a promoter of transcription. More specifically, the ortholog may retain the ability to cause, promote or initiate (directly or indirectly) the same (or similar) one or more genes (such as protease genes, or orthologs thereof)) whose transcription is/are caused, promoted or initiated by the reference promoter of transcription having a sequence selected from the group consisting of SEQ ID NOs: 102, 105, 110, 113, 116 and 117.

The orthologs of regulators of transcription encoded by a sequence selected from the group consisting of SEQ ID NOs: 103, 104, 106, 107, 111, 112, 114 and 115 may perform the same function as described above for orthologs of regulators of transcription having a sequence selected from the group consisting of SEQ ID NOs: 102, 105, 110 and 113.

Orthologs may have sequence identity with one another. For example, in some embodiments, the orthologs may have at least about 35% identity, at least about 40% identity, at least about 50% identity, at least about 60% identity, at least about 70% identity, at least about 80% identity, at least about 90% identity, at least about 95% identity, at least about 96% identity, at least about 97% identity, at least about 98% identity, or at least about 99% identity across their length with the regulator of transcription whose production, structure and/or function is being modulated. In some embodiments, the orthologs may have at least about 35% identity, at least about 40% identity, at least about 50% identity, at least about 60% identity, at least about 70% identity, at least about 80% identity, at least about 90% identity, at least about 95% identity, at least about 96% identity, at least about 97% identity, at least about 98% identity, or at least about 99% identity with the regulator of transcription whose production, structure and/or function is being modulated and they may be from about 80% to about 120% the length of the regulator of transcription whose production, structure and/or function is being modulated. In some embodiments, the orthologs may have at least about 35% identity with the regulator of transcription whose production, structure and/or function is being modulated and they may be from about 80% to about 120% the length of the regulator of transcription whose production, structure and/or function is being modulated. In some embodiments, the orthologs may be from the same genus and have at least about 90% identity with the regulator of transcription whose production, structure and/or function is being modulated and they may be from about 80% to about 120% the length of the regulator of transcription whose production, structure and/or function is being modulated.

Orthologs may comprise conserved sequences. For example, in some embodiments, an ortholog comprises a sequence having at least about 95% sequence identity with SEQ ID NO: 108. In some embodiments, an ortholog comprises the sequence of SEQ ID NO: 108.

In some embodiments, an ortholog of the regulator of transcription of SEQ ID NO: 102 may comprise a sequence having at least about 95% sequence identity with the amino acids 685-735 of SEQ ID NO: 102. In some embodiments, an ortholog of the regulator of transcription of SEQ ID NO: 102 may comprise a sequence comprising amino acids 685-735 of SEQ ID NO: 102. In some embodiments, an ortholog of the regulator of transcription of SEQ ID NO: 105 may comprise a sequence having at least about 95% sequence identity with the amino acids 753-803 of SEQ ID NO: 105. In some embodiments, an ortholog of the regulator of transcription of SEQ ID NO: 105 may comprise a sequence comprising amino acids 753-803 of SEQ ID NO: 105. In some embodiments, an ortholog of the regulator of transcription of SEQ ID NO: 110 may comprise a sequence having at least about 95% sequence identity with the amino acids 749-799 of SEQ ID NO: 110. In some embodiments, an ortholog of the regulator of transcription of SEQ ID NO: 110 may comprise a sequence comprising amino acids 749-799 of SEQ ID NO: 110. In some embodiments, an ortholog of the regulator of transcription of SEQ ID NO: 113 may comprise a sequence having at least about 95% sequence identity with the amino acids 670-720 of SEQ ID NO: 113. In some embodiments, an ortholog of the regulator of transcription of SEQ ID NO: 113 may comprise a sequence comprising amino acids 670-720 of SEQ ID NO: 113. In some embodiments, an ortholog of the regulator of transcription of SEQ ID NO: 116 may comprise a sequence having at least about 95% sequence identity with the amino acids 679-729 of SEQ ID NO: 116. In some embodiments, an ortholog of the regulator of transcription of SEQ ID NO: 116 may comprise a sequence comprising amino acids 679-729 of SEQ ID NO: 116. In some embodiments, an ortholog of the regulator of transcription of SEQ ID NO: 117 may comprise a sequence having at least about 95% sequence identity with the amino acids 749-799 of SEQ ID NO: 117. In some embodiments, an ortholog of the regulator of transcription of SEQ ID NO: 117 may comprise a sequence comprising amino acids 749-799 of SEQ ID NO: 117.

The orthologs may additionally comprise a defined sequence identity to a longer reference sequence. For example, in some embodiments, the ortholog may have at least about 35%, at least about 40%, at least about 45%, at least about 50%, at least about 60%, at least about 70%, at least about 80%, at least about 90%, at least about 95%, at least about 96%, at least about 97%, at least about 98% or at least about 99% sequence identity to the reference sequence, in addition to comprising a highly conserved sequence. For example, in some embodiments, an ortholog of the regulator of transcription of SEQ ID NO: 102 may comprise a sequence having at least about 95% sequence identity with the amino acids 685-735 of SEQ ID NO: 102 and the ortholog may comprise a sequence that is at least 35% identical to the sequence of SEQ ID NO: 102. In some embodiments, an ortholog of the regulator of transcription of SEQ ID NO: 105 may comprise a sequence having at least about 95% sequence identity with the amino acids 753-803 of SEQ ID NO: 105 and the ortholog may comprise a sequence that is at least 35% identical to the sequence of SEQ ID NO: 105. In some embodiments, an ortholog of the regulator of transcription of SEQ ID NO: 110 may comprise a sequence having at least about 95% sequence identity with the amino acids 749-799 of SEQ ID NO: 110 and the ortholog may comprise a sequence that is at least 35% identical to the sequence of SEQ ID NO: 110. In some embodiments, an ortholog of the regulator of transcription of SEQ ID NO: 113 may comprise a sequence having at least about 95% sequence identity with the amino acids 670-720 of SEQ ID NO: 113 and the ortholog may comprise a sequence that is at least 35% identical to the sequence of SEQ ID NO: 113. In some embodiments, an ortholog of the regulator of transcription of SEQ ID NO: 116 may comprise a sequence having at least about 95% sequence identity with the amino acids 679-729 of SEQ ID NO: 116 and the ortholog may comprise a sequence that is at least 35% identical to the sequence of SEQ ID NO: 116. In some embodiments, an ortholog of the regulator of transcription of SEQ ID NO: 117 may comprise a sequence having at least about 95% sequence identity with the amino acids 749-799 of SEQ ID NO: 117 and the ortholog may comprise a sequence that is at least 35% identical to the sequence of SEQ ID NO: 117.

In some embodiments, an ortholog of the regulator of transcription of SEQ ID NO: 102 may comprise a sequence having amino acids 685-735 of SEQ ID NO: 102 and the ortholog may comprise a sequence that is at least 35% identical to the sequence of SEQ ID NO: 102. In some embodiments, an ortholog of the regulator of transcription of SEQ ID NO: 105 may comprise a sequence having amino acids 753-803 of SEQ ID NO: 105 and the ortholog may comprise a sequence that is at least 35% identical to the sequence of SEQ ID NO: 105. In some embodiments, an ortholog of the regulator of transcription of SEQ ID NO: 110 may comprise a sequence having amino acids 749-799 of SEQ ID NO: 110 and the ortholog may comprise a sequence that is at least 35% identical to the sequence of SEQ ID NO: 110. In some embodiments, an ortholog of the regulator of transcription of SEQ ID NO: 113 may comprise a sequence having amino acids 670-720 of SEQ ID NO: 113 and the ortholog may comprise a sequence that is at least 35% identical to the sequence of SEQ ID NO: 113. In some embodiments, an ortholog of the regulator of transcription of SEQ ID NO: 116 may comprise a sequence having amino acids 679-729 of SEQ ID NO: 116 and the ortholog may comprise a sequence that is at least 35% identical to the sequence of SEQ ID NO: 116. In some embodiments, an ortholog of the regulator of transcription of SEQ ID NO: 117 may comprise a sequence having amino acids 749-799 of SEQ ID NO: 117 and the ortholog may comprise a sequence that is at least 35% identical to the sequence of SEQ ID NO: 117.

In some embodiments, variations in sequence between an ortholog and a reference sequence may be conservative variations. For example, in some embodiments, when the sequences of an ortholog having at least 35% identity to SEQ ID NO: 102 and comprising a sequence having at least about 95% sequence identity with the amino acids 685-735 of SEQ ID NO: 102 is aligned with the sequence of SEQ ID NO: 102, at least 50% of the variations between the ortholog and the sequence of SEQ ID NO: 102 are conservative amino acid substitutions. In some embodiments, at least 60%, at least 70%, at least 80% or at least 90% of the variations between the ortholog and the sequence of SEQ ID NO: 102 are conservative amino acid substitutions. In some embodiments, when the sequences of an ortholog having at least 35% identity to SEQ ID NO: 105 and comprising a sequence having at least about 95% sequence identity with the amino acids 753-803 of SEQ ID NO: 105 is aligned with the sequence of SEQ ID NO: 105, at least 50% of the variations between the ortholog and the sequence of SEQ ID NO: 105 are conservative amino acid substitutions. In some embodiments, at least 60%, at least 70%, at least 80% or at least 90% of the variations between the ortholog and the sequence of SEQ ID NO: 105 are conservative amino acid substitutions. In some embodiments, when the sequences of an ortholog having at least 35% identity to SEQ ID NO: 110 and comprising a sequence having at least about 95% sequence identity with the amino acids 749-799 of SEQ ID NO: 110 is aligned with the sequence of SEQ ID NO: 110, at least 50% of the variations between the ortholog and the sequence of SEQ ID NO: 110 are conservative amino acid substitutions. In some embodiments, at least 60%, at least 70%, at least 80% or at least 90% of the variations between the ortholog and the sequence of SEQ ID NO: 110 are conservative amino acid substitutions. In some embodiments, when the sequences of an ortholog having at least 35% identity to SEQ ID NO: 113 and comprising a sequence having at least about 95% sequence identity with the amino acids 670-720 of SEQ ID NO: 113 is aligned with the sequence of SEQ ID NO: 113, at least 50% of the variations between the ortholog and the sequence of SEQ ID NO: 113 are conservative amino acid substitutions. In some embodiments, at least 60%, at least 70%, at least 80% or at least 90% of the variations between the ortholog and the sequence of SEQ ID NO: 113 are conservative amino acid substitutions. In some embodiments, when the sequences of an ortholog having at least 35% identity to SEQ ID NO: 116 and comprising a sequence having at least about 95% sequence identity with the amino acids 679-729 of SEQ ID NO: 116 is aligned with the sequence of SEQ ID NO: 116, at least 50% of the variations between the ortholog and the sequence of SEQ ID NO: 116 are conservative amino acid substitutions. In some embodiments, at least 60%, at least 70%, at least 80% or at least 90% of the variations between the ortholog and the sequence of SEQ ID NO: 116 are conservative amino acid substitutions. In some embodiments, when the sequences of an ortholog having at least 35% identity to SEQ ID NO: 117 and comprising a sequence having at least about 95% sequence identity with the amino acids 749-799 of SEQ ID NO: 117 is aligned with the sequence of SEQ ID NO: 117, at least 50% of the variations between the ortholog and the sequence of SEQ ID NO: 117 are conservative amino acid substitutions. In some embodiments, at least 60%, at least 70%, at least 80% or at least 90% of the variations between the ortholog and the sequence of SEQ ID NO: 117 are conservative amino acid substitutions.

In some embodiments, when the sequences of an ortholog having at least 35% identity to SEQ ID NO: 102 and comprising amino acids 685-735 of SEQ ID NO: 102 is aligned with the sequence of SEQ ID NO: 102, at least 50% of the variations between the ortholog and the sequence of SEQ ID NO: 102 are conservative amino acid substitutions. In some embodiments, at least 60%, at least 70%, at least 80% or at least 90% of the variations between the ortholog and the sequence of SEQ ID NO: 102 are conservative amino acid substitutions. In some embodiments, when the sequences of an ortholog having at least 35% identity to SEQ ID NO: 105 and comprising amino acids 753-803 of SEQ ID NO: 105 is aligned with the sequence of SEQ ID NO: 105, at least 50% of the variations between the ortholog and the sequence of SEQ ID NO: 105 are conservative amino acid substitutions. In some embodiments, at least 60%, at least 70%, at least 80% or at least 90% of the variations between the ortholog and the sequence of SEQ ID NO: 105 are conservative amino acid substitutions. In some embodiments, when the sequences of an ortholog having at least 35% identity to SEQ ID NO: 110 and comprising amino acids 749-799 of SEQ ID NO: 110 is aligned with the sequence of SEQ ID NO: 110, at least 50% of the variations between the ortholog and the sequence of SEQ ID NO: 110 are conservative amino acid substitutions. In some embodiments, at least 60%, at least 70%, at least 80% or at least 90% of the variations between the ortholog and the sequence of SEQ ID NO: 110 are conservative amino acid substitutions. In some embodiments, when the sequences of an ortholog having at least 35% identity to SEQ ID NO: 113 and comprising amino acids 670-720 of SEQ ID NO: 113 is aligned with the sequence of SEQ ID NO: 113, at least 50% of the variations between the ortholog and the sequence of SEQ ID NO: 113 are conservative amino acid substitutions. In some embodiments, at least 60%, at least 70%, at least 80% or at least 90% of the variations between the ortholog and the sequence of SEQ ID NO: 113 are conservative amino acid substitutions. In some embodiments, when the sequences of an ortholog having at least 35% identity to SEQ ID NO: 116 and comprising amino acids 679-729 of SEQ ID NO: 116 is aligned with the sequence of SEQ ID NO: 116, at least 50% of the variations between the ortholog and the sequence of SEQ ID NO: 116 are conservative amino acid substitutions. In some embodiments, at least 60%, at least 70%, at least 80% or at least 90% of the variations between the ortholog and the sequence of SEQ ID NO: 116 are conservative amino acid substitutions. In some embodiments, when the sequences of an ortholog having at least 35% identity to SEQ ID NO: 117 and comprising amino acids 749-799 of SEQ ID NO: 117 is aligned with the sequence of SEQ ID NO: 117, at least 50% of the variations between the ortholog and the sequence of SEQ ID NO: 117 are conservative amino acid substitutions. In some embodiments, at least 60%, at least 70%, at least 80% or at least 90% of the variations between the ortholog and the sequence of SEQ ID NO: 117 are conservative amino acid substitutions.

Conservative amino acid substitutions are well known to the person of skill in the art. For example, conservative amino acid substitutions may be:
a) the substitution of any glycine, alanine, valine, leucine or isoleucine residues in the reference sequence with another amino acid selected from glycine, alanine, valine, leucine and isoleucine;
b) the substitution of any serine, cysteine, threonine or methionine residues in the reference sequence with another amino acid selected from serine, cysteine, threonine and methionine;
c) the substitution of any phenylalanine, tyrosine or tryptophan residues in the reference sequence with another amino acid selected from phenylalanine, tyrosine and tryptophan;
d) the substitution of any histidine, lysine or arginine residues in the reference sequence with another amino acid selected from histidine, lysine or arginine; and
e) the substitution of any aspartate, glutamate, asparagine or glutamine residues in the reference sequence with another amino acid selected from aspartate, glutamate, asparagine and glutamine.

In some embodiments, the ortholog may be from a Trichoderma spp., a Myceliophthora spp., an Aspergillus spp., a Penicillium spp., a Rasamsonia spp. or a Fusarium spp. In some embodiments, the ortholog may be from a Trichoderma spp., a Myceliophthora spp. or an Aspergillus spp. In some embodiments, the ortholog may be from a Trichoderma spp. or a Myceliophthora spp. In some embodiments, for example, but not limited to, embodiments relating to SEQ ID NOs: 102 to 104, the ortholog may be from a Trichoderma spp. In some embodiments, for example, but not limited to, embodiments relating to SEQ ID NO: 105 to 107 or 110 to 112, the ortholog may be from a Myceliophthora spp. In some embodiments, for example, but not limited to, embodiments relating to SEQ ID NO: 113 to 115, the ortholog may be from a Aspergillus spp. However, as noted above, orthologs may not necessarily be from the same genus as the reference transcription factor.

In some embodiments, the regulator of transcription whose production, structure and/or function is being modulated (or ortholog thereof) comprises a GATA-type zinc finger domain. GATA-type zinc finger domains bind the DNA sequence X1GATAX2 (SEQ ID NO: 109), wherein X1 is A or T and X2 is A or G. In some embodiments, the regulator of transcription whose production, structure and/or function is being modulated (or ortholog thereof) does not comprise more than one GATA-type zinc finger domain

In some embodiments, the GATA-type zinc finger domain comprises a sequence having at least about 95% identity to SEQ ID NO: 108. In some embodiments, the GATA-type zinc finger domain comprises a sequence having at least about 96%, at least about 97%, at least about 98% or at least about 99% identity to SEQ ID NO: 108. In some embodiments, the GATA-type zinc finger domain comprises the sequence of SEQ ID NO: 108.

Generally, the regulator of transcription whose production, structure and/or function is being modulated (or ortholog thereof) is a fungal protein, i.e. a protein expressed by fungi. The protein may be a protein that is found in wild-type fungi, i.e. naturally occurring fungal species. In preferred embodiments, the regulator of transcription whose production, structure and/or function is being modulated (or ortholog thereof) is a filamentous fungal protein (i.e. a protein from filamentous fungi).

In some embodiments, the regulator of transcription whose production, structure and/or function is being modulated (or ortholog thereof) is a GATA-type transcriptional activator. In some embodiments, the regulator of transcription whose production, structure and/or function is being modulated (or ortholog thereof) is an Are regulator of transcription. In some embodiments, the regulator of transcription whose production, structure and/or function is being modulated (or ortholog thereof) is Are1 or AreA, or an ortholog of Are1 or AreA. Preferably, the modification that affects the production, stability and/or function of the regulator of transcription is a partial or full deletion in a polynucleotide encoding an Are regulator of transcription, for example, a partial or full deletion in the polynucleotide encoding Are1 or AreA. More preferably, the modification that affects the production, stability and/or function of the regulator of transcription is a full deletion of the polynucleotide encoding Are1.

In some embodiments, the regulator of transcription whose production, structure and/or function is being modulated (or ortholog thereof) comprises a sequence having at least about 95% sequence identity with the amino acids 685-735 of SEQ ID NO: 102. In some embodiments, regulator of transcription whose production, structure and/or function is being modulated (or ortholog thereof) comprises a sequence comprising amino acids 685-735 of SEQ ID NO: 102. In some embodiments, regulator of transcription whose production, structure and/or function is being modulated (or ortholog thereof) comprises a sequence having at least about 95% sequence identity with the amino acids 753-803 of SEQ ID NO: 105. In some embodiments, regulator of transcription whose production, structure and/or function is being modulated (or ortholog thereof) comprises a sequence comprising amino acids 753-803 of SEQ ID NO: 105. In some embodiments, regulator of transcription whose production, structure and/or function is being modulated (or ortholog thereof) comprises a sequence having at least about 95% sequence identity with the amino acids 749-799 of SEQ ID NO: 110. In some embodiments, regulator of transcription whose production, structure and/or function is being modulated (or ortholog thereof) comprises a sequence comprising amino acids 749-799 of SEQ ID NO: 110. In some embodiments, regulator of transcription whose production, structure and/or function is being modulated (or ortholog thereof) comprises a sequence having at least about 95% sequence identity with the amino acids 670-720 of SEQ ID NO: 113. In some embodiments, regulator of transcription whose production, structure and/or function is being modulated (or ortholog thereof) comprises a sequence comprising amino acids 670-720 of SEQ ID NO: 113. In some embodiments, regulator of transcription whose production, structure and/or function is being modulated (or ortholog thereof) comprises a sequence having at least about 95% sequence identity with the amino acids 679-729 of SEQ ID NO: 116. In some embodiments, regulator of transcription whose production, structure and/or function is being modulated (or ortholog thereof) comprises a sequence comprising amino acids 679-729 of SEQ ID NO: 116. In some embodiments, regulator of transcription whose production, structure and/or function is being modulated (or ortholog thereof) comprises a sequence having at least about 95% sequence identity with the amino acids 749-799 of SEQ ID NO: 117. In some embodiments, regulator of transcription whose production, structure and/or function is being modulated (or ortholog thereof) comprises a sequence comprising amino acids 749-799 of SEQ ID NO: 117.

In some embodiments, the regulator of transcription whose production, structure and/or function is being modulated (or ortholog thereof) comprises a sequence having at least about 95% sequence identity with the amino acids 685-735 of SEQ ID NO: 102 and the regulator of transcription may comprise a sequence that is at least about 35% identical to the sequence of SEQ ID NO: 102. In some embodiments, the regulator of transcription whose production, structure and/or function is being modulated (or ortholog thereof) comprises a sequence having at least about 95% sequence identity with the amino acids 753-803 of SEQ ID NO: 105 and the regulator of transcription may comprise a sequence that is at least about 35% identical to the sequence of SEQ ID NO: 105. In some embodiments, the regulator of transcription whose production, structure and/or function is being modulated (or ortholog thereof) comprises a sequence having at least about 95% sequence identity with the amino acids 749-799 of SEQ ID NO: 110 and the regulator of transcription may comprise a sequence that is at least about 35% identical to the sequence of SEQ ID NO: 110. In some embodiments, the regulator of transcription whose production, structure and/or function is being modulated (or ortholog thereof) comprises a sequence having at least about 95% sequence identity with the amino acids 670-720 of SEQ ID NO: 113 and the regulator of transcription may comprise a sequence that is at least about 35% identical to the sequence of SEQ ID NO: 113. In some embodiments, the regulator of transcription whose production, structure and/or function is being modulated (or ortholog thereof) comprises a sequence having at least about 95% sequence identity with the amino acids 679-729 of SEQ ID NO: 116 and the regulator of transcription may comprise a sequence that is at least about 35% identical to the sequence of SEQ ID NO: 116. In some embodiments, the regulator of transcription whose production, structure and/or function is being modulated (or ortholog thereof) comprises a sequence having at least about 95% sequence identity with the amino acids 749-799 of SEQ ID NO: 117 and the regulator of transcription may comprise a sequence that is at least about 35% identical to the sequence of SEQ ID NO: 117.

In some embodiments, the regulator of transcription whose production, structure and/or function is being modulated (or ortholog thereof) comprises a sequence having amino acids 685-735 of SEQ ID NO: 102 and the regulator of transcription may comprise a sequence that is at least about 35% identical to the sequence of SEQ ID NO: 102. In some embodiments, the regulator of transcription whose production, structure and/or function is being modulated (or ortholog thereof) comprises a sequence having amino acids 753-803 of SEQ ID NO: 105 and the regulator of transcription may comprise a sequence that is at least about 35% identical to the sequence of SEQ ID NO: 105. In some embodiments, the regulator of transcription whose production, structure and/or function is being modulated (or ortholog thereof) comprises a sequence having amino acids 749-799 of SEQ ID NO: 110 and the regulator of transcription may comprise a sequence that is at least about 35% identical to the sequence of SEQ ID NO: 110. In some embodiments, the regulator of transcription whose production, structure and/or function is being modulated (or ortholog thereof) comprises a sequence having amino acids 670-720 of SEQ ID NO: 113 and the regulator of transcription may comprise a sequence that is at least about 35% identical to the sequence of SEQ ID NO: 113. In some embodiments, the regulator of transcription whose production, structure and/or function is being modulated (or ortholog thereof) comprises a sequence having amino acids 679-729 of SEQ ID NO: 116 and the regulator of transcription may comprise a sequence that is at least about 35% identical to the sequence of SEQ ID NO: 116. In some embodiments, the regulator of transcription whose production, structure and/or function is being modulated (or ortholog thereof) comprises a sequence having amino acids 749-799 of SEQ ID NO: 117 and the regulator of transcription may comprise a sequence that is at least about 35% identical to the sequence of SEQ ID NO: 117.

In some embodiments, variations in sequence may be conservative variations. For example, in some embodiments, when the sequences of the regulator of transcription whose production, structure and/or function is being modulated (or ortholog thereof) having at least about 35% identity to SEQ ID NO: 102 and comprising a sequence having at least about 95% sequence identity with the amino acids 685-735 of SEQ ID NO: 102 is aligned with the sequence of SEQ ID NO: 102, at least about 50% of the variations between the regulator of transcription and the sequence of SEQ ID NO: 102 are conservative amino acid substitutions. In some embodiments, at least about 60%, at least about 70%, at least about 80% or at least about 90% of the variations between the regulator of transcription and the sequence of SEQ ID NO: 102 are conservative amino acid substitutions. In some embodiments, when the sequences of the regulator of transcription whose production, structure and/or function is being modulated (or ortholog thereof) having at least about 35% identity to SEQ ID NO: 105 and comprising a sequence having at least about 95% sequence identity with the amino acids 753-803 of SEQ ID NO: 105 is aligned with the sequence of SEQ ID NO: 105, at least about 50% of the variations between the regulator of transcription and the sequence of SEQ ID NO: 105 are conservative amino acid substitutions. In some embodiments, at least about 60%, at least about 70%, at least about 80% or at least about 90% of the variations between the regulator of transcription and the sequence of SEQ ID NO: 105 are conservative amino acid substitutions. In some embodiments, when the sequences of the regulator of transcription whose production, structure and/or function is being modulated (or ortholog thereof) having at least about 35% identity to SEQ ID NO: 110 and comprising a sequence having at least about 95% sequence identity with the amino acids 749-799 of SEQ ID NO: 110 is aligned with the sequence of SEQ ID NO: 110, at least about 50% of the variations between the regulator of transcription and the sequence of SEQ ID NO: 110 are conservative amino acid substitutions. In some embodiments, at least about 60%, at least about 70%, at least about 80% or at least about 90% of the variations between the regulator of transcription and the sequence of SEQ ID NO: 110 are conservative amino acid substitutions. In some embodiments, when the sequences of the regulator of transcription whose production, structure and/or function is being modulated (or ortholog thereof) having at least about 35% identity to SEQ ID NO: 113 and comprising a sequence having at least about 95% sequence identity with the amino acids 670-720 of SEQ ID NO: 113 is aligned with the sequence of SEQ ID NO: 113, at least about 50% of the variations between the regulator of transcription and the sequence of SEQ ID NO: 113 are conservative amino acid substitutions. In some embodiments, at least about 60%, at least about 70%, at least about 80% or at least about 90% of the variations between the regulator of transcription and the sequence of SEQ ID NO: 113 are conservative amino acid substitutions. In some embodiments, when the sequences of the regulator of transcription whose production, structure and/or function is being modulated (or ortholog thereof) having at least about 35% identity to SEQ ID NO: 116 and comprising a sequence having at least about 95% sequence identity with the amino acids 679-729 of SEQ ID NO: 116 is aligned with the sequence of SEQ ID NO: 116, at least about 50% of the variations between the regulator of transcription and the sequence of SEQ ID NO: 116 are conservative amino acid substitutions. In some embodiments, at least about 60%, at least about 70%, at least about 80% or at least about 90% of the variations between the regulator of transcription and the sequence of SEQ ID NO: 116 are conservative amino acid substitutions. In some embodiments, when the sequences of the regulator of transcription whose production, structure and/or function is being modulated (or ortholog thereof) having at least about 35% identity to SEQ ID NO: 117 and comprising a sequence having at least about 95% sequence identity with the amino acids 749-799 of SEQ ID NO: 117 is aligned with the sequence of SEQ ID NO: 117, at least about 50% of the variations between the regulator of transcription and the sequence of SEQ ID NO: 117 are conservative amino acid substitutions. In some embodiments, at least about 60%, at least about 70%, at least about 80% or at least about 90% of the variations between the regulator of transcription and the sequence of SEQ ID NO: 117 are conservative amino acid substitutions.

In some embodiments, variations in sequence may be conservative variations. For example, in some embodiments, when the sequences of the regulator of transcription whose production, structure and/or function is being modulated (or ortholog thereof) having at least about 35% identity to SEQ ID NO: 102 and comprising amino acids 685-735 of SEQ ID NO: 102 is aligned with the sequence of SEQ ID NO: 102, at least about 50% of the variations between the regulator of transcription and the sequence of SEQ ID NO: 102 are conservative amino acid substitutions. In some embodiments, at least about 60%, at least about 70%, at least about 80% or at least about 90% of the variations between the regulator of transcription and the sequence of SEQ ID NO: 102 are conservative amino acid substitutions. In some embodiments, when the sequences of the regulator of transcription whose production, structure and/or function is being modulated (or ortholog thereof) having at least about 35% identity to SEQ ID NO: 105 and comprising amino acids 753-803 of SEQ ID NO: 105 is aligned with the sequence of SEQ ID NO: 105, at least about 50% of the variations between the regulator of transcription and the sequence of SEQ ID NO: 105 are conservative amino acid substitutions. In some embodiments, at least about 60%, at least about 70%, at least about 80% or at least about 90% of the variations between the regulator of transcription and the sequence of SEQ ID NO: 105 are conservative amino acid substitutions. In some embodiments, when the sequences of the regulator of transcription whose production, structure and/or function is being modulated (or ortholog thereof) having at least about 35% identity to SEQ ID NO: 110 and comprising amino acids 749-799 of SEQ ID NO: 110 is aligned with the sequence of SEQ ID NO: 110, at least about 50% of the variations between the regulator of transcription and the sequence of SEQ ID NO: 110 are conservative amino acid substitutions. In some embodiments, at least about 60%, at least about 70%, at least about 80% or at least about 90% of the variations between the regulator of transcription and the sequence of SEQ ID NO: 110 are conservative amino acid substitutions. In some embodiments, when the sequences of the regulator of transcription whose production, structure and/or function is being modulated (or ortholog thereof) having at least about 35% identity to SEQ ID NO: 113 and comprising amino acids 670-720 of SEQ ID NO: 113 is aligned with the sequence of SEQ ID NO: 113, at least about 50% of the variations between the regulator of transcription and the sequence of SEQ ID NO: 113 are conservative amino acid substitutions. In some embodiments, at least about 60%, at least about 70%, at least about 80% or at least about 90% of the variations between the regulator of transcription and the sequence of SEQ ID NO: 113 are conservative amino acid substitutions. In some embodiments, when the sequences of the regulator of transcription whose production, structure and/or function is being modulated (or ortholog thereof) having at least about 35% identity to SEQ ID NO: 116 and comprising amino acids 679-729 of SEQ ID NO: 116 is aligned with the sequence of SEQ ID NO: 116, at least about 50% of the variations between the regulator of transcription and the sequence of SEQ ID NO: 116 are conservative amino acid substitutions. In some embodiments, at least about 60%, at least about 70%, at least about 80% or at least about 90% of the variations between the regulator of transcription and the sequence of SEQ ID NO: 116 are conservative amino acid substitutions. In some embodiments, when the sequences of the regulator of transcription whose production, structure and/or function is being modulated (or ortholog thereof) having at least about 35% identity to SEQ ID NO: 117 and comprising amino acids 749-799 of SEQ ID NO: 117 is aligned with the sequence of SEQ ID NO: 117, at least about 50% of the variations between the regulator of transcription and the sequence of SEQ ID NO: 117 are conservative amino acid substitutions. In some embodiments, at least about 60%, at least about 70%, at least about 80% or at least about 90% of the variations between the regulator of transcription and the sequence of SEQ ID NO: 117 are conservative amino acid substitutions.

In embodiments in which the % identity threshold is indicated between a regulator of transcription and an ortholog thereof, the % identity between the full length regulator of transcription may differ. Two regulators of transcription may have a % identity as low as 35%, but still be orthologs of each other, provided they additional comprise conserved sequences, for example a sequence having at least about 95% identity to SEQ ID NO: 108 (e.g. a sequence having 100% identity to SEQ ID NO: 108). All of the regulators of transcription identified in the present invention comprise the sequence of SEQ ID NO: 108. Preferably, the orthologs perform the same function as the reference regulator of transcription. For example, the orthologs may be a fungal transcription factor that promotes the expression of one or more proteases. Generally, the orthologs are naturally occurring regulators of transcription.

The orthologs may be from the same genus, for example Trichoderma, Myceliophthora or Aspergillus, or they may be from a different genus. In some cases, different strains of the same species may comprise orthologs. Thus, the ortholog may be from the same species, but a different strain.

In some embodiments, for example when the ortholog is from a different genus, an ortholog may have at least about 35% sequence identity or at least about 40% sequence identity to the full length regulator of transcription. In some embodiments, the regulator of transcription may be a regulator of transcription from a Trichoderma spp. and the ortholog is a regulator of transcription from a Trichoderma spp. or a Myceliophthora spp. and the ortholog may have at least about 40% sequence identity to the full length regulator of transcription. In some embodiments, the regulator of transcription may be a regulator of transcription from a Myceliophthora spp. and the ortholog is a regulator of transcription from a Trichoderma spp. or a Myceliophthora spp. and the ortholog may have at least about 40% sequence identity to the full length regulator of transcription. In some embodiments, for example when the ortholog is from the same genus, an ortholog may have at least about 90% sequence identity to the full length regulator of transcription. In some embodiments, for example when the ortholog is from the same species, an ortholog may have at least about 95%, at least about 96%, at least about 97%, at least about 98% or at least about 99% sequence identity to the full length regulator of transcription. In all cases, the orthologs preferably comprise a conserved sequence, such as the sequence of SEQ ID NO: 108 (or a sequence having at least about 95% identity thereto).

In some embodiments, the regulator of transcription whose production, structure and/or function is being modulated (or ortholog thereof) is a fungal transcription factor that promotes the expression of one or more proteases, and comprises an amino acid sequence having at least about 95% identity to SEQ ID NO: 108, and optionally further comprises a sequence having at least about 35% identity to SEQ ID NO: 102, SEQ ID NO: 105, SEQ ID NO: 110 SEQ ID NO: 113, SEQ ID NO: 116 and/or SEQ ID NO: 117. In some embodiments, the regulator of transcription whose production, structure and/or function is being modulated (or ortholog thereof) is a fungal transcription factor that promotes the expression of one or more proteases, and comprises the amino acid sequence of SEQ ID NO: 108, and optionally further comprises a sequence having at least about 35% identity to SEQ ID NO: 102, SEQ ID NO: 105, SEQ ID NO: 110, SEQ ID NO: 113 SEQ ID NO: 116 and/or SEQ ID NO: 117.

In some embodiments, the regulator of transcription whose production, structure and/or function is being modulated (or ortholog thereof) is a fungal transcription factor that promotes the expression of one or more proteases, and comprises an amino acid sequence having at least about 95% identity to SEQ ID NO: 108, and optionally further comprises a sequence having at least about 40% identity to SEQ ID NO: 102, SEQ ID NO: 105, SEQ ID NO: 110, SEQ ID NO: 116 and/or SEQ ID NO: 117. In some embodiments, the regulator of transcription whose production, structure and/or function is being modulated (or ortholog thereof) is a fungal transcription factor that promotes the expression of one or more proteases, and comprises the amino acid sequence of SEQ ID NO: 108, and optionally further comprises a sequence having at least about 40% identity to SEQ ID NO: 102, SEQ ID NO: 105, SEQ ID NO: 110, SEQ ID NO: 116 and/or SEQ ID NO: 117.

In some embodiments, the regulator of transcription whose production, structure and/or function is being modulated (or ortholog thereof) is a fungal transcription factor that promotes the expression of one or more proteases, and comprises an amino acid sequence having at least about 95% identity to SEQ ID NO: 108, and optionally further comprises a sequence having at least about 95% identity to SEQ ID NO: 102 and/or SEQ ID NO: 116. In some embodiments, the regulator of transcription whose production, structure and/or function is being modulated (or ortholog thereof) is a fungal transcription factor that promotes the expression of one or more proteases, and comprises the amino acid sequence of SEQ ID NO: 108, and optionally further comprises a sequence having at least about 95% identity to SEQ ID NO: 102 and/or SEQ ID NO: 116.

In some embodiments, the regulator of transcription whose production, structure and/or function is being modulated (or ortholog thereof) is a fungal transcription factor that promotes the expression of one or more proteases, and comprises an amino acid sequence having at least about 95% identity to SEQ ID NO: 108, and optionally further comprises a sequence having at least about 90% identity to SEQ ID NO: 105, SEQ ID NO: 110 and/or SEQ ID NO: 117. In some embodiments, the regulator of transcription whose production, structure and/or function is being modulated (or ortholog thereof) is a fungal transcription factor that promotes the expression of one or more proteases, and comprises the amino acid sequence of SEQ ID NO: 108, and optionally further comprises a sequence having at least about 90% identity SEQ ID NO: 105, SEQ ID NO: 110 and/or SEQ ID NO: 117.

In some embodiments, the regulator of transcription whose production, structure and/or function is being modulated (or ortholog thereof) is a fungal transcription factor that promotes the expression of one or more proteases, and comprises an amino acid sequence having at least about 95% identity to SEQ ID NO: 108, and optionally further comprises a sequence having at least about 95% identity to SEQ ID NO: 105 and/or SEQ ID NO: 117. In some embodiments, the regulator of transcription whose production, structure and/or function is being modulated (or ortholog thereof) is a fungal transcription factor that promotes the expression of one or more proteases, and comprises the amino acid sequence of SEQ ID NO: 108, and optionally further comprises a sequence having at least about 95% identity SEQ ID NO: 105 and/or SEQ ID NO: 117.

Further reference is given to PCT Patent Application No: PCT/EP2021/071595 the entire contents of which are incorporated herein by reference.

In a further embodiment of the invention, the modified microbial host cell of the invention characterized by having been modified in a way that leads to a reduced or no protease activity in at least one endogenous protease may be further modified in a regulator of transcription, in particular a regulator of transcription that regulates the transcription of one or more protease genes encoded by the microbial host cell genome. The applicant has surprisingly found that the combination of a modification in a regulator of transcription, in particular a regulator of transcription that regulates the transcription of one or more protease genes encoded by the microbial host cell genome, together with a modification leading to a reduced or no protease activity of at least one endogenous protease, and where that at least one protease is comprised in the family of serine proteases, metalloproteases, aspartyl proteases and glutamic proteases, leads to an even further increase in production of the compound of interest from the modified microbial cell as compared to the production of the same compound of interest from a parent microbial host cell lacking the modification that leads to a reduced or no protease activity of the at least one endogenous protease.

In an embodiment of the invention, the combination of a modification in a regulator of transcription, in particular a regulator of transcription that regulates the transcription of one or more protease genes encoded by the microbial host cell genome, together with a modification leading to a reduced or no protease activity of at least one endogenous protease, and where that at least one protease is comprised in the family of serine proteases, metalloproteases, aspartyl proteases and glutamic proteases, leads to at least an additive increase in production of the compound of interest from the modified microbial cell as compared to the production of the same compound of interest from a parent microbial host cell lacking the modification that leads to a reduced or no protease activity of the at least one endogenous protease.

In a further embodiment of the invention, the combination of a modification in a regulator of transcription, in particular a regulator of transcription that regulates the transcription of one or more protease genes encoded by the microbial host cell genome, together with a modification leading to a reduced or no protease activity of at least one endogenous protease, and where that at least one protease is comprised in the family of serine proteases, metalloproteases, aspartyl proteases and glutamic proteases, leads to a synergistic (i.e., greater than additive) increase in production of the compound of interest from the modified microbial cell as compared to the production of the same compound of interest from a parent microbial host cell lacking the modification that leads to a reduced or no protease activity of the at least one endogenous protease.

In some embodiments, the modified microbial host cell of the invention characterized by having been modified in a regulator of transcription, in particular a regulator of transcription that regulates the transcription of one or more protease genes encoded by the microbial host cell genome, is further modified with a modification leading to a reduced or no protease activity of at least one endogenous protease, and where the at least one endogenous protease may be a protease selected from the family of serine proteases. For example, the at least one endogenous protease may be selected from the proteases slp2, slp6, serX-3, serX-4, serX-5, serX-9, tpp1, ser1-4, ser1-5, serX-10, sep1, slp1 and tsp1. The at least one endogenous protease may be selected from the proteases slp2, slp6, serX-3, serX-9, tpp1, ser1-4, ser1-5, serX-10, sep1, slp1 and tsp1.

In some embodiments, the modified microbial host cell of the invention characterized by having been modified in a regulator of transcription, in particular a regulator of transcription that regulates the transcription of one or more protease genes encoded by the microbial host cell genome, is further modified with a modification leading to a reduced or no protease activity of at least one endogenous protease and the at least one endogenous protease may be a protease selected from the family of metalloproteases. For example, the at least one endogenous protease may be selected from the proteases metX-11, metX-12, amp1, amp2, cpa2, cpa3, cpa5, met1-1, met1-2, met1-3, met1-6, met1-7 and vacX-1. The at least one endogenous protease may be selected from the proteases metX-11, amp1, amp2, cpa2, cpa3, cpa5, met1-1, met1-2, met1-3, met1-6, met1-7 and vacX-1.

In some embodiments, the modified microbial host cell of the invention characterized by having been modified in a regulator of transcription, in particular a regulator of transcription that regulates the transcription of one or more protease genes encoded by the microbial host cell genome, is further modified with a modification leading to a reduced or no protease activity of at least one endogenous protease and the at least one endogenous protease may be an endogenous protease selected from the family of Aspartyl proteases. For example, the at least one endogenous protease may be selected from the proteases pep1, pep2, pep3, pep4, pep5, pep8, pep9, pep11, pep12, aspX-7, and aspX-11.

In some embodiments, the modified microbial host cell of the invention characterized by having been modified in a regulator of transcription, in particular a regulator of transcription that regulates the transcription of one or more protease genes encoded by the microbial host cell genome, is further modified with a modification leading to a reduced or no protease activity of at least one endogenous protease and the at least one endogenous protease may be an endogenous protease selected from the family of glutamic proteases. In a more preferred embodiment, the at least one endogenous protease may be a protease selected from the proteases gap1 and gap2.

In a more specific embodiment, the modified microbial host cell of the invention characterized by having been modified in a regulator of transcription, in particular a regulator of transcription that regulates the transcription of one or more protease genes encoded by the microbial host cell genome, is further characterized by a reduced or no detectable protease activity in at least three proteases including either:
1. pep2, pep3 and pep4; or
2. slp1, pep1 and gap1.

In a more specific embodiment, the modified microbial host cell of the invention characterized by having been modified in a regulator of transcription, in particular a regulator of transcription that regulates the transcription of one or more protease genes encoded by the microbial host cell genome, is further characterized by a reduced or no detectable protease activity in at least five proteases including:
1. pep2, pep3, pep4, pep5 and sep1.

In a more specific embodiment, the modified microbial host cell of the invention characterized by having been modified in a regulator of transcription, in particular a regulator of transcription that regulates the transcription of one or more protease genes encoded by the microbial host cell genome, is further characterized by a reduced or no detectable protease activity in at least six proteases including:
1. pep2, pep3, pep4, pep5, gap2 and sep1;
2. slp1, pep1, gap1, pep5, gap2 and sep1; or
3. slp1, pep1, gap1, pep2, pep5 and sep1.

In a more specific embodiment, the modified microbial host cell of the invention characterized by having been modified in a regulator of transcription, in particular a regulator of transcription that regulates the transcription of one or more protease genes encoded by the microbial host cell genome, is further characterized by a reduced or no detectable protease activity in at least seven proteases including:
1. pep2, pep3, pep4, pep5, gap2, sep1 and pep1;
2. slp1, pep1, gap1, pep5, gap2, sep1 and pep2;
3. slp1, pep1, gap1, pep2, pep5, sep1 and gap1; or
4. slp1, pep1, gap1, pep2, pep5, sep1 and gap2.

In a more specific embodiment, the modified microbial host cell of the invention characterized by having been modified in a regulator of transcription, in particular a regulator of transcription that regulates the transcription of one or more protease genes encoded by the microbial host cell genome, is further characterized by a reduced or no detectable protease activity in at least eight proteases including:
1. pep2, pep3, pep4, pep5, gap2, sep1, pep1 and gap1; or
2. pep2, pep3, pep4, pep5, gap2, sep1, slp1 and gap1.

In a more specific embodiment, the modified microbial host cell of the invention characterized by having been modified in a regulator of transcription, in particular a regulator of transcription that regulates the transcription of one or more protease genes encoded by the microbial host cell genome, is further characterized by a reduced or no detectable protease activity in at least nine proteases including:
1. pep2, pep3, pep4, pep5, gap2, sep1, slp1, pep1 and gap1.

In a more specific embodiment, the microbial host cell has reduced or no detectable protease activity in at least 10 proteases including:
1. pep2, pep3, pep4, pep5, gap2, sep1, slp1, pep1, gap1 and serX-4.
2. pep2, pep3, pep4, pep5, gap2, sep1, slp1, pep1, gap1 and serX-5.
3. pep2, pep3, pep4, pep5, gap2, sep1, slp1, pep1, gap1 and metX-12.

In a more specific embodiment, the microbial host cell has reduced or no detectable protease activity in at least eleven proteases including:
1. pep2, pep3, pep4, pep5, gap2, sep1, slp1, pep1, gap1, serX-4 and serX-5.
2. pep2, pep3, pep4, pep5, gap2, sep1, slp1, pep1, gap1, serX-4 and metX-12.
3. pep2, pep3, pep4, pep5, gap2, sep1, slp1, pep1, gap1, serX-5 and metX-12.

In a more specific embodiment, the microbial host cell has reduced or no detectable protease activity in at least twelve proteases including:
1. pep2, pep3, pep4, pep5, gap2, sep1, slp1, pep1, gap1, serX-4, serX-5 and metX-12.

Preferably, the modified microbial host cell has
a) a partial or full deletion in the polynucleotide(s) encoding the at least one endogenous protease; and
b) a partial or full deletion of the polynucleotide encoding the regulator of transcription.

More preferably, the modified microbial host cell has
a) a partial or full deletion in each of the polynucleotides encoding the following endogenous proteases: pep2, pep3, pep4, pep5, gap2, sep1, pep1 and gap1; and
b) a partial or full deletion in the polynucleotide encoding Are1.

Preferably, the modified microbial host cell has
a) a partial or full deletion in the polynucleotide(s) encoding the at least one endogenous protease; and
b) a partial or full deletion of the polynucleotide encoding the regulator of transcription.

More preferably, the modified microbial host cell has
a) a partial or full deletion in each of the polynucleotides encoding the following endogenous proteases: pep2, pep3, pep4, pep5, gap2, sep1, slp1, pep1 and gap1; and
b) a partial or full deletion in the polynucleotide encoding Are1.

Preferably, the modified microbial host cell has
a) a partial or full deletion in the polynucleotide(s) encoding the at least one endogenous protease; and
b) a partial or full deletion of the polynucleotide encoding the regulator of transcription.

More preferably, the modified microbial host cell has
a) a partial or full deletion in each of the polynucleotides encoding the following endogenous proteases: pep2, pep3, pep4, pep5, gap2, sep1, slp1, pep1, gap1, serX-4, serX-5 and metX-12; and
b) a partial or full deletion in the polynucleotide encoding Are1.

The modified microbial host cell of the invention as described above is further characterized by comprising a recombinant polynucleotide encoding a compound of interest and wherein production of the compound of interest from the modified microbial cell is increased compared to the production of the same compound of interest from a parent microbial host cell lacking the modification in the regulator of transcription, in particular a regulator of transcription that regulates the transcription of one or more protease genes encoded by the microbial host cell genome and further lacking the modification that leads to a reduced or no protease activity of at least one endogenous protease, when measured under the same or substantially the same conditions.

### Identity

For comparing two or more nucleotide sequences, sequence “identity” may be used, in which the “(percentage of) sequence identity” between a first nucleotide sequence and a second nucleotide sequence may be calculated using methods known by the person skilled in the art.

The terms “sequence identity”, “% identity” are used interchangeable herein. For the purposes of this invention, it is defined here that in order to determine the percentage of sequence identity of two amino acid sequences or of two nucleic acid sequences, the sequences are aligned for optimal comparison purposes. In order to optimize the alignment between the two sequences gaps may be introduced in any of the two sequences that are compared. Such alignment can be carried out over the full length of the sequences being compared.

Alternatively, the alignment may be carried out over a shorter length, for example over about 20, about 50, about 100 or more nucleic acids/bases or amino acids. The sequence identity is the percentage of identical matches between the two sequences over the reported aligned region. The percent sequence identity between two amino acid sequences or between two nucleotide sequences may be determined using the Needleman and Wunsch algorithm for the alignment of two sequences (Needleman, S. B. and Wunsch, C. D. (1970) J. Mol. Biol. 48, 443-453). Both amino acid sequences and nucleotide sequences can be aligned by the algorithm. The Needleman-Wunsch algorithm has been implemented in the computer program NEEDLE. For the purpose of this invention the NEEDLE program from the EMBOSS package may be used (version 2.8.0 or higher, EMBOSS: The European Molecular Biology Open Software Suite (2000) Rice, Longden and Bleasby, Trends in Genetics 16, (6) pp 276-277, http:/emboss.bioinformatics.nl/).

For protein sequences EBLOSUM62 is used for the substitution matrix. For nucleotide sequence, EDNAFULL is used. The optional parameters used are a gap-open penalty of 10 and a gap extension penalty of 0.5. The skilled person will appreciate that all these different parameters will yield slightly different results but that the overall percentage identity of two sequences is not significantly altered when using different algorithms.

After alignment by the program NEEDLE as described above the percentage of sequence identity between a query sequence and a sequence of the invention is calculated as follows: number of corresponding positions in the alignment showing an identical amino acid or identical nucleotide in both sequences divided by the total length of the alignment after subtraction of the total number of gaps in the alignment. The identity as defined herein can be obtained from NEEDLE by using the NOBRIEF option and is labeled in the output of the program as “longest identity”. If both amino acid sequences which are compared do not differ in any of their amino acids over their entire length, they are identical or have 100% identity. Amino acid sequences and nucleic acid sequences are said to be “exactly the same” or “identical” if they have 100% sequence identity over their entire length.

In determining the degree of sequence identity between two amino acid sequences, the skilled person may take into account so-called ‘conservative’ amino acid substitutions, which can generally be described as amino acid substitutions in which an amino acid residue is replaced with another amino acid residue of similar chemical structure and which has little or essentially no influence on the function, activity or other biological properties of the polypeptide. Possible conservative amino acid substitutions will be clear to the person skilled in the art.

### Antibody

As used herein, the term “antibody” refers to polyclonal antibodies, monoclonal antibodies, humanized antibodies, chimeric antibodies, minibodies, diabodies, nanobodies, nanoantibodies, affibodies, alphabodies, designed ankyrin-repeat domains, anticalins, knottins, engineered CH2 domains, single-chain antibodies, or fragments thereof such as Fab F(ab)₂, F(ab)₃, scFv, a single domain antibody, a heavy chain variable domain of an antibody, a heavy chain variable domain of a heavy chain antibody (VHH), the variable domain of a camelid heavy chain antibody, a variable domain of the a new antigen receptor (vNAR), a variable domain of a shark new antigen receptor, or other fragments or antibody formats that retain the antigen binding function of a parent antibody. As such, an antibody may refer to an immunoglobulin, or fragment or portion thereof, or to a construct comprising an antigen-binding portion comprised within a modified immunoglobulin-like framework, or to an antigen-binding portion comprised within a construct comprising a nonimmunoglobulin-like framework or scaffold.

As used herein, the term “monoclonal antibody” refers to an antibody composition having a homogeneous antibody population. The term is not limited regarding the species or source of the antibody, nor is it intended to be limited by the manner in which it is made. The term encompasses whole immunoglobulins as well as fragments such as Fab, F(ab)2, Fv, and others that retain the antigen binding function of the antibody. Monoclonal antibodies of any mammalian species can be used in this invention. In practice, however, the antibodies will typically be of rat or murine origin because of the availability of rat or murine cell lines for use in making the required hybrid cell lines or hybridomas to produce monoclonal antibodies. As used herein, the term “polyclonal antibody” refers to an antibody composition having a heterogeneous antibody population. Polyclonal antibodies are often derived from the pooled serum from immunized animals or from selected humans.

“Heavy chain variable domain of an antibody or a functional fragment thereof” (also indicated hereafter as VHH), as used herein, means (i) the variable domain of the heavy chain of a heavy chain antibody, which is naturally devoid of light chains, including but not limited to the variable domain of the heavy chain of heavy chain antibodies of camelids or sharks or (ii) the variable domain of the heavy chain of a conventional four-chain antibody (also indicated hereafter as VH), including but not limited to a camelized (as further defined herein) variable domain of the heavy chain of a conventional four-chain antibody (also indicated hereafter as camelized VH).

### Methods of Culturing Filamentous Fungal Cells and Production of Heterologous Proteins

“Culturing”, “cell culture”, “fermentation”, “fermenting” or “microbial fermentation” as used herein means the use of a microbial cell to produce a compound of interest, such as a polypeptide, at an industrial scale, laboratory scale or during scale-up experiments. It includes suspending the microbial cell in a broth or growth medium, providing sufficient nutrients including but not limited to one or more suitable carbon source (including glucose, sucrose, fructose, sophorose, lactose, Avicel®, xylose, galactose, ethanol, methanol, or more complex carbon sources such as molasses or wort), nitrogen source (such as yeast extract, peptone or beef extract), trace element (such as iron, copper, magnesium, manganese or calcium), amino acid or salt (such as sodium chloride, magnesium chloride or natrium sulfate) or a suitable buffer (such as phosphate buffer, succinate buffer, HEPES buffer, MOPS buffer or Tris buffer). Optionally it includes one or more inducing agents driving expression of the compound of interest or a compound involved in the production of the compound of interest (such as lactose, IPTG, ethanol, methanol, sophorose or sophorolipids). If can also further involve the agitation of the culture media via for example stirring of purging to allow for adequate mixing and aeration. It can further involve different operational strategies such as batch cultivation, semi-continuous cultivation or continuous cultivation and different starvation or induction regimes according to the requirements of the microbial cell and to allow for an efficient production of the compound of interest or a compound involved in the production of the compound of interest. Alternatively, the microbial cell is grown on a solid substrate in an operational strategy commonly known as solid state fermentation.

Fermentation broth, culture media or cell culture media as used herein can mean the entirety of liquid or solid material of a fermentation or culture at any time during or after that fermentation or culture, including the liquid or solid material that results after optional steps taken to isolate the compound of interest. As such, the fermentation broth or culture media as defined herein includes the surroundings of the compound of interest after isolation of the compound of interest, during storage and/or during use as an agrochemical or pharmaceutical composition. Fermentation broth is also referred to herein as a culture medium or cell culture medium.

“Peptone” as used herein means a “protein hydrolysate”, which is any water-soluble mixture of polypeptides and amino acids formed by the partial hydrolysis of protein. More specifically “peptone” or “protein hydrolysate” are the water-soluble products derived from the partial hydrolysis of protein rich starting material which can be derived from plant, yeast, or animal sources. Typically, “peptone” or “protein hydrolysate” are produced by a protein hydrolysis process accomplished using strong acids, bases, or proteolytic enzymes. In more detail peptone or protein hydrolysates are produced by combining protein and demineralized water to form a thick suspension of protein material in large-capacity digestion vessels, which are stirred continuously throughout the hydrolysis process. For acid or basic hydrolysis, the temperature is adjusted, and the digestion material added to the vessel. For proteolytic digestion, the protein suspension is adjusted to the optimal pH and temperature for the specific enzyme or enzymes chosen for the hydrolysis. The desired degree of hydrolysis depends on the amount of enzyme, time for digestion, and control of pH and temperature. A typical “peptone” or “protein hydrolysate” may comprise about 25% polypeptides, about 30% free amino acids, about 20% carbohydrates, about 15% salts and trace metals and about 10% vitamins, organic acids, and organic nitrogen bases. Depending on the starting material “peptone” or “protein hydrolysate” can be completely free of animal derived products and/or GMO products. For example, “Peptone” or “protein hydrolysate” can be produced using high quality pure protein as a starting material. Alternatively, “peptone” or “protein hydrolysate” can be produce by using soya as a starting material. When soya is used as a starting material this soya can be free of animal sources. This soya can furthermore be free of GMO material. This soya can be defatted soya. Alternatively, “peptone” or “protein hydrolysate” can be produced by using casein as a starting material. Alternatively, “peptone” or “protein hydrolysate” can be produce by using milk as a starting material. Alternatively, “peptone” or “protein hydrolysate” can be produce by using meat paste as a starting material. When meat paste is used as a starting material this meat paste can be for example from bovine or porcine origin. When meat paste is used as a starting material this meat paste can be derived from organs, such as harts or alternatively for example muscle tissue. Alternatively, “peptone” or “protein hydrolysate” can be produced using gelatin as a starting material. Alternatively, “peptone” or “protein hydrolysate” can be produced by using yeast as a starting material.

“Isolating the compound of interest” is an optional step or series of steps taking the cell culture media or fermentation broth as an input and increasing the amount of the compound of interest relative to the amount of culture media or fermentation broth. Isolating the compound of interest may alternatively or additionally comprises obtaining or removing the compound of interest form the culture media or fermentation broth. Isolating the compound of interest can involve the use of one or multiple combinations of techniques well known in the art, such as precipitation, centrifugation, sedimentation, filtration, diafiltration, affinity purification, size exclusion chromatography and/or ion exchange chromatography. In some embodiments, isolating the compound of interest may comprise a step of lysing the microbial cells to release the compound of interest, for example if the compound of interest is not secreted by the microbial cells, or at least is not secreted by the microbial cells to a significant enough degree. Isolating the compound of interest may be followed by formulation of the compound of interest into an agrochemical or pharmaceutical composition.

The term “yield” as used herein refers to the amount of a compound of interest produced. When using the term “improved” or “increased” or a similar term when referring to “yield”, it is meant that the compound of interest produced by the modified microbial host cell of the invention capable of producing a compound of interest is increased in quantity, quality, stability and/or concentration either in the fermentation broth or cell culture media, as a purified or partially purified compound, during storage and/or during use as an agrochemical or pharmaceutical composition. The increase in yield is compared to the yield of compound of interest produced by a microbial host cell that has not been modified to affect the production, stability and/or function of at least one polypeptide, i.e. the parental microbial host cell.

In some embodiments, the yield is increased by at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90% or at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290% or at least about 300%, at least about 500%, at least about 1000% or at least about 1500% when a modified microbial host cell is used to produce the compound of interest, compared to a parental microbial host cell (the parental host cell only having been modified to express the compound of interest).

### Methods of Producing Compounds of Interest

The invention provides methods for the production of a compound of interest. The compound of interest may be a compound as described herein, for example an antibody or a functional fragment thereof, a carbohydrate binding domain, a heavy chain antibody or a functional fragment thereof, a single domain antibody, a heavy chain variable domain of an antibody or a functional fragment thereof, a heavy chain variable domain of a heavy chain antibody or a functional fragment thereof, a variable domain of camelid heavy chain antibody (VHH) or a functional fragment thereof, a variable domain of a new antigen receptor, a variable domain of shark new antigen receptor (vNAR) or a functional fragment thereof, a minibody, a nanobody, a nanoantibody, an affibody, an alphabody, a designed ankyrin-repeat domain, an anticalins, a knottins or an engineered CH2 domain. In some embodiments, the compound of interest is an antibody, for example a VHH.

In some embodiments, the compound of interest is a therapeutic protein, biosimilar, multi-domain protein, peptide hormone, antimicrobial peptide, peptide, carbohydrate-binding module, enzyme, cellulase, protease, protease inhibitor, aminopeptidase, amylase, carbohydrase, carboxypeptidase, catalase, chitinase, cutinase, deoxyribonuclease, esterase, alpha-galactosidase, beta-galactosidase, glucoamylase, alpha-glucosidase, beta-glucosidase, invertase, laccase, lipase, mannanase, mutanase, oxidase, pectinolytic enzyme, peroxidase, phospholipase, phytase, phosphatase, polyphenoloxidase, redox enzyme, proteolytic enzyme, ribonuclease, transglutaminase or xylanase.

In some embodiments, the compound of interest is a VHH. In more specific embodiments, the VHH may be a VHH bind a specific lipid fraction of the cell membrane of a fungal spore. Such VHHs may exhibit fungicidal activity through retardation of growth and/or lysis and explosion of spores, thus preventing mycelium formation. The VHH may therefore have fungicidal or fungistatic activity.

In some embodiments, the VHH may be a VHH that is capable of binding to a lipid-containing fraction of the plasma membrane of a fungus (for example Botrytis cinerea or other fungus). Said lipid-containing fraction may be obtainable by chromatography. For example, said lipid-containing fraction may be obtainable by a method comprising:
fractionating hyphae of a fungus (for example Botrytis cinerea or other fungus) by total lipid extract thin-layer chromatography and selecting the fraction with a Retention Factor (Rf) higher than the ceramide fraction and lower than the non-polar phospholipids fraction.

The invention also provides a polypeptide, wherein said at least one polypeptide is capable of binding to a lipid-containing fraction of the plasma membrane of a fungus (for example Botrytis cinerea or other fungus). Said lipid-containing fraction may be obtainable by chromatography.

For example, said lipid-containing fraction may be obtainable by a method comprising: fractionating hyphae of a fungus (for example Botrytis cinerea or other fungus) by total lipid extract thin-layer chromatography and selecting the fraction with a Retention Factor (Rf) higher than the ceramide fraction and lower than the non-polar phospholipids fraction.

The VHHs are generally capable of binding to a fungus. The VHH thereby causes retardation of growth of a spore of the said fungus and/or lysis of a spore of the said fungus. That is to say, binding of the VHH to a fungus results in retardation of growth of a spore of the said fungus and/or lysis of a spore of the said fungus.

The VHHs may (specifically) bind to a membrane of a fungus or a component of a membrane of a fungus. In some embodiments, the VHHs do not (specifically) bind to a cell wall or a component of a cell wall of a fungus. For example, in some embodiments, the VHHs do not (specifically) bind to a glucosylceramide of a fungus.

The VHHs may be capable of (specifically) binding to a lipid-containing fraction of the plasma membrane of a fungus, such as for example a lipid-containing fraction of Botrytis cinerea or other fungus. Said lipid-containing fraction (of Botrytis cinerea or otherwise) may be obtainable by chromatography. The chromatography may be performed on a crude lipid extract (also referred to herein as a total lipid extract, or TLE) obtained from fungal hyphae and/or conidia. The chromatography may be, for example, thin-layer chromatography or normal-phase flash chromatography. The chromatography (for example thin-layer chromatography) may be performed on a substrate, for example a glass plate coated with silica gel. The chromatography may be performed using a chloroform/methanol mixture (for example 85/15% v/v) as the eluent.

For example, said lipid-containing fraction may be obtainable by a method comprising:
fractionating hyphae and/or conidia of a fungus (for example Botrytis cinerea or other fungus) by total lipid extract thin-layer chromatography and selecting the fraction with a Retention Factor (Rf) higher than the ceramide fraction and lower than the non-polar phospholipids fraction.

In a more specific embodiment, the lipid-containing fraction may be obtainable by a method comprising:
fractionating hyphae and/or conidia of a fungus (for example Botrytis cinerea or other fungus) by total lipid extract thin-layer chromatography on a silica-coated glass slide using a chloroform/methanol mixture (for example 85/15% v/v) as the eluent and selecting the fraction with a Retention Factor (Rf) higher than the ceramide fraction and lower than the non-polar phospholipids fraction.

Alternatively, the fraction may be obtained using normal-phase flash chromatography. In such a method, the method may comprise:
fractionating hyphae and/or conidia of a fungus (for example Botrytis cinerea or other fungus) by total lipid extract normal-phase flash chromatography, and selecting the fraction with a Retention Factor (Rf) higher than the ceramide fraction and lower than the non-polar phospholipids fraction.

In a more specific embodiment, the lipid-containing fraction may be obtainable by a method comprising:
fractionating hyphae and/or conidia of a fungus (for example Botrytis cinerea or other fungus) by total lipid extract normal-phase flash chromatography comprising dissolving the TLE in dichloromethane (CH₂Cl₂) and MeOH and using CH₂Cl₂/MeOH (for example 85/15%, v/v) as the eluent, followed by filtration of the fractions through a filter.

In a more specific embodiment, the lipid-containing fraction may be obtainable by a method comprising:
fractionating hyphae and/or conidia of a fungus (for example Botrytis cinerea or other fungus) by total lipid extract normal-phase flash chromatography comprising dissolving the TLE in dichloromethane (CH₂Cl₂) and MeOH loading the TLE on to a phase flash cartridge (for example a flash cartridge with 15 μm particles), running the column with CH₂Cl₂/MeOH (85/15%, v/v) as the eluent, and filtering the fractions through a filter (for example a 0.45 μm syringe filter with a nylon membrane) and drying the fractions.

The fractions from the chromatography may be processed prior to testing of binding of the VHH to the fraction or of interaction with the fraction. For example, liposomes comprising the fractions may be prepared. Such a method may comprise the use of thin-film hydration. For example, in such a method, liposomes may be prepared using thin-film hydration with the addition of 1,6-diphenyl-1,3,5-hexatriene (DPH). Binding and/or disruption of the membranes by binding of the VHH may be measured by a change in fluorescence before and after polypeptide binding (or by reference to a suitable control).

Accordingly, in some embodiments, the VHHs may (specifically) bind to a lipid-containing chromatographic fraction of the plasma membrane of a fungus, optionally wherein the lipid-containing chromatographic fraction is prepared into liposomes prior to testing the binding of the polypeptide thereto.

Binding of the VHH to a lipid-containing fraction of a fungus may be confirmed by any suitable method, for example bio-layer interferometry. Specific interactions with the lipid-containing fractions may be tested. For example, it may be determined if the polypeptide is able to disrupt the lipid fraction when the fraction is prepared into liposomes, for example using thin-film hydration.

In methods involving chromatography, an extraction step may be performed prior to the step of chromatography. For example, fungal hyphae and/or conidia may be subjected to an extraction step to provide a crude lipid extract or total lipid extract on which the chromatography is performed. For example, in some embodiments, fungal hyphae and/or conidia (for example fungal hyphae and/or conidia of Fusarium oxysporum or Botrytis cinerea) may be extracted at room temperature, for example using chloroform:methanol at 2:1 and 1:2 (v/v) ratios. Extracts so prepared may be combined and dried to provide a crude lipid extract or TLE.

Accordingly, in some embodiments, the VHH may be capable of (specifically) binding to a lipid-containing fraction of the plasma membrane of a fungus (such as Fusarium oxysporum or Botrytis cinerea), wherein the lipid-containing fraction of the plasma membrane of the fungus is obtained or obtainable by chromatography. The chromatography may be normal-phase flash chromatography or thin-layer chromatography. Binding of the VHH to the lipid to the lipid-containing fraction may be determined according to bio-layer interferometry. In some embodiments, the chromatography step may be performed on a crude lipid fraction obtained or obtainable by a method comprising extracting lipids from fungal hyphae and/or conidia from a fungal sample. The extraction step may use chloroform:methanol at 2:1 and 1:2 (v/v) ratios to provide two extracts, and then combining the extracts.

In methods relating to thin-layer chromatography, the chromatography may comprise the steps of:
fractionating hyphae of the fungus by total lipid extract thin-layer chromatography and selecting the fraction with a Retention Factor (RD higher than the ceramide fraction and lower than the non-polar phospholipids fraction.

In some methods relating to thin-layer chromatography, the chromatography may comprise the steps of:
fractionating hyphae and/or conidia of a fungus (for example Botrytis cinerea or other fungus) by total lipid extract thin-layer chromatography on a silica-coated glass slide using a chloroform/methanol mixture (for example 85/15% v/v) as the eluent and selecting the fraction with a Retention Factor (RD higher than the ceramide fraction and lower than the non-polar phospholipids fraction.

In methods relating to normal-phase flash chromatography, the chromatography may comprise the steps of:
fractionating hyphae and/or conidia of a fungus (for example Botrytis cinerea or other fungus) by total lipid extract normal-phase flash chromatography, and selecting the fraction with a Retention Factor (RD higher than the ceramide fraction and lower than the non-polar phospholipids fraction.

In some methods relating to normal-phase flash chromatography, the chromatography may comprise the steps of:
fractionating hyphae and/or conidia of a fungus (for example Botrytis cinerea or other fungus) by total lipid extract normal-phase flash chromatography comprising dissolving the TLE in dichloromethane (CH₂Cl₂) and MeOH and using CH₂Cl₂/MeOH (for example 85/15%, v/v) as the eluent, followed by filtration of the fractions through a filter.

In some methods relating to normal-phase flash chromatography, the chromatography may comprise the steps of:
fractionating hyphae and/or conidia of a fungus (for example Botrytis cinerea or other fungus) by total lipid extract normal-phase flash chromatography comprising dissolving the TLE in dichloromethane (CH₂Cl₂) and MeOH loading the TLE on to a phase flash cartridge (for example a flash cartridge with 15 μm particles), running the column with CH₂Cl₂/MeOH (85/15%, v/v) as the eluent, and filtering the fractions through a filter (for example a 0.45 μm syringe filter with a nylon membrane) and drying the fractions.

In some embodiments, the compound of interest is VHH-1, VHH-2 or VHH-3. For example, in some embodiments, the compound of interest is a VHH comprising or consisting of a sequence selected from the group consisting of SEQ ID NOs: 118, 119, 123, 127, 131 and 132.

In some embodiments, the compound of interest is a VHH comprising:
(a) a CDR1 comprising or consisting of a sequence selected from the group consisting of SEQ ID NOs 120, 124 and 128;
(b) a CDR2 comprising or consisting of a sequence selected from the group consisting of SEQ ID NOs: 121, 125 and 129; and
(c) a CDR3 comprising or consisting of a sequence selected from the group consisting of SEQ ID NOs: 122, 126 and 130.

In some embodiments, the compound of interest is a VHH comprising:
(a) a CDR1 comprising or consisting of the sequence of SEQ ID NO: 120, a CDR2 comprising or consisting of the sequence of SEQ ID NO: 121 and a CDR3 comprising or consisting of the sequence of SEQ ID NO: 122;
(b) a CDR1 comprising or consisting of the sequence of SEQ ID NO: 124, a CDR2 comprising or consisting of the sequence of SEQ ID NO: 125 and a CDR3 comprising or consisting of the sequence of SEQ ID NO: 126 or
(c) a CDR1 comprising or consisting of the sequence of SEQ ID NO: 128, a CDR2 comprising or consisting of the sequence of SEQ ID NO: 129 and a CDR3 comprising or consisting of the sequence of SEQ ID NO: 130.

In some embodiments, the compound of interest is a VHH comprising a CDR1 comprising or consisting of the sequence of SEQ ID NO: 120, a CDR2 comprising or consisting of the sequence of SEQ ID NO: 121 and a CDR3 comprising or consisting of the sequence of SEQ ID NO: 122.

In some embodiments, the compound of interest is a VHH comprising SEQ ID NO: 118.

In some embodiments, the compound of interest is a VHH comprising SEQ ID NO: 119.

In some embodiments, the compound is a VHH disclosed in WO2014/177595 or WO2014/191146, the entire contents of which are incorporated herein by reference. More specifically the VHH comprises an amino acid sequence chosen from the group consisting of SEQ ID NO's: 1 to 84 from WO2014/177595 or WO2014/191146.

Thus, the microbial host cells of the invention can be used to produce compounds of interest, in particular VHHs, such as the VHHs disclosed herein, as well as other VHHs, such as those disclosed in WO2014/177595 or WO2014/191146. In some embodiments, the VHHs are fused to a carrier peptide.

The methods comprise providing a modified microbial host cell of the invention, which is characterized by (a) having been modified and where this modification leads to a reduced or no protease activity of at least one endogenous protease; and (b) comprising a recombinant polynucleotide encoding a compound of interest. The host cell is capable of expressing the compound of interest. The method further comprises culturing said modified microbial host cell under conditions conducive to the expression of the compound of interest. The method may further optionally comprise a step of isolating the compound of interest from the culture medium or fermentation broth.

The modified microbial host cell that is provided may already be capable of expressing the compound of interest. For example, the modified microbial host cell may be provided already comprising a polynucleotide coding for the compound of interest, and the sequence encoding the compound of interest may be operable linked to a promoter (for example a constitutive promoter or an inducible promoter). Alternatively, the method may comprise a step of transforming the microbial host cell with the polynucleotide to insert the polynucleotide into the microbial host cell.

The step of transforming the microbial host cell, if present, may occur before, after or simultaneously with the modification of the microbial host cell to modify the production, structure and/or function of the at least one polypeptide.

In some embodiments, the methods may comprise a step of inducing expression of the compound of interest by the microbial host cell. For example, if the compound of interest is encoded by a nucleotide sequence that is operably linked to an inducible promoter, the method may comprise a step of inducing the expression of the compound of interest. A common inducible promoter that may be used is the inducible cbh1 or cbh2 promoter, in which administration of lactose will initiate expression. Other inducible promoters could of course be used. If the sequence encoding the compound of interest is under the control of a constitutive promoter, no specific step of induction of expression may be required.

Fermentation or culture of the microbial host cells may occur in a solid fermentation or culture setting or a liquid fermentation or culture setting. Solid-state fermentation or culture may comprise seeding the microbial host cell on a solid culture substrate, and methods of solid-state fermentation or culture are known the skilled person. Liquid fermentation or culture may comprise culturing the microbial host cell in a liquid cell culture medium.

The method may also comprise a step of isolating the compound of interest produced by the microbial host cell, for example isolating the compound of interest from the fermentation broth or cell culture medium.

The method may further comprise a step of formulating the compound of interest into a agrochemical or pharmaceutical composition. The step of formulating the compound of interest into an agrochemical composition may comprise formulating the compound of interest with one or more agrochemically acceptable excipients. The step of formulating the compound of interest into a pharmaceutical composition may comprise formulating the compound of interest with one or more pharmaceutically acceptable excipients.

The present invention therefore provides compounds of interest obtained by a method of the present invention. The present invention also therefore provides an agrochemical or pharmaceutical composition obtained by a method of the present invention.

The present invention also provides the use of a modified microbial host cell of the invention for the production of a compound of interest, wherein the microbial host cell is characterized by (a) being modified and where this modification leads to a reduced or no protease activity of at least one endogenous protease and; (b) comprising a recombinant polynucleotide encoding a compound of interest.

Any methods comprising or requiring the culturing or fermentation of the modified microbial host cell comprise the culture or fermentation of the host cell is a suitable medium. Generally, the medium will comprise any and all nutrients required for the microbial host cell to grow. The skilled person will be aware of the required components of the cell culture media or fermentation broth, which may differ depending on the species of microbial host cell being cultured. In some embodiments, the cell culture media or fermentation broth may comprise a nitrogen source, such as ammonium or peptone.

Features of each aspect of the invention provided herein are as for each of the other aspects mutatis mutandis. For example, features which are described in the context of microbial host cells of the invention also apply to, unless context requires otherwise, the methods of the invention. It will be appreciated that certain features of the invention, which are, for clarity, described in the context of separate embodiments, may also be provided in combination in a single embodiment.

Conversely, various features of the invention, which are, for brevity, described in the context of a single embodiment, may also be provided separately or in any suitable sub-combination. All combinations of the embodiments pertaining to the modified microbial host cell are specifically embraced by the present invention and are disclosed herein just as if each and every combination was individually and explicitly disclosed.

### EXAMPLES

The following non-limiting Examples describe methods and means according to the invention. Unless stated otherwise in the Examples, all techniques are carried out according to protocols standard in the art. The following examples are included to illustrate embodiments of the invention. Those of skill in the art should, in light of the present disclosure, appreciate that many changes can be made in the specific embodiments which are disclosed and still obtain a like or similar result without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### Example 1: General Procedures for Performing a Fermentation

Fermenters are filled with medium with similar characteristics as described in Vogel, H., 1956. A convenient growth medium for Neurospora. Microbiol. Genet. Bull. 13, 42-43, or in a suitable defined medium containing ammonium sulphate (NH₄)₂SO₄ and peptone using lactose and/or sophorose as inducers. Calibration of the Dissolved oxygen (DO) levels is performed at around 28° C., 400 rpm and 60 sL/h of aeration. The pH of the medium in the fermenter is adjusted to around 5 before being inoculated in the fermenter.

Fermenters are inoculated with around 0.5%-10% inoculum density in 1980 ml medium. Incubation at around 28° C.; 1200 rpm and 60 sL/h aeration. DO lower limit at 50%. DO cascade output set as 0-40% 1200-1400 rpm of stirrer, 40-100%, 100-200 tl/h of aeration. Antifoam is dissolved as 10× in water. Ammonium hydroxide 12.5% as base. Induction with for instance lactose 20% is generally initiated after a p02 spike. The feed rate is set at approximately 9 ml/h (4.5 ml/L·h).

### Example 2: Mass Spectrometry Analysis on Fermentation Broth

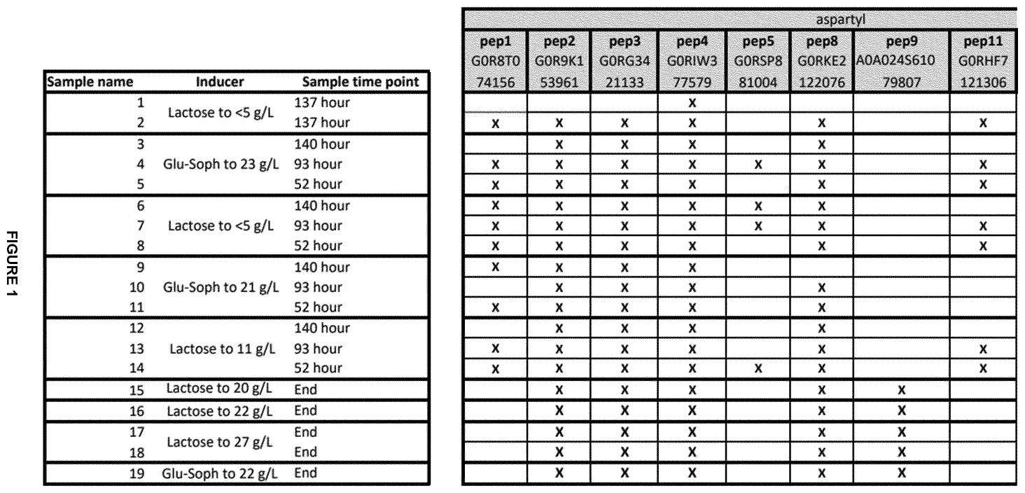

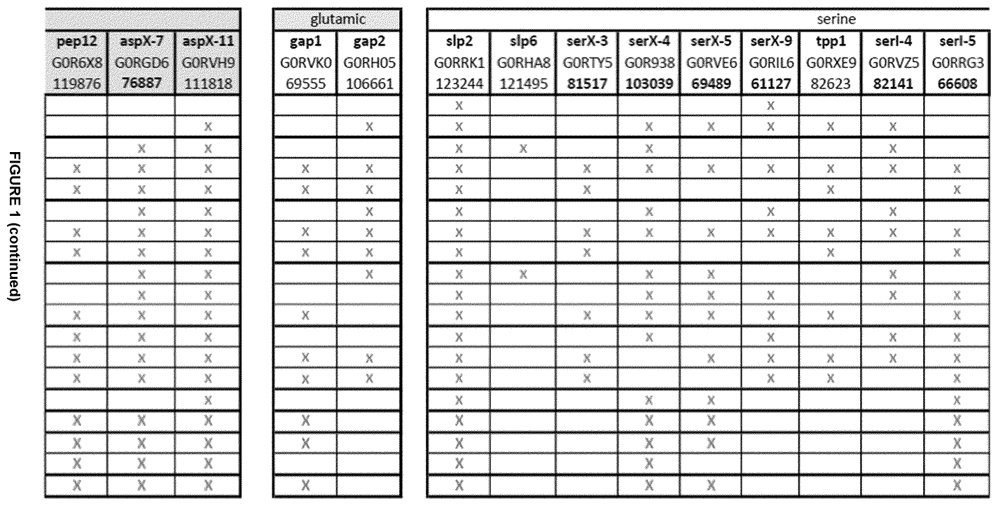

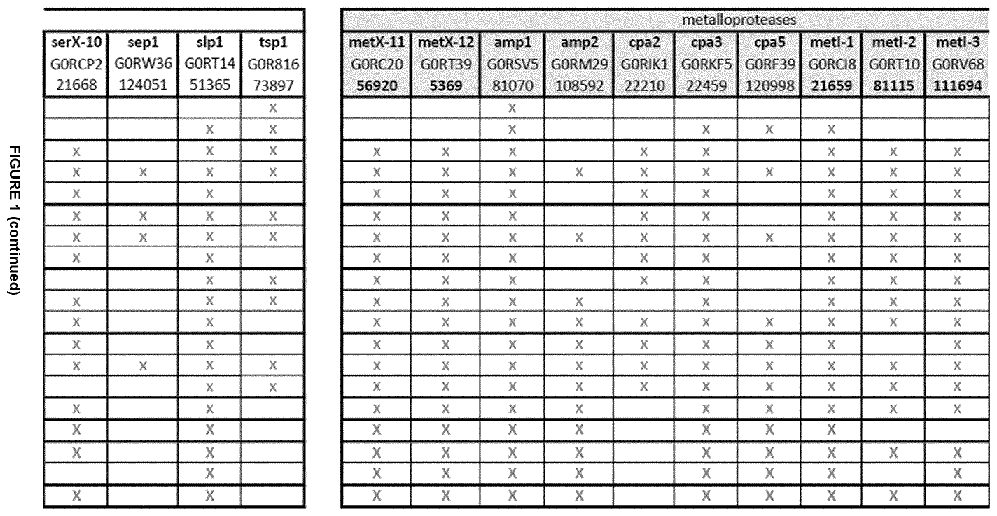

To identify the abundance and repertoire of proteins secreted by the modified microbial host cell compared to the parent microbial host cell, the cells were cultured in fermenters such as described in Example 1 using different combinations of either lactose, glucose and/or sophorose for induction of expression. Samples were taken at different timepoints during or at the end of each fermentation run. Samples were taken from the supernatant, total protein was TCA-precipitated, digested with trypsin, iTRAQ-labeled, and LC-MS/MS analyzed. MS raw files were imported into MaxQuant and proteins were identified and quantified using the MaxLFQ algorithm. The LFQ (label-free quantitation) protein values were normalized to exclude some outliers to best represent the ratio changes of different samples. These analysis identified the proteases present in a Trichoderma reesei RL-P37 fermentation broth as presented in FIG. 1.

### Example 3: Performing a Spiking Experiment

To determine the stability of a compound of interest in the fermentation broth of a modified T. reesei host cell compared to the parental T. reesei host cell a spiking experiment is performed. For this, approximately 5×10⁶ spores/mL of fresh conidia of the parent and modified T. reesei host cell are inoculated into 50 mL of Vogel's liquid medium or a defined media (with peptone or ammonium as nitrogen source) in 250 mL shake flask in duplicate and incubated at 28° C. An uninoculated control is included in all experiments. After 48 h of growth, a known concentration of purified compound of interest is spiked into fermentation media and the addition of 1000 μL of 20% lactose and/or sophorose inducer is started once a day.

During the induction process, 1 ml of homogeneous fermentation medium is taken daily from each flask to determine total soluble protein (TSP) by Bradford Protein assay kit (Thermo Scientific), according to the manufacturer's instructions.

During several days after cellulose induction the fermentation broth is sampled and all the samples are separated by SDS-PAGE electrophoresis to visualize the degradation of the compound of interest, taking 30 μL of fermentation broth, 7.5 μL sample buffer and 3.5 μL DTT, and denaturing the samples at 85° C. for 5 min. The samples are immediately transferred to ice before being loaded on SDS-PAGE gels (precast NuPAGE™ 4 to 12%, Bis-Tris, 1.0 mm, Mini Protein Gel, 12-well, Invitrogen).

### Example 4: Performing a Spiking Experiment in the Presence of Different Classes of Protease Inhibitors

To determine the stability of a compound of interest in the fermentation broth of a modified T. reesei host cell in the presence of protease inhibitors a spiking experiment similar to Example 3 can be performed in the presence of a specific protease inhibitor (for example Pepstatin A inhibiting aspartic proteases or phenylmethylsulfonyl fluoride, Aprotinin and AEBSF inhibiting serine proteases, or EDTA for inhibiting metalloproteases).

### Example 5: Deleting or Inactivating Specific Selections of Proteases

A gene can be deleted or inactivated from the genome of Trichoderma reesei according to the following protocol. To obtain the fragments necessary to assemble the deletion cassettes, genomic DNA from T. reesei is extracted using the Wizard Genomic DNA purification kit (Promega-A1120) according to the manufacturer's instructions. The pellet is resuspended in 60 μL of DNA Rehydration Solution by incubating at 65° C. for 1 hour.

To construct the donor DNAs, the 5′ and 3′ flanking fragments of target gene are amplified separately using PCR. The selection marker expression cassette comprising the hygB gene (encoding hygromycin B phosphotransferase), under the control of the oliC promoter and the trpC terminator of Aspergillus nidulans (PoliC-hph-TtrpC) is obtained via gene synthesis and amplified separately with specific primers.

The PCR amplified 5′ target gene flanking fragment, hygromycin selection marker and 3′ target gene flanking region are assembled in a cloning vector using the NEBuilder HiFi Assembly Master Mix, and E. coli DH5a competent cells are transformed with the ligation mixture to generate the plasmids containing the donor DNAs for the deletion of the target gene. The successful assembly of the donor plasmid was confirmed by restriction enzyme digestion and sequencing.

To obtain sufficient DNA for the T. reesei transformation, a PCR is performed using the donor plasmid as a template, resulting in the deletion cassette fragment.

Transformation of protoplasts from Trichoderma reesei with the deletion cassette fragment is carried out using a standard poly-ethylene glycol (PEG) mediated transformation method as described previously (Penttils et al., 1987). Successful transformants are selected on potato dextrose agar (Sigma-Aldrich) plates with 100 μg/mL hygromycin as the selective agent. The plates are incubated for 7 days until colonies could be transferred to secondary selection plates. The hygromycin resistant colonies and parent microbial host cell are grown in 500 ml Vogel's liquid medium (100 mL=2 mL Vogel's 50× stock solution, 3 mL glucose (50% stock solution), 200 mL tween-80 (10% stock solution), 9.4 mL yeast extract (80 g/L stock solution)) and 100 μg/mL of hygromycin (in the case of transformants). Genomic DNA is extracted from colonies using Phire Plant Direct PCR Kit (Thermo Fisher Scientific). The resulting genomic DNA sample is diluted into 10 μl water, debris is removed by centrifugation, and the supernatant is used as a template in subsequent PCR.

Oligonucleotides are designed outside the flanking regions of the target locus to identify the possible integration of the donor cassettes into the deletion region.

### Example 6: Assessing Protease Activity

In order to determine the protease activity present in a fermentation broth of the modified microbial host cell compared to the parent host cell, fermentations are run according to Example 1. Samples are taken during several days of the fermentation. The total protein content of the supernatant is determined using Bradford Protein assay kit (Thermo Scientific) in order to normalize different samples according to their protein content. The supernatant is thereafter analysed using the EnzChek™ Protease Assay Kit (Thermo Fisher Scientific) an assay based on the proteolytic degradations of a casein substrate leading to a fluorescent signal that is proportional to the amount of proteolytic activity present in the fermentation broth of the modified microbial host cell compared to the parent microbial host cell. Alternatively, the Pierce™ Colorimetric Protease Assay Kit is used to provide a colorimetric output proportional to the proteolytic activity present in the fermentation broth of the modified microbial host cell compared to the parent microbial host cell.

### Example 7: Cloning of a Recombinant Protein Expression Cassette

To generate a recombinant protein expression cassette, a codon-optimized version of a polynucleotide encoding the compound if interest is fused with the cellobiohydrolase I (CBHI) signal peptide coding sequence, and under the control of the cbhI or cbhII promoter sequences is synthesized. Alternatively, the catalytic domain fragment of cbhI is fused with the intact codon-optimized version of the polynucleotide encoding the compound of interest, including the KexB protease cleavage site to release the recombinant protein and CbhI carrier protein separately during protein secretion. In addition, to ensure secretion and integration of the target proteins, the same expression cassettes mentioned were readapted for their targeted integration in the cbh1 locus. The expression cassettes containing the cbhI or cbhII promoters with the target protein are flanked with 5′ and 3′ DNA homologous regions (˜1000 bp each) of cbhI locus, which results in the CbhI coding region replacement by the target gene.

To construct a selection marker cassette, a fragment of about 1.5 kbp containing nptII/neo encoding neomycin phosphotransferase gene, as well as the oliC promoter and the trpC terminator of Aspergillus nidulans (PoliC-hph-TtrpC) is obtained via gene synthesis. The co-transformation of nptII selection marker and recombinant protein expression cassette is performed as described in Example 5. After transformation, protoplasts were incubated at 28° C. for 4-6 days on selection plates containing 100 μg/mL of G418 on PDA plates. To confirm the integration of the expression cassettes, colony PCR was performed under standard PCR conditions with sequence-specific PCR primers.

### Example 8: Heterologous Protein Expression

To compare the increase in efficiency of compound of interest production, a modified microbial host cell expressing the compound of interest is compared to a parent microbial host cell expressing the same compound of interest.

The stable transformants are inoculated in production medium in shake flasks and a fermentation is run according to Example 1. Samples are taken at regular intervals and the supernatant is collected and separated by SDS-PAGE electrophoresis to visualize and quantify the compound of interest. In this way an assessment of the stability and/or production of the compound of interest from the modified microbial host cell is made in comparison with the stability and/or production from the parental microbial host cell. Additionally, the cell-free supernatant is incubated during several days to assess the stability of the compound of interest during storage.

### Example 9 Assessing Recombinant Protein Stability

To determine the stability of VHHs on the fermentation broths of modified Trichoderma strains, and the parent microbial cell host, the strains were grown in 3.8 L fermenters with defined medium and a combination of ammonium-peptone as nitrogen source.

The fermenter was filled with defined fermentation medium and trace minerals to 2000 mL. Calibration of the Dissolved oxygen (DO) levels was performed at 37° C., 1200 rpm, and 1 lpm of aeration. The pH of the medium in the fermenter was adjusted to 4.2 before inoculation of the fermenter.

Approximately 1×10⁵ fresh spores of each T. reesei strains were grown in 250 mL Erlenmeyer's containing 50 mL YPD and incubated at 30° C. and 250 rpm to obtain sufficient biomass. After 24 h of growth, the fermenters were inoculated with 1% inoculum density in 1500 mL of defined fermentation medium with peptone and trace minerals. The parameters of incubation were 37° C., 1200 rpm, 200 sL/h aeration, and DO lower limit at 50%. Antifoam was dissolved as 10× in water and added during the fermentation process. The carbon source, lactose, at a concentration of 20% was utilized for induction when glucose was depleted.

At the start of the induction, the temperature was lowered to 28° C. and 2 g/L of purified VHH-1 was spiked into fermentation media. During the induction process, 5 ml of homogeneous fermentation medium were taken daily from each fermenter to determine the concentration of spiked VHH-1 by Reverse Phase HPLC method.

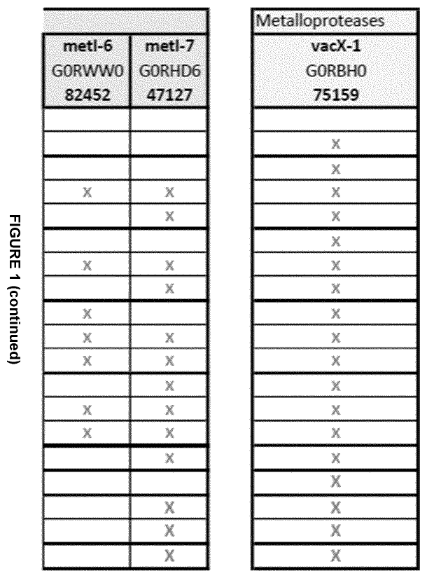

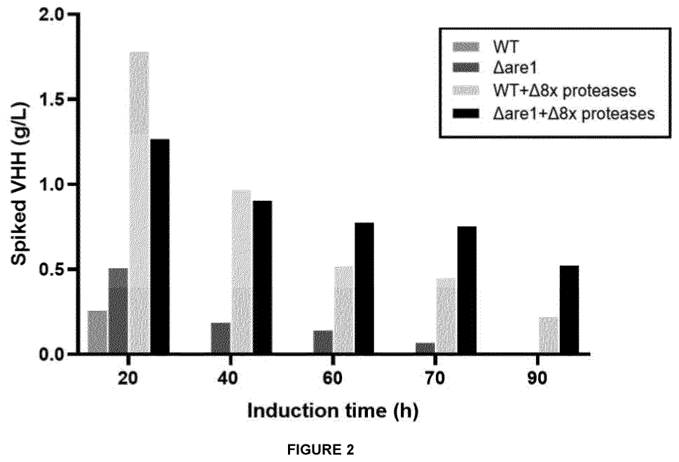

FIG. 2 shows the results of monitoring the degradation process of spiked VHH-1 during fermentation of Trichoderma reesei strains during 90 h post-lactose induction in fermentation media. Four strains were tested. A wild-type T. reesei strain showed the fastest decay of the VHH-1 falling to zero g/I by 40 hours post induction. A T. reesei strain containing the are1 deletion showed improved stability of VHH-1, with the concentration falling to zero g/I by 90 hours post induction. A Trichoderma reesei strain containing 8 protease deletions (specifically pep2, pep3, pep4, pep5, gap2, sep1, pep1 and gap1) showed a further improvement of VHH-1 stability with concentrations by 90 hours being approximately 0.22 g/l. Finally, the modified T. reesei cells containing 8 protease deletions (specifically pep2, pep3, pep4, pep5, gap2, sep1, pep1 and gap1) and the are1 deletion showed a further improvement of VHH-1 stability with the concentration by 90 hours being 0.52 g/l. Therefore, the effect of combining the are1 deletion with a set of specific protease deletions is higher than would be expected by the results of the individual modifications separately (either are1 or Δ8x proteases). The full results corresponding to FIG. 2 are provided in Table 1.

TABLE 1
concentration of VHH-1 as spiked in the
different T. reesei strain broths

| | |
|---|---|
| Time post | Concentration of VHH-1 (g/l) |

| induction | | | WT + Δ8x | Δare1 + Δ8x |
|---|---|---|---|---|
| (hours) | WT | Δare1 | proteases | proteases |
| 20 | 0.26 | 0.51 | 1.78 | 1.27 |
| 40 | 0 | 0.19 | 0.97 | 0.90 |
| 60 | 0 | 0.14 | 0.52 | 0.77 |
| 70 | 0 | 0.07 | 0.45 | 0.75 |
| 90 | 0 | 0 | 0.22 | 0.52 |

### Example 10: Deleting or Inactivating Specific Selections of Proteases: Δ9x Proteases and Δ12x Proteases

A gene can be deleted or inactivated from the genome of Trichoderma reesei according to the following protocol. To obtain the fragments necessary to generate the deletion cassettes, gene synthesis was used to clone two in-frame stop codons TAA-TAA in between the 5′ and 3′ fragments (of each 900 bp) complementary to the up and downstream regions of the target genes to be deleted by homologous recombination. To allow the selection of transformants, a selection marker expression cassette comprising the BleoR gene (encoding the phleomycin resistance marker), under the control of the oliC promoter and the trpC terminator of Aspergillus nidulans (PoliC-hph-TtrpC) was obtained via gene synthesis and cloned into an episomal plasmid. Specific primers were designed to obtain DNA material for the fungal transformation using the plasmids containing the deletion cassettes. The PCR amplification was performed employing the Phusion High-Fidelity PCR kit. Subsequently, the PCR products were purified using the Wizard DNA Clean-Up System (Promega) according to the manufacturer's instructions.

Transformation of protoplasts from Trichoderma reesei with the deletion cassettes and selection marker plasmid was carried out using a standard polyethylene glycol (PEG) mediated transformation method as described previously (Penttils et al., 1987). The parental strains P37 and Δare were used to generate 9 (specifically pep1, pep2, pep3, pep4, pep5, gap1, gap2, sep1, and slp1; referred to as Δ9x protease) and 12 (pep1, pep2, pep3, pep4, pep5, gap1, gap2, sep1, slp1, metX-12, serX-4 and serX-5; referred to as Δ12x protease) protease deletions in each strain. Successful transformants were selected on potato dextrose agar (Sigma-Aldrich) plates with phleomycin as the selective agent. The plates were incubated for 4 days until colonies could be transferred to secondary selection plates. The antibiotic-resistant colonies and parental host cells were grown in YPD liquid medium (10 g/L yeast extract, 20 g/L peptone, and 20 g/L glucose). Genomic DNA was extracted from colonies using Phire Plant Direct PCR Kit (Thermo Fisher Scientific) according to the manufacturer's instructions. The resulting genomic DNA sample was diluted into 10 μl water, debris was removed by centrifugation, and the supernatant was used as a template in subsequent PCR.

Oligonucleotides were designed outside the flanking regions of the target locus to identify the possible deletion of target genes or insertions. The expected size of the deletion was variable and target depending. Positive transformants confirmed by colony PCR were further purified to obtain single spore isolations, followed by two or three rounds of subculturing under nonselective conditions to remove the antibiotic selection plasmid from transformants.

### Example 11: Cloning of a Recombinant Protein Expression Cassette in Different Δare1, Δ9x Proteases and/or Δ12x Proteases Combination Strains

To generate a recombinant protein expression cassette, a codon-optimized version of a polynucleotide encoding a VHH was fused with the cellobiohydrolase I (CBHI) signal peptide coding sequence under the control of the cbhI promoter sequence, which was synthesized. Alternatively, the catalytic domain fragment of cbhI was fused to the intact codon-optimized version of the polynucleotide encoding the compound of interest, including the KexB protease cleavage site to release the recombinant protein and CbhI carrier protein separately during protein secretion. In addition, to ensure secretion and integration of the target proteins, the same expression cassettes mentioned were readapted for their targeted integration in the cbh1 locus. The recombinant expression cassettes containing the cbhI promoter with the VHH encoding polynucleotide were flanked with 5′ and 3′ DNA homologous regions (˜1000 bp each) of cbhI locus, which resulted in the CBHI coding region replacement by the recombinant expression cassette.

A co-transformation with the selection marker BleoR and recombinant protein expression cassette was performed as described in Example 5. The following genetic backbones were transformed with the recombinant expression cassette encoding for a VHH: P37 wild-type, P37 Δare1, P37 Δ9x protease, P37 Δare1 Δ9x proteases, P37 Δ12x proteases, and P37 Δare1 Δ12x proteases strains. After transformation, protoplasts were incubated at 28° C. for 4-6 days on selection plates. To confirm the integration of the expression cassettes was confirmed by colony PCR using standard PCR conditions with sequence-specific PCR primers.

### Example 12: Increased VHH Production in a P37 ΔAre1 Strain in Combination with Δ9x Proteases and Δ12x Proteases

To compare the stability and production of the compound of interest, the modified microbial host cells P37, P37 Δare1, P37 Δ9x proteases, P37 Δare1 Δ9x, P37 Δ12x, and P37 Δare Δ12x expressing VHH were compared. The stable transformants were inoculated in production medium in shake flasks.

Approximately 2×10⁵ spores of the selected strains were inoculated in 10 mL YPD medium in 50 mL falcon tubes and incubated for two days at 30° C. (250 rpm). After incubation, 10 mL of mycelium was added to 40 mL of the production medium. Additionally, 200 μL of Tween 80 and 1 mL of 20% lactose were added to each shake flask. Each colony was inoculated in duplicate to have two biological replicates. The shake flasks were subsequently incubated at 30° C. (250 rpm) for 7 days (168 hours). After 2, 4, and 7 days, 1 mL of the sample was taken and centrifuged for 15 minutes at max rpm. The supernatant was transferred to a new Eppendorf tube and stored at −20° C. until further processing. The remaining pellet was put in the oven at 70° C. to dry and was measured afterwards.

All the samples were separated by SDS-PAGE electrophoresis to visualize the degradation of VHH, taking 15 μL of fermentation broth, 4 μL sample buffer and 2 μL DTT, and denaturing the samples at 85° C. for 5 min. The samples were immediately transferred to ice before being loaded on SDS-PAGE gels (precast NuPAGE™ 4 to 12%, Bis-Tris, 1.0 mm, Mini Protein Gel, 12-well, Invitrogen). After SDS-PAGE, the gel was rinsed with UP water to remove the buffer salts. Next, the gel was removed from the cassette and the proteins were transferred to a nitrocellulose membrane by using the IBlot2 gel transfer device (7 min) according to the manufacturer's protocol. After the transfer, the membrane was blocked overnight in ±50 ml 4% Milk/1×PBS at 4° C. The following day, the membrane was first washed with UP water. Next, the iBind flex western system was used. The required solutions were prepared according to the manufacturer's protocol and the whole was incubated for 4 hours with Anti-camelid VHH polyclonal rabbit antibody (Genscript), and Goat anti-rabbit IgG-antibody [Alkaline Phosphatase](Sigma). Afterwards, the membrane was washed three times for 10 minutes with 0.1% Tween20/1×PBS at room temperature. Finally, the membrane was incubated with the substrate to allow AP chromogenic detection.

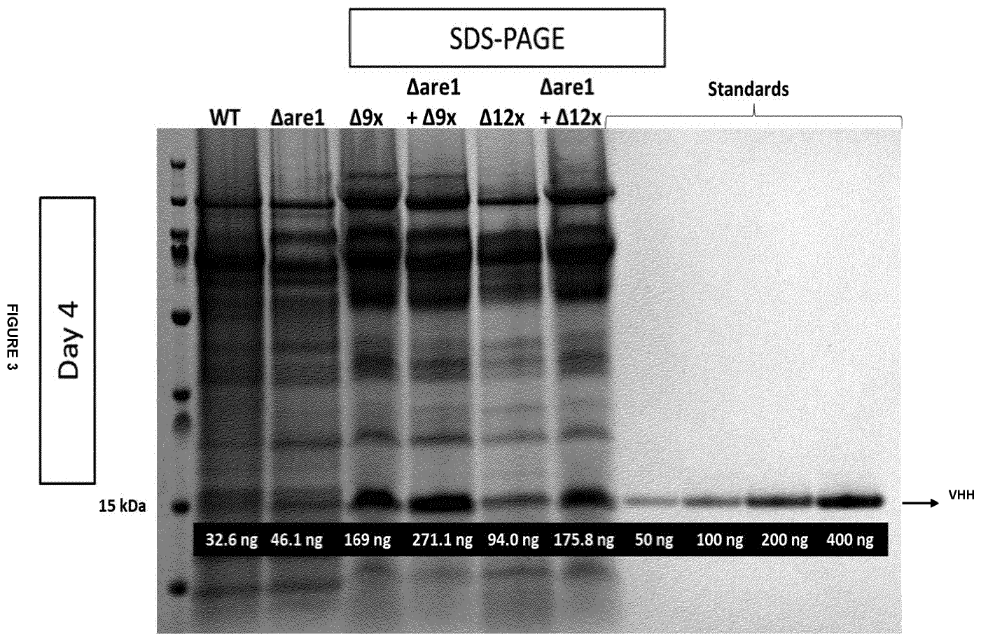

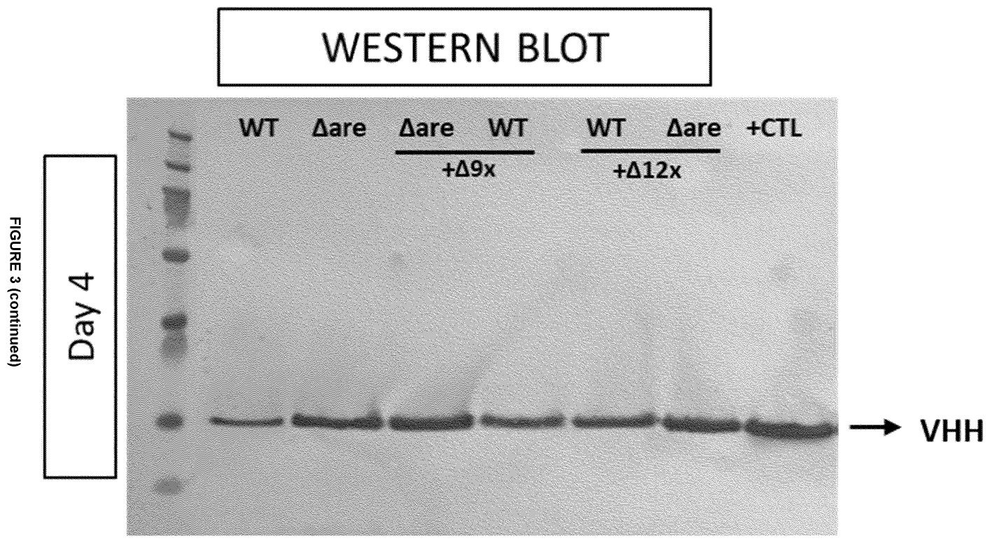

FIG. 3 shows the results at day 4 post induction. An SDS-page gel with the samples from each of the different P37 background strains expressing a VHH is shown (FIG. 3 upper panel) with a corresponding western blot shown (FIG. 3 lower panel). The strains with the Δare1 genotype showed an increased quantity of VHH at day 4 over the P37 wild-type background expressing a VHH. Furthermore the strains with Δ9x proteases or Δ12x proteases showed an increase of VHH quantity over the P37 wild-type background expressing a VHH. Combining both the Δare1 genotype with either Δ9x proteases or Δ12x proteases increased the quantity of VHH at day 4 even more. In fact, further quantification of the protein band in de SDS-PAGE (FIG. 3 upper panel) shows that the effect of combining both the Δare1 genotype with either the Δ9x proteases or Δ12x proteases genotype led to an effect that was higher than the sum of the effect created by the genotypes individually. Therefore, the effect of combining the Δare1 genotype with a set of specific protease deletions is higher than would be expected by the results of the individual modifications separately (either are1 or Δ9x or Δ12x proteases).

### EMBODIMENTS

Statements (features) and embodiments of the methods and compositions as disclosed herein are set out below. Each of the statements and embodiments as disclosed by the invention so defined may be combined with any other statement and/or embodiment unless clearly indicated to the contrary. In particular, any feature indicated as being preferred or advantageous may be combined with any other feature or features indicated as being preferred or advantageous.

The present invention provides at least the following numbered embodiments:
1. A microbial host cell which is characterized by
   a. having a modification that leads to a reduced or no protease activity of at least one endogenous protease and,
   b. comprising a recombinant polynucleotide encoding a compound of interest;
      wherein the at least one endogenous protease is selected from the proteases comprised in the family of serine proteases, metalloproteases, aspartyl proteases and glutamic proteases, and
      wherein production of the compound of interest from the modified microbial cell is increased compared to the production of the same compound of interest from a parent microbial host cell lacking the modification that leads to a reduced or no protease activity of the at least one endogenous protease.
2. The microbial host cell of embodiment 1, wherein the serine proteases are selected from the proteases slp2, slp6, serX-3, serX-4, serX-5, serX-9, tpp1, ser1-4, ser1-5, serX-10, sep1, slp1 and tsp1.
3. The microbial host cell of embodiment 1, wherein the serine proteases are selected from the proteases slp2, slp6, serX-3, serX-9, tpp1, ser1-4, ser1-5, serX-10, sep1, slp1 and tsp1.
4. The microbial host cell of embodiment 1, wherein the metalloproteases are selected from the proteases metX-11, metX-12, amp1, amp2, cpa2, cpa3, cpa5, met1-1, met1-2, met1-3, met1-6, met1-7, and vacX-1.
5. The microbial host cell of embodiment 1, wherein the metalloproteases are selected from the proteases metX-11, amp1, amp2, cpa2, cpa3, cpa5, met1-1, met1-2, met1-3, met1-6, met1-7, and vacX-1.
6. The microbial host cell of embodiment 1, wherein the aspartyl proteases are selected from the proteases pep1, pep2, pep3, pep4, pep5, pep8, pep9, pep11, pep12, aspX-7, and aspX-11.
7. The microbial host cell of embodiment 1, wherein the glutamic proteases are selected from the proteases gap1 and gap2.
8. The microbial host cell of embodiments 1-7, wherein the microbial host cell has reduced or no protease activity in the following proteases: pep2, pep3 and pep4.
9. The microbial host cell of embodiments 1-7, wherein the microbial host cell has reduced or no protease activity in the following proteases: pep2, pep3, pep4, pep5 and sep1.
10. The microbial host cell of embodiments 1-7, wherein the microbial host cell has reduced or no protease activity in the following proteases: pep2, pep3, pep4, pep5, gap2 and sep1.
11. The microbial host cell of embodiments 1-7, wherein the microbial host cell has reduced or no protease activity in the following proteases: pep2, pep3, pep4, pep5, gap2, sep1 and pep1.
12. The microbial host cell of embodiments 1-7, wherein the microbial host cell has reduced or no protease activity in the following proteases: pep2, pep3, pep4, pep5, gap2, sep1, pep1 and gap1.
13. The microbial host cell of embodiments 1-7, wherein the microbial host cell has reduced or no protease activity in the following proteases: pep2, pep3, pep4, pep5, gap2, sep1, slp1 and gap1.
14. The microbial host cell of embodiments 1-7, wherein the microbial host cell has reduced or no protease activity in the following proteases: pep2, pep3, pep4, pep5, gap2, sep1, slp1, pep1 and gap1.
15. The microbial host cell of embodiments 1-7, wherein the microbial host cell has reduced or no protease activity in the following proteases: pep2, pep3, pep4, pep5, gap2, sep1, slp1, pep1, gap1, serX-4, serX-5 and metX-12.
16. The microbial host cell of embodiments 1-7, wherein the microbial host cell has reduced or no protease activity in the following proteases: slp1, pep1 and gap1.
17. The microbial host cell of embodiments 1-7, wherein the microbial host cell has reduced or no protease activity in the following proteases: slp1, pep1, gap1, pep5, gap2 and sep1.
18. The microbial host cell of embodiments 1-7, wherein the microbial host cell has reduced or no protease activity in the following proteases: slp1, pep1, gap1, pep5, gap2, sep1 and pep2.
19. The microbial host cell of embodiments 1-7, wherein the microbial host cell has reduced or no protease activity in the following proteases: slp1, pep1, gap1, pep2, pep5 and sep1.
20. The microbial host cell of embodiments 1-7, wherein the microbial host cell has reduced or no protease activity in the following proteases: slp1, pep1, gap1, pep2, pep5, sep1 and gap1.
21. The microbial host cell of embodiments 1-7, wherein the microbial host cell has reduced or no protease activity in the following proteases: slp1, pep1, gap1, pep2, pep5, sep1 and gap2.
22. The microbial host cell according to any preceding embodiment, wherein the microbial host cell is a fungal cell, for example a filamentous fungal host cell, for example a filamentous fungus selected from the group consisting of Aspergillus, Acremonium, Myceliophthora, Thielavia Chrysosporium, Penicillium, Talaromyces, Rasamsonia, Fusarium or Trichoderma, preferably a species of Aspergillus niger, A. nidulans, Aspergillus awamori, Aspergillus foetidus, Aspergillus sojae, Aspergillus fumigatus, Aspergillus oryzae, Acremonium alabamense, Myceliophthora thermophila, Myceliophthora heterothallica, Thermothelomyces heterothallica, Thermothelomyces thermophilus, Thielavia terrestris, Chrysosporium lucknowense, Fusarium oxysporum, Rasamsonia emersonii, Talaromyces emersonii, Trichoderma reesei, Penicillium chrysogenum, Penicillium oxalicum and Neurospora crassa.
23. The microbial host cell according to embodiment 22 which is Trichoderma reesei, Myceliophthora heterothallica, Myceliophthora thermophilus, Aspergillus niger or Aspergillus nidulans.
24. The microbial host cell of any preceding embodiment, wherein total protease activity is at least about 1% less, preferably about 10% or less, more preferably about 40% less than the total protease activity of the parent microbial host cell.
25. The microbial host cell of any preceding embodiment, wherein the compound of interest is selected from the group consisting of an antibody, or functional fragment thereof, a carbohydrate binding domain, a heavy chain antibody or a functional fragment thereof, a single domain antibody, a heavy chain variable domain of an antibody or a functional fragment thereof, a heavy chain variable domain of a heavy chain antibody (VHH) or a functional fragment thereof, a variable domain of camelid heavy chain antibody or a functional fragment thereof, a variable domain of a new antigen receptor (vNAR), a variable domain of shark new antigen receptor or a functional fragment thereof, a minibody, a nanobody, a nanoantibody, an affibody, an alphabody, a designed ankyrin-repeat domain, an anticalins, a knottins or an engineered CH2 domain.
26. The microbial host cell of any preceding embodiment, wherein the compound of interest is a heavy chain variable domain of a heavy chain antibody (VHH) or a functional fragment thereof.
27. The microbial host cell of any preceding embodiment, wherein the modification that leads to a reduced or no protease activity of at least one endogenous protease is a genetic modification.
28. The microbial host cell of embodiment 27, wherein the genetic modification is in the polynucleotide(s) encoding the at least one endogenous protease.
29. The microbial host cell of embodiment 27 or embodiment 28, wherein the genetic modification is a partial or full deletion of the polynucleotide(s) encoding the at least one endogenous protease.
30. The microbial host cell of any preceding embodiment, wherein the microbial host cell has a further modification that affects the production, stability and/or function of a regulator of transcription, and wherein production of the compound of interest from the modified microbial cell is increased compared to the production of the same compound of interest from a parent microbial host cell lacking the further modification that affects the production, stability and/or function of the regulator of transcription.
31. The microbial host cell of embodiment 30 where the regulator of transcription comprises a sequence having at least about 95% or 100% identity to the sequence of SEQ ID NO: 108.
32. The microbial host cell of embodiment 30 wherein:
   a. the regulator of transcription comprises a sequence selected from the group consisting of SEQ ID NOs: 102, 105, 110, 113, 116 and 117 or a polypeptide at least about 80%, at least about 85%, at least about 90%, at least about 95%, or at least about 98% identical thereto, or an ortholog thereof;
   b. the regulator of transcription is coded for by a genomic nucleotide sequence comprising a sequence selected from the group consisting of SEQ ID NOs: 103, 106, 111 and 114 or a polypeptide at least 80%, at least about 85%, at least about 90%, at least about 95%, or at least about 98% identical thereto, or an ortholog thereof; or
   c. the regulator of transcription is coded for by a nucleotide sequence comprising a sequence selected from the group consisting of SEQ ID NOs: 104, 107, 112 and 115 or a polypeptide at least about 80%, at least about 85%, at least about 90%, at least about 95%, or at least about 98% identical thereto, or an ortholog thereof.
33. Use of the microbial host cell of any preceding embodiment in a method of producing the compound of interest.
34. A method of producing a compound of interest, comprising
   a. providing a microbial host cell of any one of embodiments 1-32,
   b. culturing the cell such that the compound of interest is produced, and
   c. optionally isolating the compound of interest.
35. A compound of interest produced by the method of embodiment 34.
36. A method of increasing production of a compound of interest from a microbial host cell, comprising
   a. providing a microbial host cell comprising a recombinant polynucleotide encoding a compound of interest, and
   b. modifying the microbial host cell such that the modification leads to a reduced or no protease activity of at least one endogenous protease;
      wherein the at least one endogenous protease is selected from the proteases comprised in the family of serine proteases, metalloproteases, aspartyl proteases and glutamic proteases, and
      wherein production of the compound of interest from the modified microbial cell is increased compared to the production of the same compound of interest from a parent microbial host cell lacking the modification that leads to a reduced or no protease activity of the at least one endogenous protease.
37. The method of embodiment 36, wherein the method comprises further modifying the microbial host cell such that the further modification affects the production, stability and/or function of a regulator of transcription and wherein production of the compound of interest from the modified microbial cell is increased compared to the production of the same compound of interest from a parent microbial host cell lacking the further modification that affects the production, stability and/or function of the regulator of transcription.
38. A method of producing a modified microbial host cell, comprising
   a. providing a microbial host cell, and
   b. modifying the microbial host cell such that the modification leads to a reduced or no protease activity of at least one endogenous protease, thereby obtaining the modified host cell;
      wherein the at least one endogenous protease is selected from the proteases comprised in the family of serine proteases, metalloproteases, aspartyl proteases and glutamic proteases, and
      wherein production of a compound of interest expressed by the modified microbial cell is increased compared to the production of the same compound of interest from a parent microbial host cell lacking the modification that leads to a reduced or no protease activity of the at least one endogenous protease.
39. The method of embodiment 38, wherein the method comprises further modifying the microbial host cell such that the further modification affects the production, stability and/or function of a regulator of transcription and wherein production of the compound of interest from the modified microbial cell is increased compared to the production of the same compound of interest from a parent microbial host cell lacking the further modification that affects the production, stability and/or function of the regulator of transcription.
40. The method of embodiments 38 or 39, wherein the microbial host cell provided in step (a) comprises a recombinant polynucleotide encoding a compound of interest.
41. The method of embodiments 38 or 39, further comprising modifying the microbial host cell to introduce a recombinant polynucleotide encoding the compound of interest.
42. A kit:
   a. comprising:
      i. a microbial cell; and
      ii. a vector or donor DNA construct for homologous recombination, for example for effecting a full or partial deletion of a gene encoding at least one endogenous protease in the microbial cell; and optionally further comprising
      iii. a vector comprising a nucleotide sequence coding for a compound of interest, wherein the nucleotide sequence is operably linked to a promoter;
   b. or comprising:
      i. a microbial cell having a modification that leads to a reduced or no protease activity of at least one endogenous protease, optionally wherein the modified microbial host cell is a microbial cell according to any one of embodiments 1 to 32; and
      ii. a vector comprising a nucleotide sequence coding for a compound of interest, wherein the nucleotide sequence is operably linked to a promoter;
   c. or comprising:
      i. a vector or donor DNA construct for homologous recombination, for example for effecting a full or partial deletion of a gene encoding at least one endogenous protease in the microbial cell; and
      ii. a vector comprising a nucleotide sequence coding for a compound of interest, wherein the nucleotide sequence is operably linked to a promoter.
43. A kit:
   a. comprising:
      i. a microbial cell; and
      ii. a vector or donor DNA construct for homologous recombination, for example for effecting a full or partial deletion of a gene encoding at least one endogenous protease in the microbial cell and for effecting a modification that affects the production, stability and/or function of a gene encoding a regulator of transcription in the microbial cell; and optionally further comprising
      iii. a vector comprising a nucleotide sequence coding for a compound of interest, wherein the nucleotide sequence is operably linked to a promoter;
   b. or comprising:
      i. a microbial cell having a modification that leads to a reduced or no protease activity of at least one endogenous protease and a further modification that affects the production, stability and/or function of a regulator of transcription, optionally wherein the modified microbial host cell is a microbial cell according to any one of embodiments 1 to 32; and
      ii. a vector comprising a nucleotide sequence coding for a compound of interest, wherein the nucleotide sequence is operably linked to a promoter;
   c. or comprising:
      i. a vector or donor DNA construct for homologous recombination, for example for effecting a full or partial deletion of a gene encoding at least one endogenous protease in the microbial cell and for effecting a modification that affects the production, stability and/or function of a gene encoding a regulator of transcription in the microbial cell; and
      ii. a vector comprising a nucleotide sequence coding for a compound of interest, wherein the nucleotide sequence is operably linked to a promoter.
44. The kit of embodiment 42 or 43 wherein the kit further comprises instructions for use and/or wherein the components of the kit are disposed separately in different containers.

1. A microbial host cell which is characterized by
a. having a modification that leads to a reduced or no protease activity of at least one endogenous protease and a further modification that affects the production, stability and/or function of a regulator of transcription; and,
b. comprising a recombinant polynucleotide encoding a compound of interest;
wherein the at least one endogenous protease is selected from the proteases comprised in the family of serine proteases, metalloproteases, aspartyl proteases and glutamic proteases, and
wherein production of the compound of interest from the modified microbial cell is increased compared to the production of the same compound of interest from a parent microbial host cell lacking the modification that leads to a reduced or no protease activity of the at least one endogenous protease and lacking the further modification that affects the production, stability and/or function of the regulator of transcription.

2. The microbial host cell of claim 1, wherein the serine proteases are selected from the proteases slp2, slp6, serX-3, serX-4, serX-5, serX-9, tpp1, ser1-4, ser1-5, serX-10, sep1, slp1 and tsp1.

3. The microbial host cell of claim 1, wherein the metalloproteases are selected from the proteases metX-11, metX-12, amp1, amp2, cpa2, cpa3, cpa5, met1-1, met1-2, met1-3, met1-6, met1-7, and vacX-1.

4. The microbial host cell of claim 1, wherein the aspartyl proteases are selected from the proteases pep1, pep2, pep3, pep4, pep5, pep8, pep9, pep11, pep12, aspX-7, and aspX-11.

5. The microbial host cell of claim 1, wherein the glutamic proteases are selected from the proteases gap1 and gap2.

6. The microbial host cell of any one of claims 1 to 5, wherein the microbial host cell has reduced or no protease activity in the following proteases:
pep2, pep3 and pep4; or
pep2, pep3, pep4, pep5 and sep1; or
pep2, pep3, pep4, pep5, gap2 and sep1; or
pep2, pep3, pep4, pep5, gap2, sep1 and pep1; or
pep2, pep3, pep4, pep5, gap2, sep1, pep1 and gap1; or
pep2, pep3, pep4, pep5, gap2, sep1, slp1 and gap1; or
pep2, pep3, pep4, pep5, gap2, sep1, slp1, pep1 and gap1.

7. The microbial host cell of any one of claims 1 to 6, wherein the microbial host cell has reduced or no protease activity in the following proteases: pep2, pep3, pep4, pep5, gap2, sep1, slp1, pep1, gap1, serX-4, serX-5 and metX-12.

8. The microbial host cell of any one of claims 1 to 5, wherein the microbial host cell has reduced or no protease activity in the following proteases:
slp1, pep1 and gap1; or
slp1, pep1, gap1, pep5, gap2 and sep1; or
slp1, pep1, gap1, pep5, gap2, sep1 and pep2; or
slp1, pep1, gap1, pep2, pep5 and sep1; or
slp1, pep1, gap1, pep2, pep5, sep1 and gap1; or
slp1, pep1, gap1, pep2, pep5, sep1 and gap2.

9. The microbial host cell according to any preceding claim, wherein the microbial host cell is a fungal cell, for example a filamentous fungal host cell, for example a filamentous fungus selected from the group consisting of Aspergillus, Acremonium, Myceliophthora, Thielavia Chrysosporium, Penicillium, Talaromyces, Rasamsonia, Fusarium or Trichoderma, preferably a species of Aspergillus niger, A. nidulans, Aspergillus awamori, Aspergillus foetidus, Aspergillus sojae, Aspergillus fumigatus, Aspergillus oryzae, Acremonium alabamense, Myceliophthora thermophila, Myceliophthora heterothallica, Thermothelomyces heterothallica, Thermothelomyces thermophilus, Thielavia terrestris, Chrysosporium lucknowense, Fusarium oxysporum, Rasamsonia emersonii, Talaromyces emersonii, Trichoderma reesei, Penicillium chrysogenum, Penicillium oxalicum and Neurospora crassa.

10. The microbial host cell according to claim 9 which is Trichoderma reesei, Myceliophthora heterothallica, Myceliophthora thermophilus, Aspergillus niger or Aspergillus nidulans.

11. The microbial host cell of any preceding claim, wherein total protease activity is at least about 1% less, preferably about 10% or less, more preferably about 40% less than the total protease activity of the parent microbial host cell.

12. The microbial host cell of any preceding claim, wherein the compound of interest is selected from the group consisting of an antibody, or functional fragment thereof, a carbohydrate binding domain, a heavy chain antibody or a functional fragment thereof, a single domain antibody, a heavy chain variable domain of an antibody or a functional fragment thereof, a heavy chain variable domain of a heavy chain antibody (VHH) or a functional fragment thereof, a variable domain of camelid heavy chain antibody or a functional fragment thereof, a variable domain of a new antigen receptor (vNAR), a variable domain of shark new antigen receptor or a functional fragment thereof, a minibody, a nanobody, a nanoantibody, an affibody, an alphabody, a designed ankyrin-repeat domain, an anticalins, a knottins or an engineered CH2 domain.

13. The microbial host cell of any preceding claim, wherein the compound of interest is a heavy chain variable domain of a heavy chain antibody (VHH) or a functional fragment thereof.

14. The microbial host cell of any preceding claim, wherein the modification that leads to a reduced or no protease activity of at least one endogenous protease is a genetic modification, optionally wherein the genetic modification is in the polynucleotide(s) encoding the at least one endogenous protease, further optionally wherein the genetic modification is a partial or full deletion of the polynucleotide(s) encoding the at least one endogenous protease.

15. The microbial host cell of any preceding claim, wherein the regulator of transcription comprises a sequence having at least about 95% or 100% identity to the sequence of SEQ ID NO: 108.

16. The microbial host cell of any preceding claim, wherein:
a. the regulator of transcription comprises a sequence selected from the group consisting of SEQ ID NOs: 102, 105, 110, 113, 116 and 117 or a polypeptide at least about 80%, at least about 85%, at least about 90%, at least about 95%, or at least about 98% identical thereto, or an ortholog thereof;
b. the regulator of transcription is coded for by a genomic nucleotide sequence comprising a sequence selected from the group consisting of SEQ ID NOs: 103, 106, 111 and 114 or a polypeptide at least 80%, at least about 85%, at least about 90%, at least about 95%, or at least about 98% identical thereto, or an ortholog thereof; or
c. the regulator of transcription is coded for by a nucleotide sequence comprising a sequence selected from the group consisting of SEQ ID NOs: 104, 107, 112 and 115 or a polypeptide at least about 80%, at least about 85%, at least about 90%, at least about 95%, or at least about 98% identical thereto, or an ortholog thereof.

17. Use of the microbial host cell of any preceding claim in a method of producing the compound of interest.

18. A method of producing a compound of interest, comprising
a. providing a microbial host cell of any one of claims 1 to 16,
b. culturing the cell such that the compound of interest is produced, and
c. optionally isolating the compound of interest.

19. A compound of interest produced by the method of claim 18.

20. A method of increasing production of a compound of interest from a microbial host cell, comprising
a. providing a microbial host cell comprising a recombinant polynucleotide encoding a compound of interest, and
b. modifying the microbial host cell such that the modification leads to a reduced or no protease activity of at least one endogenous protease and further modifying the microbial host cell such that the further modification affects the production, stability and/or function of a regulator of transcription;
wherein the at least one endogenous protease is selected from the proteases comprised in the family of serine proteases, metalloproteases, aspartyl proteases and glutamic proteases, and
wherein production of the compound of interest from the modified microbial cell is increased compared to the production of the same compound of interest from a parent microbial host cell lacking the modification that leads to a reduced or no protease activity of the at least one endogenous protease and lacking the further modification that affects the production, stability and/or function of the regulator of transcription.

21. A method of producing a modified microbial host cell, comprising
a. providing a microbial host cell, and
b. modifying the microbial host cell such that the modification leads to a reduced or no protease activity of at least one endogenous protease and further modifying the microbial host cell such that the further modification affects the production, stability and/or function of a regulator of transcription, thereby obtaining the modified host cell;
wherein the at least one endogenous protease is selected from the proteases comprised in the family of serine proteases, metalloproteases, aspartyl proteases and glutamic proteases, and
wherein production of a compound of interest expressed by the modified microbial cell is increased compared to the production of the same compound of interest from a parent microbial host cell lacking the modification that leads to a reduced or no protease activity of the at least one endogenous protease and lacking the further modification that affects the production, stability and/or function of the regulator of transcription.

22. The method of claim 20 or 21, wherein the microbial host cell provided in step (a) comprises a recombinant polynucleotide encoding a compound of interest, or wherein the method further comprises modifying the microbial host cell to introduce a recombinant polynucleotide encoding the compound of interest.

23. A kit:
a. comprising:
i. a microbial cell; and
ii. a vector or donor DNA construct for homologous recombination, for example for effecting a full or partial deletion of a gene encoding at least one endogenous protease in the microbial cell and for effecting a modification that affects the production, stability and/or function of a gene encoding a regulator of transcription in the microbial cell; and optionally further comprising
iii. a vector comprising a nucleotide sequence coding for a compound of interest, wherein the nucleotide sequence is operably linked to a promoter;
b. or comprising:
i. a microbial cell having a modification that leads to a reduced or no protease activity of at least one endogenous protease and a further modification that affects the production, stability and/or function of a regulator of transcription, optionally wherein the modified microbial host cell is a microbial cell according to any one of claims 1 to 16; and
ii. a vector comprising a nucleotide sequence coding for a compound of interest, wherein the nucleotide sequence is operably linked to a promoter;
c. or comprising:
i. a vector or donor DNA construct for homologous recombination, for example for effecting a full or partial deletion of a gene encoding at least one endogenous protease in the microbial cell and for effecting a modification that affects the production, stability and/or function of a gene encoding a regulator of transcription in the microbial cell; and
ii. a vector comprising a nucleotide sequence coding for a compound of interest, wherein the nucleotide sequence is operably linked to a promoter.

24. The kit of claim 23, wherein the kit further comprises instructions for use and/or wherein the components of the kit are disposed separately in different containers.
